SEQUENCE LISTING (193 sequences)

SEQ ID NO: 1 - DNA, 1661 bp, Arabidopsis thaliana
  misc_feature (1)..(1661): Ceres CLONE ID no. 28635
gttttcatcgatgaataaattaacaaacaaaactaaaaaataggatattcgctggccgaatcgcgtcgatccacatcgcaggtgagggttcctgcaatttatccctcgtggtctctgaatctcagatcgtcgtcaacgaatccaccattttctgaatcaaaattttctggaaacaatggggagcttggggacgatgctgagatatcccgatgatatatatccgctcctgaagatgaaacgagcgattgagaaagcggagaagcagatccctcctgagccacactggggtttctgctattcgatgctccacaaggcttctcgaagcttttctctcgttattcagcaactcaacaccgagctccgtaacgccgtgtgtgtgttctacttggttctccgagctcttgatactgttgaggatgatactagcataccaactgatgaaaaggttcccatcctgatagcttttcaccggcacatatacgatactgattggcattattcatgtggtacgaaggagtacaagattctaatggaccaatttcaccatgtttctgcagcttttttggaacttgaaaaagggtatcaagaggctatcgaggaaattactagaagaatgggtgcagggatggccaagtttatctgccaagaggtagaaactgttgatgactacgatgaatactgccactatgttgctgggcttgttggtttaggtttgtcgaaactcttcctcgctgcaggatcagaggttttgacaccagattgggaggtgatttccaattcaatgggtttatttctacagaaaacaaacattatcagagattatcttgaggacattaatgagataccaaaatcccgcatgttttggcctcgcgagatttggggcaaatatgctgacaagcttgaggatttaaaatacgaggagaacacaaacaaatccgtacagtgcttaaatgaaatggttaccaatgcgttgatgcatattgaagattgcctgaaatacatggtttccttgcgtgatccttccatatttcggttctgtgccatccctcagatcatggcgattggaacacttgcattatgctataacaatgaacaagtattcagaggcgttgtgaaactgaggcgaggtcttactgctaaagtcattgatcgtacaaagacaatggctgatgtctatggtgctttctatgatttttcctgcatgctgaagacaaaggttgacaagaacgatccaaatgccagtaagacactaaaccgacttgaagccgttcagaaactctgcagagacgctggagttcttcaaaacagaaaatcttatgttaatgacaaaggacaaccaaacagtgtctttattataatggttgtgattctactggccatagtctttgcatatctcagagcaaactgagtgatccatgtaagcgagtctgattgtatcaccatcattcaagatgttcagagcaaatttgagtgatgaagtaatctaggttgattcttattcacgccactgaatcctaagcaagattgtttccagaacaaacagagtttaagcatggtttagtctaaaaccatggattctattttagttactaccttcgttgtctaaacgtgcatttgttcatctatttttattccttgtgtttaaagttctttctttgttt

SEQ ID NO: 2 - protein, 410 aa, Arabidopsis thaliana
  misc_feature (1)..(410): Ceres CLONE ID no. 28635
MGSLGTMLRYPDDIYPLLKMKRAIEKAEKQIPPEPHWGFCYSMLHKASRSFSLVIQQLNTELRNAVCVFYLVLRALDTVEDDTSIPTDEKVPILIAFHRHIYDTDWHYSCGTKEYKILMDQFHHVSAAFLELEKGYQEAIEEITRRMGAGMAKFICQEVETVDDYDEYCHYVAGLVGLGLSKLFLAAGSEVLTPDWEVISNSMGLFLQKTNIIRDYLEDINEIPKSRMFWPREIWGKYADKLEDLKYEENTNKSVQCLNEMVTNALMHIEDCLKYMVSLRDPSIFRFCAIPQIMAIGTLALCYNNEQVFRGVVKLRRGLTAKVIDRTKTMADVYGAFYDFSCMLKTKVDKNDPNASKTLNRLEAVQKLCRDAGVLQNRKSYVNDKGQPNSVFIIMVVILLAIVFAYLRAN

SEQ ID NO: 3 - protein, 413 aa, Glycine max
  misc_feature (1)..(413): Ceres CLONE ID no. 515962
MGSLGAILKHPDDFYPLLKLKMAARNAEKQIPPEPHWAFCYTMLHKVSRSFALVIQQLGIELRNAVCIFYLVLRALDTVEDDTSIETDVKVPILIAFHRHIYDRDWHFSCGTKEYKVLMDQFHHVSTAFLELGKNYQEAIEDITKRMGAGMAKFICKEVETIDDYDEYCHYVAGLVGLGLSKLFHASGSEDLAPDDLSNSMGLFLQKTNIIRDYLEDINEIPKSRMFWPRQIWSEYVNKLEDLKYEENSVKAVQCLNDMVTNALMHPEDCLKYMAALRDPPIFRFCAIPQIMAIGTLALCYNNIEVFRGVVKMRRGLTAKVIDRTKTMADVYGAFFDFASMLEPKVDKNDPNATKTLSRLEAIQKTCRESGLLSKRKSYIVNDESGYGSTMIVILVIMVSIIFAYLSANHHNS

SEQ ID NO: 4 - protein, 415 aa, Centella asiatica
  misc_feature (1)..(415): Public GI no. 55710094
MGSLGAILKHPDDFYPLLKLKMAARHAEKQIPPEPHWAFCYSMLHKVSRSFGLVIQQLGPQLRDAVCIFYLVLRALDTVEDDTSISTEVKVPILKAFHRHIYDNNWHFSCGTKEYKILMDEFHHVSNAFLELGSGYKEAIEDITMRMGAGMAKFICKEVETIDDYDEYCHYVAGLVGLGLSKLFHASGAEDLASDSLSNSMGLFLQKTNIIRDYLEDINEIPKSRMFWPRKIWNKYVNKLEDLKYEENSVKAVQCLNDMVTNALLHVEDCLKYMSDLRDPAIFRFCAIPQIMAIGTLALCYNNLQVFRGVVKMRRGLTAKVIDRTNKMSDVYGAFYDFSCMLKTKVDNNDPNATKTLSRLEAIQKKCKESGVITPNRKSYVLENDSGYNLVLIAILFIILALVYAYLSSNLSNNR

SEQ ID NO: 5 - protein, 413 aa, Glycyrrhiza glabra
  misc_feature (1)..(413): Public GI no. 2144186
MGSLGAIVRHPDEVYPLLKLKMAARHAEKQIPAEPHWAFCYTMLLKVSRSFALVIQQLDPQLRNAVCIFYLVLRALDTVEDDTSIATDVKEPILIAFHRHIYDRDWHFSCGTKEYKVLMDQFHHVSTAFLELEKNYQAAIENITKEMGAGMAKFICNEVETVDDYDEYCHYVAGLVGLGLSKLFHASGKEDLAPDHLSNSMGLFLQKTNIIRDYLEDINEIPKSRMFWPRQIWSKYVNKLEDLKYEENSVKAVQCLNDMVTNALLHAEDCLKYMSALRDMSIFRFCAIPQIMAIGTLALCYNNIEVFRGVVKMRRGLTAKVIDRTKTMADVYGAFFDFSCMLKSKVDKNDPNATKTLSRLEAIQKTCRESGLLSKRKPYILRNESTNSSTMVLILVIILSIIFAYLSAKRQDN

SEQ ID NO: 6 - protein, 413 aa, Lotus corniculatus var. japonicus
  misc_feature (1)..(413): Public GI no. 28208268
MGSLGAIVKHPDDLYPLLKLKMAARHAEKQIPSEPHWGFCYSMLHKVSRSFALVIQQLDTDLRNAVCIFYLVLRALDTVEDDTSIATEVKVPILKAFHRHIYDRDWHFSCGTKEYKVLMGQFHLVSTAFLELAKNYQEAIEDITDRMGAGMAKFICKEVETIDDYDEYCHYVAGLVGLGLSKLFHASGKENLAADSLSNSMGLFLQKTNIIRDYLEDINEIPKSRMFWPRQIWSKYVNKLEDLKYEENSVKAVQCLNDMVTNALMHAEDCLKYMSDLRDDSIFRFCAIPQIMAIGTLAICYNNVEVFRGVVKMRRGLTAKVIDRTKTIADVYGAFFDFASMLESKVDKNDPNATKTLSRLVAIQKTCRESGLLNKRKSYILRKENGYGSTLIIILVLLFSIMFAYSSATRHSN

SEQ ID NO: 7 - protein, 415 aa, Panax ginseng
  misc_feature (1)..(415): Public GI no. 41224629
MGSLGAILKHPEDFYPLLKLKFAARHAEKQIPPEPHWAFCYSMLHKVSRSFGLVIQQLGPQLRDAVCIFYLVLRALDTVEDDTSIPTEVKVPILMAFHRHIYDKDWHFSCGTKEYKVLMDEFHHVSNAFLELGSGYQEAIEDITMRMGAGMAKFICKEVETINDYDEYCHYVAGLVGLGLSKLFHASGAEDLATDSLSNSMGLFLQKTNIIRDYLEDINEIPKSRMFWPRQIWSKYVDKLEDLKYEENSAKAVQCLNDMVTDALVHAEDCLKYMSDLRGPAIFRFCAIPQIMAIGTLALCFNNTQVFRGVVKMRRGLTAKVIDQTKTMSDVYGAFFDFSCLLKSKVDNNDPNATKTLSRLEAIQKTCKESGTLSKRKSYIIESESGHNSALIAIIFIILAILYAYLSSNLLLNKQ

SEQ ID NO: 8 - protein, 413 aa, Medicago truncatula
  misc_feature (1)..(413): Public GI no. 27475614
MGSIKAILKNPDDFFPLLKLKIAARNAEKQIPPEPHWGFCYSMLHKVSRSFGLVIQQLGPELRDAVCIFYLVLRALDTVEDDTSIETDVKVPILIDFHRHIYDNDWHFGCGTKEYKVLMDQFHHVSKAFLELGKNYQDAIEDITKRMGAGMAKFICKEVETVDDYDEYCHYVAGLVGLGLSKLFYASGKEDLATDKLSNSMGLFLQKTNIIRDYLEDINEIPKSRMFWPRQIWSKYVSKLEDLKYEENSVKAVQCLNDMVTNALLHADDCLQYMSALRDSSNFRFCAIPQVMAIGTLAMCYNNIGVFRGVVKMRRGLTAKVIDRTKTMADVYGAFFDFASVLESKVDKNDPNATKTSSRLEAIQKTCRESGLLTKRKSYVLRNESGYGSTMILLLVILFSIIFAYLSANRHNN

SEQ ID NO: 9 - protein, 411 aa, Solanum tuberosum
  misc_feature (1)..(411): Public GI no. 5360655
MGTLRAILKNPDDLYPLIKLKLAARHAEKQIPPEPHWGFCYLMLQKVSRSFALVIQQLPVELRDAVCIFYLVLRALDTVEDDTSIPTDVKVPILISFHQHVYDREWHFACGTKEYKVLMDQFHHVSTAFLELGKLYQQAIEDITMRMGAGMAKFICKEVETTDDYDEYCHYVAGLVGLGLSKLFHASGTEDLASDSLSNSMGLFLQKTNIIRDYLEDINEVPKCRMFWPREIWSKYVNKLEDLKYEENSVKAVQCLNEMVTNALSHVEDCLTYMFNLRDPSIFRFCAIPQVMAIGTLAMCYDNIEVFRGVVKMRRGLTAKVIDRTKTMADVYGAFFDFSCMLKSKVNNNDPNATKTLKRLDAILKTCRDSGTLNKRKSYIIRSEPNYSPVLIVVIFIILAIILAQLSGNRS

SEQ ID NO: 10 - protein, 411 aa, Capsicum annuum
  misc_feature (1)..(411): Public GI no. 4426953
MGTLRAILKNPDDLYPLIKLKLAARHAEKQIPPEPHWGFCYLMLQKVSRSFALVIQQLPVELRDAVCIFYLVLRALDTVEDDTSIPTDVKVPILISFHQHIYDREWHFSCGTKEYKVLMDQFHHVSTAFLELGKNYQQAIEDITMRMGAGMAKFICKEVETTDDYDEYCHYVAGLVGLGLSKLFHASGKEDLASDSLSNSMGLFLQKTNIIRDYLEDINEVPKCRMFWPREIWSKYVNKLEELKYEENSVKAVQCLNDMVTNALSHVEDCLIYMSNLRDPAIFRFCAIPQVMAIGTLAMCYDNIEVFRGVVKMRRGLTAKAIDRTRTMADVYGAFFDFSCMLKSKVNNNDPNATKTLKRLEAILKTCRDSGTLNKRKSYVIKSEPTYSPVLIFVIFIILAIILAHLSGNRS

SEQ ID NO: 11 - protein, 411 aa, Nicotiana tabacum
  misc_feature (1)..(411): Public GI no. 1552717
MGSLRAILKNPDDLYPLVKLKLAARHAEKQIPPSPHWGFCYSMLHKVSRSFALVIQQLPVELRDAVCIFYLVLRALDTVEDDTSIPTDVKVPILISFHQHVYDREWHFSCGTKEYKVLMDQFHHVSTAFLELRKHYQQAIEDITMRMGAGMAKFICKEVETTDDYDEYCHYVAGLVGLGLSKLFHASGKEDLASDSLSNSMGLFLQKTNIIRDYLEDINEVPKCRMFWPREIWSKYVNKLEELKYEDNSAKAVQCLNDMVTNALSHVEDCLTYMSALRDPSIFRFCAIPQVMAIGTLAMCYDNIEVFRGVVKMRRGLTAKVIDQTRTIADVYGAFFDFSCMLKSKVNNNDPNATKTLKRLEAILKTCRDSGTLNKRKSYIIRSEPNYSPVLIVVIFIILAIILAQLSGNRS

SEQ ID NO: 12 - protein, 411 aa, Nicotiana benthamiana
  misc_feature (1)..(411): Public GI no. 1184109
MGSLRAILKNPEDLYPLVKLKLAARHAEKQIPPSPNWGFCYSMLHKVSRSFALVIQQLPVELRDAVCIFYLVLRALDTVEDDTSIPTDVKVPILISFHQHVYDREWHFSCGTKEYKVLMDQFHHVSTAFLELRKHYQQAIEDITMRMGAGMAKFICKEVETTDDYDEYCHYVAGLVGLGLSKLFHASEKEDLASDSLSNSMGLFLQKTNIIRDYLEDINEVPKCRMFWPREIWSKYVNKLEELKYEDNSAKAVQCLNDMVTNALPHVEDCLTYMSALRDPSIFRFCAIPQVMAIGTLAMCYDNIEVFRGVVKMRRGLTAKVIDRTRTIADVYGAFFDFSCMLKSKVNNNDPNATKTLKRLEVILKTCRDSGTLNKRKSYIIRSEPNYSPVLIVVIFIILAIILAQLSGNRS

SEQ ID NO: 13 - DNA, 1092 bp, Arabidopsis thaliana
  misc_feature (1)..(1092): Ceres CLONE ID no. 23439
ttctctttctctcacactctctgcctatctccgccgcggagatccatcgtgttttctacttctgttggtatccatggattctctcgttgaatccggttggaagtaccttgttacgcatttcagtgactttcaactggcgtgtattgggagttttatacttcatgaaagcgtgtttttcttgtctggactcccttacattttcctagaaaggactggttttctgagcaactacaaaattcagaccaaaagtaatactcctgaagcccagggaaaatgcattgctcgactattactttaccattgctgcgtaaacttgcccctcatgatggcgtcgtatcctgtattcagattcatgggcatggaaagcagttttcctctgccgtcctggaaagtggtgtctgcccagatcttattctacttcatcattgaggattttgtattctattggggtcacaggatcttgcatactaaatggctctacaagaacgtgcacagtgtgcatcatgagtacgcgacaccgtttggtttgacatcagaatatgctcatcccgctgaaattctgttccttggttttgctaccattgttggtccggctctcaccgggcctcacctgatcaccctttggttatggatgatgctcagagttattgagacagttgaggcacattgtggttatcatttcccatggagcccctcgaattttcttcctctatacggcggatctttaatattaatgtgggaatcatttgcttacagtgctgacttccatgactaccatcatcgattactctacacaaagtctggcaactactcatcaacgtttgtttacatggactggatctttggcaccgataaaggttacagaaaacttaaggccctaaaagaaacctgacaacaaacaaacgtgaaattcgattcccgagagaggtcttgttctcaaaaaaccttctttcttttgttttgtctctaccatttcctcaactttagtcataagaacatcattatttgctacaatttgggttgtacaatcaaagctgctagtctgaagtcgattcgatgttgaaatttatacgtgacctcgtttctattggc

SEQ ID NO: 14 - protein, 272 aa, Arabidopsis thaliana
  misc_feature (1)..(272): Ceres CLONE ID no. 23439
MDSLVESGWKYLVTHFSDFQLACIGSFILHESVFFLSGLPYIFLERTGFLSNYKIQTKSNTPEAQGKCIARLLLYHCCVNLPLMMASYPVFRFMGMESSFPLPSWKVVSAQILFYFIIEDFVFYWGHRILHTKWLYKNVHSVHHEYATPFGLTSEYAHPAEILFLGFATIVGPALTGPHLITLWLWMMLRVIETVEAHCGYHFPWSPSNFLPLYGGSLILMWESFAYSADFHDYHHRLLYTKSGNYSSTFVYMDWIFGTDKGYRKLKALKET

SEQ ID NO: 15 - protein, 269 aa, Gossypium arboreum
  misc_feature (1)..(269): Public GI no. 27448145
MAAIIESGWLYLITHFSDFQLASLGSFFLHESVFFLSGLPFIYLERAGLLSKYKIQTKNNSLAAQEKCITRLLLYHIGVNLPLMIASYPFFRFMGMKSSLPFPSWKVVLSQIIFYFILEDFVFYWGHRILHTKWLYKHVHSVHHEYATPFGLTSEYAHPAEILFLGFATIIGPAITGPHLITLWLWMTLRVLETVEAHCGYHFPWSLSNFLPLYGGADFHDYHHRLLYTKSGNYSSTFVYMDWIFGTDKGYRKLKALKRDGVEEEAKQT

SEQ ID NO: 16 - protein, 271 aa, Glycine max
  misc_feature (1)..(271): Ceres CLONE ID no. 664026
MASLIESGWQYLITHFSDFQLACLGSFFLHEGVFFLSGLPFIWLERAGWMSKYKIQAKNNTPAAQEKCIVRLLLYHFGVNLPVMIFSYPVFTYMGMRSSLPLPSWKVVLIQIIFYFILEDFIFYWGHRILHTKWLYKHVHSVHHEYATPFGLTSEYAHPAEILFLGFATIFGPAITGPHLITLWLWMVLRVLETVEAHCGYHFPWSLSNFLPLYGGADFHDYHHRLLYTKSGNYSSTFTYMDRIFGTDIGYRKLKALKSIGVEDSGEQKKQ

SEQ ID NO: 17 - protein, 252 aa, Brassica napus
  misc_feature (1)..(252): Ceres CLONE ID no. 977729
MDSLVESGWQYLVSNFSDFQLACIGSFIVHETVFFLSGLPYILLERTGLLSNYKIQTKHNTPEAQGKCIAWLLFYHSCVNFPLMMFSYRVFKFMGMQSSFPLPSWKVVSAQILFFFIIEDFVFYWGHRILHTKWLYKNVHSVHHEYATPFGLTSEYAHPAEILFLGFATFIGPALTGPHLITLWLWLVLRVIETVEAHCGYHFPWSPSIFLSMAVLTSMTTITVYSTQSLGTTHQLLSTWIGYLVPTRVTEN

SEQ ID NO: 18 - protein, 242 aa, Nicotiana benthamiana
  misc_feature (1)..(242): Public GI no. 34978966
MASMIESAWTYLISNFSDFQLTCLGGFVLHESIFFLSGLPFILFERAGWFGKYKIQKKNNSPEAQEKCITRLLMYHLCVNLPILLGSYPVFKFMGTRSSLPLPSWKVISTQIIFYFILEDFVFYWGHRILHTKWLYKHVHSVHHEYATPFGLTSEYAHPAEILFLGFATIIGPAITGPHLITLYLWVSLRVLETVEAHSGYHFPWSPSNFLPLYGGADFHDYHHRLLYTKSGNYSSTFVYMD

SEQ ID NO: 19 - protein, 268 aa, Oryza sativa subsp. japonica
  misc_feature (1)..(268): Public GI no. 51963234
MMAAAGLESAWEYLITHFSEFQLASIGTFLLHESVFFLSGLPSLLFERLGLFSKYKIQKKSNTPDYQNRCVVRLVLYHVCVNLPLTILSYRTFKFMGLRSTLPLPHWTVVVSQVLFFFVLEDFIFYWGHRALHTKWLYQHVHSVHHEYATPFGLTSEYAHPAEILFLGFATVAGPALTGPHLFTLWVWMVLRVLETVEAHSGYHFPWSPSNFLPLYGGAEFHDYHHRVLYTKSGNYSSTFIYMDWLFGTDKDYRKTKALEEKERTKHL

SEQ ID NO: 20 - protein, 265 aa, Zea mays
  misc_feature (1)..(265): Ceres CLONE ID no. 217004
MAVPMSAIESAWQLLIANFTEFQLATVITFLLHETVFFLSGLPSLLFERFGLFAKYKIQKRSNTSAYQNRCVLRLILYHVSVNLPVMILSYPAFKFMGLRSSLPLPHWTVVVSQVLFYFVLEDFIFYWGHRALHTKWLYKHVHSVHHEYATPFGLTSEYAHPAEILFLGFATVVGPALTGPHLFTLWLWMVLRVLETVEAHSGYHFPWSPSNFLPLYGGSDFHDYHHRVLYTKSGNYASTFVYMDWLFRTDNGYRKAKRPLRSKK

SEQ ID NO: 21 - protein, 268 aa, Zea mays
  misc_feature (1)..(268): Ceres CLONE ID no. 245428
MAAPMSAIDSAWQLLIANFTEFQLATVVTFLLHETVFFLSGLPSLLFERFGLFAKYKIQKKSNTSAYQNRCVLRLILYHVCVNLPVMIFSYPAFKFMGLRSSLPLPHWSVVVSQVLFYFVLEDFIFYWGHRALHTKWLYKHVHSVHHEYATPFGLTPEYAHPAEILFLGFATVVGPALTGPHLFTLWLWMVLRVLETVEAHSGYHFPWSPSNFLPLYGGSDFHDYHHRVLYTKSGNYASTFVYMDWLFGTDKDYRKAKTIEEKEGKNL

SEQ ID NO: 22 - DNA, 1147 bp, Arabidopsis thaliana
  misc_feature (1)..(1147): Ceres CLONE ID no. 39378
tcactcactcacactactcaagtcaacgtttctaagattctccggcggagcgattatgtcaggatcttcttcaccgagcttgtggctggcaccgaatccgagcaagagatggggagagctcttcttcctcttctacactcctttttggctcactctctgtttaggcatcgttgttccttacaagctttacgagacattcacggagttggagtatctgcttctagctttggtttcagctgttcctgctttcgttatacccatgttactcgttggaaaggccgacagaagtttatgttggaaggaccgctattgggttaaggcaaatctctggataattgttttcagctatgtcggaaactacttttggactcactatttctttaaagttcttggagcgtcctatacttttccatcatggaaaatgaataatgtgcctcacacaacattcttcctaacacatgtttgcttcctcttttaccacgttgcatcgaacattactcttcggaggctacgccattccactgctgatttaccagattctctgaaatggtgttttgaggctgcatggatacttgcgctttcttatttcattgcatacttggagactattgctatcgcaaattttccttactatgagtttgtggaccgaagtgccatgtacagagttggatgtcttttctatgccatttacttcattgtgagcttcccaatgttcttcaggatggatgagaaatcaactgatgaatgggatttatctcgagtggctgttgatactttgggtgctgctatgttggtaacaatcattcttgatctatggcgtctcttcttgggacctatagttcccttaccggagggacaaaactgccttcagtctggattaccatggttctccaattgaagattgaaagatcggttacttccaccaattcagggaattgacatgggactgagagagaaacctgcaaaggaatttccattaacactttacggaattgggaagcagttaatgcaggccatgtttctaaggcttttcttctccaactgctgatttgtttgttagttgtttttggttttcagtttctcttgatgaactttcccaatgtaacctttcaagttatcctttatagaacagaatggaacttcgttt

SEQ ID NO: 23 - protein, 280 aa, Arabidopsis thaliana
  misc_feature (1)..(280): Ceres CLONE ID no. 39378
MSGSSSPSLWLAPNPSKRWGELFFLFYTPFWLTLCLGIVVPYKLYETFTELEYLLLALVSAVPAFVIPMLLVGKADRSLCWKDRYWVKANLWIIVFSYVGNYFWTHYFFKVLGASYTFPSWKMNNVPHTTFFLTHVCFLFYHVASNITLRRLRHSTADLPDSLKWCFEAAWILALSYFIAYLETIAIANFPYYEFVDRSAMYRVGCLFYAIYFIVSFPMFFRMDEKSTDEWDLSRVAVDTLGAAMLVTIILDLWRLFLGPIVPLPEGQNCLQSGLPWFSN

SEQ ID NO: 24 - protein, 285 aa, Glycine max
  misc_feature (1)..(285): Ceres CLONE ID no. 716942
MAASASSSPWLAPNPSKRWGELFFLLYTPFWLTLCLGIVIPFNLYEKFTELEYLLIGLVSAVPAFVVPLLLVGKADRSISWKDRYWVKASLWIIIFSYVGNYFWTHYFFTVLGASYTFPSWKMNNVPHTTFLLTHVCFLFYHVSSNMTLRRLRHFIAGLPEKLQWATEAAWILALAYFIAYLETLAISNFPYYQFVDRDSMYKVGSLFYAIYFIVSFPMFLRIDEKPGDKWDLPRVAVDALGAAMLVTIILDLWRIFLGPIVPIADTKQCPQVGLPWFTGHANLK

SEQ ID NO: 25 - protein, 297 aa, Zea mays
  misc_feature (1)..(297): Ceres CLONE ID no. 285554
MAAARRPGAKPRGGGGAGKRSAWLAADGSKRWGEAFFLLYTPFWLTLCLGVVVPFKLYERFTELEYLILGLVSTVPAFVIPLFLVGKADSVRNFKDRYWVKANIWIIIFSYVGNYFWTHYFFTVLGASYTFPSWRMNNVPHTTFLLTHACFLFYHMASNMTLRRLRHSTAHLPQSICWLFEAAWILALSYFIAYLETLAIANFPYYEFVDRDIMYKVGSLFYAIYFIVSFPMFSRIDEKDEKWSLSRVAVDSLGAAMLVTIILDLWRIFLGPIVPVPESRQCGQPGLAWFQAQNGST

SEQ ID NO: 26 - protein, 326 aa, Oryza sativa subsp. japonica
  misc_feature (1)..(326): Public GI no. 62732798
MAAPRRPAAAKARNGGGAGRTSRWMAADGSKRWGETFFLLYTPFWLTLCLGVVVPFKLYERFTELEYLVVGLVSTVPAFVIPLFLVGKADSVRSLKDRYWVKANIWIIIFSYVGNYFWTHYFFTVLGASYTFPSWRMNNVPHTTFLLTHACFLFYHMTSNMSLRKLHHSTAHLPQFLRWSFEAAWVLALSYFIAYLETLAIANKICGNAFQSGQIPLDRPSGYTTFEHWEKFPYYEFIDRDIMYKVGSLFYAIYFIVSFPMFSRIDENEEKWSLSRVAVDALGAAMLVTIILDLWRIFLGPIVPIPESRRCGQPGLAWFQVQNESV

SEQ ID NO: 27 - DNA, 928 bp, Arabidopsis thaliana
  misc_feature (1)..(928): Ceres CLONE ID no. 2121
agtaagtcatcatcactcagatctagtagttccacaaactcacattgggtcggatcgttgaccagaaaaacacacagagaaaggagaaaaacatgaaggagttggcgcatccgtacgttccgagagatctgaacctacccggatacgtaccaatctcaatgtcaatgtcctccatcgtctctatctacctcggttcttccctccttgttgtctccctcgtctggcttctcttcgggaggaagaaagctaaacttgataagttgcttatgtgttggtggacattcactggtctcactcatgttattctcgagggctatttcgttttctcccctgagtttttcaaggacaacacttctgcttatcttgctgaagtttggaaagaatatagcaaaggtgattcgagatacgtaggtagagattctgcagttgtatctgttgaagggatcactgctgttatcgttggcccagcttctctcctagctatttatgccattgctaaggagaagtcgtatagctacgtgcttcagcttgcgatttcggtttgccagctctacggatgtttggtttatttcattactgctatcttggaaggagacaactttgccacaaactctttctactattactcctactacatcggggctaactgttggtgggtcttgataccttcactcatttctttccgttgctggaaaaagatttgtgcagctgctgccattgccaacaacaacgtcgagacaaagacgaagaagaaaacccgttgaagcaaggatcaaactctcttttatctcttcaggttactgctgctatagcaaatgaacatgagtctccttttgcggataatttatccattttaaaaactttagctatgttgtctgtctctcttacagcaataaacttgtgaggataatttatccatttttcatac

SEQ ID NO: 28 - protein, 223 aa, Arabidopsis thaliana
  misc_feature (1)..(223): Ceres CLONE ID no. 2121
MKELAHPYVPRDLNLPGYVPISMSMSSIVSIYLGSSLLVVSLVWLLFGRKKAKLDKLLMCWWTFTGLTHVILEGYFVFSPEFFKDNTSAYLAEVWKEYSKGDSRYVGRDSAVVSVEGITAVIVGPASLLAIYAIAKEKSYSYVLQLAISVCQLYGCLVYFITAILEGDNFATNSFYYYSYYIGANCWWVLIPSLISFRCWKKICAAAAIANNNVETKTKKKTR

SEQ ID NO: 29 - protein, 220 aa, Brassica napus
  misc_feature (1)..(220): Ceres CLONE ID no. 959258
MEEAAAHPYVPRDLKLPGYVPISMSMSSILAVYLGASLFVVTFVWFLLGRKKTHLDRLLMCWWAFTGLTHMVLEGYFVFSPEFYKDNTSCYLAEVWKEYSKGDSRYAGRDSAVIAVEGITAVIEGPACLLAVYAISKGKSYSYVLQLAISLGQLYGCLVYFITAFLEGDNFATNSFYYYSYYIGANGWWVLIPLXISYRCWNKICESANGVETKMKKKIR

SEQ ID NO: 30 - protein, 219 aa, Glycine max
  misc_feature (1)..(219): Ceres CLONE ID no. 592262
MEAHPYVPRDLHLPGYAPCFLSMSNILSVFASSSLLIVTLVWIFSGRFKKTKVDRVLMCWWAFTGLTHIILEGYFVFSPEFFKDKTGFYLAEVWKEYSKGDSRYAGRDAGVVTVEGITAVLEGPASLLAVYAIATGKSYSYILQFAISLGQLYGTAVYYITAILEGDNFSTNSFYYYAYYIGANASWIVIPLIIAIRCWRKICAAFRVQGGQTKKPKVR

SEQ ID NO: 31 - protein, 221 aa, Zea mays
  misc_feature (1)..(221): Ceres CLONE ID no. 282337
MAAAASMGHPYAPAELDLPGFVPLKLSQVEILVSYLGASVFVFLAVWLVSGRCVRLSKTDRLLMCWWAFTGLTHIMIEGPFVFTPDFFKKENPNFFDEVWKEYSKGDSRYVARDTATVTVEGITAVLEGPASLLAVYAIASRKSFSHILQFAVCLGQLYGCLVYFITAYLDGFNFWVGPFYFWAYFIGANSFWIWIPMLIAIRSWKKTCAAFQAEKVKKTK

SEQ ID NO: 32 - protein, 219 aa, Oryza sativa subsp. japonica
  misc_feature (1)..(219): Public GI no. 50900588
MGHPHPHPYAPAELHLPGFVPLQLSQAQILVPYLATSLFLLLAVWLISGRCSRRLSDTDRWLMCWWAFTGLTHIIIEGTFVFAPNFFSNQNPSYFDEVWKEYSKGDSRYVARDPATVTVEGITAVLEGPASLLAVYAIASGKSYSHILQFTVCLGQLYGCLVYFITAYLDGFNFWTSPFYFWAYFIGANSSWVVIPTMIAIRSWKKICAAFQGEKVKTK

SEQ ID NO: 33 - protein, 216 aa, Triticum aestivum
  misc_feature (1)..(216): Ceres CLONE ID no. 703736
MGAHPYVPASLDLPGYVPLRLTQLEILGAYLGTSLFVLVAVWLLSGRCRRLSGTDRLLMCWWAFTGLTHILIEGPFVFTPDFFTKTNPNFFDEVWKEYSKGDSRYVARDTATVTVEGITAVLKGPASLLAVYAIASRKSYSHILQFAVCLGQLYGCIVYFTTAYLDGFNFWASPFYFWAYFIGANSSWVVIPLLIATRSWKRICAAIHQSEKIKTK

SEQ ID NO: 34 - DNA, 704 bp, Arabidopsis thaliana
  misc_feature (1)..(704): Ceres CLONE ID no. 31252
gatcgaatcataaaccaaacaaatctttcttttaaaagaagcggacacaaaccctaaccaccaaccctctccgtcgtcaaatttcccggcgaaagatgaaacgaattgttcgaatttcattcacagacatggaagcaaccgattcttcaagcagcgaagacgagtcaccaccgtcatcacgccgtcgaggtaagaagctagtcaaggaaatcgtaatcgatcattctgatcccccggaggtcggaaaaactcggttcaaaatcaggattccggcgagtttacttgctgcgaggaatacgacggcgaataagaagaagttccgtggcgtgaggcagagaccatgggggaagtgggcggctgagattagatgtggtagagttaaaggaagacctgaacgaatttggcttgggacttttgaaacagctgaagaagctgctcttgcttatgataacgctgcgattcagttgattggacctgatgcgccgactaattttggccgtcctgatgttgattctgcggtggtgaaaaagcaagattctgatgctagtggtggtgcttctgaagaagttgtttgattttcttgtttataactaactttgcaaggttacttttgtttatcatcatcttttgtaaatttgatcatcttgctctttgctttttaacttctcttgtacatgtttgaattataaaagttctaatttttc

SEQ ID NO: 35 - protein, 159 aa, Arabidopsis thaliana
  misc_feature (1)..(159): Ceres CLONE ID no. 31252
MKRIVRISFTDMEATDSSSSEDESPPSSRRRGKKLVKEIVIDHSDPPEVGKTRFKIRIPASLLAARNTTANKKKFRGVRQRPWGKWAAEIRCGRVKGRPERIWLGTFETAEEAALAYDNAAIQLIGPDAPTNFGRPDVDSAVVKKQDSDASGGASEEVV

SEQ ID NO: 36 - DNA, 1558 bp, Arabidopsis thaliana
  misc_feature (1)..(1558): Ceres CDNA ID no. 23389731
aaaaccaaaacgatggcgtttcatcacaatcatctctcacaagacctctccttcaatcatttcaccgaccaacaccaacctccacctccgcaaccgcctcctcctcctccgcaacagcaacaacatttccaagaagcaccgcctcctaattggttaaacacagcgcttcttcgttcctcagataacaacaataacttcctcaacctccacacagccaccgctaacaccacaaccgcaagcagctccgattctccttcctccgccgccgccgccgccgctgctaaccagtggctatctctctcctcctctttcctccaacgaaacaacaacaacaacgcttccatagtcggagatgggatcgatgatgtcaccggaggagcagacactatgattcagggagagatgaaaaccggcggtggagaaaacaaaaacgacggcggaggagctacggcggcggatggagtagtgagctggcagaatgcgagacacaaggcggagatcctttcgcatcctctttacgagcagcttttgtcggcgcacgttgcttgtttgagaatcgcgactccggttgatcagcttccgagaatcgatgctcagcttgctcagtctcaacacgtcgtcgctaaatactcagctttaggcgccgccgctcaaggtctcgtcggcgacgataaagaacttgaccagttcatgacacattatgtgttgctactgtgttcatttaaagagcaattgcaacaacatgtgcgtgttcatgcaatggaagctgtgatggcttgttgggagattgagcagtctcttcaaagcttaacaggagtgtctcctggagaagggatgggagcaacaatgtctgacgatgaagatgaacaagtagagagtgatgctaatatgttcgatgggggattagatgtgttgggttttggtcctttgattcctactgagagtgagaggtcgttgatggaaagagttagacaagaacttaaacatgaactcaaacagggttacaaggagaagatagtagacataagagaggagatattaaggaagagaagagctgggaagttaccaggagataccacctctgttctcaaagcttggtggcaatctcattccaaatggccttaccctactgaggaagataaggcgaggttggtgcaagagacaggtttgcagctaaaacagataaacaattggttcatcaatcagagaaagaggaactggcatagcaatccatcttcttccactgtattgaagaacaaacgcaaaagcaatgcaggtgacaatagcggaagagagcggttcgcgtagaaacaacaaacatatgatgtgaattggggaggtggaagatgggatttgaaagcagggttttagggatttaaagttgagaattttatggaggagtttggattatacagagagaggggacagtattagaaagtaactttttgtgcaattacatagtaacgtagtttggttatgtgattatgcccatatattttattaagtagcacacaaaccaaaaagaaaatatgaaaactgaagatgcaggcttttgttc

SEQ ID NO: 37 - protein, 431 aa, Arabidopsis thaliana
  misc_feature (1)..(431): Ceres CDNA ID no. 23389731
MAFHHNHLSQDLSFNHFTDQHQPPPPQPPPPPPQQQQHFQEAPPPNWLNTALLRSSDNNNNFLNLHTATANTTTASSSDSPSSAAAAAAANQWLSLSSSFLQRNNNNNASIVGDGIDDVTGGADTMIQGEMKTGGGENKNDGGGATAADGVVSWQNARHKAEILSHPLYEQLLSAHVACLRIATPVDQLPRIDAQLAQSQHVVAKYSALGAAAQGLVGDDKELDQFMTHYVLLLCSFKEQLQQHVRVHAMEAVMACWEIEQSLQSLTGVSPGEGMGATMSDDEDEQVESDANMFDGGLDVLGFGPLIPTESERSLMERVRQELKHELKQGYKEKIVDIREEILRKRRAGKLPGDTTSVLKAWWQSHSKWPYPTEEDKARLVQETGLQLKQINNWFINQRKRNWHSNPSSSTVLKNKRKSNAGDNSGRERFA

SEQ ID NO: 38 - protein, 393 aa, Arabidopsis thaliana
  misc_feature (1)..(393): Public GI no. 1045044
MAFHNNHFNHFTDQQQHQPPPPPQQQQQQHFQESAPPNWLLRSDNNFLNLHTAATAAATSSDSPSSAAANQWLSRSSSFLQRGNTANNNNNETSGDVIEDVPGGEESMIGEKKEAERWQNARHKAEILSHPLYEQLLSAHVACLRIATPVDQLPRIDAQLAQSQNVVAKYSTLEAAQGLLAGDDKELDHFMTHYVLLLCSFKEQLQQHVRVHAMEAVMACWEIEQSLQSFTGVSPGEGTGATMSEDEDEQVESDAHLFDGSLDGLGFGPLVPTESERSLMERVRQELKHELKQGYKEKIVDIREEILRKRRAGKLPGDTTSVLKSWWQSHSKWPYPTEEDKARLVQETGLQLKQINNWFINQRKRNWHSNPSSSTVSKNKRRSNAGENSGRDR

SEQ ID NO: 39 - protein, 393 aa, Arabidopsis thaliana
  misc_feature (1)..(393): Public GI no. 26451634
MAFHNNHFNHFTDQQQHQPPPPPQQQQQQHFQESAPPNWLLRSDNNFLNLHTAASAAATSSDSPSSAAANQWLSRSSSFLQRGNTANNNNNETSGDVIEDVPGGEESMIGEKKEAERWQNARHKAEILSHPLYEQLLSAHVACLRIATPVDQLPRIDAQLAQSQNVVAKYSTLEAAQGLLAGDDKELDHFMTHYVLLLCSFKEQLQQHVRVHAMEAVMACWEIEQSLQSFTGVSPGEGTGATMSEDEDEQVESDAHLFDGSLDGLGFGPLVPTESERSLMERVRQELKHELKQGYKEKIVDIREEILRKRRAGKLPGDTTSVLKSWWQSHSKWPYPTEEDKARLVQETGLQLKQINNWFINQRKRNWHSNPSSSTVSKNKRRSNAGENSGRDR

SEQ ID NO: 40 - protein, 419 aa, Arabidopsis thaliana
  misc_feature (1)..(419): Public GI no. 9795158
MAFHNNHFNHFTDQQQHQPPPPPQQQQQQHFQESAPPNWLLRSDNNFLNLHTAASAAATSSDSPSSAAANQWLSRSSSFLQRGNTANNNNNETSGDVIEDVPGGEESMIGEKKEAERWQNARHKAEILSHPLYEQLLSAHVACLRIATPVDQLPRIDAQLAQSQNVVAKYSTLEAAQGLLAGDDKELDHFMTHYVLLLCSFKEQLQQHVRVHAMEAVMACWEIEQSLQSFTGVSPGEGTGATMSEDEDEQVESDAHLFDGSLDGLGFGPLVPTESERSLMERVRQELKHELKQGYKEKIVDIREEILRKRRAGKLPGDTTSVLKSWWQSHSKWPYPTEEDKARLVQETGLQLKQINNWFINQRKRNWHSNPSSSTVSKNKRRRYMESLNLEGFLYEAMQVKTAEETVEIKLACRDPKAL

SEQ ID NO: 41 - protein, 427 aa, Malus x domestica
  misc_feature (1)..(427): Public GI no. 1946222
MAYHNHLSQDLPLHHFTDQTHHQHQQYQSDQPDPNSKPPEPHHSFQPAPNWLNSALLRNFTNTDTNPTNSNNANNNGGGVSNFLNLHVTASDSAASQASNQWLSQSHRPILHRNHSDVNDDVTVAGDSMIAAALSHDSADLKPDSILNKNEGGGGDGGVMNWQNARHKAEILAHPLYEPLLSAHVACLRIATPVDQLPRIDAQLAQSQNVVAKYSALGNGMVGDDKELDQFMRNYVLLLCSFKEQLQQHVRVHAMEAVMACWEIEQSLQSLTGVSPGEGTSATMSDDEDDQVDSDANLFDEGMEGHDSMGFGPLIPTESERSLMERVRQELKHELKQGYKEKIVDIREEILRKRRAGKLPGDTTSVLKAWWQSHSKWPYPTEEDKARLVQETGLQLKQINNWFINQRKRNWHSNPSTSTVLKSKRKR

SEQ ID NO: 42 - protein, 345 aa, Glycine max
  misc_feature (1)..(345): Ceres CLONE ID no. 515966
MAFHDHLQHEIAFQRFTEEQELTENRDMQQRLPPPTWLNNVNARQQNFLDTEKSVDRNNRSESNCESEDLREYKADILGHPLYDQLLSAHVSCLRIATPVDQLPRIDAQLQQSQRVVEKYSALAHNGVVDEKELDQFMTHYVLLLCAFKEQLQQHVRVHAMEAVMACWDLEQSLQSLTGVSPGEGTGATMSDDEDDQAESNANLYEGSLDGGETLGFGPLVPTESERSLMERVRHELKHELKQGYKEKIVDIREEILRKRRAGKLPGDTTSLLKAWWQSHSKWPYPTEEDKARLVQETGLQLKQINNWFINQRKRNWHTNNPSSSSNSKSKRKSSAGEASNQSFM

SEQ ID NO: 43 - protein, 426 aa, Lycopersicon esculentum
  misc_feature (1)..(426): Public GI no. 6016226
MEFQDHFSQEMVLHQQQQQQQQQQNAVLRSMLPESPHHDARKSPPTWLNTSLLRQQHSQFGNASSPSSAAAAAAVAGGNNFLHLQTSNSDSSNSNQWLSPTAAAGGGSNGGGSGHNDELSESMNFAKKMSQQHSGGGEENNNNNNNNNNNNNEEENSWEREKCKADILNHPLYDQLLSAHVSCLRIATPVDQLPRIDAQLAQSQNVVAKYSVLGQGQPPLDDKDLDQFMTHYVLLLSSFKEQLQQHVRVHAMEAVMACWELEQSLQSLTGVAPGEGTGATMSDDDDDQADSDTNFLDGGFDGPDSMGFGPLVPTESERSLMERVRQELKHELKQGYKEKIVDIREEILRKRRAGKLPGDTTSVLKAWWQSHSKWPYPTEEDKARLVQETGLQLKQINNWFINQRKRNWHSNPSTSSSQKSQTQECR

SEQ ID NO: 44 - protein, 422 aa, Nicotiana tabacum
  misc_feature (1)..(422): Public GI no. 7446245
MAYNPNHMSQEMDMHHFSDENSAVLRSILPEQLAQSSPDVKPLDHQQPPTWLNSAILRQESHYTGTGGRGVGENFLNLHSNSESSAAASQASNQWLSRSILRRNVSDVQTSNNSSAVIAAADLKNDDGNNNNDNGNNNAGGQLTDSEVVGGGGTDGILNWQNAGYKAEILAHPLFEQLLSAHVACLRIATPVDQLPRIDAQLAQSQQVVAKYSTLGQNIGDDKELDQFLTHYVLLLCPFKEQLQQHVRVHAMEAVMACWEIEQSLQSLTGVSPGEGTGATMSDDEDDQVDSEANLFDGSLDGHDGMAFGLPTESERSLMERVRQELKHDLKQGYKEKLVDIREEILRKRRAGKLPGDTTSVLKAWWQSHAKWPYPTEEDKAKLVQETGLQLKQINNWFINQRKRDWHSNASSSTTSKSKRKR

SEQ ID NO: 45 - protein, 374 aa, Oryza sativa subsp. japonica
  misc_feature (1)..(374): Public GI no. 1805618
MAFHGHLPHEMTMQALGADDAAVAAAAAAGGVGAGGAPAWMRYNDGSFLHLQTTSDSSASPSGAAAAAAAAAAAAAAGVQQWMGGGGGGEDAVAAAMGGGGEADAARCKAEILAHPLYEQLLSAHVACLRIATPVDQLPRIDAQLAQSQGVVAKYSALAAAAAGDDGRELDQFMTHYVLLLCSFKEQLQQHVRVHAMEAVMACWELEQNLQSLTGASPGEGTGATMSDGEDDQADSEANMYDPSLDGADNMGFGLPTESERSLMERVRQELKHELKQGYKEKLIDIREEILRKRRAGKLPGDTTSTLKAWWQSHAKWPYPTEEDKARLVQETGLQLKQINNWFINQRKRNWHSNPSSSTSVKTKRKSNAGDNNS

SEQ ID NO: 46 - protein, 375 aa, Oryza sativa subsp. japonica
  misc_feature (1)..(375): Public GI no. 1805617
MAFHGHLPHEMTMQALGADDAAVAAAAAAGGVGAGGAPAWMRYNDGSFLHLQTTSDSSASPSGAAAAAAAAAAAAAAGVQQWMGGGGGGEDAVAAAMGGGGEADAARCKAEILAHPLYEQLLSAHVACLRIATPVDQLPRIDAQLAQSQGVVAKYSALAAAAAGDDGRELDQFMTHYVLLLCSFKEQLQQHVRVHAMEAVMACWELEQNLQSLTGASPGEGTGATMSDGEDDQADSEANMYDPSLDGADNMGFGLPTESERSLMERVRQELKHELKQGYKEKLIDIREEILRKRRAGKLPGDTTSTLKAWWQSHAKWPYPTEEDKARLVQETGLQLKQINNWFINQRKRNWHSNPSSSTSVKTKRKRAGGIHPKQ

SEQ ID NO: 47 - protein, 436 aa, Ceratopteris richardii
  misc_feature (1)..(436): Public GI no. 11463943
MEDKSYVESKLGAPTWLSGAVQQQQVRASQQGTTNSAGSSFVNEATQQQQSHDTPRKWCGCEAGGQCVQCSFMHLSGGGAGGGGSSVAGNGIAMTANHNQQHSAAEAAGLLAIARSGGDLAQSGQGGRGNLLDLHSDTANSSDLVEGEHGGGEHGGGGHNQQDSQVLWQNARLKADITMHPLYDQLLAAHVACLRIATPVDQLPRIDAQIAQASQIVAKYAVLGQNNLLVGEEKDELDQFMAHYVLLLCTFKEQLQQHVKVHAMEAVMACWELEQSLLTLTGVSPGEGTGATMSDDDDDPAESDPSIYDPAFDTHDSGAFGPLIPTETERTLMERVRQELKNELKNGYKDRIVDVREEILRKRRAGKLPGDTTSVLKAWWHAHSKWPYPTEDEKARLVQETGLQLKQINNWFINQRKRNWHSNPSSTAAMKTKRKR

SEQ ID NO: 48 - DNA, 1667 bp, Arabidopsis thaliana
  misc_feature (1)..(1667): Ceres CDNA ID no. 23644306
gttaagagatccttctccctcttcgaagatgagcggtacggtgaaggatatcgtttcaaaggcggagcttgataacttgcgccagagcggcgcaccagtcgtgcttcacttctgggcttcttggtgtgatgcttcgaagcagatggatcaagttttctctcatctcgctactgatttccctcgtgctcacttctttagggttgaagctgaggaacatcctgagatatctgaggcttactctgttgctgctgtgccttatttcgtcttcttcaaggatggtaaaactgtggatacacttgagggtgcagatccatcaagtttagctaataaggttggcaaagttgctggttctagtacttctgcggagcctgctgctcctgcaagcttagggttggctgctgggccaacgattcttgaaactgtgaaggagaatgcgaaagcttctttacaagaccgagctcagcctgtatctaccgccgatgctctcaagagccgtttggaaaagctcactaattctcaccctgtcatgttattcatgaaaggtattcctgaagagcctaggtgtgggtttagcaggaaagtagttgacattttgaaagaggttaacgttgattttggaagttttgacatactatcggataacgaagtgcgagagggtttgaagaaattctctaactggccaacgtttcctcagctgtactgcaacggagagcttcttggtggagctgatatcgcaatagcgatgcacgagagcggtgaactaaaagatgctttcaaagatcttgggatcacgacagttggttcaaaagaaagtcaggatgaagctggaaaaggaggaggggttagttcaggaaacacaggcttaagtgagaccctccgagctcggctcgaaggtctggtcaattccaaaccagttatgctgttcatgaaaggaagaccagaagaaccaaagtgtgggttcagtgggaaagtggttgaaatcctcaaccaagaaaaaatcgagtttgggagtttcgatatcctcttagatgacgaagttcgccaaggccttaaagtgtattcaaactggtcaagctatcctcagctttacgtgaaaggcgagcttatgggtggatcagacattgtcttggagatgcaaaagagcggtgagctgaaaaaggtcttgaccgagaaagggatcactggagaacagagtcttgaagatagattgaaggcactgatcaattcctcggaagtaatgctattcatgaaaggttcaccagatgaaccgaaatgcggatttagctccaaagttgtgaaagcattgagaggagaaaacgtgagtttcggatcgtttgatatcttgactgatgaagaagtaaggcaagggattaagaatttctcaaactggccaacttttcctcagctatactacaaaggtgagttaattggaggatgtgatatcattatggagctaagtgagagtggtgatctcaaagcaactctatccgagtaagtaatatatacaagtctctgtctgctggtttgccttggtgagagagaacatttcagttatggtaataatatgttttaggtgttacaaacattgatattgttgcttctcaagctttgtcttgttatttctattgctgagtctattagattcataactatttttctctctttgt

SEQ ID NO: 49 - protein, 488 aa, Arabidopsis thaliana
  misc_feature (1)..(488): Ceres CDNA ID no. 23644306
MSGTVKDIVSKAELDNLRQSGAPVVLHFWASWCDASKQMDQVFSHLATDFPRAHFFRVEAEEHPEISEAYSVAAVPYFVFFKDGKTVDTLEGADPSSLANKVGKVAGSSTSAEPAAPASLGLAAGPTILETVKENAKASLQDRAQPVSTADALKSRLEKLTNSHPVMLFMKGIPEEPRCGFSRKVVDILKEVNVDFGSFDILSDNEVREGLKKFSNWPTFPQLYCNGELLGGADIAIAMHESGELKDAFKDLGITTVGSKESQDEAGKGGGVSSGNTGLSETLRARLEGLVNSKPVMLFMKGRPEEPKCGFSGKVVEILNQEKIEFGSFDILLDDEVRQGLKVYSNWSSYPQLYVKGELMGGSDIVLEMQKSGELKKVLTEKGITGEQSLEDRLKALINSSEVMLFMKGSPDEPKCGFSSKVVKALRGENVSFGSFDILTDEEVRQGIKNFSNWPTFPQLYYKGELIGGCDIIMELSESGDLKATLSE

SEQ ID NO: 50 - protein, 499 aa, Zea mays
  misc_feature (1)..(499): Ceres CLONE ID no. 280200
MASGGAVREVGSAAELQAAVAGARAAAVHFWASWCEASKQMDEVFAHLAVDFPHAAFLRVEAEEQPEISEAYGVTAVPYFVFCKEGKTVDTLEGANPASLANKVAKVAGPASVAESAVPASLGVAAGPAVLENIQKMAQRNGSSAVESISSGSTEDALNKRLEQLVNSHPVFLFMKGTPEQPRCGFSRKVIDILKQEGVKFGSFDILTDNDVREGMKKFSNWPTFPQLYCKGELLGGCDIVVAMHESGELKDVFEEHNITLKPQGSKNEEAGEPESATEKGSAVSESIKLTDAQKTRLESLINSSPVMVFIKGTPEEPKCGFSGKLVHILKQENIPFSSFDILSDDEVRQGLKVFSNWPSYPQLYIKGELVGGSDIVMEMHKSGELKKILSEKGVRQKGNLEDRLKSLISSAPVMLFMKGTPDAPRCGFSSKVVNALKKEGVSFGSFDILSDEEVRQGLKTYSNWPTFPQLYYKSELIGGCDIILEMEKSGELKSTLSM

SEQ ID NO: 51 - protein, 491 aa, Oryza sativa subsp. japonica
  misc_feature (1)..(491): Public GI no. 22165075
MAAVREVGSKAELEAAAGGARAAAVHFWAAWCEASKQMDEVFAHLAVDFSHAVFLRVEAEEQPEISEAYGVTAVPYFVFLKEGKTVDTLEGANPASLANKVAKLAGPASVAESAVPASLGVAAGPAVLEKVQEMAQQNGASATSSAEDALNKRLEQLVNSHPVFLFMKGTPEQPRCGFSRKVVDVLKQEGVEFGSFDILTDNDVREGMKKFSNWPTFPQLYCKGELLGGCDIVIAMHESGELKDVFKEHNIPLQPQGSKNEEAVKAKPDTEKSGAVSEPALLTAAQKERLESLVNFSTVMAFIKGTPEEPKCGFSGKLVHILKQEKIPFSSFDILTDDEVRQGLKLLSNWPSYPQLYINGELVGGSDIVMEMHKSGELKKVLSEKGIVAKESLEDRLKALISSAPVMLFMKGTPDAPRCGFSSKVVNALKQAGVSFGAFDILSDEEVRQGLKTYSNWPTFPQLYYKSELIGGCDIVLELEKSGELKSTLSE

SEQ ID NO: 52 - DNA, 761 bp, Arabidopsis thaliana
  misc_feature (1)..(761): Ceres CDNA ID no. 23543586
atggattctgaacccggccagccagagaacggtactgatgctgagaaatctgttgtaggaaaatgttctgagacgatcagtgactcagaacctggccagcctgagaacggcactgaagccgagaaatctgttgtgcaaaaatgttctgagaagatcgatgaatctgaagccggccagcctgagaacagtactgaagccgagaaattcgttgtgcggaaatgttctgagaagatcgatggctctgaaaatgttcctgcagctggttgtgttattaggactgatctaaactcgtgtcctgagtttgagaagaaacctttgtttttgaccaaaaactggaggaacatcctatgcagatgcgaaaagtgccttgagatgtataagcagagaaaggtaagctatctacttgatgcagaggacacaattgttgaatacgagaagaaggcgaaggaaaaaagaacagagaaactggagaaacaagaaggtgaagcacttgatcttctgaataatctagaccacgtatccaaagttgagctccttcacggaatcaaagacttccaagacggactccagggtttaatggtatatattgtcaccatctatgaaagaaatttgattacaattctcaggaaccacttcatttaaaaagacttgattcgttgttggttttgtaggagtctgctgggccatcaaaggcgataacttctgcagatatcgagcaaatgttttcaaaactgaaaaacaaacgtaaaaggatggagtga

SEQ ID NO: 53 - protein, 222 aa, Arabidopsis thaliana
  misc_feature (1)..(761): Ceres CDNA ID no. 23543586
MDSEPGQPENGTDAEKSVVGKCSETISDSEPGQPENGTEAEKSVVQKCSEKIDESEAGQPENSTEAEKFVVRKCSEKIDGSENVPAAGCVIRTDLNSCPEFEKKPLFLTKNWRNILCRCEKCLEMYKQRKVSYLLDAEDTIVEYEKKAKEKRTEKLEKQEGEALDLLNNLDHVSKVELLHGIKDFQDGLQGLMESAGPSKAITSADIEQMFSKLKNKRKRME

SEQ ID NO: 54 - DNA, 918 bp, Arabidopsis thaliana
  misc_feature (1)..(918): Ceres CDNA ID no. 23361365
aaaagaaagaaaaaaatgccttcttttgcttttggatctcatcaccatttggcgaatcctacagactcgccgccgtactccgtcgaaattagcatcgacggtgactcctccgacttggattctttgtctcaggtcgatttagagtccggcggtgtaccggcgccggagaaacagctacattccggtggtaagaagaggagaactaggaggagaaagaggaggaagaagaagaagaagaagaaaggtggaagagattgcaggatctgtcatcttcctttagagactaacaaagaagctgaagatgaagatgaagaagaagaagatgattctgatgatgatgaagatgaagaagatgaagaagaagaagaagaagaagaagaatattatggtttgcctttgcaattaggttgctcttgtaaaggtgatttgggtgttgctcatagtaagtgtgctgagacttggtttaagatcaaaggaaacatgacatgtgagatatgcggcgcaatggctctaaacgtggctggtgaacaatctaatccggagagcactgcttctacacattcacaagcagctgcgggacaatctctaactcagacagagccacgaggaatctggcatggtcgccctgttatgaacttcttacttgctgctatggtcttcgccttcgttgtttcttggctttttcacttcaaagtcctcaagtgaaacgttttccatctctctctctctctctctttcgcccacccgcacgagctcactcttctcctgttcttgagccgcttgcaatcgctgaatcaatccctcctgttgcttgattattcacacgttttgtagctgtaaaaacttggtagtgtttagattgttgtaacttgtgagatttacattttgtataatattgttctctgctccggcttcgtg

SEQ ID NO: 55 - protein, 229 aa, Arabidopsis thaliana
  misc_feature (1)..(229): Ceres CDNA ID no. 23361365
MPSFAFGSHHHLANPTDSPPYSVEISIDGDSSDLDSLSQVDLESGGVPAPEKQLHSGGKKRRTRRRKRRKKKKKKKGGRDCRICHLPLETNKEAEDEDEEEEDDSDDDEDEEDEEEEEEEEEYYGLPLQLGCSCKGDLGVAHSKCAETWFKIKGNMTCEICGAMALNVAGEQSNPESTASTHSQAAAGQSLTQTEPRGIWHGRPVMNFLLAAMVFAFVVSWLFHFKVLK

SEQ ID NO: 56 - protein, 197 aa, Arabidopsis thaliana
  misc_feature (1)..(197): Public GI no. 9759231
MPSFAFGSHHHLANPTDSPPYSVEISIDGDSSDWDSLSQVDLESGGVPAPEKQLHSGGKKRRTRRRKRRKKKKKKKGGRDCRICHLPLETNNGLPLQLGCSCKGDLGVAHSKCAETWFKIKGNMTCEICGAMALNVAGEQSNPESTASTHSQAAAGQSLTQTEPRGIWHGRPVMNFLLAAMVFAFVVSWLFHFKVLK

SEQ ID NO: 57 - protein, 207 aa, Glycine max
  misc_feature (1)..(207): Ceres CLONE ID no. 642012
MLVTEDKSHVAVAIDNDGCCHRSSAGGEGCSDASDRTDKEQRRSSHVSGTEIVGVCEERGSECSVEVDLVPEVKVHLANEERDCRICHLSMDMTNHESGTPIELGCSCKDDLAAAHKQCAEAWFKIKGNKTCEICGSVARNVAGAIEIQMTEQWNEANDASTAPSSGPAPLAETQNFWQGHRFLNFLLACMVFAFVISWLFHFNVPS

SEQ ID NO: 58 - protein, 207 aa, Glycine max
  misc_feature (1)..(207): Ceres CLONE ID no. 518866
MLVTEDKSHVVVAIDNDGCCHRSSAGGEGFADAGERSDEEQRSSHDSGTEIVGVCEKERGSECSVEVDLVPEVKVHLANVERDCRICHLSMDMTNHESGTSIELGCSCKDDLAAAHKQCAEVWFKIKGNKTCEICGSVARNVAGAIEIQMTEQWNEANDASTAPPSGPAPPTETRNFWQGHRFLNFLLACMVFAFVISWLFHFNVPS

SEQ ID NO: 59 - protein, 246 aa, Triticum aestivum
  misc_feature (1)..(246): Ceres CLONE ID no. 766557
MVGSGAQSASGAEVEDIELGERRRADEFVDDDEEGSQYFTDAEDRSWPSHSRQESAAFEDCISRCASTRASSCGGADSDADIEAGGGHFRKSSCVSECSLDDVDLEAGFGGESAKGSPDPEKAEKNCRICHLGLESAAAESGAGITLGCSCKGDLSYSHKQCAETWFKIRGNKTCEICSSTACNVVVLGDPEFVEQSNESNTTAAGHTFPNETRRFWQGHRFLNFLLACMVFAFVISWLFHFNVPG

SEQ ID NO: 60 - protein, 243 aa, Zea mays
  misc_feature (1)..(243): Ceres CLONE ID no. 246572
MDHTGAKDLERGELRRGAPEFADGDDGDGEESQYFSDAEDRSWPSHSRHDSTAYEDYVSPCVSARASSVDADADADADGEAVRQHCRKSSCVSEGSLDDVDLEAGLSEIIKASPEKAEQNCRICHLGLESAAAESGAGITLGCSCKGDLSYAHKQCADTWFKIRGNKVCEICSSTASNVVVLGDPEFSDQWSETNSAAAAAQAPPPPAEPRRFWQGHRFLNFLLACMVFAFVISWLFHFNVPG

SEQ ID NO: 61 - protein, 244 aa, Oryza sativa subsp. japonica
  misc_feature (1)..(244): Public GI no. 55733851
MAGGGGQSAIAAEDDDLERGAERDEEEEEGSQYFTDAEDHQSWASHSRHNSTAYEDYISTCASVRASSYDGEAEEEEDAGGGGGGVEHHRRKSSCVSECSLDDVDLEAGLAEVIKGSPEKAERCCRICHLGLETAAAESGAGITLGCSCKGDLSYSHKQCAETWFKIRGNKICEICSSTACNVVGIGDSESVEQWNESNSTAPAQAPPAETQRFWQGHRFLNFLLACMVFAFVISWLFHFNVPG

SEQ ID NO: 62 - DNA, 1069 bp, Arabidopsis thaliana
  misc_feature (1)..(1069): Promoter Construct YP0092 with Candidate Id 13148193 as reported in Report Number # 13
aaagattgagttgagagagatggtggagacgcagaacagacaaagggagtttaccatatagtgctctaaagggcaatgagattgcagtgatgtggctatccggggaatcatcgcaggttattccttcccatgagcaacaatcaatggatgggttccaattcagaggagaaacagaagaagaaacgtttccagagaaccacagtagggattctcgatcttgcgagttgcagagagcctctgaaactgcaatagaaaggacactgatgaaaagaacacactgaaggagtatgccaatcatgtgaaaactcagagcttgtattggtcttgtggttgatgaagttctcacaaaacctttggctttgaatctcccctcattagtcatggtgagaacaagaacaagacgagaaacagacaaagaagatgaaaaaacttgttggccagtgttgactaagggggaatagccccagacataacaaaattagacttgtcgtacatctttaatatttttttatctgtttctttgtcctgacgctttcattattcctgtgatcaattttctcataccattggtccatcgttaatcctttcttaatttcattttctacgtaacatgagaggagaccaagtcctatgagaacagttgacgtaacagtggttgttaagttaagttaaaaagaggaagctagtgagagtgaccgttaggtagagaagtgagatctttaaccactcttctttctctctctctctgcttttttcgtcgtctttcacatctactgttcgcaaactctcttatgcttccaataatggtgataccaattgagacttgcaggagaatctcctcttctccacactctatcaactggtcagccatggaatggtcgtttcagtttcaatattcctggattctttttaaggattcctgtttctcttctgttcctggtatattcttaacgacgaaattagtatcggatcctggtaatacattttgaagcttttaagtaccattgcactgggatccaacaatgtcctccgactcgtccaagatcaagaggaagcggaaccgcatccc

SEQ ID NO: 63 - DNA, 1004 bp, Arabidopsis thaliana
  misc_feature (1)..(1004): Promoter Construct YP0128 with Candidate Id 13148257 as reported in Report Number # 26
gataaactgataatggaaaagaacaaagaaaccagtttttaactatttgcatatgtaatttatttgttgcaaattatatttagttaaaatgtttcctctatttatatatatatatatcagtcaagcactatgtataagaaatgtcaatttataaatttttacatgtcctttaacagaaagaaaatgaatttttacatgtcattcatagagagtcactcgtttatttcttatatagagaataacacactcacatgcatatgcatgcaatatgatacattttatgacaaagataatcaacggaaacggtcaagacataatttgataaacaacttgcacgatgcacagatctgatcaaatatataactctttaacatatccaaaatattcaaaaagaaaaactcgatccaaactagcaacatcacgctcacgcggtaggctaaaaatttattaatctccaaaagtctttcttatgaacactgcaaacacaacaacttgaaaagtcatataggtttagatgatgacgcgtattggctatcgcttaccggagtggctcataaatacaataaacaatacgtaaaagtcaaagtcaaatatatttagtcaactataaccattaatcgggcaaaacctttagctgtcaaaacaacgtgaaaacgatatttgtatatatcatcaagaatcagtagataagagaatgatttaatcccctgactattacaattttggtgtaataaacagtctctattggtttttattctttgttttaatttctcatgacctatagagagaattaggtagtttcgaaaattggctaatcaacttttgaaaactactgtctactttgcttaaattctctacacttagtttcggataagataattgtcggactaatagttaatcccttgacaatctttgatattataaaaggtttagttaatctcttctctatataaatattcatacaccagctttcaaaaatatataatccaaacaccaaaaacaaa

SEQ ID NO: 64 - DNA, 1000 bp, Arabidopsis thaliana
  misc_feature (1)..(1000): Promoter Construct YP0119 with Candidate Id 13148175 as reported in Report Number # 80
taccaaaaataaggagtttccaaaagatggttctgatgagaaacagagcccatccctctccttttccccttcccatgaaagaaatcggatggtcctccttcaatgtcctccacctactcttctcttctttctttttttctttcttattattaaccatttaattaatttccccttcaatttcagtttctagttctgtaaaaagaaaatacacatctcacttatagatatccatatctatttatatgcatgtatagagaataaaaaagtgtgagtttctaggtatgttgagtatgtgctgtttggacaattgttagatgatctgtccatttttttcttttttcttctgtgtataaatatatttgagcacaaagaaaaactaataaccttctgttttcagcaagtagggtcttataaccttcaaagaaatattccttcaattgaaaacccataaaccaaaatagatattacaaaaggaaagagagatattttcaagaacaacataattagaaaagcagaagcagcagttaagtggtactgagataaatgatatagtttctcttcaagaacagtttctcattacccaccttctcctttttgctgatctatcgtaatcttgagaactcaggtaaggttgtgaatattatgcaccattcattaaccctaaaaataagagatttaaaataaatgtttcttctttctctgattcttgtgtaaccaattcatgggtttgatatgtttcttggttattgcttatcaacaaagagatttgatcattataaagtagattaataactcttaaacacacaaagtttctttattttttagttacatccctaattctagaccagaacatggatttgatctatttcttggttatgtattcttgatcaggaaaagggatttgatcatcaagattagccttctctctctctctctagatatctttcttgaatttagaaatctttatttaattatttggtgatgtcatatataggatcaa

SEQ ID NO: 65 - DNA, 999 bp, Arabidopsis thaliana
  misc_feature (1)..(999): Promoter Construct YP0275 with Candidate Id 11768836 as reported in Report Number # 142
aaacattaatatgtagtaactatgggcgtatgctttactttttaaaatgggcctatgctataattgaatgacaaggattaaacaactaataaaattgtagatgggttaagatgacttatttttttacttaccaatttataaatgggcttcgatgtactgaaatatatcgcgcctattaacgaggccattcaacgaatgttttaagggccctatttcgacattttaaagaacacctaggtcatcattccagaaatggatattataggatttagataatttcccacgtttggtttatttatctattttttgacgttgaccaacataatcgtgcccaaccgtttcacgcaacgaatttatatacgaaatatatatatttttcaaattaagataccacaatcaaaacagctgttgattaacaaagagattttttttttttggttttgagttacaataacgttagaggataaggtttcttgcaacgattaggaaatcgtataaaataaaatatgttataattaagtgttttattttataatgagtattaatataaataaaacctgcaaaaggatagggatattgaataataaagagaaacgaaagagcaattttacttctttataattgaaattatgtgaatgttatgtttacaatgaatgattcatcgttctatatattgaagtaaagaatgagtttattgtgcttgcataatgacgttaacttcacatatacacttattacataacatttatcacatgtgcgtctttttttttttttactttgtaaaatttcctcacttttaagacttttataacaattactagtaaaataaagttgcttggggctacaccctttctccctccaacaactctatttatagataacattatatcaaaatcaaaacatagtccctttcttctataaaggttttttcacaaccaaatttccattataaatcaaaaaataaaaacttaattagtttttacagaagaaaagaaaaca

SEQ ID NO: 66 - DNA, 1000 bp, Arabidopsis thaliana
  misc_feature (1)..(1000): Promoter Construct PT0613 with Candidate Id 13148297 as reported in Report Number # 163
ttaatactaacattgtagaaagccacaaaaaagaaattgaaatgtgagtagatgctgagtcagaggtttggtcaatacacaacagctaattgagataatattatacacgtcacgatgacttgttttttctcctcccaacttgttaatttctttattcttaaaattaaaccatcgcaaaaacagaagaacacagctgtttttctcgactcccaatttctattttgctgctaaggacatttcatttcattatttcccaattcaggactccttagattttcctaaatttgttttcctaacttgctctctctcattctaacattttctcatttttttagattatcttgtactttttagtagattattttatcaggttttacaaacatacattgacattctaaaaagggcttctaaaaattcagtgtggaatgctgatatactaaaaaaaggtcatgcaaaattatctacgatttatctaaaattagataatttgccatatataactattaactaataatcgatcctttgattttttgtttagataaaacgaaacagctatatcttttttttttgttatcggattttaatcgaataaaagctgaaaaataacagttatatcttcttcttttttaactaatgaaacagttatatcttaaacaaacaacagaaacagtaaaatattaatgcaaatccgcgtcaagagataaattttaacaaactaataacaattgagataagattagcgcaaaagaaactctaattttagagcgtgtaaacacaaacacgtcttgaaagtaaacgtgaattacacgcttctaaaacgagcgtgagttttggttataacgaagatacggtgaagtgtgacacctttctacgttaatttcagtttgaggacacaactcaagttatgtttgatatctaaggacttgcactgtctccaaatctgcaggaaggactttttgattggatcaatataaataccatctccattctcgtctccttc

SEQ ID NO: 67 - DNA, 351 bp, Arabidopsis thaliana
  misc_feature (1)..(351): Promoter Construct PT0625 with Candidate Id 13148207 as reported in Report Number # 166
gatcatgatcagtttcaactcgctgtgcccacgtgtcgagagatcggcacgtgcctgagctctcagccgctcataaatacacttgtttagtagcaacagtatactatagtagtcctctcctgtttggcttttagcttgcatcgatggatggatggatggatcgcatgagagggcttcgcgaaggtacggaaccttacacaacgcgtgtcctttctacgtggccatcgtgtaggcgtctcgccatgctacgtgtcccggaggatgtctcgatgccaacccttataaatactgttccattccaatcccatcgccacagccagtgcaaatctgatcgatcaagataatcgagca

SEQ ID NO: 68 - DNA, 999 bp, Arabidopsis thaliana
  misc_feature (1)..(999): Promoter Construct PT0672 with Candidate Id 15295940 as reported in Report Number # 171
cagccgtaaatcctccataaatttattttgcaagttttgctcattatataatgagcggaatttatgatataatcgtttgtaataatgttatgttttgatcaaaatttgaaattaaaagtaggtgagaacttgttatacagtgtagataaggtggatcttgaatataaaaataaaatttataagatgtatttaaagcagaaaagcataaaactttagataaaataatgtaaaaatgtgttagcatcaatgttgggatattggccgacccgaacttaatcaatgtcggaagccattacttctctcccaaaagacctttttccttcggagaactaggaacttcctcactacctttcgcttaacgtgaaagccataaatttcatatattcataaaaatcagaaaatctaaaactgtttagtatcacctgtttttggtatagactattggttttgtgttacttcctaaactatatgatttcgtacttcattggatcttatagagatgaatattcgtaaaaagataagttatctggtgaaacgttacttcagtcatgttgggtctagatttacatactactatgaaacattttaagataataattatcctagccaactatatgttctatattatgggccaagaagatatagaactaaaagttcagaatttaacgatataaattactagtatattctaatacttgaatgattactgttttagttgtttagaataaatagtagcgtgttggttaagataccatctatccacatctatatttgtgtgggttacataaaatgtacataatattatatacatatatatgtatatttttgataaagccatatattactccttgacctctgcccccatttccttttactataaataggaatactcatgatcctctaattcagcaatcaacaccaacgaacacaaccttttccaaagccaataataaaagaacaaaagcttttagtttcatcaaagacgaagctgccttagaa

SEQ ID NO: 69 - DNA, 998 bp, Arabidopsis thaliana
  misc_feature (1)..(998): Promoter Construct PT0678 with Candidate Id 15295964 as reported in Report Number # 178
aattaaatgaaaccgcccctaaattaggagggatttgggtaagtggtaacacattcactggaaacatgtgaagaaaggaggatgtcaagtagctgaaaactcagtatagtaaccaacggcttctcaccaacctttcattaataatttggtcatccctatatttttattcaacattttgtttttcaatagcttagagcaccttaatacctttcagtgtttttttataaaaaaaacaaaaattgggattaatcatcaatccccaaatgtaacgtttacttagattatgttcatttttctatacacacaaatcatattcttttgttttaatcttcgaaaaacgagaggacattaaatacccctaaaaaaggaggggacattactaccaacgtacattaacatgtttgatagcaaacgatttattttgttcgttttgaaaaggggaaagtaatgtgtaaattatgtaaagattaataaacttttatggtatagtaacattttcgaataataagagagggaaaacactcgccattgtcggcaatttagaaccaatattagaagggtttttttagagaaaaaggacttaaaagtttagagaccttaacaacaacttatttagaaatagacatgcttaagttgacaacagcgagtttattttctatatcgaagaaaaatacgaactttttcttaattagatttcgaatgcatgcactatcgagaatcgaccgtcacaagaaaaaactaatatacatactgtacatatctatattcaatattggtggggatgggtttaatgtgtatttataattcatggataaattcacacaataaggtccatgaaactagaaggtaccaaaaataagcattaatgactctttgccacttatatatatgattctctcatagtaccattttattctcccaaacctatcttcttcttcctctcttgtctctctcgctctctctcttctacattgtttcttgaggtcaatctattaaaa

SEQ ID NO: 70 - DNA, 1000 bp, Arabidopsis thaliana
  misc_feature (1)..(1000): Promoter Construct PT0688 with Candidate Id 15371509 as reported in Report Number # 183
acgttcagaggcatcgcttttgtacaaattgaagcgggtttgttcaatatttaaaataacacaggaaacattcaaatgtattattgatgttgcttaggtttgtgaaatgatatgaaccatatcgtatatattactagatttttcttatatgttttaagggtagtggggctgacctatcattctgtttggcattaccaatcagactatcagagtattcaccattcaggattccataactagaaaaagaaggggtttacattttctcatactgtataattttctactatcagagattttatcgattacattaatctcatagtgattattctgatttataaaaaagttgacaaaataattaaaaccagtattttataacaagattgtctctctcccatggccattattttgacctctgacttatttaaatcttaattaacagcataatactgtattaagcgtatttaaatgaaacaaaataaaagaaaaaaagaacaaaacgaaagagtggaccacatgcgtgtcaagaaaggccggtcgttaccgttaaggtgtgtcgaactgtgattgggccacgttaacggcgtatccaaaagaaagaaagggcacgtgtatagatctaggaaaaaagaaagaatggacggtttagattgtatctaggtaccaggaaatggaacgtcacaccaaacggtacgtgtcggatcctgcccgttgatgctgacggtcagcaacttccccttattcatgcccccctgcccgttaattacgtgtaacccttccatgcgaaaatcaaacccttttttttttttgcgttcttcttcaacttttctttttaaatcaaaccttttctttttaaaatcacattgcatttcctaacgctcaacaaaatctctctctactaatatctctctctctctctctctattgttgaagaagactcataatcggagattgtttgtttttggtttgctctgtaaattggagaagttttgttagagatcaa

SEQ ID NO: 71 - DNA, 998 bp, Arabidopsis thaliana
  misc_feature (1)..(998): Promoter Construct PT0660 with Candidate Id 15224257 as reported in Report Number # 186
caagtcaagttccaatattctaaggagaaataatagtatactaaacatacattagagaggttaaacttctttttggatttaagtgtgtatgcataggctatttattcttaagtataactattaactgtagctagatttatacaagaaatacataaaactttatgcatgtgaggtagccatgaatatacgtacatgttgcaatcgattatacatgttgtatttggatttctctatacatgttttaacttgtcattctctaagtatatacataccattaatactgtgggcatgagtttatgataagacttttcttttggagaccagttttgttttcctttccacctatatttgtctataggcttcacggtacactagtttacaagtgtttttatatgttctaaataaaattgagattttccggaacggtatgatctgtttgcaaataaggacgtatatataacagtatcaaatatatttgttgttataaggcaataatatattttctgagatattgcgtgttacaaaaaagaaatatttgttaagaaaaaaaaagatggtcgaaaaaggggagtaggtgggggcggtcggcttttgattagtaataaaagaaaccacacgagtgacctaccgattcgactcaacgagtctaccgagctaacacagattcaactcgctcgagcttcgttttatgacaagttggtttttttttttttttttaattttttcatcttcttgggtttggttgggtcactcttcaggtcaggtgtgtaaaaaagaaagaaagaaaagagagattgttgtgttgtaacccctttgactaaaatctaatgaacttttttaacacaacaaaactccttcagatctgaaagggttcttcttctctcttagtctcttcgtccttttattctccgtcgtcgtttcatgatctgactctctggtcttctcttcttcttcttcttcttctattttttcttacttcgtcactgttgtgtctgaac

SEQ ID NO: 72 - DNA, 1000 bp, Arabidopsis thaliana
  misc_feature (1)..(1000): Promoter Construct PT0676 with Candidate Id 15295958 as reported in Report Number # 205
aagatagtacagtttcagtgttttgagaaaaaaagctgaactaaaactaaaatgtttaaggacacaatatttagtttcaattagataattcaacagtttgaacaattttttttttttttttttgaagtcatttatttatacaatgttttaaaacgcattaagcatttaggcagccgacaaacgcctattgtctaactgtaaataggcgcttccacttaggttcatattgcatatttactatatgtgtatagtgacaaaaaccaatatttctcttattttggatgaaggtatagtagttgttaaatgttcaatataattaagcattaatgacaaataaaataaaattaatttagttgataaaaagataatcttataaaaagatcgatgaatagatataatggtttactgaattctatagctcttaccttgcacgactatgtcccaaggagaggaagtaccttaactataattctgaacataattttgtctatcttggtgagtattatatgacctaaaccctttaataagaaaaagtataatactggcgtaacgtaataaattaacacaatcataagttgttgacaagcaaaaaaacatacataatttgtttaatgagatatattagttatagttcttatgtcaaagtacaattatgcctaccaaaattaattaatgatttcaacaggaagtctgagatgatgggccgacgtgtagttacgtttcttgaattgtgagagatggtatttattatactgaagaaaacattatttactaaataaattttcatttcacatcttctgtaatcaatgcgggtagatgaagaagttgttaatacgatggccaaccatatggatctcttttttggcgtttctatatatagtaacctcgactccaaaggcattacgtgactcaataaaatcaagtcttttgtttccttttatccaaaaaaaaaaaaaagtcttgtgtttctcttaggttggttgagaatcatttcatttca

SEQ ID NO: 73 - DNA, 1000 bp, Arabidopsis thaliana
  misc_feature (1)..(1000): Promoter Construct PT0683 with Candidate Id 15295991 as reported in Report Number # 209
gattgaatgatgagtgtgcacccttgtattactaataaaaaatttagcaacagttataagctaacgtcatccatgagtcattcattagattcactatttgcgttctcaaaaatcgaattgttaaaatttgagaagctctaatatacgagtcaatgagatgtggcaaaagcatgtccttgaccataaaatttcgaggggtcaactcattagataaggacaagaatcaaccaattgaaggcgtcttctataacaagtttctttattactaatattaaagtccaatggggtgagggggagaagaacttaaataaaaggaaataattggtaagtgaataaaatctaaatacgatactagatgattgatttgtgctagtgcatggtattagatcagatatgtgttactattcgaattcaaattggcatattccatgttgttgataagaaaattgtagaagtgtaaaagctgagttactatattcaaactagtggtttacataaagtgagacaacaactgtttcacaaaaatgactataaaatagtaagtagtattaggtcaattgattttaaaattttaatcaaattcaaatttgtgatataatcaaatttgtttatagaaaatgttaagaaatcaattttggcagaactaattcagtgagaaacaatcatttacaaaaacaattttaacattatttaacagtaagatttgacatttaacccgttcgtgtgaacccatcatatctaacatggctctacccatgacgcctccatgccatggacaattttgacagatcagaagttctgaacgtggacgaggtaagaacaccatgatgatacgattggagttagttatgtcgccaccgacatcactgccaatctcattaataaaagtggtactaaatctctaatctctattaactataaatataacaaagaaccaaaagaaagtttcttatctctcttatctttcataatttccaagaaacacaaaccttttctacta

SEQ ID NO: 74 - DNA, 1000 bp, Arabidopsis thaliana
  misc_feature (1)..(1000): Promoter Construct PT0708 with Candidate Id 15371629 as reported in Report Number # 235
gtttccaaaactagtattctttatttgctctattcattatatttttatatttgtaacgtcccgaccgtctttattaggtttcgacaatcacttctcggaaggtcgtccatcctgaaattactctatcctaaacatgtttaactataaaattctctcgaaacttttgtaacgtatataaccacataaattctcttaaacttatttgcatacaccattatatttctgaaatcgatatgttacaatattatttaatatttagattacttttactgaatcgaattaaatatcaaatcgaaacaaatctaatctaccaaaaataattttgttataaacatttcttgcctagttctacctcatatacattttagttaaagaaagaaatcacaacaattcccataattcaataattaaatccacaaaatcttggagtaagtaagagaaataaaaagatagtatcttaacataaacaattcaaagatgctctctcacacaattcacacacacttacaaaacaaaagacagaaacaatgttttcattcaaatcaaaagaagttataacactagtacaaaaaaagctcaaattctaatagtaactctttttatttcccaattacccaaagattctctctcacttcacaaaactagctttgagagtcgtgttccacaaaatccattaaagctgaaacggttttgctcaccattcaaacaaatacaaaattgcaaaaccccaaattataacaaaataatataaaaattaaaccgctaaaaagagtgaaccaacaaaaatcgccgaatgtgtgtgtaatgagaaaaccgacccatcatcccaatcatctcttcccgtgtcactctcttcctctcccacgtttcttctctcttccctttatgggttttaacttctccttcttcttcttcttcaatcttcagttttcaaattcaacaacaattcacattttgatttcttcatcatctctctctctctcgcttctctctcaaatcg

SEQ ID NO: 75 - DNA, 1000 bp, Arabidopsis thaliana
  misc_feature (1)..(1000): Promoter Construct PT0758 with Candidate Id 15371866 as reported in Report Number # 243
agctagccacatcagtgaccaaaaaagataattaacaaaccaaataaaataacaaattttgatcatttggaataaaatttataaaaggaacgaaagcgccttctcacgggtcccatccattgaaatatattctctctttttgctctatataataataacgcgtactaatttgtagtatatattattacaaagtcgatatttgattgttttgtgaacgttgatatattaattttcttggatgatgacaaaaaaagtcatagaaagtaacgtgtgaacatagcattaacaaaatacaaacataatatataaccaaatatatgaaaataggataaaatctcattgaatagatcttcttctattcaaatatataaatatttgtttgtctataaaattaacagagcattcacattatctaaaataatagtaaaatcaaaataaaactaaataaaaataactctggttttataacgattgattttaaatattagtttttgttgtaaagagatcattatatatgtctgtaatatttttatactgagttacatgatatttagttattatagcgtaattaactaagataagaaattaactaaagtgatattctgattattattatttttgttaggacacgtacgtggaaaaactaaacactataggttacaaaacggtataataaactcaccattactggaaaatgtttgcatttgactcaataagtaacttattataagttactgatataatgcatagttttgaaattcttaaataaattattttggtttcgcatgaaaatatgaaaggagagaaatttattattgtcacttatatatatatacatcgtaatcattttttcgtgaataattctctctcccattccattatttctcagtatctctctttctttcccttactttattgttgcttttaaaccttcaatttgctcataaaccaaatatataatatcaaaacaaacaaacaaaaaatcagaattcccctaata

SEQ ID NO: 76 - DNA, 763 bp, Arabidopsis thaliana
  misc_feature (1)..(763): Promoter Construct PT0837 with Candidate Id 15371899 as reported in Report Number # 250
aactacaagggagacataatatcaccatctggttcctgttatcatctgaagatttcttgttttaccttccagtgataaaatgatccttataatacatatagatatattaaattgctgtattttaagattatagatatataaggtacatgagagtgtttatttaaaaaaattcacttggaattcatgttttgtgatacgttagattggaatccatttgggaaaagaagaatcatctgttcttatgtctcaaattttgacttcattcacttttcttcttgtcttttaagaaagcttccacaatctaactgttcgatgtgaaaactgagattcgagtaagaaaatgtgaactgtgttatactgttttttaattagataatttagattgcactcagataaattaataacattcctcgaatacttttatgtgattggatatattaggtatatctgccaaccaaccaataaactgctatgtttaaacaaattaaataaattagtatatgtttactcaagaataaagaagatagaaaagaaaattctatatgagctaaatttgctggaggaggcatcggacgtgggtaccagacctttccaagcacacgagtagtgcttagccatgtcatgctaacatacaccatttggttcatacaaaatccaaatcaaaatctatttttaaaatcttttgcacacgtctttgaaaaacacctctcatactatagctacggaagcttcaatttcaaggtttgtctaaaagctaacgatt

SEQ ID NO: 77 - DNA, 1823 bp, Arabidopsis thaliana
  misc_feature (1)..(1823): Ceres Promoter 21876
gtctcttaaaaaggatgaacaaacacgaaactggtggattatacaaatgtcgccttatacatatatcggttattggccaaaagagctattttaccttatggataatggtgctactatggttggagttggaggtgtagttcaggcttcaccttctggtttaagccctccaatgggtaatggtaaatttccggcaaaaggtcctttgagatcagccatgttttccaatgttgaggtcttatattccaagtatgagaaaggtaaaataaatgcgtttcctatagtggagttgctagatagtagtagatgttatgggctacgaattggtaagagagttcgattttggactagtccactcggatactttttcaattatggtggtcctggaggaatctcttgtggagtttgatatttgcgagtataatctttgaacttgtgtagattgtacccaaaaccgaaaacatatcctatataaatttcattatgagagtaaaattgtttgttttatgtatcatttctcaactgtgattgagttgactattgaaaacatatcttagataagtttcgttatgagagttaatgatgattgatgacatacacactcctttatgatggtgattcaacgttttggagaaaatttatttataatctctcataaattctccgttattagttgaataaaatcttaaatgtctcctttaaccatagcaaaccaacttaaaaatttagattttaaagttaagatggatattgtgattcaacgattaattatcgtaatgcatattgattatgtaaaataaaatctaactaccggaatttattcaataactccattgtgtgactgcatttaaatatatgttttatgtcccattaattaggctgtaatttcgatttatcaatttatatactagtattaatttaattccatagatttatcaaagccaactcatgacggctagggttttccgtcaccttttcgatcatcaagagagtttttttataaaaaaatttatacaattatacaatttcttaaccaaacaacacataattataagctatttaacatttcaaattgaaaaaaaaaatgtatgagaattttgtggatccatttttgtaattctttgttgggtaaattcacaaccaaaaaaatagaaaggcccaaaacgcgtaagggcaaattagtaaaagtagaaccacaaagagaaagcgaaaaccctagacacctcgtagctataagtaccctcgagtcgaccaggattagggtgcgctctcatatttctcacattttcgtagccgcaagactcctttcagattcttacttgcaggttagatattttctctctttagtgtctccgatcttcatcttcttatgattattgtagctgtttagggtttagattcttagttttagctctatattgactgtgattatcgcttattctttgctgttgttatactgcttttgattctctagctttagatccgtttactcgtcgatcaatattgttcctattgagtctgatgtataatcctctgattaattgatagcgtttagttttgatatcgtcttcgcatgttttttatcatgtcgatctgtatctgctctggttatagttgattctgatgtatttggttggtgatgttccttagatttgatatacctgttgtctcgtggtttgatatgatagctcaactggtgatatgtggttttgtttcagtggatctgtgtttgattatattgttgacgttttggttgttgtatggttgatggttgatgtatttttgttgattctgatgtttcgatttttgtttttgttttgacagct

SEQ ID NO: 78 - DNA, 1000 bp, Arabidopsis thaliana
  misc_feature (1)..(1000): Ceres Promoter PT0668
atagagttttactatgcttttggaatctttcttctaatgtgccaactacagagaaatacatgtattaccactaggaatcggaccatatcatagatatcaggattagataactagttctcgtcgctatcacttcgcattaagttctagtaattgttaaagattctaattttttactaaacaaaaactaaatcaacatcaaatatgcaaagtgtgtgttgtccacacaagtgactcaaagtatacgcaggtgggattggaccatattattgcaaatcgtttccgaaccactcatatttctttttttctctcctttttttatccggagaattatggaaccacttcatttcaacttcaaaactaattttttggttcagtgatcaaatacaaaaaaaaaaaaaaagttatagatattaaatagaaaactattccaatcttaaaaatacaaatgaaaccataattttaatttatacaaaactatttaattagctaagggttgtcttaacgtttagaaaataaaaaattatgattgtctgtttaaaattacaatgaatgaataaaaaaaatatgcaatgaatgaaagaataaattttgtacatccgatagaatgagaaaatgaattttgtacaaaccactcaagaattcaaaacaattgtcaaagttttcttctcagccgtgtgtcctcctctcctagccgccacatctcacacactaatgctaaccacgcgatgtaaccgtaagcgctgagtttttgcatttcagatttcacttccaccaaacaaaactcgccacgtcatcaatacgaatcattccgtataaacgtctagattctttacagcctacaatgttctcttctttggtcggccattatttaacgctttgaacctaaatctagcccagccaacgaagaagacgaagcaaatccaaaccaaagttctccattttcgtagcttctttaagctttttcagtatcatagagacacttttttttttttgattagaa

SEQ ID NO: 79 - DNA, 1000 bp, Arabidopsis thaliana
  misc_feature (1)..(1000): Ceres Promoter PT0535
ttagtgaaattatgacattaagtaaggttttcttagttagctaatgtatggctattcaattgttatgttaggctattttagttagtatatgaatttaggcagtctatgcaaatgatttcgttttcattttttcatatgtaaacatcaagatcaagtaacgccattcgagttgatattttttttttaaattagtgtgtgtaaattttggaccgcttatttgagtttgctaatgaagttgcatatatattacgttaaaccataggcaaactaatttgaaacatccgattcgatttcctgtaatttttcttggttaattgaccaaaatcaagatcttcagaaataaaataaaagacgaaagaaagctgtcgcaaagcagattgtgttaaaaaaaagtggattgggctcaaacgcaacttgtccagcccgtgacaattaccctatacgcaagtaagagtaacgtatcactggcaaaagttggtattagttacgatatctttgtcatgggggcatgcatgggcatggcttaagagttaagccttaagaagagtcccacactcgtgactctcatgatcacttgttgtttcttacgggcaaatacatttaactttattcttcatttattcacctatattcttttggataataacttttctctatataaaataacaaacatcgtacgtttcatttatttacaacaagcgatgagaattaaaaggagaccttaattgatgatactcttcttttctctcggttacaacgggattattacagataatgataatctatatggatgctgacgtggaaaaacaaaatttggtgaaacacgtcaattaagcacgacttttccatggctagtggctaagatcgtttcatcacatggctatatcatataatacttggatgaattcaaaataaacgactgagaaaatgtccacgtcacggcgcaccgctttggacttaagtctcctataataaatacaacaccaaacattgcattcca

SEQ ID NO: 80 - DNA, 999 bp, Arabidopsis thaliana
  misc_feature (1)..(999): Ceres Promoter PT0585
tgaagtcatttaatatgagtttgacattaggtaaacctaatctatgagattatagaatgtagcaaaactatcaatgtttcttttccaaaatattttgtggtttttctttttggttcattatgttttgttatttgtgaattattttaatatgaagtaattatattgattttatatgatatacatattattttgatataaaatttaacacttatccattaaaatagcatgggcataatcaaaatcgggactattacgatgaaaaagatagttaaattgtatgataaaataaaatgtgtaagattaaaattttgggttttagaaaattactaaacaaaatatagacaaagtatgttgactattatttaaaatttaaatatcatcaataagatatagttaaagtcattaagtgtatagcaaaatgaaaattctaagattaaaattcgattaaaattttttttactaaattaaatatttaaaaatagggattatcatttactatttacaattctaatatcatgggtaaaaattgataactttttttaaacccgcctatctaggtgggcctaacctagtttactaattactatatgattaacttattaccacttttacttcttcttttttggtcaaattactttattgttttttataaagtcaaattactctttgcattgtaaataatagtagtaactaaaatcttaaaacaaaatattcaacctttcccattattggaatggtaatgtcttcaacaccattgaccaacgttaaggaatgtcttttaatatttttggaacctaaatgctaatactgtataccacaatcacttatgagtattgaagttgagatagaggaggtacaaggagaccttatctgcagaagacaaaaagccatttttagcaaaactaaagaaagaaaaaagattgaaacacaaatatgcgccactcgtagtccacccctatctctttggcaaaagccacttcactctttttccctttttat

SEQ ID NO: 81 - DNA, 1022 bp, Arabidopsis thaliana
  misc_feature (1)..(1022): Ceres Promoter PT0633
cccgatcggccttaatctgagtcctaaaaactgttatacttaacagttaacgcatgatttgatggaggagccatagatgcaattcaatcaaactgaaatttctgcaagaatctcaaacacggagatctcaaagtttgaaagaaaatttatttcttcgactcaaaacaaacttacgaaatttaggtagaacttatatacattatattgtaattttttgtaacaaaatgtttttattattattatagaattttactggttaaattaaaaatgaatagaaaaggtgaattaagaggagagaggaggtaaacattttcttctattttttcatattttcaggataaattattgtaaaagtttacaagatttccatttgactagtgtaaatgaggaatattctctagtaagatcattatttcatctacttcttttatcttctaccagtagaggaataaacaatatttagctcctttgtaaatacaaattaattttcgttcttgacatcattcaattttaattttacgtataaaataaaagatcatacctattagaacgattaaggagaaatacaattcgaatgagaaggatgtgccgtttgttataataaacagccacacgacgtaaacgtaaaatgaccacatgatgggccaatagacatggaccgactactaataatagtaagttacattttaggatggaataaatatcataccgacatcagtttgaaagaaaagggaaaaaaagaaaaaataaataaaagatatactaccgacatgagttccaaaaagcaaaaaaaaagatcaagccgacacagacacgcgtagagagcaaaatgactttgacgtcacaccacgaaaacagacgcttcatacgtgtccctttatctctctcagtctctctataaacttagtgagaccctcctctgttttactcacaaatatgcaaactagaaaacaatcatcaggaataaagggtttgattacttctattggaaagaaaaaaatctttggaaaaggcctgcaggg

SEQ ID NO: 82 - DNA, 1000 bp, Arabidopsis thaliana
  misc_feature (1)..(1000): Ceres Promoter PT0650
catacttaattctaaaaaaacaacacttatagtttataagcagctcttatgataaaaatctttctgagttatagctctgttaaacttgtattcaccccaaaaacggatgtttcatttcttattttttacttggagtattttattgtaatttgtaaaaaaaaatgtaaagtgggggatatcatgaaaaacaacgtcactttgtttggtcacaatatacatttgataaaataatggtcgtcgcgtgatttagttgatttttgttttatcaaccacgtgtttcacttgatgagtagtttatatagttaacatgattcggccacttcagatttgggtttgcccacatatgacataccgacatagaaggttaaatccacgtgggaaatgccaatattcaatgtttggttttcaaaagagaatcatttctttatatgatctcaaaagtatggaattgaaatgactaatgagcacatgcaattggtgctatcttaaaaaccgaacgtctttgaatttaatttgtttttcaccaaaggtacctaatgaaaccctttcattaaaaaataaaggtaacaaacaaaattttgtattggaaaaaacattttttggaatatataatttggtaatagaattatgagcaaaaaagaaaaagaaaagaaagaataatgagcataataaagcctttacagtattactaattgggccgagcagttttgggctcttgatcatgtctagtaatcttaaacagacgataaagttaactgcaatttagttggttcaggtgagctaccaaatccaaaaatacgcagattaggttcaccgtaccggaacaaaccggatttatcaaaatccttaagttatacgaaatcacgcttttccttcgatttctccgctcttctccactcttcttctctgttctatcgcagacatttttgtttatatgcatacataataataatacactcttgtcaggatttttgattctctctttggttttctcggaaaa

SEQ ID NO: 83 - DNA, 1000 bp, Arabidopsis thaliana
  misc_feature (1)..(1000): Ceres Promoter PT0665
aaaaaggatgggtaatgggacctattttccccaacatcccacatgcacacttccctctccattctctcacatttatttctttcattctaatttatccattccgtgtgtaacatattcactaataatctcatctcactaactcattcattgattgtgatatgtttatctagaattagtgttttaacactgtgtctacatatgatttccttttcattgtatgtgaacatgttaactcactaatcattttgtattttcgagttaacatgagtctccacttcggtagactaaagtaaagataggtttgagtataataaagtttaaaatttgctttaaaatcaatatttataaataagtttttatcataagtgatttttgtatgttatattggaccttgtataaacagactacagaagaaaattatttatgagaacttgtaatgttagagtggacctcgtataaactaattatgtgggcttttaccataaactatttatgaaaattattatggcccacaccactataactaaagcccacatatttagcagcccagtttcattgtaagagacatgttcgctctggaactagaattttctggtttttgggtatttgttttcttatgtgtagagaaatgatggtaacgattaaatgttgtgtattacaatttacaatggtaagacgattaatatatttacacacaattttgttgttgctgtaacacgttagtgtgtgtgatgatagaatttcataaagctttaactacgaggggcaaaatgttaattctaaatagttgacagcagaaaaagatatgtatacataatataaggattaaaacgtaaataataataaataaggcgagttaaattaaaaccctgttaaaaccctagcttgaaacacatgtataaaaacacttgcgagcgcagcttcatcgccatcgccattctctctctcatcaaaagcttttctccttgattttcgcattctttagagtcttaacgcaaag

SEQ ID NO: 84 - DNA, 1000 bp, Arabidopsis thaliana
  misc_feature (1)..(1000): Ceres Promoter PT0695
aacattttctttaacttactcttaaattttaatagtaagttgatgcatgttatgttgatccgtcttgatcacaaatattgttttatggacgaattctttgacagtaaatggctatagtgactcagcttggagcatcccgatatgaaaacaaagtgcagtattgtgtcgtggtcatcactaacgcactttcctagaactatcgcgcgtgtttgacctatgcaacacaccagatgtcatgaacgtatacttaaatagaaacaatgatatagacaattggctatattctgtcatggaacgcaaaccggataacatgtctattagattcatcggacttgatcatggttatgtcttaatagacgaattctttgttaacgattggttaaaacggctcacgttagagcatcctactatgacttcaaaattgataaatattacatggaaatcactttaattttagttagaaggtagttaatttagatattcttatttaataaattaaaaaatagaagaaaaaaagatgagaagagtttttgtttataaaataagaaatatcttttattgtaattttaaaattaaacaaatttaatttatattaaaattatctttgttttattgttaaggcaataattatttttttggtgggaattgttaaaacaataattagtatactgttaagtggtcctttaataataagataacgtgatttaaaaaagaacgagacaggctaatatagtagagaggaaaaaatacaatttaggcccaataaagcccaatatagagttgtgctcaaacacaggtcttcgccagatttcctatgacgccgtgtgtcaatcatgacgccaagtgtcattcaagaccgtcacgtggcgttgtttctacacataggcgatccatacaaatcagtaacaaacacgaaaagagcattcatatgtacgaaagtagaaaagaagagactctttgtgataaaactaagtaagaaatagcataaaagtaaaaggga

SEQ ID NO: 85 - DNA, 1000 bp, Arabidopsis thaliana
  misc_feature (1)..(1000): Ceres Promoter PT0710
tagtgcgcgtggggagagggaatggtgaaaccttagtggttaagttatgaggaaaatgataaaaggataaaacaatcaaatgcagcttgaaacggccataacataaagtaccttatggtggtgcgaatatttttgtgtttctttcactcttttattgctgaaagctacgacacttgtcttaatatattgtttccgcaagtcacatgatctactttttatttaacgtctagaaacgccgagatatatgatgattagtatatcacgtctatgcaaattgttagttcgtgtttggccaaaagatatcgagacatgtctgaagaaccgagtctggttttgagatatttcttcaagcattactatacaatagaaaaaggagacacgcgaatatgataatagcaaaaggcataaaaaggcgaaaattaaagaaaaacgtaaagtgatttggcctcaatcaacgggaacgtatcttaattttagaggttcttcttttacttttgagacgagagagtttgcgtctttgcgagctgctttggttgactaaacattatcatattgaaaaccaaaatacaacggaggaatatttgtcacagtttcactttcacattgtttccttaacgtttaatcaaccttgttcaaaatttctatagttgtaatcatcattgtttacaaaattttcgttcaaagatgattttaaataaaattgtgaaagaaaaccttttctgaaataaggattggatgatagtgttaaaagaaaaatatgaactgaggcaaaaagaggagtggtccccggaagattgtgaaatgtgtcatctaaaccagccagacgtagtcacgtgttctctctagctttatgaacttccttagccagcaccatcattgtgattgtagtatatatgtaaccctaccttcatctctcccattttccattctccatatagactcctttacaatatacaaaacctatccaaaagcgaagaagccaagcaaacatattataaaa

SEQ ID NO: 86 - DNA, 1002 bp, Arabidopsis thaliana
  misc_feature (1)..(1002): Ceres Promoter PT0723
gtcatatcttatcaacacgtcaacgatcaaaacctttagcctattaaattcaacggcttagatcaaaacgaaactaggtgggtcccacttttaatatcgtggctgcataacatttcctcgataactgaagccgttgtggtctttctcagaatctggtgcttaaacactctggtgagttctagtacttctgctatgatcgatctcattaccatttcttaaatttctctccctaaatattccgagttcttgatttttgataacttcaggttttctctttttgataaatctggtctttccatttttttttttttgtggttaatttagtttcctatgttcttcgattgtattatgcatgatctgtgtttggattctgttagattatgttattggtgaatatgtatgtgtttttgcatgtctggttttggtcttaaaaatgttcaaatctgatgatttgattgaagcttttttagtgttggtttgattcttctcaaaactactgttaatttactatcatgttttccaactttgattcatgatgacacttttgttctgctttgttataaaattttggttggtttgattttgtaattatagtgtaattttgttaggaatgaacatgttttaatactctgttttrcgatttgtcacacattcgaattattaatcgataatttaactgaaaattcatggttctagatcttgttgtcatcagattatttgtttcgataattcatcaaatatgtagtccttttgctgatttgcgactgtttcattttttctcaaaattgttttttgttaagtttatctaacagttatcgttgtcaaaagtctctttcattttgcaaaatcttctttttttttttgtttgtaactttgttttttaagctacacatttagtctgtaaaatagcatcgaggaacagttgtcttagtagacttgcatgttcttgtaacttctatttgtttcagtttgttgatgactgctttgattttgtaggtcaaa

SEQ ID NO: 87 - DNA, 1001 bp, Arabidopsis thaliana
  misc_feature (1)..(1001): Ceres Promoter PT0740
tgtggccactaaagatttacccttaaccgggcccatataagcccacgtcaagtggcgcttatacgctctccgtaagagagccaacatttggtatgtaatgttgcaaattattcttcaagacaataaattcaaatataattcaatattgtccaaatatagtgatgtacttcagttgtgcacatagaaactccactaaaccaacttttagatagatgcattcacaaattttcaacaatgtcgcgaaagtctaatccatcaccagattctaacattttaattattatatttaactatacatactctaatcagcatgagtcaaacgtgtacaatagcccaagcatataataagaccaaagtcaaactcaaataaatgtctccaaactcaaaacttgaaaaagacctaattattacatggtagatatgactttgtcgacaagtaaaccaactaatcctcgaagctaccttctcttcccagttattatgtgtgatcgatttataaatctcttcttctaataacacctatatttttcttatgatgtgaataaatataaaacttttaactttaaaacatatttatccgaaatattgcacttagatttcaaatagataaataatagtactatctaactgatattgaaaagacctaacacggaaaacagttttataaaaaatcccaaatgtgggtaattatcttgatttcttgggggaaacagaaaatggattaagattaatcggagtcgtgtcaagcagctcgttaataactgtagcaagttgactgagtaagcatcaacgtgtcatctccgtaaagcccattatttctagtctcgccgcgtcttctcttccacgtagcacttcactttttctctccttttgtttcctttggaacacaaacgtttctatttataggaataattacgtcgtccgtatctgtgtcggaacatagatccaaattaaaagcgacttacttaattacatatcgttcgtgtttttttcttcaaaaa

SEQ ID NO: 88 - DNA, 1024 bp, Arabidopsis thaliana
  misc_feature (1)..(1024): Ceres Promoter PT0743
tcgattggcccgatcggccccaaaatcaagctgagccgcttcaaacttcagcttttgaaatcacccccaaactcatgtcctcttatcattataactaaaggatctttcattttatttaactcatcgtcttgcactacccaacccaaaggttccaactatacccgaagctttctaaaggtccaaagactttttttttcgagccagactattcaagccaagaaaagccaaaccccacaagccagtacttttcaattccatattataaacttatctgtcttgttttagtcccactaaaaacaacagaatttaatttaggttgagctaaaacccttgacaaaagtgtatagtcgtcgattcagtagcacactcatcactcatcagatttgatagttgacctaaagtatgactactccatttcaactaacaaatgaaaataaaagagacctaagggttagaggattgaaactatactctcaagtcttttatcactaggctactaccagctagttaacttgatggatttaagcaagaaaacgtagaatttatattcgagcagattgtttagctaaaaaagcttgggtttgaaattgccttttctcccatataagcacgtcggttcctaaataactctttctagcggagagtgtctttccaataatttaataaaaatggtgtttgtatatcaaaaaaaaaagaaaaaagaaactgatcgagatagaacgtttgcagttttataaacaatttaaaaaacaaaaaaaattaaactcaatgtattttttattaattcacaaacaataataaatcataggatcgaatatttacacggtatcaaaacctactcgccgctactatataaaaattgaagtcaaatatcaaccgcaattattaaaccagcaagacaataattcataaacttaatataaacataaataaattaatgttacacaacgatatatggtgagggttattactatcttcttcctctcaaaacacatctcctaaccttaagctttagacggcctgc

SEQ ID NO: 89 - DNA, 921 bp, Arabidopsis thaliana
  misc_feature (1)..(921): Ceres Promoter PT0829
aaagttttgaattattgggaatcaatttcgaagttttgtaattctttgggggctaataggatattttattttcttggtttcgtctattgttgtttttctatttatggttgggcttttagaactctggacaggcccatgtcatatgttttcccttctccttatatttttcatttttcattttgttaaattaatgcataatatccaaaaacaatttaaatttttgaaggaaccctttagttacggctccgaagctttcacaagtgagaatgtgagatcaaagaaggcaaatggaggattttaaaagttaaaatcatcttttatctgcaaaagttgacaatttttttgtatcaaatctaaatcatcaaactctcttaaactacaagagcataacaacctctatgtaatccatgaaataatctgcttgaaggacataacataaatcattatggctagagtgactaacttcaatcaaatcctcttaactctagctcccttacaatggtatcgtaaaacattatgcattagggattgttgtcctaggaaaataaaataaaaatccccacagaccaactaccattttaacttaaaaataagcttcgtccgcgacgaattgttttccatcctaaaaatagaatggtgtaatctgctaatggtttagttccattaacttgcaagttctattgaaagcctaaatgtcaataaagatattaaaattcggagtcaaaagacaaatgaatcaaaagcaacaagacaagtcagctccattcttcactacccatcttttacaataaatcatctctcttttcacaaatttcaaactactctcattgccctttagctttgttatagagccaacactacagagagactcacacacttgtttcaataattaaatctgaatttggctcttcttataaacta

SEQ ID NO: 90 - DNA, 751 bp, Arabidopsis thaliana
  misc_feature (1)..(751): Ceres Promoter PT0838
atactggtatgcttaaggttgaagccaagatctctgtcttacccaagtaaccactttctattagaagggatcaacactaagaatatggagatttaagcctaagggctaaggcggttctcaacaatacatgatgtgaatacaatcacagacgatttactgaggtttgttgataagatcttgatcagtctctgcatcatctgttcaacaatctcaatctttgactgtttgctttcggagccataaacagaggaatcccttattccctgttataggagcaatacaccaagtattatttccatggctgaaattctcttatggaaacctaattgttccattgaagctgtaaaatcgaatctggtgaatattctcgagcaaagccgcatgctaattatgtcaattcagaagagtttgattaggagactcgaagcgagtttgatgatctttcttgatgttcaactccgattgtaagggtataattgacttttcatgtattacggctccaccacctgacactaaggcactctttgtccatctcgttggtatcatcggattcggatggtaaaaataaaaagagcagaggaaacttgttactcatgcaagcttctcaggtgccacgtcactccattacgtgtcatcttcacacaccatctcgctcaaaaccgatctcatttttcaaaccttaaaggcagaagcaactgattaagttaacactcttgagaagctctcgattaagcttgaacttggaggatca

SEQ ID NO: 91 - DNA, 669 bp, Arabidopsis thaliana
  misc_feature (1)..(669): Ceres Promoter PT0848
tctctttaaatcagttaactaaccgtttatatatttacgataaggtttgaagagattattgataaaataatacatttcataatcccgcgttcaaccgtttaaagtaacatttaagttgactatatctaattttttttccattaaatatggagctggtaaactttatcaacttctaaaaagtgtaacaacaaaaattaggtcaatcacaattctgttttttttattattttggattgacttccaattgcaaatagtcttagtgatcaccattatcatacatatatacatcaagtaggtttcatcatgatataccacaaagtatttgacaagccatatggttttggatcaaaaagtcggtccaaaattaatgttttatgtgcaagaaccgacccattgtacacacgtgttaacatcttcaagactttcatctctatttttcttttggtcattaagatacccattgatccgaatctgttacattcccacctacttttttaatttttactatccactccaaattaaacacaaccgatgattttaataattggaagcttttaaaaatatttcaaaacaagcctctttgtgtttgtctatatatatacacgtaataagaaggtgaatgaatctcacagcttacttgttctaaggcttccaataacgaaaacagta

SEQ ID NO: 92 - DNA, 702 bp, Arabidopsis thaliana
  misc_feature (1)..(702): Ceres Promoter PT0863
cgggaaacgacaatctgatctctagtccagtcgattggcccgatcggccgattataaacttacatgagacaagtataaataattattataaacttattaagtttaagatcaaggcttttgtgcaatgtatcaatgaatgttagatgtgatatgatgaaagcaatgttttaaacacatacatagtcattgatcggaatgtgtgttattagaaatgcatgcctaagccgatagggttatctatgtttggtcttggacattatagccaaatttcgaatctaattcttccaatatatatttttttttttttgcttagggccactactagtattgcttatcaattttaagagctcatgaaaatgcaacaatatagtagttgcaaatccttgtttcaagagaaatcaaagggccacttgtgaattgaataataataatatttgcaaataacctttcactaaaccataccaacaaaaccacacagatttggcaaagacataacctttgggagacgtgaaaaggctcaaaatttgacaattgtccttacaaattcgctcattagtgcaattgtgagatttgtttgcatccaaatccaattcataactcacactcgtctcaaattcgaaaaggcctgcagggccagtgcactgggatccaacaatgtcctccgactcgtccaagatcaagaggaagcggaaccgcaccgcg

SEQ ID NO: 93 - DNA, 435 bp, Arabidopsis thaliana
  misc_feature (1)..(435): Ceres Promoter PT0879
ttctaggaagactggtcaagctaagctgtttctgttttttgtttttgtactttactttttgtttgctagtgggaactgggtttattgggccttgaagttgataaaagatgaataaaagacatatcgcctaaagcccatatgagaagcagaagacaaaaacctccaactttgggcataaattttgattatagttaaaagtccagacccaatttggcacctggcttagttacgattctaaggcatgacacctgcctaatatgtttattacagaaaataaagagaatcagctaggtgtcccttattgaacacattaacaaactccaacgacactacgtgtcttcgtgactcttactatatccaaaaacctatagctaaagctgaattttccatgattagtatagtcccaaccaaaaaaatactgaagaaggcataagc

SEQ ID NO: 94 - DNA, 397 bp, Arabidopsis thaliana
  misc_feature (1)..(397): Ceres Promoter PT0886
agtgtatttgaaaacgacattgaagaattaatatatttttttttaattttagttttttatagtacaaatattaaaacaaacaatcctaccatatcataacatttgtaaataacattttaagttttgttttgagttttaattaattttctatgacaaaaaaatgaagtcaatagactaagtgaatcatatagtataaataaacacaatttaaatagtttcaaataaatttagaaagaataaaacaaatagaaatcagaaggtgtctgtttcctcctcgcaacatacgatcaaagagaaacaacttgaccctttacattgctcaagagctcatctcttccctctacaaaaatggccgcacgtctccaaccttctcccaactccttcttccgccatcatc

SEQ ID NO: 95 - DNA, 1024 bp, Arabidopsis thaliana
  misc_feature (1)..(1024): Ceres Promoter YP0007
agcagaacaactatatttattgtgtcacataaatctgagatcatttataaccaccaaagaacctatacacagtaaatgacaaatgtatctccctctatctctattgcccatatgtagatgctaaagtaagatttctcttttttttaatgtacttttttttgtataaagtatattccataagaaaaaggaaaagcttgtttatggatcaattgaccccaaaaaaagtttttagatcaaagcccaatataaaaaaaaaacacagtagtgacacaaaggaacttaaataaaccatgaattgatctataaacagtagagatcgataaggcgaacattttccatgtgaagtgtcttctttcatctataatatttttgacatccaataatttcctctataatatcattcacataattgatagaaacattatgttagaattgtccacatcatttgagctgtaatatattctgttttaacaaattatatggtagttgcttaatcttatgtccatcttcttctatgcatcgttttcgcgcctagttgtccagtccatttcaactacctacctctaattcttatcttaaaacaacattttttaatttaagtattatgctcaaagactaactagatagaaaaccgttattaaacattaaacgaattaaaagtcttacatggaaaatgtaggtttataaaccacgagttatgattgacaataaaaaaaatgcaaatcatcaatcaaaagagacttgagtgcgactctatatcaaccattgcaattaaaattatctatcacaaaaattttagacagattaagttaatttagtctaaattcactaatttattttctataattagtaattaactatatttatttatttacacattttctgataatttagaaatttgcatgaataacaaatataagattttggaaattagtagcaaatttaattaataattatttttgcctaaatgaaccaaactataaaacctccacatacaccagtcatcaaatttacagagacaaca

SEQ ID NO: 96 - DNA, 1000 bp, Arabidopsis thaliana
  misc_feature (1)..(1000): Ceres Promoter YP0008
ctcgagagatgaagtcttagtaatgtttgaacaaacaataatcacgttttccatcaaattcgagcatttaaagtttatattactacatgccccaagatgataccgtccatctcatccgaaaatatttctgaaattgcgctaagacaacaatgtttgctcaaattcgatcatttaaagtttacaaatctctcatcaatcttacaaacttctcacactaaacagaggtacatattttcttataaagacaaaaggttcgaacagctggcttctcaactcgagttgtttgtcagggcctctcttcactaactacaagttggtacttcaaatattggtggctagcttcacgtgatattgtctacaaattaaacccatgaaaaagctgcattaattgttccaagtgaaccctgaggagtgtcaatagtctttgctttagtgtgatcattaaaccaaatctctaaattcctaatttgtactaacatttggaacgtatttcctactcttctccctgctccaactcccaaaaataagattagttagatttctataactaatatacatgtatactcccaaaaacagtaaaaccatattaataaagctaattttgcatagatttatttcggtaaaccggcggttcaagttggggaaaaaaaagacaaacggtctaaagtcatccaaagacaaaaaaccaaagacaagttgagagagacgagaccaatcacaacattgcttcgtagattgcgtgacatcatccttgacggctactttcatttgtgtcttatttggataaaacgcacgtgtttaattcacgaaccttcatagcaataagaaatttccattactttcatattttcaactttttttattacccattacatgcttaaaatattaattcacaagtctttgtcaaaattcaatattttccaggttcatgaaccctttttatctcaatctactctataatatctccctataaattacaacaaaacctctttatttttca

SEQ ID NO: 97 - DNA, 999 bp, Arabidopsis thaliana
  misc_feature (1)..(999): Ceres Promoter YP0019
gatataagtagaatcattttttgccgccgtttctcgctaacacaccgaaaactgaatcaaatctcctagctcttctacgcaaaatcgagtgcatcgacaatggcggaacgtggtgtcgaacgtggtggagatcgcggcgatttcggacgtggattcggtggtcgcggcggtggaagaggtggtccgagaggtcgtggtcgccgtgcaggtcgtgctccagaggaggagaaatgggtgccagtgactaagcttggtcgtctcgtaaaggaaggtaagatcacaaagattgagcagatctacctccattctctcccagtcaaggagtaccagatcatagatttactcgtcggtccttcattgaaagacgaagtgatgaaaatcatgccggttcaaaaacaaaccagagccggtcagagaacgagattcaaggccttcatcgtcgtcggagatagtaacggtcacgtcggattaggagtcaaatgctccaaggaagttgcgacggcgatcagaggcgcgatcattctcgcgaaattgtctgtggttccgatacgaagaggttattggggtaacaagattggaaaaccacatacggttccgtgtaaggtaaccgggaaatgtggatctgttactgtacgtatggttccagctccgagaggttctggtattgtggcggctagagttcctaagaaggttcttcaattcgctggaattgatgatgtctttacttcttctagaggatccaccaaaactcttggaaacttcgtcaaggtatgtactttcacaatggctgttttggtttgatgaactctgaattaggcagtgaaaaagtaatcattaccagttaagtgaatttatattgaagattaggatttagctgattgtattggtttgagcatgtgagtttgtgtttaagattgcttgaattgaaatgctttaggttgtttgattacgctaaattctgactaatgtaattcaaattgttgttgtttttttttggtc

SEQ ID NO: 98 - DNA, 1024 bp, Arabidopsis thaliana
  misc_feature (1)..(1024): Ceres Promoter YP0028
gtcagtgaagtcgattggtagtacttgaaacacttggttggtttcatgtatttggcctatatataaacaaacatcgtaattatatacggatttttttcggaattttacgccatatctgtaagtatatataacatgcatgtcgttttcaaattcatatgatgaacgatccacgtaagtgctactactcctacaatattgcatgagagagatatgtatttataaattttattttgaagaagaaataagagggaaggttacttgggtggatcgatgtgaaaacaaaagaagaaaaagcgaaacccactaagccattacatgatatcgaccttcttatctttttcctctttattttatttttctcatcttctttttgtcaggacttttttctacttaatgaaacctccaaactatctaactaatacactcccatgtagaataaagaaaattatataagatattgttgatattttgtaactagaaaatatatttgctctgtaatttttcgtaagttaaatcaacattttaaagtagaaacaaatattactgcaaaaagtaggatcattatttttgtccaaaatctcagttagctatagggttgtagtaaaaacaaaacacattcttgatttgccccaaaaaataaagagagagaagaatattgttcaaaagtggtctcttctctctctaattatgttttcactaaacccaattagattcaaacagtctacaaagtccaaaagataaacatgggacaacaattcgatgcaaaaaatcctcttttcatgctctttttttattctctagtcttttaaattactaataaaaactcacaaatccaccaaacccattctctacaactcaccttcatctagatttacccactcccaccgagaaacacaagaaaaaaaatatacatatataaatatacaagacaacacatgatgctgatgcaatatacacaacaaagtattaaatcttagatattgtgggtctccctttcttctattcattttcttattcattaaaa

SEQ ID NO: 99 - DNA, 1024 bp, Arabidopsis thaliana
  misc_feature (1)..(1024): Ceres Promoter YP0039
ccgttcgagtatttgaaaatttcgggtacacccgcctaaataggcggaccttatctagtatatatatacatttgaactatattgtttactttttagttgatttaggctatgtcatgacattgacataaatctacctgttatttatcacgtgtaattcgtgtaaagtgtaaactagaaagttcaaatacgtatttgtttttgttctgttatataggattgtcatagttgtaaatctacaatttattacaacatgaataagtacacaagcaatgtaattggatttaattgctaaactctttacatggtcaatctaaatttgataagaaatacgtcacatattactaagactgatagtttttttgttgtcaccaattatttttgttaaattgacgaaaacaattccaaaaactcaaatgtacaaaatcatacagtctcacaaacatctcatagagaaagatataaatctcccatatgggaacgataacacgaggtcgaaatactattcgtaaaactaaaacgccttagttataaatcgttagttgtaaccgcggtcgagaatacatacagatccacgaaactactactacacatgctgctgaattggaatttggaaaagaccatcttctttaggaagagctcacccaatgagtgacaaaggtgtcggtggcttgttttctacccatatgtatacatcaaatggtagtttcattaacgtttggttttgagaaaagtaagactttggctagtagctaggttcgtatataataaactcttttgagaaagttcatcactggtggaaaatgttaaaccggttttttctcattttttccgccatgttaaccaccggtttaaaaagaccgtaacacattgaaagattaataagggtatatttgtaattacggtttgctggcaatttttaattattattttaattagagaaaatagagaagccctatcaatgtacatggtatatatataaaaggcaaaaccctagaaaacgatactattcgactcagccgtcctt

SEQ ID NO: 100 - DNA, 1024 bp, Arabidopsis thaliana
  misc_feature (1)..(1024): Ceres Promoter YP0050
aatctgatctctagtccagtcgattggtacttgagggaaacatcatatttttaaaccttgtctcagtaagctaacacacaccccttgtgattacttatccatgtttatccacaagaatgcagttggattgagatattttcttctttgttgaaatcaggcctcaaggtgttcatgtggtctgcaaaaaaattcccaaaaataaagatagtgacatctgaaatcgataatggattagacgaagagtttcgtgttattccttggtatgggcgggtttggggacagatattttggcacagacgaggactaggccactgtggtcctgcagcattaggtgtcccttccatgtcctgcattacattttattgatggattcatcaccctatctactacaacggctacacaaactatgaagagttttgtttactaataaatgcccaagtgaggggtcgatcgaacccgggacacgtttttcagtttaccatatagaattatccttggaacccttgatactccatagaacatcaccacctctgttgtcatctcaggaatccaggttcaaacctagtctctctctccctagtgggaggtatatggccactgggccaatgatgacaaaatgcaaaaaaaataaaatacatttgggttcattatctaaaatatctcttgtgtttgtaagttttggttgcacactcgtgtggttgaagtgtgtgtgagaggtactatacaatacactctgcttttgttttgtacctatctctttctcttctccacatatccaagactttggggataaagctgagatcattggttgccatttggttgtgtagaagcaatcacccatttgctttatccgaggttgataaatttcctcgggttctccttctgacacgtatgacaaattctaatagtatattcctcgtagatattacctatatattctcaatagttgcaggtacttaaggctttgtcttggcatcctcgtcctcttcagcaaaactcgtctctcttgcactccaaaaagcaa

SEQ ID NO: 101 - DNA, 999 bp, Arabidopsis thaliana
  misc_feature (1)..(999): Ceres Promoter YP0086
cttatcctttaacaatgaacaggtttttagaggtagcttgatgattcctgcacatgtgatcttggcttcaggcttaattttccaggtaaagcattatgagatactcttatatctcttacatacttttgagataatgcacaagaacttcataactatatgctttagtttctgcatttgacactgccaaattcattaatctctaatatctttgttgttgatctttggtagacatgggtactagaaaaagcaaactacaccaaggtaaaatacttttgtacaaacataaactcgttatcacggaacatcaatggagtgtatatctaacggagtgtagaaacatttgattattgcaggaagctatctcaggatattatcggtttatatggaatctcttctacgcagagtatctgttattccccttcctctagctttcaatttcatggtgaggatatgcagttttctttgtatatcattcttcttcttctttgtagcttggagtcaaaatcggttccttcatgtacatacatcaaggatatgtccttctgaatttttatatcttgcaataaaaatgcttgtaccaattgaaacaccagctttttgagttctatgatcactgacttggttctaaccaaaaaaaaaaaaatgtttaatttacatatctaaaagtaggtttagggaaacctaaacagtaaaatatttgtatattattcgaatttcactcatcataaaaacttaaattgcaccataaaattttgttttactattaatgatgtaatttgtgtaacttaagataaaaataatattccgtaagttaaccggctaaaaccacgtataaaccagggaacctgttaaaccggttctttactggataaagaaatgaaagcccatgtagacagctccattagagcccaaaccctaaatttctcatctatataaaaggagtgacattagggtttttgttcgtcctcttaaagcttctcgttttctctgccgtctctc

SEQ ID NO: 102 - DNA, 1024 bp, Arabidopsis thaliana
  misc_feature (1)..(1024): Ceres Promoter YP0088
tcgattgggattactacttcatctagtaaggttctgaaaacgtttgttgttgataaggaagattcgtctcaggttattactgttgatcttcaaggtttgtgattgtgacgcttatacatgtgctgaaactgtggtgtttatttattgaaaacaaaaaaaaagtctctcttgtagtttcattgtactaaatagaaaacaagaaacgtttttttctttaatcttctacattgataatattggatcaaaggattgtttctgcaagacacaacacaaacatacttatactagtttacttctactaagtactaactacatacccatacacacacttgcacctagactttacttctagacatcattaccctaaggtagaaccaagcttacaagcaagttttaccgacaactcttacattacaactctagtctgtagtctttaacgtagacttactaactagtcattagtggtttaattttttaaattttcatccatatgtttttgttgtagatataaactaaagtcggtcacatttaataattgtcattatgtccgcgtaaaagtcaattcagctattggacatttatgaaatgtaagattttctctctcatttccccgtgcgtgaagacatgcattggtttttctgtaataatcaacaaatccaaaccccttttcgatctttatttggacattgttagagacaaaatttctctatagtctttttcctaatttgataccatgtttttgtttctgcacaaatttactcactggtttaactaactatccacttatttatgattttaccattaggcgtcagctagccctagtcaaatttgtaaacaagccaagctatctacataaatcgagatgtcattaacgttaatcgtcgttaattcgaatttgaaaacatagatagctttagcagtacaatgggcaatggtaagaagaatagcaaaaggcccaatatttggtttgcagaaattaaagccttaaaaaaaagcccacagatatttgtcaaagaaccctaat

SEQ ID NO: 103 - DNA, 1020 bp, Arabidopsis thaliana
  misc_feature (1)..(1020): Ceres Promoter YP0096
gaggtcagtgagtcgattggtgcaaaattgaaaaattgaagggtgaaacaaatttaaagataatatctattaaatcctctaattttaaaaatttagcaaaaattgtattttcttatggatctgttagttcacacgtatcttaattagtaccaaatcatatctaatgattagtgataaaactagttagatatctatatgtgtctttaccatttaacttgaatccttcttcttttttttacgtaaacaacttgaatccttcgttaatacataaatttaaagcattttttctttaattctattgatcggtatatatttactataagttttagctcatatgcaatttcaaatgatatgcttttaaattttgtctaggtgtgatagttgtatctttaacataaatcttatagcaaaattatacttgatattctaaatttatctatttgctcttgtgaacctcatattagtctagagaaactttgaaatcctttcaattagttgtatgtccaatacatttttactaacatttattagtctttttaattaagattattgttagaaaaaaaaagattttttaaaaataaataatatgttttagatacaatgtgagttaggcttcttatattttaaaaaataaatttatttcatacttaaaaatagtttggaatttcaatttatttggctgaataccataaaatatgtcaatttgaaccttatacccattgactatttggtgttagaaaccctttaacaaaaaaaaactatttggtgttagatatcaaaataaaaaaagtttaaccattggtttcttatattgaattggatattgttacatgtattaaagtttttttggtttaattttgaaacgttgatagaaactattaagtttaagtttggtagtatatttatttgtggaaaatttaattgccattaaatataacgtcaactttttttggttttttttgagaagttacgttgtgattttgatttcctatataaaagttagattacgtcattttttaa

SEQ ID NO: 104 - DNA, 1000 bp, Arabidopsis thaliana
  misc_feature (1)..(1000): Ceres Promoter YP0097
ttcatctttatatttaagagtttaaaaactgcaacttttgtttttctttcactaagtcttatggccacagttaattaaaagcagatgaaaggtggtccaatggaaaaggagaatgtgattgggctagttgggagagttctgatgtctagtgttgggtacacgtgtccgtcagttacacatagcattaaatcagacggcatgtcattattcaaatctagttcacatagtacgactaatagctgataaattaatgattatacagcatatgaattatgaattcaaaaaaaaaaaaaaattgaaaatgttaaggagatgctatattttacaaaattcatcgcaatgctttctactaatttgctaagtggtcttctccagttagtcttgtcgattccaagcgatattattaaatcttgaagcatcgctcaaagcattatagcttaagataaccaaattgttattaaaaacacctagtgaaatttttaaattaaaacaattttgatatctttgtaatatctaatactactctttctgtgtctaaaaggattaattttcaaaaatttcacacatattaaaaaaaaaaaaaaattactagctaaacaattttcaataatcataaaacaatagtaacttaataatttttttttattttcaaaatagtccttcaagtttacaattcattttagtattataatcaacaaaatttgtattaaaaagttggaaaattaatctttgtggaacaaaaaaatctagaaatcattttttagaattagagagaggtttgataaaaaaaaataaaaaaaaatagagagaggtagtacatactaaacgatgtgatactactattgacaaaatcttaattctcagtttagtagaataaactagaaggaatgaatgaagtaaatgcgaatccaactactaacaaaccctacttagtcatcatattttcccatatgaaatccctatataaacccatcatcatctcccacttttttcatatcca

SEQ ID NO: 105 - DNA, 1004 bp, Arabidopsis thaliana
  misc_feature (1)..(1004): Ceres Promoter YP0101
ttctcgttctctagaatattgctggaccggattaggtcaatattattgggccagattagatattgaattgtcgacgttgcttacgttacgttatatcttgtttaagaattaaacctatcgacttagtcttaattaagaaaacattgccttaaattctctggtctgcgaccgtttttttgaccgttaacccctaattaaagaaacaaaataattatagaaagagcactgaaatgtgattattttaacagtactcttatgagaaaattcgtactttttagttttttttttgtacaaatctctaagaaaaacactactactaattaagaaacgtttcaaacaattttattttcgttggctcataatctttctttctcggtccgggactaaccgttggcaaaaaaaaaaaaaaagttgacaataattattaaagcgtaaatcatacctctcaaataaaaacttgaatttggaaacaaagacaactaaaaaactcgaatttaagagaattcctaaaatcaagtgaagtatcatcacttggtaaaatttcataaccgttggcttctatttctatgtgtgccttggtttgcaggagataatatttcatttccaaccaatgatattcgtacacatagtcaaacaaatgtttgtctttgttattatattgagaaagaaacaagaaagagagagagagatagataagacgaaggaagtgaagcttccaagcgcccaccgttaaaaatctcgtgtgcaagtttcaaatacaagtggccggtggtctccataatttgatcgtcatccaattaaaaaggaagaaaaagcgtgttttatacaagaaaactcattaaaatagcaagtctagaaatatctcaacactaatctaccacgtctattacacacacacacacacacacttgatcttaatttattttcaagattcaagaaaatacccattccattaccacaacttgaccacacgcctatatataaaacataaaagccctttcccc

SEQ ID NO: 106 - DNA, 1000 bp, Arabidopsis thaliana
  misc_feature (1)..(1000): Ceres Promoter YP0102
atttggttgataacgttttcactcgactaattatatacttcagaaggatagtaatagaataccaaaataattaaatgattggttagtgccttagtggagactttttaaccgattctaatagactaatgatgtagctaagcatttatttgggatcatcactgtttgaaaacgtgaaatgtgataaaagttatgaaacgattaaaatataaaataaccgtacaaaacattatgtaccgtttttttctctgttcttttggcgatttggtttagttcgttacactctaaatgttattgcagatatatatataatgatgcatttgcatctgaggaacatataattccggttaacacttccaaatcttatatccgtctaggtagggattttataaatcatttgtgtcatcatgcgttatgcttgtcggctttgaccataacgcagagatatagaactagcttttacttaacttttagatttattatttgatctagagttaagtggagatatatagtgtttttgttagattattggtggatgtgagagtttgtctttagtttcaagttgagaatataaggcaagaggagactctgaggcaatcagaggttttgattggcaaaatatccaaaaggcccaaaccaagtcgaagcccatctcgtacaaaaaaagaaagagatctgtaagaaaaaatattctttgatattcttacaaaaataagtgtaaaacttttattagtcaaaatcttcaatctttaaaaactctcatcactcctacgaaagcgcgtgagagttatgagacattccttaatagcattactcacaagtcacaagttcaaaacgtctgactgaaacagaaacaagcctttgttgaagtcttgaagaagagacattagtactcgtcgtatagccataaaaggtaatatacgaaatttcttcgctaatctcttcaccttcctctacgcgtttcactttcactttataaatccaaatctcccttcgaaaacat

SEQ ID NO: 107 - DNA, 1004 bp, Arabidopsis thaliana
  misc_feature (1)..(1004): Ceres Promoter YP0103
gttttgaagaacaatctggatcgaaatctaacataaggtcatcgtattcaagttacgcagtcaaggacttgacatcatcctactctggtctgaggttaccacttccaaagatgggatttttcgactcggtatgcttcctaagaaattcgttttattgaacctagcaaatatcttgtaatgtaagattcctgagatgatgaagaaaaaacaaacttttgttacagcaggagaacggagagaaagaaaacagagaaccaaatgctcttgaagcaaacagaagaagaagacacaaatccaaacttgagacttcttctacaccagaaaaccgcagcattctgggacaacgcaaaacacgaaagtgaaacgggcaatgatatatatgtcttgggtgcgttacaaggcatcgtttgcaactgttgagttggataagtcaactgtcttcttttcctttggttgtagtagctgccttttttttcctttgttgctttaagaaatagcccgaaaaaaagaatgttctacatttcggagcagaaaactaaccgaatgagtttttggtcggatcatcggatcgatcagatatattttgagttacgaactgttataaaaaaagccataattttgtgttgagtttgcaaaataccttataacttgttatttgagattgcacctccatatatattaattcgtaagagtatttattaagtaagctttagtataaatccttttttcctttaaagtaagttaatgttctactaaataatagtaaagttgaagaaccgctccgtttttacaccatgcacgtgttatctaacaaagaaaatatggtacacctaatggctaatgcaaaggacaacacaatgaaactaacttgactctgtgttatagaaacccatagacatctgcatacatcctagtatttgtataaattggactcaaattcctgaggacaatcatagcaaacaatcacatcatcgcaatatacataaacaaaagaggaagaaaaa

SEQ ID NO: 108 - DNA, 1003 bp, Arabidopsis thaliana
  misc_feature (1)..(1003): Ceres Promoter YP0107
taacaatccttgggaacattgcatccatagatatccggttaagatcgatctttgaactcataaaaactagtagattggttggttggtttccatgtaccagaaggcttaccctattagttgaaagttgaaactttgttccctactcaattcctagttgtgtaaatgtatgtatatgtaatgtgtataaaacgtagtacttaaatgactaggagtggttcttgagaccgatgagagatgggagcagaactaaagatgatgacataattaagaacgaatttgaaaggctcttaggtttgaatcctattcgagaatgtttttgtcaaagatagtggcgattttgaaccaaagaaaacatttaaaaaatcagtatccggttacgttcatgcaaatagaaagtggtctaggatctgattgtaattttagacttaaagagtctcttaagattcaatcctggctgtgtacaaaactacaaataatctattttagactatttgggccttaactaaacttccactccattatttactgaggttagagaatagacttgcgaataaacacattccccgagaaatactcatgatcccataattagtcggagggtatgccaatcagatctaagaacacacattccctcaaattttaatgcacatgtaatcatagtttagcacaattcaaaaataatgtagtattaaagacagaaatttgtagacttttttttggcgttaaaagaagactaagtttatacgtacattttattttaagtggaaaaccgaaattttccatcgaaatatatgaatttagtatatatatttctgcaatgtactattttgctattttggcaactttcagtggactactactttattacaatgtgtatggatgcatgagtttgagtatacacatgtctaaatgcatgctttgtaaaacgtaacggaccacaaaagaggatccatacaaatacatctcatagcttcctccattattttccgacacaaacagagca

SEQ ID NO: 109 - DNA, 1024 bp, Arabidopsis thaliana
  misc_feature (1)..(1024): Ceres Promoter YP0110
gggatgcggttccgcttcctcttgatcttggacgagtcggaggacattgttggatcccagtgcaatggtaatataaaacaagaaaacaagagattttataggacaatcactaaatgacatttaattgattaaacatttattcattaataattgtatgttactaacttcaacatttaataattttgtttaagatacgtttacatcagagactattaatatttttacaggttgtaactttaaactttgtcttgaatcgaacatgactatagattttgggcaaacttaaagataacaacatttccgttttttttcaaattattacaaatcaaactgatatattagacacaacacgattacacgtaatgaaaaaagaaaaagataaaaagataaaagaagggatcgattctgtttggtctggtttagtgagattcaaagttaagctcttcctttcaagacatgccttcttaaaccgggaatgtgaacgtttgtaatgtagtccgtccagttaatgcttccaacatcaaatccaaattctctcttctcgtcctctgacatattctccattaatctctggggtattgctgttatcaaatctgtaaaagaaaccaaaaaaaaaagatgaaaactttgcgggtaccggttttgtctgctctaagaattagaatgttaatgagttctgtcttaccttccaccatagaaagtgtatggctcataaatagtagcaaggtgtttggcttgttcaacagatttcttgcatataaactttagcttctgcatcatcttactatccactgaactcataccactcatcaacccactccgttcttgagcatctctccacaaatgatccgagaaatcatcaacggaattgaaaagtttcatcaaacgcaccataataggatcacctttagagtccatgcatggagatgttttgtagtggttataaagaagctccgctaagtcttcgaaaaccagcgggtttatcgccgaagaagcgatctgatacacgtttatttcaggttcc

SEQ ID NO: 110 - DNA, 1024 bp, Arabidopsis thaliana
  misc_feature (1)..(1024): Ceres Promoter YP0111
cgattggatttagtctatacattatagggcgcaagtttgtggatttaagaattatataaaaacttgaaatatatagtttttatgcattctcctcttgtgtaatacataaaccaaatatgagataggttaatctgtatttcagataatattaaattccaaacaatatttttacttgttataagaaggcaattaatatctctctgttaatggcaagtggtaccaagtagtattaaactattaatgcaatggaagagtactgttggaaattataatcctctatcacacattcaaacagatctcctgaaatcttctcttccaaacttgtacttctctgatccaaatgtaggctccaaaatatagacatttaccatttactaagtccacaactcctttcttgtctccttcaaaaatgactcttgtgtaaccaccatatgactccgacagttcggcattgccatgatgagagcttaaaaattcaccttcctgagcatttcaagtcttcactcccttagcttgacctgaaccaagataaaatgcctttgtcgtcccgtaatatccatcctgctttggacggcatcatagttacattcgatccatcctatttacaatgttattttagtattaaaaacatgacaataaatttgttgttaaacatattcaaatacaatatgattggatttataagtaattgtaatatgaaatgtccttagtaatatgttaaaaaatacatagatacacacacgtactaaaagaggcaacgcgggagatgtcattagaggaagaactaggaagcagagcgttcatgcaaaatgctaccaaaaacgttaatgcaatatctcaactaatcagcacagtccatttcatactgagaatgtaaaaaccaatcagcatcgtccattttttcatctaattatttgttaactcttaattggccacaacttccaaccacatgacgctctttctattccctttatatattcccatctcaaatgttcttggagacacaaaatatcataaacatata

SEQ ID NO: 111 - DNA, 996 bp, Arabidopsis thaliana
  misc_feature (1)..(996): Ceres Promoter YP0115
gtcgattggatgatgaacattctacatatataattattatgtttaagcacttagacagcataaattctttctaattatataaatctaaccttgttacattgtacatctataaattacttgaagaaataacgagttctatttctttttaaaaattaaaaatactataccatatctcagtgattaagttgaaccaaaaggtacggaggagaaacaagcatttgattcttccttattttattttattcatctctcactaatgatggtggagaaaaaaagaaaatacctaacaaacaaatatatattgtcatacaaaaatatttctatatttttagttaattagtttatattcctcacttttcagggcttatataagaaagtgagcaaacacaaatcaaaatgcagcagcaaatactatcatcacccatctccttagttctattttataattcctcttctttttgttcatagctttgtaattatagtcttatttctctttaaggctcaataagaggaggtactattactacacttctctctacttttacttgtattttagcattaaaatcctaaaatccgttttaaattcaaaaataaacttagagatgtttaatctcgattcggtttttcggctttaggagaataattatatgaaattagtatggatatctttactagtttccattcaaatgattctgatttcaatctaatactctcactctttaattaaactatatgtagtgtaatttcacactgttaaatttctaccatgtcatgtatattagagttgcatagaaaattgtaaaacatccatttgaattcgaatgaaacaaaatgttttaaaataaaattttggtttttaaaagaaaaatctaaaactgaattatatcgtttaaccaagttgtaaaagtcataaaacgtagtatcttgtaaatcgctcttccacggtccaaatagacttctagtaataaacaagtaaaactaattttggtttcttac

SEQ ID NO: 112 - DNA, 1024 bp, Arabidopsis thaliana
  misc_feature (1)..(1024): Ceres Promoter YP0117
gtcagtgagtcgattggatcacagtcctttatgataaaacaaactcataattattccaccgacaacatgcgttttaaattattttttcttaaattatattatattatattgatatcaacctagctaaaataattcggatggcgaaatcggacaatttttaatagaaaaaatgggtatgaagatagtctatgattccgttcttagcgactagagggacctgctcaaatctcccgggtgatacgcgatgtcaagctcaatagaaccccacaaccgacgagaccgagaaatccttgatttgggctagaagattttgaaataaatttaatatattctaagtaacttgcttaaattttttttcaaactctaaagacataactaacataaagtaaaaaaaaaaaagttaatacatgggaagaaaaaaattaaactaatgattagctctctaacgtgtttaatctcgtatcaagtttttttttaaaaattatattgctattaaaacattgtactattgtttctattttgtttagctattattcttgtgaaatgaaaagttgtgtttattcaattactaaatggcaatatttatcttggaaaactatacctctaattggattaggccctagacatcctctttagcttattgacgttaaaattattcccaaaactattaaagtttagtagtttgaaagatgcatcaagacctactcagataggtaaaagtagaaaactacagttagtgtgattatattttaaaatatataaaacaatcttattaaactaaatattcaagatatatactcaaatggaagataaaaacatttagtctgttaccactaccagcctagctagtcactaatagtcactttggaactgagtagatatttgcatcttgagttaccatggactcaaaagtccaaaaagagaccccgagtgaaaatgctaccaacttaataacaaagaagcatttacagcggtcaaaaagtatctataaatgtttacacaacagtagtcataagcaccattg

SEQ ID NO: 113 - DNA, 999 bp, Arabidopsis thaliana
  misc_feature (1)..(999): Ceres Promoter YP0120
tagtttttgatttaatctacgtttttcttaatcataaatgggtaattattagtttttgcaaaatcaaaatccaaaaattgttctaaacactgcaaccatttaaggcctatatcactcagaaaatttctggtgggagaactaatcgtttgtcctttctaaatctcacatattagaatttagaattagtgtgctacataagaatattagttcagctcggaacaactattttttggtaaaacagagaacttaaacaaatgcattattttatcaacatgcattttgaattgaatataaaatttcataattgtaaagacataaattacataaaattttacatgaaaaaatagatatagaaagaaaatgaaactaactgatgatatgctctctaaattttttaatctcataacaagaattcaaattaattagttcatatttttggttaatataacatttacctgtctaagttggaactttcatttttttctgttttgtttagtcagtattcttaatgtgaaacggaaagttgaatttattcaaacttaaattcaatagcattaattaaaggcgaaagctattatctctacatgtggttcaaactagacatccaatttaattagcttattgacgttgaaatgttttccaaaactactatagtttggcaatttgaaagatgcatcagaactactcagacaggtaaaagtagaacctctagctgtgtgaattgtatgttagtccataaagaacatcttgtaaacttcatacttaagatatatattacaatatatacttgaatggtagataaaaacgattagtctgattgctagcatactcacaactatttggaaatgagtaagatattggcattctagagttactactatggagacaaaagtcgaataaaagagacctcacgtgaaaatgttacgagctagtaaaaaaagcatttacactaacggtaaaaaaagtatctataaatgtttacacaaggtagtagtcatt

SEQ ID NO: 114 - DNA, 999 bp, Arabidopsis thaliana
  misc_feature (1)..(999): Ceres Promoter YP0121
ttggattttttttttgttgagtcagcagaccatctaatctctctttttccaccacagcctgctttctatgaagcatttgggcttacggttgtggaatcaatgacttgtgcactcccaacgtttgctacctgtcatggtggacccgcagagattatcgaaaacggagtttctgggttccacattgacccatatcatccagaccaggttgcagctaccttggtcagcttctttgagacctgtaacaccaatccaaatcattgggttaaaatctctgaaggagggctcaagcgaatctatgaaaggttggcccattctccttgacaggcttaacaatacaacttgtatcgcttcaacaagatgatggcttaataaggatttttgcatgtataggtacacatggaagaagtactcagagagactgcttaccctggctggagtctatgcattctggaaacatgtgtctaagctcgaaaggagagaaacacgacgttacctagagatgttttactcattgaaatttcgtgatttggttagtgtaacccactgttattcttttgatgtctacatctactttacttacattattcttttcttcggtttgcaggccaattcaatcccgctggcaacagatgagaactgatcatgacagggtaggattttatttcctgcactttctttagatcttttgtttgtgttatcttgaataaaaattgttgggttttgtttccttcagtggtttgattttggacttatttgtgttaatgttgttttggctgttctcttaatatcaataacaaataaatttactggttggtatctaagatctaacaatagttactatttttagaggtaaagacaccaaccttgttatattggtcagagagctaaaaccttgacttgttgggaaaacaaaactctaatgacagaaaatctgacatgatgccttataattcacagcctcatgttctacataaatcctaacaatagcactttgtttct

SEQ ID NO: 115 - DNA, 1001 bp, Arabidopsis thaliana
  misc_feature (1)..(1001): Ceres Promoter YP0137
gtggcacatgctgaaaccccgagcatctctccggaagacacgcgtcgttcgctccaaagaaaacagtcacagctgccggagaatctccgccgtcttcttctgccaccggaaaaactctctccaccactttcagtgcccacctcgtgttatatccactgtatcctcgtagcaccatatcagcctaataaaattttatgtatcaaattttaagacatagccgaaactacactatactagacaataataatatgatttgtttcctgaaaaattatggtttcatgagaaacattaatcatctataaaacaaattagctatggcatcgaagagttatcaatcaaaactgatgaatctttacttaatatatacaacatatctttaccttgcggcggagaagatcggcgagagaagcaccccagccaccgtcactaaaggattcttcagtgatggaatcaccaaagagaaaaaccttccgtctcatcatcttccacacaatcttcttgagaaaatctgagagataagaaaggtgtagtggttttgctgaagtgatcgtgtttgatttagtaaagaaatgctttatttattgttgggggaaacataaataaataaagtaaaagtggatgcactaaatgctttcacccactaatcaccgacctttcatggtttattgtgaaatacactcatagatagacatacaataccttatgtacgtaaataacattttatttgtcgacacttatgtaagtaacgcatagattattttctatgtgattgccactctcagactctcagtttcaaccaataataacaataactacaacaacattaatcataaacatatgctctggtttacaattaaagcttagattaagaaactgtaacaacgttacagaaaaaaaatgttatttacgttttgtaagattagtctctagaatcatcaccgttttttatatattaatgattctttcttatatataaaacctttctcgaaatacccatgaaa

SEQ ID NO: 116 - DNA, 1001 bp, Arabidopsis thaliana
  misc_feature (1)..(1001): Ceres Promoter YP0143
atacaacagatggcagatatcgagttaaatacgtgaatcagccgttacgatattttaaaactagaaaattatttaaaaatattgcaaaataccatttaatttcattgttcataaaaaaaagaaattcaaaaacttaaaaactgattcaaaaatttggattaattctcattaacagtcttcaacactacaacaacatgtttctaatttattttatattttaataattaaacaatatatacgtctgcacattgttgctccgacataatctagtataaaaatagttgcagcatatgtgaaaagcaagcagcatttatcactcaatacttttaattttatctgttgtatgtattaaggttttgtagctttaagaaaacgcttataatataaaataacttctaaaagatatttcatgcgtatacaataaatatttgtgaaaaaacatttcgaaaacgtgtacaatatataaactattgtgttatcttttgacattcaaacaaatgttgacaatgtaattttatccatgatatgattggccaattagctgcgaggtaaaaatccgtatacgagtaaaagtaagataaaatttcgcaagaagatttttagcaggaaatctaagacaagtgtcatgaacgtgtcaatcaacaaacgaaaaggagaattatagaatccagattcgacgtaccacattaataaatatcaaaacattttatgttattttatttttgctctggcagttacactctttttcattgctccaataaaaaaatcactcgcatgcatgcatatatatacaccatagtaaactccgcctcttcttcattttaaaagtatcagtttacactgacacaatccttaactattttcctttgttcttcttcatctttattacacatttttttcaaggtaacaaataatctttttaagtcacttttatactctttaaatcttagattgatatatgaatgcatgttaatatttcaagatttataggtctaccaaac

SEQ ID NO: 117 - DNA, 1003 bp, Arabidopsis thaliana
  misc_feature (1)..(1003): Ceres Promoter YP0144
aaacgttgcaagattattgattgtgagaaagagtgctcaaggtagtactgatttctgtaaagctcacggtggtgggaaacgatgttcttggggagatgggaaatgtgagaaaatttgctagaggaaagaagcggtttatgcgctgcgcataacactattatgtctcgggagaacaaagatggaagcaagagcggtttgattggaccgggactctttagtggccttgtttttggctctacttctgatcattctcagtctggagctagcgctgtctctgattgtactgattctgttgaacgaatacagtttgagaataggcagaagaacaagaagatgatgataccgatgcaggttctagtaccttcatcaatgaaatctccaagtaattcacatgaaggagaaacaaacatctatgacttcatggttccggaggagagagttcacggcggtgggctagtaatgtctttacttggtggctccattgatcgaaactgaaagccatttatggtaaaagtgtcacattctcagcaaaaacctgtgtaaagctgtaaaatgtgtgggaatctccgaatctgtttgtagccggttacgttatgctggatcaaaaactcaagatttgttggatattgttatgctggatcggtggtgaaaccacttcccggttgctaaataaataaacgtttttgttttataatctttttcactaaacggcagtatgggcctttagtgggcttcctttaagcgaccaatacaatcgtcgcaccggaatctactaccatttataggtttattcatgtaaaacctcggaaaatttgagagccacaacggtcaagagacaaaaacaacttgaagataaagggataaggaaggcttcctacatgatggacaacatttctttccacacaaattctcataataaaaatcttataatacaaatacttacgtcataatcattcaatctagtccccatgttttaaggtcctgtttcttgtctgatacaaat

SEQ ID NO: 118 - DNA, 1004 bp, Arabidopsis thaliana
  misc_feature (1)..(1004): Ceres Promoter YP0156
ttggtttgcattgtgaagatttgtattaactatagaacattgaattgatggtgttaagttcttacacaagcgtgcttctcggtttgaactgtttcttttgtatgttgaatcagagcttagtttataggaaccagagtatctacttagtcattctctgatgctaagtgctaaggttctacctagttgccctctaggcccttatgttattgataacttatgaagctatttgaacacttgattcttaggagacctaagttggtacagccagatagagtgtatgttcttgttctctatgtgacaggatcaagctgccacacatagttcaagggtatgctctgtgtgggtttgctcagattgaggacaaatctatacaaggaagtagagtctttgacattttgatgttgtatgataagaagaagaaaggagagtaataaagaaagagaaaagggaaacagaaacacgtgggagaacatcccaaagaggaagcacacgcggatcttcatgcaaagctccccgattctcccatgtggtccctttctccctttgtccccctcctctttcttcttttctcattttactcctttttttaccattatacaacgaatcttttttatcataattttttggttttggtttattttccaataacactttcttggttacttcccattctcactttttcatataagaaactcactttgggaaacttatgtttgagaatgacaagtctttttagagaaagtgatgtaacaaatctaaagtgattatataataaccttgcacaatgtttttgattttttgtaagattcgaatattaggtttattattcgtagggaataaacttactttcaaaagcgttcataagttaatactttcatatatgatcataagtacggacactattgttttttgtttgtttgtgtttattctaaaagaaagtagcttttaattgaaatgtcctcggaggcacagtttaaagttcgagtgtaacagtttctaaggca

SEQ ID NO: 119 - DNA, 1000 bp, Arabidopsis thaliana
  misc_feature (1)..(1000): Ceres Promoter YP0158
ttattagattaatagattgcattgcattgcttgtgctttcaatttacaaattgtctcccaactccatcgacacatctctttttgtgtatataagattcagacttgttatattttttttataaatatgttattagcatcttaagttaaattgattttttatatctgcattaaggattacacgactatatttgcgattgtgtgttggttaaaatataatttaggattgtctttaactacatttaggattatatgactatatttggttaaatataaaatctagctgtgattattagtattcaaaaataagtagcctaaccaattaaaacaacggctattggggcaaattagaacattttagtgtgtccaaaatataatggtcattaggtcatattcctcctagcttcatcgcagcataattgaatgattgccttatttagaagagcttttccactttcccaaaatctaggtgggatctttttgttttgaccttcatttttcttgtttaccatttttagctaaattatttacgattacaaaagatatcaaaagttggatcataatacaatttatagacttactgtagaaaattcgtatgtacaagtacaacaaattcttcataataaattttgaaaattctattacaaatgttgtaagaaatagaatttgaaatatatataaactaaggagaaaaaaaaagagaacatgcattgctctagtcagagtggaccaacatcaacgagataagataacataaaaaccaactcaccataactaaaaacatcccaagagatccaacgattcatatcaaacacaaaaacatcgaacgatcagatttaaaccatctctggtatctccaaaacacaaacacttttttttttcttttgtctgaatggaacaaaagcatgcgacatctctgtgtctttatcttctctctcctcttcttgaaaaactgaacctttaattctttcttcacatctcctttagctttctgaagctgcta

SEQ ID NO: 120 - DNA, 1005 bp, Arabidopsis thaliana
  misc_feature (1)..(1005): Ceres Promoter YP0188
gattggtatgaaatttcggagaccaacaaaaaaaactttattgagcttggagtgaagctatatatatggggcaagatcataatatgtttatatcggccttttcgttaactgaaaataatagttttgagaaatatatcaaatggtaaacagacatcatctttgaaaaataccatcaatgaagttaatattgttattggcatatggtttacccatcttaattttaatgcaaccaaacaaacaagaaacaaaaactgtataagatacaaggtgttttacgattttccgtcttaaaaccgaaatatttttgttcctacgactttaaacggactttgcttaagttgtgtgcatgtaagctcgtcgtccctcgattgtcatcaacattcaccaatatcagcctctatcacacgagtgaaggtggtgattcggcttaatgaaaacagagaaatatttcaatatgattcctattaaattttaaatcttttttctcaatctctagattttcattaaaagcatcatgatttttttccactatgttcatatatctctatcacagttttaggtacattgtagaaattggataagatacgtcatacgtctaacatgaatttggtctagcaaggaaggtttgagataataagtgaaaagaaaacacaagataataaattataatttataaatgctttatagtattgaaaaataagatgattttttttttttttaataccggattggctgatccacttatgatgactcaaatgttattaagtttcaagacaatttatgatgacacaaatcacaatgagtcaatagtagccacgaagccagaaaaaaaaaatgtactacaaaaagataatgatagtacaaaatgatacgtcgtactgccacatgtacgacacaactcgattaccaaaaagcagagccatccaaccataaaactcaaaacacacagattccactggcgtgtgctctcctcacttcactcgtccttgaaacttgaggtactga

SEQ ID NO: 121 - DNA, 1002 bp, Arabidopsis thaliana
  misc_feature (1)..(1002): Ceres Promoter YP0190
taaatagtgacattggtaagaagaaaaaaaacactattaaatagtgaaaaaatggtttataactctcttaattaacattacttattattgctagcacctaaaatctcccacaaaatatttgttgtaaaacacaaatttacaaaatgattttgtttttaaattagtaacacatgttcatatatacgttaataagaacataccctatatgattttatataaaaaaatttctttgagacgtcttattcttttttctttaataatatgcaattgtgagagtttggatttgaatggtagcattagaagcaaacttgaaccaaacatatttcatgaagtcaaacttgaaccaatgtgatcactaatcacagtgttcgcagtgtaaggcatcagaaaatagaagaagggacatagctatgaatcatataatcttgacacatgttttataggttttaggtgtgtatgctaacaaaaaatgagacagctttcttctaatagacttaatatttgggctaaatgtaccacagttgtgaatttcttacaaaaatgggccgagctacaaaaaactacaggcccactctcaactcttatcaaacgacagcgttttacttttttaaaagcacacactttttgtttggtgtcggtgacggtgagtttcgtccgctcttcctttaaattgaagcaacggttttgatccgatcaaatccaacggtgctgattacacaaagcccgagacgaaaacgttgactattaagttaggttttaatctcagccgttaatctacaaatcaacggttccctgtaaaacgaatcttccttccttcttcacttccgcgtcttctctctcaatcacctcaaaaaaatcgatttcatcaaaatattcacccgcccgaatttgactctccgatcatcgtctccgaatctagatcgacgagatcaaaaccctagaaatctaaatcggaatgagaaattgattttgatacgaattagggatctgtgtgttgaggac

SEQ ID NO: 122 - DNA, 995 bp, Arabidopsis thaliana
  misc_feature (1)..(995): Ceres Promoter YP0212
agtcgattggtacactcttaatttaattagagtaagagatcaacaaaaatatagaattttctttatatcgaagtgctacgaccttatatatatagaaaaaaaagcataggtgaatctctaaattgagattgtgctgtagtaaacatattaagtttttagtttttttaagaaatgaatctttttgttgattaattcaaactagtagtcattaagattccggagattccaatttagaaaagtcaaagattcaaagaacaagtccaggtccacatgttgaatccgattcatcatccactcatccttcatatcttcctccaccgtctccgcccaaaaaatcaataacaataaaaaatcctaaaaaaacatatttgattttgaaaaaactttatcatatattatattaattaaatagttatccgatgactcatcctatggtcagggccttgctgtctctgacgtccttaattatcattatttttaaatttgtctctctcagaaaattacgccacaatcttcctctttcccttttccgaaaacagctaatatttgtggacctaaactaaataacgtagcctctagattttatataattactaatactatatgctactacttgttattatttactccaatcatatatgataccaatcaagaatcactacataagtagaaaactttgcaatgagtccattaattaaaattaagaataaacttaaaattttatggtattttaagattccctttggattgtaatgacaagaaatcagcaaattagtcgtaactcgtaagaataaacaagatcaatttttactttctttacaaagattccgttgtaattttagaaatttttttttgtcactgtttttttatagattaatttatctgcatcaatccgattaagaagtgtacacatgggcatctatatatatctaacaggtaaaacgtgtatgtacatgcataaggttttacgtgcttctataaatatatgtggcagt

SEQ ID NO: 123 - DNA, 1024 bp, Arabidopsis thaliana
  misc_feature (1)..(1024): Ceres Promoter YP0214
ccagtcgattggcgcctcgcatgcctatcatatttaaccgtcaataatggatttggcggttttggtaggccgggtcaaccggattaaaagaaaacggtttggagtccttccttgcaattgaattttcacacattcgggttttgtgatttctctgtcataatgggcccggcacatatggttcataacccatgtgggcctatggtataatttttccaattaaaactattgttaggtcgataaaacaaaaaacaataaaaacgagtggaatacacataccaaaaagaatgtgatgaacattagtaattttattttgatggttaatgaaaaacaaaataaatgcatcttggcatcttccgttggaaagcgcaaatagggcagattttcagacagatatcactatgatggggggtgagagaaagaaaacgaggcgtacctaatgtaacactacttaattagtcgttagttataggactttttttttgtttgggcctagttataggatcataaggtaaaaatgaagaatgaatattagattagtaggagctaatgatggagttaagtatgcacgtgtaagaactgggaagtgaaacctcctgtatggtgaagaaactatacaacaaagccctttgttggtgtatacgtattaatttttattcttttatcacaagcgatacgtatcttaagacataataaatatatatcttactcataataaatatcttaagatatatatacagtatacacctgtatatatataataaataggcatatagtagaaattaatatgagttgttgttgttgcaaatatataaatcaatcaaaagatttaaaacccaccattcaatcttggtaagtaacgaaaaaaaagggaagcaagaagaaccacagaaaagggggctaacaactagacacgtagatcttcatctgcccgtccatctaacctaccacactctcatcttctttttcccgtgtcagtttgttatataagctctcactctccggtatatttccccattgcactgga

SEQ ID NO: 124 - DNA, 911 bp, Arabidopsis thaliana
  misc_feature (1)..(911): Ceres Promoter YP0263
atctagctgtggattccaccaaaattctggcagggccatgatctaaaaactgagactgcgcgtgttgttttgcagtgatttgtatttcatatttgcaccatcctacacagtccacttggtatcgtaaccaaacataaggagaacctaattacattattgttttaatttcgtcaaactggtttttaccttttagttacatagttgattcttcatttgttttagtagttatggagcacaataatgtgcaacaaagaaagatcatagtggattaatatgttgagaggtcagaaattcttggttaacaaaaaaaagttacaaggactgagattttgggtgggagaaagccatagcttttaaaacatgattgaacttaaaagtgatgttatggtttgaggggaaaaaggttgatgtcaactaagatagttgaagtaatgtcttaaactaaagtaaaccaccggtccaaccgtggtccggaagcatctctggtatgatttatcctaaaaatcaaaatagtagaaacatactttaaatatatacattgatcggacgaaaattgtaaactagtatagtttcaaaaactagttgaacaggttatgtaccttaaacatttatttcaaacttaaacactaaagaacatatatgaatagaagtttatataaattactatatatctaccataaatctcttataattatgatgtcacgatgaggaagtgttgaaacgttaaaatgccaaaatataagcatgcgacggaattttggcagaagattgtagagttgtaatctgtcgcaatcattactcgtgctagcatttttcattttcccttcatttgtggataacgcacgatataacattctacacaccaacaagattctataaaaacgcaaaggttgtctccatagaatatcgtc

SEQ ID NO: 125 - DNA, 981 bp, Arabidopsis thaliana
  misc_feature (1)..(981): Ceres Promoter YP0285
gggattatatatgatagacgattgtatttgcgggacattgagatgtttccgaaaatagtcatcaaatatcaaaccagaatttgatgtgaaaacactaattaaaacatataattgacaactagactatatcatttgttaagttgagcgttgaaagaaaatgaaagagtgtagactgtagtacgtatgagtttcccaaaagatggtgcttgaatattattgggaagagactttggttggttcggttgaatgaagatttttacctgccatgttgatagagaaaggcaaataaatgtaggggtcgatgtctaacgtaaagactggatcaaccaagagtcctcctcctcgtcttcaccaaaaaaaaagagtcctcctcgtggaaacttatttcttctccagccaagatctcatctcatctcttcactctatgaaatataaaggaatcttatggtttttctaaaaactatagtacgtctatataccaaaggaaacaatataaaatcagttaatctgataaattttgagtaaataataaagttaactttgtacttacctatatcaaactaattcacaaaataaagtaataataacaaagaatttttagtagatccacaatatacacacacactatgagaaatcataatagagaattttaatgattttgtctaactcatagcaacaagtcgctttggccgagtggttaaggcgtgtgcctgctaagtacatgggctctgcccgcgagagttcgaatctctcaggcgacgtttcttttgttttcggccataaaggaaaaagcccaattaacacgtctcgcttataagcccataaagcaaacaatgggctgtctctgtctcactcacacacgcgttttcctactttttgactatttttataaccggcgggtctgacttaattagggttttctttaataatcagacactctctcactcgtttcgtcaacattgaacacagacaaaaccgcgt

SEQ ID NO: 126 - DNA, 996 bp, Arabidopsis thaliana
  misc_feature (1)..(996): Ceres Promoter YP0286
gaaaacaatcataggttacgctattatcatcgaaaggtatgtgatgcatattcccattgaaccagatttccatatattttatttgtaaagtgataatgaatcacaagatgattcaatattaaaaatgggtaactcactttgacgtgtagtacgtggaagaatagttagctatcacgcatacatatatctatgaataagtgtgtatgacataagaaactaaaatatttacctaaagtccagttactcatactgatttcatgcatatatgtattatttatttatttttaataaagaagcgattggtgttttcatagaaatcatgatagattgataggtatttcagttccacaaatctagatctgtgtgctatacatgcatgtattaattttttccccttaaatcatttcagttgataatattgctctttgttccaactttagaaaaggtatgaaccaacctgacgattaacaagtaaacattaattaatctttatatgagataaaaccgaggatatatatgattgtgttgctgtctattgatgatgtgtcgatattatgcttgttgtaccaatgctcgagccgagcgtgatcgatgccttgacaaactatatatgtttcccgaattaattaagttttgtatcttaattagaataacatttttatacaatgtaatttctcaagcagacaagatatgtatcctatattaattactatatatgaattgccgggcacctaccaggatgtttcaaatacgagagcccattagtttccacgtaaatcacaatgacgcgacaaaatctagaatcgtgtcaaaactctatcaatacaataatatatatttcaagggcaatttcgacttctcctcaactcaatgattcaacgccatgaatctctatataaaggctacaacaccacaaaggatcatcagtcatcacaaccacattaactcttcaccactatctctcaatctctcgtttcatttcttgacgcgtgaaaa

SEQ ID NO: 127 - DNA, 1000 bp, Arabidopsis thaliana
  misc_feature (1)..(1000): Ceres Promoter YP0337
taatttttttatttttggaactaacacttattagtttaggtttccatcacctatttaattcgtaattcttatacatgcatataatagagatacatatatacaaatttatgatcatttttgcacaacatgtgatctcattcattagtatgcattatgcgaaaacctcgacgcgcaaaagacacgtaatagctaataatgttactcatttataatgattgaagcaagacgaaaacaacaacatatatatcaaattgtaaactagatatttcttaaaagtgaaaaaaaacaaagaaatataaaggacaattttgagtcagtctcttaatattaaaacatatatacataaataagcacaaacgtggttacctgtcttcatgcaatgtggactttagtttatctaatcaaaatcaaaataaaaggtgtaatagttctcgtcatttttcaaattttaaaaatcagaaccaagtgatttttgtttgagtattgatccattgtttaaacaatttaacacagtatatacgtctcttgagatgttgacatgatgataaaatacgagatcgtctcttggttttcgaattttgaactttaatagttttcttttttagggaaactttaatagttgtttatcataagattagtcacctaatggttacgttgcagtaccgaaccaattttttacccttttttctaaatgtggtcgtggcataatttccaaaagagatccaaaacccggtttgctcaactgataagccggtcggttctggtttgaaaaacaagaaataatctgaaagtgtgaaacagcaacgtgtctcggtgtttcatgagccacctgccacctcattcacgtcggtcattttgtcgtttcacggttcacgctctagacacgtgctctgtccccaccatgactttcgctgccgactcgcttcgctttgcaaactcaaacatgtgtgtatatgtaagtttcatcctaataagcatctcttaccacattaattaaaaa

SEQ ID NO: 128 - DNA, 1000 bp, Arabidopsis thaliana
  misc_feature (1)..(1000): Ceres Promoter YP0356
ttagttcattgaaacgtcaactttttacttgcaaccactttgtaggaccattaactgcaaaataagaattctctaagcttcacaaggggttcgtttggtgctataaaaacattgttttaagaactggtttactggttctataaatctataaatccaaatatgaagtatggcaataataataacatgttagcacaaaaaatactcattaaattcctacccaaaaaaaatctttatatgaaactaaaacttatatacacaataatagtgatacaaagtaggtcttgatattcaactattcgggattttctggtttcgagtaattcgtataaaaggtttaagatctattatgttcactgaaatcttaactttgttttgtttccagttttaactagtagaaattgaaatttttaaaaattgttacttacaataaaatttgaatcaatatccttaatcaaaggatcttaagactagcacaattaaaacatataacgtagaatatctgaaataactcgaaaatatctgaactaagttagtagttttaaaatataatcccggtttggaccgggcagtatgtacttcaatacttgtgggttttgacgattttggatcggattgggcgggccagccagattgatctattacaaatttcacctgtcaacgctaactccgaacttaatcaaagattttgagctaaggaaaactaatcagtgatcacccaaagaaaacattcgtgaataattgtttgctttccatggcagcaaaacaaataggacccaaataggaatgtcaaaaaaaagaaagacacgaaacgaagtagtataacgtaacacacaaaaataaactagagatattaaaaacacatgtccacacatggatacaagagcatttaaggagcagaaggcacgtagtggttagaaggtatgtgatataattaatcggcccaaatagattggtaagtagtagccgtctatatcatccatactcatcataacttcaacct

SEQ ID NO: 129 - DNA, 1000 bp, Arabidopsis thaliana
  misc_feature (1)..(1000): Ceres Promoter YP0374
aagacacccgtaaatgttgtcatgtagaagaaactagaaacgttaaacgcatcaaatcaagaaattaaattgaaggtaatttttaacgccgcctttcaaatattcttcctaggagaggctacaagacgcgtatttctttcgaattctccaaaccattaccattttgatatataataccgacatgccgttgataaagtttgtatgcaaatcgttcattgggtatgagcaaatgccatccattggttcttgtaattaaatggtccaaaaatagtttgttcccactactagttactaatttgtatcactctgcaaaataatcatgatataaacgtatgtgctatttctaattaaaactcaaaagtaatcaatgtacaatgcagagatgaccataaaagaacattaaaacactacttccactaaatctatggggtgccttggcaaggcaattgaataaggagaatgcatcaagatgatatagaaaatgctattcagtttataacattaatgttttggcggaaaattttctatatattagacctttctgtaaaaaaaaaaaaatgatgtagaaaatgctattatgtttcaaaaatttcgcactagtataatacggaacattgtagtttacactgctcattaccatgaaaaccaaggcagtatataccaacattaataaactaaatcgcgatttctagcacccccattaattaattttactattatacattctctttgcttctcgaaataataaacttctctatatcattctacataataaataagaaagaaatcgacaagatctaaatttagatctattcagctttttcgcctgagaagccaaaattgtgaatagaagaaagcagtcgtcatcttcccacgtttggacgaaataaaacataacaataataaaataataaatcaaatatataaatccctaatttgtctttattactccacaattttctatgtgtatatatatacccacctctctcttgtgtatttg

SEQ ID NO: 130 - DNA, 998 bp, Arabidopsis thaliana
  misc_feature (1)..(998): Ceres Promoter YP0377
tataaaccattcctataacaccatatttaaacataacaatgaattgcttggatttcaaactttattaaatttggattttaaattttaatttgattgaattatacccccttaattggataaattcaaatatgtcaactttttttttgtaagatttttttatggaaaaaaaaattgattattcactaaaaagatgacaggttacttataatttaatatatgtaaaccctaaaaagaagaaaatagtttctgttttcactttaggtcttattatctaaacttctttaagaaaatcgcaataaattggtttgagttctaactttaaacacattaatatttgtgtgctatttaaaaaataatttacaaaaaaaaaaacaaattgacagaaaatatcaggttttgtaataagatatttcctgataaatatttagggaatataacatatcaaaagattcaaattctgaaaatcaagaatggtagacatgtgaaagttgtcatcaatatggtccacttttctttgctctataacccaaaattgaccctgacagtcaacttgtacacgcggccaaacctttttataatcatgctatttatttccttcatttttattctatttgctatctaactgatttttcattaacatgataccagaaatgaatttagatggattaattcttttccatccacgacatctggaaacacttatctcctaattaaccttactttttttttagtttgtgtgctccttcataaaatctatattgtttaaaacaaaggtcaataaatataaatatggataagtataataaatctttattggatatttctttttttaaaaaagaaataaatcttttttggatattttcgtggcagcatcataatgagagactacgtcgaaaccgctggcaaccacttttgccgcgtttaatttctttctgaggcttatataaatagatcaaaggggaaagtgagatataatacagacaaaacaagagaaaaga

SEQ ID NO: 131 - DNA, 999 bp, Arabidopsis thaliana
  misc_feature (1)..(999): Ceres Promoter YP0380
acaagtaccattcacttttttacttttcaatgtatacaatcatcatgtgataaaaaaaaaaatgtaaccaatcaacacactgagatacggccaaaaaatggtaatacataaatgtttgtaggttttgtaatttaaatactttagttaagttatgattttattatttttgcttatcacttatacgaaatcatcaatctattggtatctcttaatcccgctttttaatttccaccgcacacgcaaatcagcaaatggttccagccacgtgcatgtgaccacatattgtggtcacagtactcgtcctttttttttcttttgtaatcaataaatttcaatcctaaaacttcacacattgagcacgtcggcaacgttagctcctaaatcataacgagcaaaaaagttcaaattagggtatatgatcaattgatcatcactacatgtctacataattaatatgtattcaaccggtcggtttgttgatactcatagttaagtatatatgtgctaattagaattaggatgaatcagttcttgcaaacaactacggtttcatataatatgggagtgttatgtacaaaatgaaagaggatggatcattctgagatgttatgggctcccagtcaatcatgttttgctcgcatatgctatcttttgagtctcttcctaaactcatagaataagcacgttggttttttccaccgtcctcctcgtgaacaaaagtacaattacattttagcaaattgaaaataaccacgtggatggaccatattatatgtgatcatattgcttgtcgtcttcgttttcttttaaatgtttacaccactacttcctgacacgtgtccctattcacatcatccttgttatatcgttttacttataaaggatcacgaacaccaaaacatcaatgtgtacgtcttttgcataagaagaaacagagagcattatcaattattaacaattacacaagacagcgagattgtaaaagagtaagagagagag

SEQ ID NO: 132 - DNA, 1000 bp, Arabidopsis thaliana
  misc_feature (1)..(1000): Ceres Promoter YP0381
cacggtcaaagtattgctaacatggtcattacattgaaaaagaaaattaattgtctttactcatgtttattctatacaaataaaaatattaaccaaccatcgcactaacaaaatagaaatcttattctaatcacttaattgttgacaattaaatcattgaaaaatacacttaaatgtcaaatattcgttttgcatacttttcaatttaaatacatttaaagttcgacaagttgcgtttactatcatagaaaactaaatctcctaccaaagcgaaatgaaactactaaagcgacaggcaggttacataacctaacaaatctccacgtgtcaattaccaagagaaaaaaagagaagataagcggaacacgtggtagcacaaaaaagataatgtgatttaaattaaaaaacaaaaacaaagacacgtgacgacctgacgctgcaacatcccaccttacaacgtaataaccactgaacataagacacgtgtacgatcttgtctttgttttctcgatgaaaaccacgtgggtgctcaaagtccttgggtcagagtcttccatgattccacgtgtcgttaatgcaccaaacaagggtactttcggtattttggcttccgcaaattagacaaaacagctttttgtttgattgatttttctcttctctttttccatctaaattctctttgggctcttaatttctttttgagtgttcgttcgagatttgtcggagattttttcggtaaatgttgaaattttgtgggatttttttttatttctttattaaacttttttttattgaatttataaaaagggaaggtcgtcattaatcgaagaaatggaatcttccaaaatttgatattttgctgttttcttgggatttgaattgctctttatcatcaagaatctgttaaaatttctaatctaaaatctaagttgagaaaaagagagatctctaatttaaccggaattaatattctccgaccgaagttattatgttgcaggct

SEQ ID NO: 133 - DNA, 999 bp, Arabidopsis thaliana
  misc_feature (1)..(999): Ceres Promoter YP0384
tttaaaaaattggataaaacaccgataaaaattcacatttgcaaattttattcagtcggaatatatatttgaaacaagttttgaaatccattggacgattaaaattcattgttgagaggataaatatggatttgttcatctgaaccatgtcgttgattagtgattgactaccatgaaaaatatgttatgaaaagtataacaacttttgataaatcacatttattaacaataaatcaagacaaaatatgtcaacaataatagtagtagaagatattaattcaaattcatccgtaacaacaaaaaatcataccacaattaagtgtacagaaaaaccttttggatatatttattgtcgcttttcaatgattttcgtgaaaaggatatatttgtgtaaaataagaaggatcttgacgggtgtaaaaacatgcacaattcttaatttagaccaatcagaagacaacacgaacacttctttattataagctattaaacaaaatcttgcctattttgcttagaataatatgaagagtgactcatcagggagtggaaaatatctcaggatttgcttttagctctaacatgtcaaactatctagatgccaacaacacaaagtgcaaattcttttaatatgaaaacaacaataatatttctaatagaaaattaaaaagggaaataaaatatttttttaaaatatacaaaagaagaaggaatccatcatcaaagttttataaaattgtaatataatacaaacttgtttgcttccttgtctctccctctgtctctctcatctctcctatcttctccatatatacttcatcttcacacccaaaactccacacaaaatatctctccctctatctgcaaattttccaaagttgcatcctttcaatttccactcctctctaatataattcacattttcccactattgctgattcatttttttttgtgaattatttcaaacccacataaaaaaatctttgtttaaatttaaaacca

SEQ ID NO: 134 - DNA, 998 bp, Arabidopsis thaliana
  misc_feature (1)..(998): Ceres Promoter YP0385
actcaacaataggacaagccaaaaaaattccaattattgtgttactctattcttctaaatttgaacactaatagactatgacatatgagtatataatgtgaagtcttaagatattttcatgtgggagatgaataggccaagttggagtctgcaaacaagaagctcttgagccacgacataagccaagttgatgaccgtaattaatgaaactaaatgtgtgtggttatatattagggacccatggccatatacacaatttttgtttctgtcgatagcatgcgtttatatatatttctaaaaaaactaacatatttactggatttgagttcgaatattgacactaatataaactacgtaccaaactacatatgtttatctatatttgattgatcgaagaattctgaactgttttagaaaatttcaatacacttaacttcatcttacaacggtaaaagaaatcaccactagacaaacaatgcctcataatgtctcgaaccctcaaactcaagagtatacattttactagattagagaatttgatatcctcaagttgccaaagaattggaagcttttgttaccaaacttagaaacagaagaagccacaaaaaaagacaaagggagttaaagattgaagtgatgcatttgtctaagtgtgaaaggtctcaagtctcaactttgaaccataataacattactcacactccctttttttttctttttttttcccaaagtaccctttttaattccctctataacccactcactccattccctctttctgtcactgattcaacacgtggccacactgatgggatccacctttcctcttacccacctcccggtttatataaacccttcacaacacttcatcgctctcaaaccaactctctcttctctcttctctcctctcttctacaagaagaaaaaaaacagagcctttacacatctcaaaatcgaacttactttaaccaccaaatactgattgaacacacttgaaa

SEQ ID NO: 135 - DNA, 1000 bp, Arabidopsis thaliana
  misc_feature (1)..(1000): Ceres Promoter YP0396
catagtaaaagtgaatttaatcatactaagtaaaataagataaaacatgttatttgaatttgaatatcgtgggatgcgtatttcggtatttgattaaaggtctggaaaccggagctcctataacccgaataaaaatgcataacatgttcttccccaacgaggcgagcgggtcagggcactagggtcattgcaggcagctcataaagtcatgatcatctaggagatcaaattgtatgtcggccttctcaaaattacctctaagaatctcaaacccaatcatagaacctctaaaaagacaaagtcgtcgctttagaatgggttcggtttttggaaccatatttcacgtcaatttaatgtttagtataatttctgaacaacagaattttggatttatttgcacgtatacaaatatctaattaataaggacgactcgtgactatccttacattaagtttcactgtcgaaataacatagtacaatacttgtcgttaatttccacgtctcaagtctataccgtcatttacggagaaagaacatctctgtttttcatccaaactactattctcactttgtctatatatttaaaattaagtaaaaaagactcaatagtccaataaaatgatgaccaaatgagaagatggttttgtgccagattttaggaaaagtgagtcaaggtttcacatctcaaatttgactgcataatcttcgccattaacaacggcattatatatgtcaagccaattttccatgttgcgtacttttctattgaggtgaaaatatgggtttgttgattaatcaaagagtttgcctaactaatataactacgactttttcagtgaccattccatgtaaactctgcttagtgtttcatttgtcaacaatattgtcgttactcattaaatcaaggaaaaatatacaattgtataattttcttatattttaaaattaattttgatgtattacccctttataaataggctatcgctacaacaccaataac

SEQ ID NO: 136 - DNA, 1514 bp, Arabidopsis thaliana
  misc_feature (1)..(1514): Ceres Promoter p13879
tttcgatcctcttcttttttaggtttcttgatttgatgatcgccgccagtagagccgtcgtcggaagtttcagagattaaaaccatcaccgtgtgagttggtagcgaattaacggaaagtctaagtcaagattttttaaaaagaaatttatgtgtgaaaagaagccgttgtgtatatttatataatttagaaaatgtttcatcattttaattaaaaaattaataatttgtagaagaaagaagcattttttatacataaatcatttaccttctttactgtgtttttcttcacttacttcatttttacttttttacaaaaaagtgaaaagtaaattacgtaattggtaacataaattcactttaaatttgcatatgttttgttttcttcggaaactatatcgaaaagcaaacggaaagaacttcacaaaaaaccctagctaactaaagacgcatgtgttcttcttattcttcatatatcctctgtttcttgtgttctgttttgagtcttacattttcaatatctgactctgattactatatctaaaagggaacatgaagaacttgagaccatgttaaactgtacaatgccttcaaacatggctaactaaagatacattagatggctttacagtgtgtaatgcttattatctttaggttttttaaatcccttgtattaagttatttaccaaattatgttcttgtactgcttattggcttggttgttgtgtgctttgtaaacaacacctttggctttatttcatcctttgtaaacctactggtctttgttcagctcctcttggaagtgagtttgtatgcctggaacgggttttaatggagtgtttatcgacaaaaaaaaaatgtagcttttgaaatcacagagagtagttttatattcaaattacatgcatgcaactaagtagcaacaaagttgatatggccgagttggtctaaggcgccagattaaggttctggtccgaaagggcgtgggttcaaatcccactgtcaacattctctttttctcaaattaatatttttctgcctcaatggttcaggcccaattatactagactactatcgcgactaaaatagggactagccgaattgatccggcccagtatcagttgtgtatcaccacgttatttcaaatttcaaactaagggataaagatgtcatttgacatatgagatatttttttgctccactgagatatttttctttgtcccaagataaaatatcttttctcgcatcgtcgtctttccatttgcgcattaaaccaaaaagtgtcacgtgatatgtccccaaccactacgaattttaactacagatttaaccatggttaaaccagaattcacgtaaaccgactctaaacctagaaaatatctaaaccttggttaatatctcagcccccttataaataacgagacttcgtctacatcgttctacacatctcactgctcactactctcactgtaatcccttagatcttcttttcaaatttcaccattgcactggatg

SEQ ID NO: 137 - DNA, 1954 bp, Arabidopsis thaliana
  misc_feature (1)..(1954): Ceres Promoter p326
gtgggtaaaagtatccttctttgtgcatttggtatttttaagcatgtaataagaaaaaccaaaatagacggctggtatttaataaaaggagactaatgtatgtatagtatatgatttgtgtggaatataataaagttgtaaaatatagatgtgaagcgagtatctatcttttgactttcaaaggtgatcgatcgtgttctttgtgatagttttggtcgtcggtctacaagtcaacaaccaccttgaagttttcgcgtctcggtttcctcttcgcatctggtatccaatagcatacatataccagtgcggaaaatggcgaagactagtgggcttgaaccataaggtttggccccaatacggattccaaacaacaagcctagcgcagtcttttgggatgcataagactaaactgtcgcagtgatagacgtaagatatatcgacttgattggaatcgtctaagctaataagtttaccttgaccgtttatagttgcgtcaacgtccttatggagattgatgcccatcaaataaacctgaaaatccatcaccatgaccaccataaactcccttgctgccgctgctttggcttgagcaaggtgtttccttgtaaagctccgatctttggataaagtgttccactttttgcaagtagctctgacccctctcagagatgtcaccggaatcttagacagaacctcctctgccaaatcacttggaagatcggacaatgtcatcatttttgcaggtaatttctccttcgttgctgctttggcttgagcacggtgcttctttgtaaagctccgatctttggataagagcggatcggaatcctctaggaggtgccagtcccttgacctattaatttatagaaggttttagtgtattttgttccaatttcttctctaacttaacaaataacaactgcctcatagtcatgggcttcaaattttatcgcttggtgtatttcgttatttgcaaggccttggcccattttgagcccaataactaaatctagccttttcagaccggacatgaacttcgcatattggcgtaactgtgcagttttacctttttcggatcagacaagatcagatttagaccacccaacaatagtcagtcatatttgacaacctaagctagccgacactactaaaaagcaaacaaaagaagaattctatgttgtcattttaccggtggcaagtggacccttctataaaagagtaaagagacagcctgtgtgtgtataatctctaattatgttcaccgacacaatcacacaaacccttctctaatcacacaacttcttcatgatttacgacattaattatcattaactctttaaattcactttacatgctcaaaaatatctaatttgcagcattaatttgagtaccgataactattattataatcgtcgtgattcgcaatcttcttcattagatgctgtcaagttgtactcgcacgcggtggtccagtgaagcaaatccaacggtttaaaaccttcttacatttctagatctaatctgaaccgtcagatatctagatctcattgtctgaacacagttagatgaaactgggaatgaatctggacgaaattacgatcttacaccaaccccctcgacgagctcgtatatataaagcttatacgctcctccttcaccttcgtactactactaccaccacatttctttagctcaaccttcattactaatctccttttaaggtatgttcacttttcttcgattcatactttctcaagattcctgcatttctgtagaatttgaaccaagtgtcgatttttgtttgagagaagtgttgatttatagatctggttattgaatctagattccaatttttaattgattcgagtttgttatgtgtgtttatactacttctcattgatcttgtttgatttctctgctctgtattaggtttctttcgtgaatcagatcggaa

SEQ ID NO: 138 - DNA, 2016 bp, Arabidopsis thaliana
  misc_feature (1)..(2016): Ceres Promoter p32449
gatcggccttcttcaggtcttctctgtagctctgttacttctatcacagttatcgggtatttgagaaaaaagagttagctaaaatgaatttctccatataatcatggtttactacaggtttacttgattcgcgttagctttatctgcatccaaagttttttccatgatgttatgtcatatgtgataccgttactatgtttataactttatacagtctggttcactggagtttctgtgattatgttgagtacatactcattcatcctttggtaactctcaagtttaggttgtttgaattgcctctgttgtgatacttattgtctattgcatcaatcttctaatgcaccaccctagactatttgaacaaagagctgtttcattcttaaacctctgtgtctccttgctaaatggtcatgctttaatgtcttcacctgtctttctcttctatagatatgtagtcttgctagatagttagttctacagctctcttttgtagtcttgttagagagttagttgagatattacctcttaaaagtatccttgaacgctttccggttatgaccaatttgttgtagctccttgtaagtagaacttactgggaccagcgagacagtttatgtgaatgttcatgcttaagtgtcgaacgtatctatctctactatagctctgtagtcttgttagacagttagttttatatctccatttttttgtagtcttgctagttgagatattacctcttctcttcaaagtatccttgaacgctcaccggttatgaaatctctacactatagctctgtagtcttgctagatagttagttctttagctctctttttgtagcctagttctttagctctccttttgtagccttgctacagagtaagatgggatattacctccttgaacgctctccggttatgaccaatttgttgtagctccttgtaagtagaacttaggatagagtgagtcaactttaagaaagaacctagtatgtggcataaccagattgcaggctctgtctcggctacagtaacgtaactctatagctctttgttttgttcagaaagaaccagtgattggatgattcgtccttagaaactggacctaacaacagtcattggctttgaaatcaagccacaacaatgcctatatgaaccgtccatttcatttatccgtttcaaaccagcccattacatttcgtcccattgataaccaaaagcggttcaatcagattatgttttaattttaccaaattctttatgaagtttaaattatactcacattaaaaggattattggataatgtaaaaattctgaacaattactgattttggaaaattaacaaatattctttgaaatagaagaaaaagcctttttccttttgacaacaacatataaaatcatactcccattaaaaagattttaatgtaaaattctgaatataagatattttttacaacaacaaccaaaaatatttatttttttccttttttacagcaacaagaaggaaaaacttttttttttgtcaagaaaaggggagattatgtaaacagataaaacagggaaaataactaaccgaactctcttaattaacatcttcaaataaggaaaattatgatccgcatatttaggaagatcaatgcattaaaacaacttgcacgtggaaagagagactatacgctccacacaagttgcactaatggtacctctcacaaaccaatcaaaatactgaataatgccaacgtgtacaaattagggttttacctcacaaccatcgaacattctcgaaacattttaaacagcctggcgccatagatctaaactctcatcgaccaatttttgaccgtccgatggaaactctagcctcaacccaaaactctatataaagaaatcttttccttcgttattgcttaccaaatacaaaccctagccgccttattcgtcttcttcgttctctagttttttcctcagtctctgttcttagatcccttgtagtttccaaatcttccgataaggcct

SEQ ID NO: 139 - DNA, 667 bp, Arabidopsis thaliana
  misc_feature (1)..(667): Ceres Promoter PD1367
acagttttcttttctcatcttacaacaagtttccaggaggatagagacataaacgaagctcggattgtatcgttctttttagcttttattcacatccgaaagtcctgtagtttagattctgttatcttgcggttttgagttaatcagaaacagagtaatcaatgtaatgttgcaggctagatctttcatctttggaaatttgtttttttctcatgcaatttctttagcttgaccatgagtgactaaaagatcaatcagtagcaatgatttgatttggctaagagacatttgtccacttggcatcttgatttggatggttacaacttgcaagacccaattggatacttgctatgacaactccaactcaagagtgtcgtgtaactaagaaccttgactaatttgtaatttcaatcccaagtcatgttactatatgtttttttgtttgtattattttctctcctacaattaagctctttgacgtacgtaatctccggaaccaactcctatatccaccatttactccacgttgtctccaattattggacgttgaaacttgacacaacgtaaacgtatctacgtggttgattgtatgtacatatgtacaaacgtacacctttctcctctttcacttcatcacttggcttgtgaattcattaattcctgcgaa

SEQ ID NO: 140 - DNA, 1836 bp, Oryza sativa
  misc_feature (1)..(1836): Ceres Promoter p530c10
gcctctcgaccacgagtttagcacttgtgcaacatatatgcgtgcgatgaacatctactgatgcgccatgcgaattttagcgttcgttcatgacgcttccaacggcacagaggctgagcagcagcatgcatgcatggctcttgtgaaaacaaaaaaggttactggtaaatgacatgctgctgtagctagctagcagaatgcaaggcccatgcatatgcaatgctatgcgacaagtacagtaccagcatgtatggtagccagctaactaatctatcagcagaggcagcaagctcgtgcatggtgtgatgcacttctctccagtaatctagtggtaattttcacccaaagcgttgctcatatggacagtaattagtaatattaccaaggttcacaatcccgttacctgaccaaatactactcacgaatggtatctctggttttcgttaaaaccgttggtaaaccagcaaaaatagacaaaatttgtcaaaattttaaattttagttttttttttttaacttagccgggaaaccttgaagtttgtgctgtcgagctgtcctgggaaggacggttttggttgggattgtgaaccctggttactgcacttcatttttgaacagatattagtgcaacagacaaatgccaacgcatttttttctgtttaccggcaagctgaagcttttacgatccccatacagccgttgctgcaaacctgccaagaaagagcagcagaaacaggtgtcattttgtggtggaaagccaagtaaagtaaacagaagatggaagatagtgaggaccagggagtgaggcaggggacacatggcccacgcctccctgcacattttcgtgtataaatacaggtggatgcatcgctctcccagcatccatcggttctctgctctgttcatccatagagtttcctcctcttctcctttagtgcaaggtagagaagagcatgtgtgtgtgtgtgtgtgtgtgaactgtgaagtgcagagtgcttctgtagttctgtgttatgtccatagtgatcttgttaggattgttgctatggatgcatgatgttatggttgatctctgaattacagtagggacttttctgagatctctggattagtggggggtgctaaatttttttctggttgcatcagcttgggtttctggtattggtgtgggttcttgctctgaattttggttcagaatgtcgatttgtttgtgtttgttctctgaagttgagagtagctatgatccatccagcacagaactgcaggtcctgcctgccggctgcatatacaggacatgccattttgcaagctctgggcttatggtttctcttttggagttcttcttcttgcatgatctgtgttctctaacaaaggaagcaagatttagcaactttattcagagacaagaaaaggatctggcaaccttttgtttctgttttatcctactcgtaaagattgttatttaagcaaaaatttcccaaaagttttaaatataatttccatgatgtgccactctcatgtccttgaacctggcactcattatgggctcctcagaagtgctgtagctaatgtcactaatcttttgtatctttgttcatagtcttgtattttatgatgcttatccctttgtgctttccatgtttgatgtccaaatgtcatggcaatgtttttgacttctagtaggggttttagtacctttttgttagataagtacatccaaattctgtttatttattcaaaaatcattctgtttattcactgaaaacatttgtccattcaatggactcataaactgtctgtgtttttcaggcttgaggatccatctagaagatagca

SEQ ID NO: 141 - DNA, 3000 bp, Oryza sativa
  misc_feature (1)..(3000): Ceres Promoter pOsFIE2-2
gcttaacacatgaactaccaaaatatactgatcactttgttctagtcatacataccttaagtcattttattctgcagtgtttggattggagggagcattctagcatcccttgggtcgttccagcaaatgtggttctccaaagcagagtaagcacaacacagtattttaggttatgtttcccctatctcgtcacggacagctcacaagttaatgtgatttatctcactatagatacgaagaacatggagtatcctacatccaaaggaagtgcccatgaagttgtggagcatcgctacgatttgtgaccaaatttgggtgcatgtgggcaatcgtattacagccaccctgttgttgatctatatcgactattatccgacgatatttatcattatattatgactagttagtttgtagattttgagagggcaacataagaagcaatccagcttaacctgttatgttcttgatggtagattctagttcatgtgttgaatctgttctccctgctgtagaatgtatcgagttgctgctctctactctgtacttttagaataccttttcaatcatttggagtcagctgattgttgtactacttatacgccacctgattagtcatgtcaacaattaaacttgagcactggttaagttaagagtggcctgattgtagttgataatcacattttattcgtagacattgtatgctggatctttatcagccaccgtcagatcatcctctgtaataaatcttcatcagacgtgtgtgccaatcgcaaggaacacgaaatgcatccgaaatgttactctgagttaatcaatactataattcttggtcaaattaattatttatatctataaagtttaaattaaatttaggaaaatgaattcatgcaaatcttgtggtaagttgtcaatttcataaaaaatccagcttactactccctttttaggagtgtgttgtggctgcacacttctgccttttgatatatacggttctattctcggtgtactcctttattattattaaaacaatcccagttacttggtaagtgctaatcacgaatcaaagtcaacataacaaatcatgtgcgtacagctataactcgattacacaaacaacaaaattcatatttgaacataaatccagttgtagcatatctggtagtataaagttttttttttgtatagaagagttttaatttctgtaagttttggaaagcatttaatcctagaaattgtagtgtagctcaactaaaaaataaatgaacttgaatcgaaattgggttgtatcataaatctttaccactcaaacgaatatttatcctaaaccacaaatgactcttttcatcaaggaatgttttgttttcagcattttaaaaaaaaacttttctaatatggttttcatgtttcgttcttttgaaatttaacatctatttaatttgcacggctccataaattcaacggatacatattctgaataattactaaggaggcatatatcggctctcttaatacaaccgcttgtttctcaaaatttattttgagttttgtctacacattctcaaggacggtacaaacacactatagatgttcacaattttttttttctaaagttgattgatggacaaatgtttgaacatataaacatataagcactgaatatttgcttatgcaggaggtatttatatcaagttcgatactttactaccatagtccctaggacactaaaatgccttcaatgatctgatgaagcctaagagagaatattgatcagtggagcgacttgcaactacacatggcacaagtagactagacacggtatatattcatattaacttgttaaaattttactacttaacagttcacttgtggtgcatccatatcaattcttacttacacaatatttgtaaaaacaacctaacactataggatgacctagacaacctttatgtcaatcacacttagaagatgatcgtctttttaataaataatgtgtactacacaccatgctctccatatagatcaagatctacaaacccttccacttataaaccttaccaccaaaaactcattaagttgcttcatttatctatgctattaagaaaaaaacttatttcgtttatgccatttctagaaatggctagtcacactattcacaatattatataataaataaaagtttcaaatattcatccaccaaaaatcatcaagtcgtgggacttatatgttaattagagaagtccctttgggtgcaatcgattttggaaaccctaaattttttctatacatagaagagagagatgtctagttgcaattgcttttgcgatgtgccaaccacccttctagctttcatccacgtctacttaattgccattcttcttcttctttttcttcactattactacctcctatcttagcgaatcttcttcttcttcactattactacctcccaccttagtgaattcatcctcattgttcacaatgacattgctaagttaactaggtatgctaagtacacaattagaatataacctagagcctttgtttccatcatacttaaaagatgacatttttatatagataaagtgtgctactcacaaggcttactatatatatgtatgatacacacaaactccacaacccaaaactctttcaagttgtgtggcccatctatgctattaaaaagcccatttagcccatccaacatgagaaaccctagggttttttccctataaaagatacctaggttattgttgcttttccaccccgcccgccgccgctccctattcctatttaatcccatctctcttcctcatcaccgctctcctctctccaggcaagaggtacgcactttttgtttcggatttgaaatctttgcttcgttttactatcattggtcataagttcttttttgaagatgtttgagaataagtttatcattgagattatcgtcacttgtgataggaagtacgcaacctcaagccggacaagacgtgagcaaag

SEQ ID NO: 142 - DNA, 2023 bp, Oryza sativa
  misc_feature (1)..(2023): Ceres Promoter pOsMEA
gagagcagaacatagtagccgctgttttctgggggtgcaatttgtgcaagatcgctatccttatggaccatgcaagcaccaagcaatattaagccaggtccaacagcggtcttggggaattcagaaatgagcttaaaaacctccttgagctggccagctcagccaaggaggtccatcatgcatgtgcatgctcaatacttggaattattgcaaaatgatcggtcattgactggaagactttgcgcccttcctcagccaaccttatgtggctgcatgcatagagtaccaacaggaaggtagcgtttgttggaataaggtttgcatccagcatgtccttgtagagcttcaaagcctcagcaccttggcccatgaaggccatatccagctaattgcattccatgagaccacattcttgctatccatactgttgaagtgaagatgctccgagcttcggaaatgcttccacactatgcatacatgtcaatgagcactgtcatgacataaacattgggccccaagtcctcctcagcgataatcctatgcagccactttcccagggacaaagctccaagctgtgcacacgctgaaagagagctagaaatgatgattggatttggtcacacgctaagtaccagcatttgctcaaagagggcaattgccatctccgtccagccattctaggcataccctggtattattgctttccatgattccgattccgtggtcttctatggcatcgcattgaaggccttccttgcagactccatatcatttaacctacagtacaatatggtaattgctgtcgacactggagaattcgcagtaaatccagacttgagaggaccatgtaagcattgatcaagcagttcattcccaaacagactatacgggatcagtgccagtgctcgagtttggcttcaattccaaggccatcaacccaataaacagattaactgatgaaccaaccatgcaattcgccgagcaaacatagattaagcattgtaggcaaccaaatctggattctccatcaagtcaaagagacgccatgcagaattccacatccccgctgtatacaccgagatcaaccggtcagaacatgctcatactccgccaaccctctcttcagaacatgctcatactccgccaaccctctcttctctgcaagaggcatcctccccaattccccattgttatatctgttgctggtaagaccgttgccagcgtggttgtgtcagaccgaacagactctgcactcgccatcctcacgaacgactccagggcctccgaaccaggaagcccggccggccatcagcgtgttccacataacggtatccggcgactgcacagtgtcgaacaccttgcgtgcgtggtcacctctggacagcatgaagcgtacaggctacagcttggccaatgcggacgccacgaacgtgtcggcggcgtaacccgcgcgtgcagcgcgccgcgcgcgggctgcggagtcggttggagacgacacgccgccgccatgagagcaatgagcgaggtggcggcgaaggcgaaggagaagtagtcgaggcaagcggaagagaaggcggcagcggagaaagcgatcggggcggcggaggaggtgggtgggagggagggacgcgtagcggaggtcggaggaggagggagctgaggtttccggggcgggggtcgagagggtagtgtacggaggcgagggacacggcgaggatctggtcgaggtagcgcagtgtgaaggaaagcgcgatgaggcggagggcgccggcgaagagcggcgcggcggatagcgggaggaggcggcgccggcggggtctcatccgattggaaacagattgggaagggggagggggtaggaatacgtggcgtcggcagtattaggtagagagagaaaccctttccatcctttgtctcttagccccgaaggagagagaaaaatcagaaaaaaaaaaccctccgcgtgtgggggaagcagagctccggacgctggcgccgctcgcgccaccgcacccgcaccgcc

SEQ ID NO: 143 - DNA, 2034 bp, Oryza sativa
  misc_feature (1)..(2034): Ceres Promoter pOsYp102
gaacgacccaaacgcgtaaatggtggtactggtttccctgctttgccgagtaccagcagccacgaagaacgttacacaatcgagtacaaaatctataagagcaagtttaatagcatagccaaatactacctctaaatcatctatagccaatttaatagttcatttattcaataattacttataaacatatactacaatcattaatatatggtcttacttcttatacacataatattttggagtccgtgttacagctggctataaatataagggattttggttggatgtggtacatcctattataatgaatctagacatgaaacctgtccaaattcatcgtgctaggatacgccacatctaaccaaaatctcttatctttagggatggagagagtaataattaaatgaagctaggtagagtttcccggtcaatacgcttgcgtgtgcttataagagcatggccaacagtttcccgatactcttcccaatatcagttttgaggagttttgttggaaaaaatcgctccaacagtagacctaaatcacccctaaaagcttggcgtttccaaacccgcatatttcgttctccacttgtagggaagagactcggcgcccaatccttcaaccgcatgcacttcgcgcgcgctgtgtgaaaattttcctaccaggttcttctttgtgcgttcgtctacctgtgagtcaatccatcacgccagcagcctcatcttccccgcagctgtctgggaaagcagccatggctcccccaagcttccccagcgtcgacatttttttctcagcggcagcgccagacccatctccaacccaattgggcggaccttcgtcggcgctcccccagcaccaccaccgactcgaatcggccgtcgcccctattcatctccaatcgtccctcgaccctaccgcatcctgcagcacagcctgtctctcgcgtcagactggcgctgcgctccccccggtaatgtgcaggcgacaaaggccccatgcgatgcgaccagcagccggcgacaaccggaggtgcccagtcgctggccttcatcgaatcatcgtgcacctcggtcggagtcgatttctgattgttgctgctgctcaaatctggagcttgctattgctgagaactgcttggtggtggtactggaaatttgttgtttgctggctgatgaaaactgttgttctttgctgctaaaaactgctgcttgctagtactgaaaagtactattgcagctgctgaaatatcttgctgcttgctgctgaaaacttcaagttgttaacaccgttcacactaaaaaagctgaaattttttttctgggctgaaaaccccattgttgatgattgcagaaccaatatttttccatgtaaaatacaggagatcgtggtaataatcaagtgaaatatcattttggggcaaatactcagatcgtacctgaagccaatggaaacattgttcaatgcttaaactgtcagttatgatgtcaaagagattgatcactgaatgtcctgaaaggagccgtgaggaggatgcagcattgcagcgtgcgcgagcgtgagtggaggagaggaatgacgattctgttggtagttgtcgatgtggcctactttttttgttttgaggattaaattttgggaatctcttggagataaaaggtattctcataccttaaatcctttttagagatctaaaaaaaatgatttaggggattgaattttgggtggctgttggtgatgctctaagttgcacatcctggggaaaaacctccctaatccatcagcaaaccgatcaaccacccacgacaagtcgacgccaccgttttttttttctccctcctaagtcctaaccccacaaaaatcccgcgaactttcgtctcaccacgcgccgcgtgccccctacaaataccaaacaacacccaccacgtccactcacaaaccacgcaggaaacctcagaaaatcaccgtacgcgacgcgggcccaagaaaaccccgacagaaaccgcgcagcagcaacaccaccaccggcgtcggag

SEQ ID NO: 144 - DNA, 1877 bp, Oryza sativa
  misc_feature (1)..(1877): Ceres Promoter pOsYp285
ggcccgagttaaacgatcttccacgtgtcagcgaatcctagtcgttcgatgaatctgaatctgacttgtggtggttggacggccacgtgttaaaaaagggaaacgtccgcatcacccgatgctgggacatttgcaatttcgatccagctgtagattgaccagttgttactctcttttttttaacaccatacaaacgtaatactccctctgtcccaaaatataagtattttttttaacctcggttcagtcttcgaggtgctactttgaccaataatatttataaaaataagatgttttaaataaagagagttgcatattatgatagctcgtttaatgataaacaaagtaccatcaaatttacatgattaatctttttaatttatttgctattaatagttaaaatttaaaaagtttgacttcacactgttctaaaaatacttatattttgggacggagggagtacacattagagcaggtacaatagcagactagtagccagctataaacatattttaatgagataaaagatgagagagaacagcgggctacagatctgtagccagctgcagcacggactccaagacattgtgtgtgtatgacaggtgggaccatatattaatagtacagtaagtaactattgtatgaattggctattagattagctataggtgaattgtagctagtagtgggctatactattgaacttactcttatatctctcaatatctccagaaaactaggacgatatatattgatattaacaaagtcatcatagatatctcgctatcgacatatatattacctatcactgaaaaaataattaatcataaatgcaagcacatatactacgttcaacactgaatgtaggtagattggtagacgggttccaccgcaagaaaagcattgcaccagtgaagaaagaaacatcggaatttgtatgtagtttgttgtttgatgaattcttttgattaaaaaaaactaaaatcagagttgattcagttaatggtgttgcctacgatatacttccatatcatgatatcactgtagactatgaatcatatctttaattaaaactaaatcaagaaattaagtatgagacctcaactcaatgaagaatttctagttgaaaaacattcctagtgtgcgttcggatggaggtagggatcttctctccgttcatataaaaccggatggttcattagaacatgattaattaagcaacagttaatctaaaaataaattaatattttttaagaaatttttgtatagagatcttttgaaaaaaatacattggttagaaagcatactaataaaaagagaaaaataagaacatagtactatagtagaaaatgagaacttggagtatttgagaggatgggaaataagaagattaagaagatgcgtaaagtgaacggttaacgcatgattgattaattaaatattaattattttaaatttggaaaataaattagtatgatttttaagcaacatatatatatatatatatatatatatagaaaaacatagttttagaaaatataagcgtgtaaaacgatatgcaggaacgaaacgttgagcattcaaaatttcaaattgaacatatgaatcaagagagaataaaaaaagaggccttctaggctggcatggacaattggacatgttttcaactagggtttcaagcttcgagcatccacttttgtccttgcaaactttatacggcaaggcccgtgaatctagccccccacaccaccccacccgcccgcgccgcgcggccgcctcgcctcccctcccttctcctcctctccgcccccgccgccaggccgtccacctccgccgtctcctcccccattcgcacccaaggcgctggcgcggaaggc

SEQ ID NO: 145 - DNA, 1000 bp, Arabidopsis thaliana
  misc_feature (1)..(1000): Ceres Promoter PT0565
caccaaatatagtgttatttcaatactaaaatggtgttatggttggagatgccctaaagataaacatgacgagacacgagatttattaatttcttgatcaaccataacttaataacttaatattaatttcacttaataatttccaattaagtgaatctttacttcaccaaaagttcctaacgaactcttattttctagcatcaatattaccatgaactagcatcaatactatcatgaaaaattcctacttcctatccaactcttaataacaatgctagtcttaacaatattcatcaaaaacttgatatagaccttctaacttagccacgactagtatcggtgaataccaaaattaatgtattcatgagaacttgagatttctctaatgtattcttgttactaaacaagtaacaacactcaagaaatatcatgatcaaatattttactcataaactccatatttcacattttgaaaattttaaacagcaaatcacattgaattttcgtggtaaaagtatttaaaattgaaaaatagcagctcctgatttcaatgtataaatttatctttatatggtttatgtctccaacttattttaaaaaagagagaaagagcacccaaaaggtgaccgtttgaaattcgaatttatttccgtttgaaattcgaattcaaaaaaagtaaaccgaaccgagtctcgttactgactgtcacacattgtttccctaaaagctaattaacccatacgtggcgtaatataacaggtcagtgatcaatactaaataacagacatacacctttaaaattcgtgcacgctccaaaacaaaatctacacttcaaaatcaacggtcacgatcattcctcaaatttcaaaaaattatttaacctcacttccttcgctttgtttttaaaacctctctctctttctctttctctttcgccattaaaactctgtttcctttttcagagattctcagagaagattcattttaccctaagaaaaaa

SEQ ID NO: 146 - DNA, 999 bp, Arabidopsis thaliana
  misc_feature (1)..(999): Ceres Promoter YP0015
ttgagccttattgttgttattgacttttagccaatagaaagagatggaaattcaataattatccacaaaattccaaatcattggtgtacaaaaagatctaaggctgttatattttcaaaaaagaaagaaaagaaatgcaacaaatatggattaaactgtggtttgtaaattgagctttgcatgaaaactttatcactatgatttcactactccatatttattgactaaagtggcactaatgaatttcttaatcatgaaatcttgtatcaaaaagtactaaaataaacatgacattggcaattaggaaaattctaaattagaaattagtaaaaatgaaaggtgaaagggaaagatgatgatatgaattggttggtgaccaggagaaatgtatcccgatttttgcagacactttcagtgtccccattcatataattatggcccacctcgttaagatttttcattcaccaccataacaagatctaagcttagatttcatgtaattaaacatataatatacttgccaatactatctaataaagtatacttaagcaaaaattattactctagtgtaaggcgatgaaatataagtttagttgaaaatttatgtcgatataacaaagtataatgaattaagaccttggttttcgattaacaaactaattaaacactagttttgcctaataaaaccgggaatcgtattcaaaaccgaacgacaaaacaagggacaagttgagagacaaaaccaaatcagcatctttcttccagaaatgtcatgaccacatgacgtcatcttgacccttcttcattgtgatatctgtggataaagcgcacgtgtttaattcacgaaccttcgtagtaacgaaaaatccacaactttcatattttttaattacccactaaactaaaacaaatttggaaaaacatgaaaaactttttctttttttccaggttcgtgaacctcgtaccctctatataaacctcttaaccaccttccacata

SEQ ID NO: 147 - DNA, 999 bp, Arabidopsis thaliana
  misc_feature (1)..(1000): Ceres Promoter YP0087
tgaattgagtaaaatgtgttttcaaacagttaggtggtagaaggtaaaggtaataacatcatgatcttactaaaagaattgttgcatactaactatcaatattctcaacaacataatataatgtttttttaggtaattttccattttaattttttgtgattaaacaattaaacaactcgaatgatgatgataaaaaaaaaaaattaacaactcgaataagttaaagtagcaatacacatgtcgttcaattcaaccaataaagtaagacttatatttttaagaagttgactaatagcttaataagttggaaaacttgtgtagtttcttaattcccacgtgcagtaagaaataaaaatgaaaaaaattattatatccttcccactctgcgacttttcttttattttatcaaatattaaaaagattcatatcacagtttacacattgaaatcataaacgataattatgtattttgtaataaaaagttagttctgaagctcatactttggatagtcgctagtcgctaatatgctccttgtaataattaaagtcactacgacgcacgtcaaagccgatatttagggcttaattgatgcgtgtttttcttttcatataatagtaatataaattagtactaataaagtatgatggatggttgagacagaaaagaaaaaagatgactgtatggtcatcattacaaagaagaatgtattcttcatgttcttaagaataataaaatgtcacttgtaaatcaagttggtaagcattttgagaactttgttcgatgcaacgtatgatgatttatgtagacaaaagataaaaccgtatcttcaactattgccaagaaaagataaaacctaatctagtcagtctctcaacataaatacaacccaatagccaaactgtgtccaattcggagagaaactaaactaaaacaaaacacaaaagcccaacataagcccaataaaacccattttataaacagaacattactaacactca

SEQ ID NO: 148 - DNA, 1000 bp, Arabidopsis thaliana
  misc_feature (1)..(1000): Ceres Promoter YP0093
atgatgaacattctacatatataattattatgtttaagcacttagacagcataaattctttctaattatataaatctaaccttgttacattgtacatctataaattacttgaagaaataacgagttctatttctttttaaaaattaaaaatactataccatatctcagtgattaagttgaaccaaaaggtacggaggagaaacaagcatttgattcttccttattttattttattcatctctcactaatgatggtggagaaaaaaagaaaatacctaacaaacaaatatatattgtcatacaaaaatatttctatatttttagttaattagtttatattcctcacttttcagggcttatataagaaagtgagcaaacacaaatcaaaatgcagcagcaaatactatcatcacccatctccttagttctattttataattcctcttctttttgttcatagctttgtaattatagtcttatttctctttaaggctcaataagaggaggtactattactacacttctctctacttttacttgtattttagcattaaaatcctaaaatccgttttaaattcaaaaataaacttagagatgtttaatctcgattcggtttttcggctttaggagaataattatatgaaattagtatggatatctttactagtttccattcaaatgattctgatttcaatctaatactctcactctttaattaaactatatgtagtgtaatttcacactgttaaatttctaccatgtcatgtatattagagttgcatagaaaattgtaaaacatccatttgaattcgaatgaaacaaaatgttttaaaataaaattttggtttttaaaagaaaaatctaaaactgaattatatcgtttaaccaagttgtaaaagtcataaaacgtagtatcttgtaaatcgctcttccacggtccaaatagacttctagtaataaacaagtaaaactaattttggtttcttactaattttcacaga

SEQ ID NO: 149 - DNA, 999 bp, Arabidopsis thaliana
  misc_feature (1)..(1000): Ceres Promoter YP0108
ttagctgaaccaggaaattgatctcttataccagtttccgggtttagattggtttgatggcgatttgattaaacccccgaaattttatgtcgtagttgtgcatagtattattattctttgcggacaatagacgtatcgggaccaagttctgtagcaaaattgtataagcttaagtttgatgaaatttaaaggtaatcactaaaacccaaatgggacaataaaccggtgaagatttagagtttttaattttgactcatgaatctggagaaagagccctcgttaaaaggagtgaatcaatccataggggaaaaagttttgtctttttaaaaactaaagaaccaaaccttaatagaagcagctcaatgtgtgacaactttccactggcactaagataaagtgactagcgatgagtgcaattattgaaatagtagatggtaaatattacatacaagagtaaaaatatctttatgtcaatgcttaattcagtgtttctggttaacaagagaaacttctctaactttcgtaattgggtcttataaaattttatgcaattatgattttacccttttactacttttcattagctttcacgaatctattttgacaagagaaatcattagaggtaaacatgctttttggtcaagggccttaacagttccaccaatcaagctcaaaagttgtacttaaccgacatcttctgtgaaaacatataattacatgtacaaatcaaaactaccttatgaaataaatagaaatattgcagttcatttctaatttaacctcttcaacttttaaaactatttacatttctttatgtcatttctagtcattttgatgcaaattgtaccatttatggattatcttcacaaatttttaagttggtgaaaactttttggtgggtagttaaaacttgaaatagaaatttactttaccaaaataaactaatgaaaagtaatcactccactccctataataagatttccaacgttcccactaagc

SEQ ID NO: 150 - DNA, 999 bp, Arabidopsis thaliana
  misc_feature (1)..(1000): Ceres Promoter YP0022
tagttccattacaatttccaaatgatttgttacaaagctacaagattattcgaaataggatttcatccataagagagaatggtgtggtcgacgctacaatgttgatttattggttgtggtttgcatcttggggatgtcaaatcctaagtttcaagttcttgtaaaaacgttttcaggtttctttaatatattttaatattaatgtaaaaagaaaagatatagcttttgtacaaaaaaatttgtttaatcactatgtaggaggatgcgatcaaattcatggaatgatgtattattagcttttctatcctcactctaaaaacaatactatagtgagttaaataatttgatcatttcaatgtagattaaaattttattaaaagaagaaaaatttaaaagcctataacaaaataaaaaaggaggctcgaggtatgatgggtgtagcagaagagctggcaacagctatcgactgagtgattacgaactcagtactcagtgttctcagctcacacactctttttttgttctctttcttttggacagctttcattttctcttttcttttttctattttgtttcaaaattccatccatattaaaataggcctgatcatgagaataaaggaaatactaatgatgagtttctcaataatgcaataagatgcaattattatgagctatttactattgaaaatgagcaaataaatgtcaaaacacaatctggttaagttagagcaactccattgtataggattcatgtagtttctaagaaaacaaaatgtattaatattttacttttacatccaaaaaaccaacttatatgagtaatagaaacgatcctaatattaggaattttagagattttctctcatctgtttcttaacttttcaatatttttattttttaaaattgtatgagtttctactaagaaactactgctggagttggtcttagcttcccaatgcttctccacctatatatatgcatatctccttcttaaaac

SEQ ID NO: 151 - DNA, 999 bp, Arabidopsis thaliana
  misc_feature (1)..(1000): Ceres Promoter YP0080
aagcggcaatttagtaagaagtactcaaagtatcatttaccaaaagtatatggttttgggaagagttgttagggatgtattctttctaaacagatgatatgacgatgttcttgaaaactaatgttaaagacggaatctctggcatcttcactcgggagatatattaaaccgttgattgtagttagccatgtacttagcttagtgcacaaataatctgctgcaagaaatctttttctattataatatctctcatttaaacattagaacatattgtttaacttgttcttctagaaataaaactgctaatttcttatggtaaactattttcctttagattgcacaatcgaactcgaaaatctagtggagactatgtgactatgtttatatatatgaaacctaaatcaaattatcccaataattgggagacacaaaagaaaaattacgaaagaaaacaggaaatcaaatcaaaagataaagagaaggtaaaaaaaggcaagaagcactaatgtttaatatttatagttttctccattaaagaaaaagcgatgatgtgtgttctcatcttttgtgaaagtatatatattgcttttgcttttctcaaaagcaaaagactcatccaacaagaacaaaaaaaaaaactaaagctcaatccaaaagacgaagaatgcattggatactacaacttctttttcacttttctttcaaatttacaattatgattttcacaatacagtttattcaaaaataaataaaaaaacgaggcatgaaaataatgattatcctcttcacttattaagccactcactataagcagagcaactccagaacatagtgagcccccaaaacattaaagcatgatgatgtctaatgatgatgatcttcttcgttccatttctctaaatttttgggatttctgcgaagacccttcttctctttctcttctctgaacttcaagattcgtgtcggacaaatttttgtttttatttttctgatgttaca

SEQ ID NO: 152 - DNA, 3000 bp, Arabidopsis thaliana
  misc_feature (1)..(1000): Ceres Promoter PR0924
atctataacgagttaacatgttgccagtttgaatcaagaagcttggatgatgaatgaatggatcggtttgtggtacaattcttaaaattgtagtagaggagacagagaaaaaacatgataagactttggtatttacaacttgacggagacaagacagtaagccaaatctgtcacaaaaacactcaaactcttttctcagtgttttgagtttaaagagagacttattcacttcccctttcgtaacacttatttgtctcccaaccaaacagtttctgtcctttcccttgtcctcccacgtgcatctttatatctcatgacttttcgtttctagatcttgaataatgtcttagtggattaggtttgttgtcggtaaattaggtgaccgtttttttcttatatttggaagatcgcgggatgaagcagatactgagtttcagggcatacacacctaatttgaaaatcattgttagtccaatttcactttaatcttgtttacaaaaaaattgatctgaaaatgttgatgggataagtaaaaatgtaagttttgctagtagtcatgatataataatagcaaaaccagatcaattttgagcaaaaggaagaaacaaaaaacagatcgatcccacgagcaagactaagtgtaaagtggttcccacaagagccatatggatatggtccttcaacttttaaagcccattacttcagtggtcgacccgacattacgccacgagtagtcacgcacgcacgactccgttcacgtgacattcacgttgatatttccccctctactctcttctgcttggttgatctaaaaaacatgaagagaccaacctaatttcatattaatatatgatatagacttcatactcaacagtcactttcgtaatccaaatccatatcttacgaaattagttcttaataaaggttgtggattaagttataatattgtgttaagagttaagacacagcatataaccttgtaccaacagtgctttattcttaaatggaaacaaaacatatgtcaatgtcaagcatacagctaaaatatcattatctaatattaagagtaaaacaagataattaaaaattgaaacaacaccatatttttatagctttacttatcgtatttttctagtcttcatggtaattgtgttgctttattttgtttataaatgaatttggttcgaccagatagtctaatatcagtttttaaacactggttttaataaaatcatatgtcggcaattcaacctgttacgttgtatgattgtatcctagtcaaataggggaggaggtactagtcgtttcaattagtttacgtaatcaatccaaagaaactataagctataaagatcctcaatttgttggttacaataaaaacaacagttgtcaaaatttatgtttataaaaagtaataactatgttccttcccatatagagcaaagtacctcaggataggcaaaccgtacttaatagcccttattcataatttgatccaactcttccccacaaaattgcaactgatgaagtcaatacttgtatagtgagtcaagctataaatgtctagtgatagttttgtctcttaaaaggttaacaaaagttatgacaagctgaaaaatcagagtttgctaggagtattacttacagttatcagtttaagtatcacatttatagtattgtatacaatgattcttaaattccaccttttccgtgcgaaaccaaattttctattggaaacatagaatgtaaacaaaaatatgggacgttgtccgttccaacattaaccaaacttgtctattactaatattcgtgttggtttgatgttggatgtctaaattcgttgaatcatgtgtctcttgacgaaatatgcatcttcttatttcttagtatagatgcactttatcattcttttagtacatgcttaatttttttttttaaaatatgttgattgtcatattgccaaaagtatgaattaaagacgcacatctaacacaagttagcagccgtaaatccttccataaatttattttgcaagttttgctcattatataatgagcggaatttatgatataatcgtttgtaataatgttatgttttgatcaaaatttgaaattaaaagtaggtgagaacttgttatacagtgtagataaggtggatcttgaatataaaaataaaatttataagatgtatttaaagcagaaaagcataaaactttagataaaataatgtaaaaatgtgttagcatcaatgttgggatattggccgacccgaacttaatcaatgtcggaagccattacttctctcccaaaagacctttttccttcggagaactaggaacttcctcactacctttcgcttaacgtgaaagccataaatttcatatattcataaaaatcagaaaatctaaaactgtttagtatcacctgtttttggtatagactattggttttgtgttacttcctaaactatatgatttcgtacttcattggatcttatagagatgaatattcgtaaaaagataagttatctggtgaaacgttacttcagtcatgttgggtctagatttacatactactatgaaacattttaagataataattatcctagccaactatatgttctatattatgggccaagaagatatagaactaaaagttcagaatttaacgatataaattactagtatattctaatacttgaatgattactgttttagttgtttagaataaatagtagcgtgttggttaagataccatctatccacatctatatttgtgtgggttacataaaatgtacataatattatatacatatatatgtatatttttgataaagccatatattactccttgacctctgcccccatttccttttactataaataggaatactcatgatcctctaattcagcaatcaacaccaacgaacacaaccttttccaaagccaataataaaagaacaaaagcttttagtttcatcaaagacgaagctgccttagaa

SEQ ID NO: 153 - DNA, 1000 bp, Arabidopsis thaliana
  misc_feature (1)..(1000): Ceres Promoter YP0388
agaagtattcacgcaccaaggttatatttgtagtgacatattctacaattatcacatttttctcttatgtttcgtagtcgcagatggtcaattttttctataataatttgtccttgaacacaccaaactttagaaacgatgatatataccgtattgtcacgctcacaatgaaacaaacgcgatgaatcgtcatcaccagctaaaagcctaaaacaccatcttagttttcactcagataaaaagattatttgtttccaacctttctattgaattgattagcagtgatgacgtaattagtgatagtttatagtaaaacaaatggaagtggtaataaatttacacaacaaaatatggtaagaatctataaaataagaggttaagagatctcatgttatattaaatgattgaaagaaaaacaaactattggttgatttccatatgtaatagtaagttgtgatgaaagtgatgacgtaattagttgtatttatagtaaaacaaattaaaatggtaaggtaaatttccacaacaaaacttggtaaaaatcttaaaaaaaaaaaaagaggtttagagatcgcatgcgtgtcatcaaaggttctttttcactttaggtctgagtagtgttagactttgattggtgcacgtaagtgtttcgtatcgcgatttaggagaagtacgttttacacgtggacacaatcaacggtcaagatttcgtcgtccagatagaggagcgatacgtcacgccattcaacaatctcctcttcttcattccttcattttgattttgagttttgatctgcccgttcaaaagtctcggtcatctgcccgtaaatataaagatgattatatttatttatatcttctggtgaaagaagctaatataaagcttccatggctaatcttgtttaagcttctcttcttcttctctctcctgtgtctcgttcactagttttttttcgggggagagtgatggagtgtgtttgttgaatagttttgacgatca

SEQ ID NO: 154 - DNA, 283 bp, Arabidopsis thaliana
  misc_feature (1)..(1000): Ceres Promoter PD0901
caaagtatttgacaagccatatggttttggatcaaaaagtcggtccaaaattaatgttttatgtgcaagaaccgacccattgtacacacgtgttaacatcttcaagactttcatctctatttttcttttggtcattaagatacccattgatccgaatctgttacattcccacctacttttttaatttttactatccactccaaattaaacacaaccgatgattttaataattggaagctttttaaaatatttctccacgtgcctctttgtgtttgtctatata

SEQ ID NO: 155 - DNA, 1000 bp, Arabidopsis thaliana
  misc_feature (1)..(1000): Ceres Promoter PT0623
aaagttattgacattttgaaaggaccgtaaatattaccaaaaaactgacggagttaggatcggccacgtagaaagggacaaagagagaacagtcacggactcggccagactaagtatgggcctgtctgaatccaaactcagctaagttccaaaagcataaagagagatgtgtaatgaaatgaacgtattctagaaacgaaagcaatgttatgctttgtttttgagccacatgtttttgggagatggagagaatcttttttacgtttttaacctaacccacttggcacttggccaaaaaagtgagaagaaactgtggcgaatgagtaggccacgccatggactttgttccttgtccttcaaaagttaaatttatgttatgcgtggggacaatctaagcaacgtggttcctttaaatatcgcagcttcctcttttacacttttggagcctacgtgttttgttttggaccggccaaatacacgagtcagtcagtttagaaataatttggatgtccaaaaatcttggagatccaaataaaataattagcatgttttagttcataagaatatgaaatgtagataaactgtctatattaatttttccatagaattggctttttatcgaggtgatgtacttaatgactttgttgattactactcgtataacaataaagaatatgatactatgtgagacttataatgaatttggtgtgtgttaattaatccagttgaaacagtttaataacaaatcagaataaaaattgtagtaagaaaatttgaacgctgatccttcaacctagatagtgaacctttcaaatactatatgattcacgtgtaatgtttttgaccgttggttatttttgtgtgaactatattaacttatcaatatcgaaaggctaaataagtaaataactaaaagaaagttcaggaaacaactcgacctaatgacctatcatttctgatcacccgtcctataaatacatacgtaagatcattcgttact

SEQ ID NO: 156 - DNA, 1710 bp, Gossypium hirsutum
  misc_feature: Ceres Clone ID no. 1920025
aatcccaaaaatccaaattcagatgtcattttcccaaactcttttcttcaaaattaaaccccaagatcgattttgaaaacccaccaaaacatttgcacttcaataaaaaaatttcaatacagctctttttttttttttgtatggtagtgacaccagaaaaaaaactatcaaatgcaagaatccaatgcgccatcatcttcttaaagatagaaatctaataaccttcttcgatccttaaaatcgatattgttttttgttgttttggatctgattttgaagcgtttaaaaatttcaaaaacaaaatggggagtttgggagcactgatgaaacatccagatgatttttatccattgttgaaattgaaaatggcggcgagaaatgccgagaaacagatcccatcggagcctcactggggtttttgtttctctatgcttcacaaagtttctcgtagtttcgctcttgttattcagcagctcgatacagagcttaggcatgcagtttgcatattttatttggttcttcgagctcttgatactgttgaggatgatactagtgttgcagcagatgtcaaagttccaattcttatagatttttatcgtcacatatatgatcccgactggcacttttcttgtggtacaaaggaatacaaagttctcatggatcagtttcatcacgtgtctgctgcttttctggaactcgaaaaaggttatcaggaggcaattgaggatattacactaagaatgggagcagggatggcaaaatttatttgcaaggaggtcgaaacagttgatgactatgatgaatattgtcactatgtagccggacttgtcggattaggtttgtccaagctattccatgcctatggatcagaagatttggctccggaatcgctctccaattcaatggggttgtttcttcagaaaacaaatattatccgagattatctagaggatatcaatgaaataccgaagtcacgcatgttttggccacgccagatttggagtaaatacgtcaagaaacttgaggacttgaaagacgaggaaaactcggtcaaggcggtgcagtgcttgaatgacatggtcactaacgctttactacatgttgatgattgcctgaaatacatgtccgctttacgtgaccctgcaattttccgattttgtgcaatccctcagatcatggctatcggaacactagccttgtgttacaacaacatcaaagtcttccgaggtgttgtgaaaatgaggcgcggtttaactgcgaaagtcattgatcgaacaaaatcaatgactgatgtctatggtgctttttatgatttttcttgcattttgaaagccaaggtcgacaaaaatgatcctaatgcacaaaaaaccgtgagccgactcgattcaatcctgaagacttgccgagactcgggtgtgctaaacaaaaggaaatcatacataatcgagaatcagtccaattacactccgtttgtggttgttttgcttttcatcatattcgccatctttttggttaacctgaacccaaactggcctaataactagtaagttcgatttctaaataatgatgcgatagaattatgttgcttggattgaagtgtcagtgtgatttgatcagattctttttgaatattttcgtgtaattaaagaatatttggatgttttattctcatattaatgtgccagatgcaagtattgaacgtaggtactt

SEQ ID NO: 157 - protein, 413 aa, Gossypium hirsutum
  misc_feature: Ceres Clone ID no. 1920025
MGSLGALMKHPDDFYPLLKLKMAARNAEKQIPSEPHWGFCFSMLHKVSRSFALVIQQLDTELRHAVCIFYLVLRALDTVEDDTSVAADVKVPILIDFYRHIYDPDWHFSCGTKEYKVLMDQFHHVSAAFLELEKGYQEAIEDITLRMGAGMAKFICKEVETVDDYDEYCHYVAGLVGLGLSKLFHAYGSEDLAPESLSNSMGLFLQKTNIIRDYLEDINEIPKSRMFWPRQIWSKYVKKLEDLKDEENSVKAVQCLNDMVTNALLHVDDCLKYMSALRDPAIFRFCAIPQIMAIGTLALCYNNIKVFRGVVKMRRGLTAKVIDRTKSMTDVYGAFYDFSCILKAKVDKNDPNAQKTVSRLDSILKTCRDSGVLNKRKSYIIENQSNYTPFVVVLLFIIFAIFLVNLNPNWPNN

SEQ ID NO: 158 - DNA, 1242 bp, Populus balsamifera subsp. trichocarpa
  misc_feature: Ceres ANNOT ID no. 1503464
atggggagtttaggagcaattttgaaacacccagttgacatatacccattgctgaagctgaaaatggcagctaagcatgctgaaaaacagatcccacgtgaacctcactgggctttctgttattctatgctccctagagtctctcgtagctttgctctcgttatccaacaacttgacacagaactccgtaacgctgtatgcgtattttatttggttcttcgagcccttgacactgttgaggatgatacaagcatacctacagatgtcaaagtgcctatcctgatagcttttcaccgccacatatatgatcatgactggcatttctcatgtggtaccaaggagtacaaggttctcatggaccagtttcataatgtttcaaatgcttttctggagcttggaaaaggttatcaggaggcaatcgaggatattaccaaaagaatgggtgcaggaatggcaaagtttatcttgaaggaggtggaaagcattgatgactatgatgaatattgccactatgtagcaggacttgttggactgggcctgtccaaactcttccatgcatctggattagaagatttggcaccagatagcatctccaattcaatgggtttgtttcttcagaaaacaaacattattcgtgattatttggaggacataaacgagatacctaagtcacgcatgttttggcctcgcgagatttggagcaaatatgtcaacaaacttgaggacttgaaatatgaagagaactcggtgaaggcagtgcagtgcttgaatgatatggttaccaatgccttgatacatatggatgattgcttgaagtacttgtctgaattgcgggatcctgctatatttcggttttgtgctattcctcagatcatggcgattggaactctagcactgtgctacaacaatgtcaatgtcttcagaggtgtagtgaagatgaggcgaggtcttaccgctcaagttattcatcaaacgaaaacaatggatgatgtctatggtgctttcttcgacttctcttgtatgctgaagttcaaggttgacaacagtgatcctaatgcaataaaaaccttgagcaggctggaagcagcacaaaaatcttgcagggaatctggggctctaaacaaaaggaaatcttacataattaggaatgagccaaaatataattctgttctgatcgtcctacttttcattatattgtctattattttcgcgtatctctctgccaacagatcgagttactag

SEQ ID NO: 159 - protein, 413 aa, Populus balsamifera subsp. trichocarpa
  misc_feature: Ceres ANNOT ID no. 1503464
MGSLGAILKHPVDIYPLLKLKMAAKHAEKQIPREPHWAFCYSMLPRVSRSFALVIQQLDTELRNAVCVFYLVLRALDTVEDDTSIPTDVKVPILIAFHRHIYDHDWHFSCGTKEYKVLMDQFHNVSNAFLELGKGYQEAIEDITKRMGAGMAKFILKEVESIDDYDEYCHYVAGLVGLGLSKLFHASGLEDLAPDSISNSMGLFLQKTNIIRDYLEDINEIPKSRMFWPREIWSKYVNKLEDLKYEENSVKAVQCLNDMVTNALIHMDDCLKYLSELRDPAIFRFCAIPQIMAIGTLALCYNNVNVFRGVVKMRRGLTAQVIHQTKTMDDVYGAFFDFSCMLKFKVDNSDPNAIKTLSRLEAAQKSCRESGALNKRKSYIIRNEPKYNSVLIVLLFIILSIIFAYLSANRSSY

SEQ ID NO: 160 - DNA, 1221 bp, Populus balsamifera subsp. trichocarpa
  misc_feature: Ceres ANNOT ID no. 1514021
atggggagtttgggagcgatattaaaacacccagctgatttatacccactattgaagctgaaaatggctgctaaacatgctgcaaaacagatcccgtctgaacctcactgggctttctgttattccatgcttcatcgggtctctcgtagttttgctttcgttattcaacagcttggcacagaactccgtaacgctgtgtgcattttttacttggttcttcgagcccttgacactgttgaggatgatacaagcatacctacagatgtcaaagtacctattctgatagcttttcaccgccacatttatgatcgcaattggcatttctcatgtggtaccaatgactacagggttcttatggaccagttccatgatgtttcaactgcttttctagagcttgaaaaaggttaccaggaggcaattgaggatattaccaaaagaatgggtgcagggatggcaaagtttatctgcaaggaggtggaaaccattgatgactatgatgaatattgccactatgtagcaggacttgttggactgggcttgtccaagcttttccatgcctctgaattagaagatttggcttcagatagcatctccaattcaatgggattgtttcttcagaaaacaaacattattcgtgattatctggaggacataaatgagatacctatgtcacgcatgttttggcctcgcgagatttggagtaaatatgtcaacaaacttgaggacttgaaatatgaagagaactcggtcgaggcagtacagtgcttgaatgacatggttaccaattccttgatacatgtggatgattgcttgaaatacatgtctgcattgcgggaacctgctatatttcggttttgtgctatccctcaggtcatggctatcggaaccctagcaatgtgctacaacaacatcaatgtcttcagaggtgtagtgaagatgagacgaggtctcaccgctcaaatttttcatcgaacgaaaacaatggccgatgtctatggagctttctttgacttctcttgtatgctgaagtccaaggttgacaggaacgatcctaatgcaacaaaaacattgagcagtctggaagcagtacaaaaaacttgcagggaatccggggctttaaacaaaaggatggtcgatttcttgaattccccagtgtgcgtatggatgatggctggctacacaagtgcctgttctattccatggaagctgcttttcttataa

SEQ ID NO: 161 - protein, 406 aa, Populus balsamifera subsp. trichocarpa
  misc_feature: Ceres ANNOT ID no. 1514021
MGSLGAILKHPADLYPLLKLKMAAKHAAKQIPSEPHWAFCYSMLHRVSRSFAFVIQQLGTELRNAVCIFYLVLRALDTVEDDTSIPTDVKVPILIAFHRHIYDRNWHFSCGTNDYRVLMDQFHDVSTAFLELEKGYQEAIEDITKRMGAGMAKFICKEVETIDDYDEYCHYVAGLVGLGLSKLFHASELEDLASDSISNSMGLFLQKTNIIRDYLEDINEIPMSRMFWPREIWSKYVNKLEDLKYEENSVEAVQCLNDMVTNSLIHVDDCLKYMSALREPAIFRFCAIPQVMAIGTLAMCYNNINVFRGVVKMRRGLTAQIFHRTKTMADVYGAFFDFSCMLKSKVDRNDPNATKTLSSLEAVQKTCRESGALNKRMVDFLNSPVCVWMMAGYTSACSIPWKLLFL

SEQ ID NO: 162 - DNA, 1170 bp, Gossypium hirsutum
  misc_feature: Ceres Clone ID no. 1850745
gtatctatacctgtctctctatctctctctgtttttctctctcccgcaaggatttaattttttaattaaaaacccattttggattctcagtgttttgcttgtcacttccaagcatcactctcatggcttccatcattgaatctggttggctgtatctgatcacgcatttcagcgacttccaactagctagtatgggaagtttccttcttcatgaaagtgttttcttcttatctgggcttccatttatatatcttgaaagagctggattgctaagcaaatacaagattcagacaaaaaacaatacccctactgctcaagaaaaatgtatcactcgccttcttttgtatcattttagcgtcaatttaccgcttatgattgtctcgtatcccgtcttcagatgcatgggcatgagaagtagtctaccattgccatcctggaaagtggttctaactcagataatattctacttcattctggaggattttgtgttttactggggacatcgtgttttacatacaaaatggctgtacaagcatgtacacagtgtccatcatgaatatgcaacaccattcggactgacatcggaatatgctcacccagctgagatattgttccttggctttgccacaattgttggtcctgccatcaccggcccacatctgattactctctggttgtggatggttcttagagtcctcgagacagttgaggcacattgtggttaccattttccatggagcctctcgaactttcttcctttatatgggggtgctgattttcatgactatcatcatcgtttgctttacacgaaatctggcaactactcatccactttcatttacatggactggatattcggtaccgataaaggctacagaaagctgaaagcactaaaacacaacggagtcggagtcgaagatgacagcgagcaaacataatggagagaaaactagtttcgagtttgagggttcgggatgatttattaatcgtttaatgactttaggtataatttatcttttttatttcaacagtaataaagtgtcatgttattgtttccataggtaataatttggggtaaaaaaaaattaaaagtagtgaaatataattttaattaaatatgcctttaaaatatgttttcaaataaataaaaaatatatatattcagagtcg

SEQ ID NO: 163 - protein, 271 aa, Gossypium hirsutum
  misc_feature: Ceres Clone ID no. 1850745
MASIIESGWLYLITHFSDFQLASMGSFLLHESVFFLSGLPFIYLERAGLLSKYKIQTKNNTPTAQEKCITRLLLYHFSVNLPLMIVSYPVFRCMGMRSSLPLPSWKVVLTQIIFYFILEDFVFYWGHRVLHTKWLYKHVHSVHHEYATPFGLTSEYAHPAEILFLGFATIVGPAITGPHLITLWLWMVLRVLETVEAHCGYHFPWSLSNFLPLYGGADFHDYHHRLLYTKSGNYSSTFIYMDWIFGTDKGYRKLKALKHNGVGVEDDSEQT

SEQ ID NO: 164 - protein, 269 aa, Gossypium arboreum; identical to SEQ ID NO: 15
  misc_feature: Public GI no. 27448145

SEQ ID NO: 165 - DNA, 801 bp, Populus balsamifera subsp. trichocarpa
  misc_feature: Ceres ANNOT ID no. 1492747
atggttttctttttgatgcagtatttgatcacgcacttcagtgattttcagttggcatgtcttggaagtttctttcttcatgaaagcgtcttcttcttgtctggacttcctttcatatatctcgaaagggctggatggctgaaaaagtacaaaattcagatgaaaaacaacacccctgcagctcaggagaaatgtattgttcgcttactcttgtatcattttggtgttaatctaccagttatgctagcctcctatcctgtcttcagacacatgggcatgcaaagtagtcttccattcccgtcctggaaagtaattctaatgcagataacattctacttcatcctggaagattttatattctattggggacaccggttcttacatacaaaatggctgtacaagcatgtgcacagtatccatcatgaatatgctacaccatttggattaacttccgaatatgctcaccctgctgagatactgttccttggttttgctactattgttggtcctgccatcacaggcccccatctggtaactttgtggttatggatggtactaagggtcctggagacagttgaagcacattgtggttatcatttcccctggagcctctccaattttttacctttgtatggaggtgctgattttcatgactaccatcaccgcttgctgtatactaaatctggaaactactcatctactttcacgtacatggactgggtatttggtaccgataaaggttacagaaagctgcaagcattgaagaatgctggagtggaaaatggcggcaagcaaacctaa

SEQ ID NO: 166 - protein, 261 aa, Populus balsamifera subsp. trichocarpa
  misc_feature: Ceres ANNOT ID no. 1492747
MQYLITHFSDFQLACLGSFFLHESVFFLSGLPFIYLERAGWLKKYKIQMKNNTPAAQEKCIVRLLLYHFGVNLPVMLASYPVFRHMGMQSSLPFPSWKVILMQITFYFILEDFIFYWGHRFLHTKWLYKHVHSIHHEYATPFGLTSEYAHPAEILFLGFATIVGPAITGPHLVTLWLWMVLRVLETVEAHCGYHFPWSLSNFLPLYGGADFHDYHHRLLYTKSGNYSSTFTYMDWVFGTDKGYRKLQALKNAGVENGGKQT

SEQ ID NO: 167 - DNA, 721 bp, Gossypium hirsutum
  misc_feature: Ceres Clone ID no. 1940503
aattagcttcttctttttttcacttccaagctctcatggctgccattattgaatctgggtggctgtatctgatcacacatttcagcgacttccaactagcaagtctaggaagtttctttcttcacgaaagtgttttcttcttatccggacttccatttatatatctggaaagagctggattgctgagcaaatacaagattcagacgaaaaacaacagccttgctgctcaggaaaaatgtatcacccgcctgcttctgtatcacattggtgttaacctaccgcttatgattgcatcttatcctttcttcagattcatgggcatgaaaagtagtctgccatttccatcctggaaagtggttctatcacagataatattctatttcatcctggaggattttgtgttttactggggacatcgtattttacatacaaaatggctgtacaagcatgtgcacagtgttcatcatgagtatgcgactccatttggactgacatccgagtatgctcaccctgctgagatattgttccttggctttgcaacaataattggtcccgccatcactggcccacatcttattactctctggttatggatgactcttagagtcctagagacagttgaggcacattgtggttaccattttccatggagcctctcaaactttctacctttatatgggggtgctgattttcatgactatcatcatcgtttgctttatac

SEQ ID NO: 168 - protein, 228 aa, Gossypium hirsutum
  misc_feature: Ceres Clone ID no. 1940503
MAAIIESGWLYLITHFSDFQLASLGSFFLHESVFFLSGLPFIYLERAGLLSKYKIQTKNNSLAAQEKCITRLLLYHIGVNLPLMIASYPFFRFMGMKSSLPFPSWKVVLSQIIFYFILEDFVFYWGHRILHTKWLYKHVHSVHHEYATPFGLTSEYAHPAEILFLGFATIIGPAITGPHLITLWLWMTLRVLETVEAHCGYHFPWSLSNFLPLYGGADFHDYHHRLLY

SEQ ID NO: 169 - protein, 271 aa, Medicago truncatula
  misc_feature: Public GI no. 92875070
MASIFESGWQYLITHFSDFQLACLGSFFLHESVFFLSGLPFVWIERAGWLSKYKIQAKNNSPEAQDKCIVRLLLYHFGVNLPVMIFSYPVFRYMGMQSSLPLPSWNIILTQIMFYFILEDFIFYWGHRILHTKWLYKHIHSVHHEYATPFGLTSEYAHPAEILFLGFATIVGPAITGPHLITLWLWMVVRVLETVEAHCGYHFPWSPSNFLPLYGGSDFHDYHHRLLYTKSGNYSSTFTYMDRIFGTDVGYRKLKALKSREFEDSYEQKKQ

SEQ ID NO: 170 - DNA, 810 bp, Populus balsamifera subsp. trichocarpa
  misc_feature: Ceres ANNOT ID no. 1461748
atggcctccctcatcgaatcttgctggctgtatttgatcacgcatttcagcgattttcagttggcatgtcttggaagtttctttcttcatgaaagcatcttcttcttgtctggtattcctttcatatatcttgaaagggctggatggctgaaaaagtacaaaatccagatgaaaaacaacactcctgcatctcaagagaaatgtattattcgcctacttttatatcattttggtgttaacctaccagttatgctggcctcctatcctgtcttcagacacatgggcatgcaaagtagtcttccattcccgtcctggaatgtaattctaacgcagataacattctacttcatcctggaagattttatattctactggggacatcggattttacacacaaaatggctgtacaagcatgtgcacagtgttcatcatgaatatgctacaccatttggattaacttctgaatatgctcaccctgctgaaatactgttccttggctttgctaccattattggtcctgccatcactgggccccatctgcttactttgtggttatggatggtactaagggtcctggagacggttgaagcacattgtggttatcatttcccatggagcctctccaacttcttacctttgtatggaggtgctgattttcatgactaccatcaccggttgctgtatactaaatctggaaactactcatctactttcacctacatggactgggtattcggtaccgataaaggttacagaaagttgcaagcattgaagaatgctggagtggaaaatggcggcaagcaaatgtaa

SEQ ID NO: 171 - protein, 269 aa, Populus balsamifera subsp. trichocarpa
  misc_feature: Ceres ANNOT ID no. 1461748
MASLIESCWLYLITHFSDFQLACLGSFFLHESIFFLSGIPFIYLERAGWLKKYKIQMKNNTPASQEKCIIRLLLYHFGVNLPVMLASYPVFRHMGMQSSLPFPSWNVILTQITFYFILEDFIFYWGHRILHTKWLYKHVHSVHHEYATPFGLTSEYAHPAEILFLGFATIIGPAITGPHLLTLWLWMVLRVLETVEAHCGYHFPWSLSNFLPLYGGADFHDYHHRLLYTKSGNYSSTFTYMDWVFGTDKGYRKLQALKNAGVENGGKQM

SEQ ID NO: 172 - DNA, 1031 bp, Panicum virgatum
  misc_feature: Ceres Clone ID no. 1723971
aggagaccagaccaccaccaccaccactccacccccatcacagaaacagagagagccctgcctacaatggcggcggccctcgagtctggctggcaggtcctgaccgccaacttcaccgagttccagctcgccaccgtcttcaccttcctgatccacgaggccgccttcttcctctccggcctcccctccctcctcttcgagcgcttcgggctcttcgccaagtacaagattcagaagaagagcaacacctctgcttatcaaaacagatgtgtcctgcgtctcattctctaccatgtctgtgtgaacttgcctgtcgtgattttctcctaccctgccttcaaattcatgggccttaggagctctcttcctctgccacactggacggttgtcgtatctcaaattctattctactttgtactggaggattttgtattctattgggggcaccgggcactgcacaccaaatggctatacaagcatgtccacagcgtccaccacgaatatgctacaccctttgggttaacttctgaatacgcccaccccgctgaaattttgttcctgggattcgccacagttgttggccctgctcttactggccctcatttgttgaccctgtggctttgggtggtgttgagggtattggagacagttgaagctcacagcggctaccacttcccatggagcccctcaaatttcctgccactgtatggcggctcggacttccatgactaccatcaccgtgtgctctacaccaagtcagggaactattcctcgacttttgtgtacatggactggttgtttgggacggacaaggattaccgcaaggtgaaggccatggaggagaaggaagggaacaaggatatgtagatgatgcatgcatgggatctgcgaataatgaagacaggaggctattagcagcacgtactgagtactacaatcgtacgtaatcctggggatttgtgttagttcctgctttaatttgtttgggcgggatcggatgtatatacatgggatgggtttggatcggagct

SEQ ID NO: 173 - protein, 266 aa, Panicum virgatum
  misc_feature: Ceres Clone ID no. 1723971
MAAALESGWQVLTANFTEFQLATVFTFLIHEAAFFLSGLPSLLFERFGLFAKYKIQKKSNTSAYQNRCVLRLILYHVCVNLPVVIFSYPAFKFMGLRSSLPLPHWTVVVSQILFYFVLEDFVFYWGHRALHTKWLYKHVHSVHHEYATPFGLTSEYAHPAEILFLGFATVVGPALTGPHLLTLWLWVVLRVLETVEAHSGYHFPWSPSNFLPLYGGSDFHDYHHRVLYTKSGNYSSTFVYMDWLFGTDKDYRKVKAMEEKEGNKDM

SEQ ID NO: 174 - DNA, 1139 bp, Panicum virgatum
  misc_feature: Ceres Clone ID no. 1953785
gactaccaccacaccgtcacccccatcacagaaagaaagagagagagccctacctacaatggcggcggccctcgagtctgcctggcaggtcctgaccgccaacttctccgagttccagctcgccaccgtcttcaccttcctgatccacgagaccgccttcttcctctccggcctcccctccctcctcttcgagcgcttcggactcttcgccaagtacaagattcaaaagaagagcaacacctctgcttatgaaaacagatgtgtcctgcgcctcatactctaccatgtctgtgtgaacttgcctgtcatcattttctcctaccctgccttcaaattcatgggccttaggagctctcttcctctgccacactggacggttgttgtatctcaagttgcattctactttgtactggaggattttgtattctactgggggcaccgggcactgcacaccaaatggctatacaagcatgtccacagcgtccaccacgaatatgctacaccctttgggttaacttctgaatacgcccaccctgctgagattttgttcctgggattcgccacagttgttggtcctgctcttactggccctcatttgttgaccctgtggctttgggtggtgttgagggtattggagacagttgaagctcacagcggctaccacttcccatggagcccctcaaatttcctgccactgtatggaggctcggacttccatgactaccatcaccgtgtgctctacaccaagtcagggaactatgcctcgacttttgtgtacatggactggttgtttgggacggacaaggattaccgcaaggcgaaggccatggaggagaaggaagggaacaaggatacgtagatcgatcatgcatgggagctgcgaataatgaagacaggcggagctagcaatacgtactgtctgtgtactacaatcgtacgtacgtaatccttggggtttgccgtttgcgttcctgctttgtttaatttggccgggatcggatgtatatatacatttggatcggagctagctagtagcaagatggtcgatgaggttgatgtgaccttagtttgcttgtgtgatgaattaacaataatgtcagcaattaaataaagccacaactatagccggcctgttcagc

SEQ ID NO: 175 - protein, 266 aa, Panicum virgatum
  misc_feature: Ceres Clone ID no. 1953785
MAAALESAWQVLTANFSEFQLATVFTFLIHETAFFLSGLPSLLFERFGLFAKYKIQKKSNTSAYENRCVLRLILYHVCVNLPVIIFSYPAFKFMGLRSSLPLPHWTVVVSQVAFYFVLEDFVFYWGHRALHTKWLYKHVHSVHHEYATPFGLTSEYAHPAEILFLGFATVVGPALTGPHLLTLWLWVVLRVLETVEAHSGYHFPWSPSNFLPLYGGSDFHDYHHRVLYTKSGNYASTFVYMDWLFGTDKDYRKAKAMEEKEGNKDT

SEQ ID NO: 176 - DNA, 1176 bp, Panicum virgatum
  misc_feature: Ceres Clone ID no. 1804072
agctacagcttaaacacaagaagaagaggagaggagaggagaggagtggagtggagaggagactaccaccacaccgtcacccccatcacagaaagaaagagagagagccctacctacaatggcggcggccctcgagtctgcctggcaggtcctgaccgccaacttctccgagttccagctcgccaccgtcttcaccttcctgatccacgagaccgccttcttcctctccggcctcccctccctcctcttcgagcgcttcggactcttcgccaagtacaagattcagaagaagagcaacacctctgcttatcaaaacagatgtgtcctgcgtctcatactctaccatgtctgtgtgaacttgcctgtcatcattttctcctaccctgccttcaaattcatgggccttaggagctctcttcctctgccacactggacggttgttgtatctcaagttgcattctactttgtactggaggattttgtattctactgggggcaccgggcactgcacaccaaatggctatacaagcatgtccacagcgtccaccacgaatatgctacaccctttgggttaacttctgaatacgcccaccctgctgagattttgttcctgggattcgccacagttgttggtcctgctcttactggccctcatttgttgaccctgtggctttgggtggtgttgagggtattggagacagttgaagctcacagcggctaccacttcccatggagcccctcaaatttcctgccactgtatggaggctcggacttccatgactaccatcaccgtgtgctctacaccaagtcagggaactatgcctcgacttttgtgtacatggactggttgtttgggacggacaaggattaccgcatggtgaaggccatggaggagaaggaagggaacaaggatatgtagatgatgcatgcatgggatctgcgaataatgaagacaggcggagctagcaatatgtactgtctgtgtactacaatcgtacgtacgtaatccttggggtttgtgttcctgctttgtttaatttggccgggatcggatgtatatatacatttggatcggagctagctagtagcaagatggtcgatgaggttgatgtgaccttagtttgcttgtgtgatgaattaacaataatgtcagcaattaaataaagccacaactgc

SEQ ID NO: 177 - protein, 266 aa, Panicum virgatum
  misc_feature: Ceres Clone ID no. 1804072
MAAALESAWQVLTANFSEFQLATVFTFLIHETAFFLSGLPSLLFERFGLFAKYKIQKKSNTSAYQNRCVLRLILYHVCVNLPVIIFSYPAFKFMGLRSSLPLPHWTVVVSQVAFYFVLEDFVFYWGHRALHTKWLYKHVHSVHHEYATPFGLTSEYAHPAEILFLGFATVVGPALTGPHLLTLWLWVVLRVLETVEAHSGYHFPWSPSNFLPLYGGSDFHDYHHRVLYTKSGNYASTFVYMDWLFGTDKDYRMVKAMEEKEGNKDM

SEQ ID NO: 178 - DNA, 663 bp, Populus balsamifera subsp. trichocarpa
  misc_feature: Ceres ANNOT ID no. 1500813
atggaggggcagccgcatccatacgcaccaagagatctgaaactgcctggctatgtacctaatttcctcactcaatccaccattgtcggcgtctatttactcacctcccttcttgtcgtctctctcatctggatcctctctggaaggtctcgtaagataacaaaaatgaataggttgctcatgtgctggtgggctttcactggtctcacacacttgattctcgagggttattttgctttctctccagaattttacaaggacaagactgctcattatctggctgaagtttggaaagaatatagcaaaggtgattcaagatatgcagcaagggatgctgcaacagttactgttgaaggattgactgctgttctcgagggaccagctagcctcctggcagtgtatgctattgcttcaggaaaatcgtacagctacatacttcagtttgctgtttgtttgggacagctctatggaacagctgtgtatttcttaactgcctacttggaaggtgatcattttgctaccagtccgtaccactattacgtatactatattggtgcaaatgcctcctgggttgtaattccctcgctcatcgctatgcgttgttggaaaaagatttgctcagcagtccaagttcatggccagaaaaggaccaaaactcgttga

SEQ ID NO: 179 - protein, 220 aa, Populus balsamifera subsp. trichocarpa
  misc_feature: Ceres ANNOT ID no. 1500813
MEGQPHPYAPRDLKLPGYVPNFLTQSTIVGVYLLTSLLVVSLIWILSGRSRKITKMNRLLMCWWAFTGLTHLILEGYFAFSPEFYKDKTAHYLAEVWKEYSKGDSRYAARDAATVTVEGLTAVLEGPASLLAVYAIASGKSYSYILQFAVCLGQLYGTAVYFLTAYLEGDHFATSPYHYYVYYIGANASWVVIPSLIAMRCWKKICSAVQVHGQKRTKTR

SEQ ID NO: 180 - DNA, 630 bp, Populus balsamifera subsp. trichocarpa
  misc_feature: Ceres ANNOT ID no. 1491102
atggaggggcagccgcatccatacgcaccaacagatctcaaactgcctggctatgttcctaatttcctcactcaattcaccattgtctctgtctatggactctcctctctcatctggatcctctctggaaggtctcgtaaattagccaaaattgatagattgctcatgtgctggtgggctttcactggtctcacacacgtgattcttgagggttattttgttttctctccagaattttacaaggacaagactgctcattatctagctgaagtttgtgattcaagatatgctgcaagggatgctgcaacggttactgttgaaggagtgactgttgtttttgagggtcgagctagtctcctggcagtgtgtgctattgcttcaggaaaatcatacagctacatacttcagttttccgtttctttgggacagctctatggaacagctgtgtatttcctaaccgcctacttggaaggtgatcactttgctgcaagttcatatcactgttatgtgtactatatcggtgcaaatgcctcctgggttgtaataccctcactcatcgctattcgttgttggaacaagattttttcagcagtccaagttcaaggccagaaaaagaccaaaagtcactga

SEQ ID NO: 181 - protein, 209 aa, Populus balsamifera subsp. trichocarpa
  misc_feature: Ceres ANNOT ID no. 1491102
MEGQPHPYAPTDLKLPGYVPNFLTQFTIVSVYGLSSLIWILSGRSRKLAKIDRLLMCWWAFTGLTHVILEGYFVFSPEFYKDKTAHYLAEVCDSRYAARDAATVTVEGVTVVFEGRASLLAVCAIASGKSYSYILQFSVSLGQLYGTAVYFLTAYLEGDHFAASSYHCYVYYIGANASWVVIPSLIAIRCWNKIFSAVQVQGQKKTKSH

SEQ ID NO: 182 - DNA, 1992 bp, Gossypium hirsutum
  misc_feature: Ceres Clone ID no. 1918478
aacatatatcctctcttaaagccgtagcctttattttttttgctagacaaagagtgtatcttttaagttaatgagactgtaatttttttttctgaaagctgtctttgtcatcattttcttcgtttttctctctctctctatcagaccatgaattcataatcatcgtcgtcgtcgtctccttcatcgatatatctctttcgaatacgtttagaggaaaaaggaaaaggaaaaagggtacatacatggcgtttcaccatagcagcacccacctttctcaagaccttcctctccatcactttaccgaccagcaacagcaacagcaacaacccaaccaaacccagcaaggcgaccaacttcaggaaaccgccgctcccaattggctaaactccgcccttctccgtccccaacagccgcacccaccgcaaccccacccgcacttctccgaccctaactttcttaaccttcatactactacaactgcttccgactccaccgcggcttcccaagctcctaacccgatgttctcccgttcatcctcgtcgcttcttcatcgaaaccacggcaacgtcatcgacgacgcggctgctgcggcagcagctgcggtaggaggaggtgttatggccgtggaatcgggtgatttgaagaacagcatcagcgagactatgaataacaataagagcgaaggcgtggtggtggagagtggaggaggaggaggcggagatgggattgtgaattttcagaatgcgagatacaaggcagagattctagctcacccattttacgagcaactattgtcagcacacgtggcgtgccttaggatcgccacgccagtggatcagcttccgaggatcgacgctcagctggctcagtcgcagcatgtggtggctaaatactcagctctcggtggagggtcgcagggcttggttagtgatgacaaagaactcgatcagttcatgacgcattatgttctgttgttatgctcgtttaaagaacaattacaacaacatgttcgtgttcatgcgatggaagcagtgatggcttgctgggagattgaacaatccttacaaagcttaacaggtgtttccccaggggaaggaacaggtgctacaatgtctgatgatgatgatgatgaccaagtcgacagtgatgccaacttgtttgatacaagtttggatggtacagattcaatggggtttggacctttgatcccaacagaaagtgaaaggtctttgatggagcgtgtgaggcatgaactcaaacatgaactcaaacagggttacaaggagaagatcgtggacataagggaggaaattttgcgaaaaagaagggccggaaaacttcccggtgatacaacatcggttttaaaagcttggtggcagtcacattccaagtggccttaccctactgtaagttctacttcaccatgatctctttaattaaactctaaatgttaactaatttttcttttgcaggaggaagataaggcaaggttggttcaagaaacaggtttacagttaaaacagataaacaattggttcatcaatcaaaggaagaggaactggcatagcaatccatcgacttccaccgtctccaagaccaaacgtaaaaggtgaaaacataaggtgatcggttcgtgttaaaccaaatcaaagattaaacccaacattacttcttacatagatcacaagatatataggcttttagtggaactgcagcagaggcttaaaaggccattttgcaggtatgcgagattggaggtattggtagggaagtttaggaaaattataattagtgtaacttttaattggatgaaattgtagttgcgtcagtagattatgatttttggtatgtatgtctctttcatatagatgtgaataaattgggagtgtacagtgtaataagctttatacttggttacggccatggtagatcattgtagcctttttgacattgtaatgcagagtaaaattaggtctttgctctttctctttt

SEQ ID NO: 183 - protein, 394 aa, Gossypium hirsutum
  misc_feature: Ceres Clone ID no. 1918478
MAFHHSSTHLSQDLPLHHFTDQQQQQQQPNQTQQGDQLQETAAPNWLNSALLRPQQPHPPQPHPHFSDPNFLNLHTTTTASDSTAASQAPNPMFSRSSSSLLHRNHGNVIDDAAAAAAAAVGGGVMAVESGDLKNSISETMNNNKSEGVVVESGGGGGGDGIVNFQNARYKAEILAHPFYEQLLSAHVACLRIATPVDQLPRIDAQLAQSQHVVAKYSALGGGSQGLVSDDKELDQFMTHYVLLLCSFKEQLQQHVRVHAMEAVMACWEIEQSLQSLTGVSPGEGTGATMSDDDDDDQVDSDANLFDTSLDGTDSMGFGPLIPTESERSLMERVRHELKHELKQGYKEKIVDIREEILRKRRAGKLPGDTTSVLKAWWQSHSKWPYPTVSSTSP

SEQ ID NO: 184 - DNA, 1359 bp, Populus balsamifera subsp. trichocarpa
  misc_feature: Ceres ANNOT ID no. 1531214
atggcctatcaccataacttgtcatcacaagacctccctcttcaccacttcacagaccaacaagcaacagagaaccacacagcaccaccgaattggctgaacactgccctcctccgctctcaacaaccaccacagcaacaaactcaccaccacttcactgataacaacaacacaaacaatttcttaaacctccacaccgccaccactaccgccaccgccaccacttctgactcaaactctcataacccagttcaatggctctcccggtcctcctcctccctcctaaaccgcaaccacagtgacgtcatcgacgacgtcgccgccggcggggaccacgccatcataactagtatatcgcaagaatcgtcggaattgaagaatatgaataagggtgaaggtgaagccatggatagtggtggaggagagtcggtggtgaattggcaaaatgcaaggtataaagcggacatattgacgcatccgttgtacgatcaattgctgtcggcacacgtggcgtgtttaaggattgccacgccagttgatcagttgccgaggattgatgcacagttggctcagtcacagcaagtagtaactaagtactctgctcttggaagtcaccaaggattggttcctgatgataaagagcttgatcagtttatgacacattattttcttttgctctgttccttcaaagaacaattgcaacaacatgttcgagttcatgcaatggaagcagtgatggcgtgctgggagatagagcaatccctacagagtttaacgggagtttctccaggtgaaggtacaggcgcaacaatgtccgatgacgatgaagaccaagttgacagtgatgccaatttgttcgttggaagtttggagggtgcagatacactggggtttggtcccttggtccctacagagagtgagagatctttgatggagcgtgtgagacaagaattgaagcatgaattaaaacagggttacaaagaaaaaattgttgacattagagaggaaattctgcgaaagagaagagcaggaaagcttcctggggacacaacctcagtcttaaaagcttggtggcaatcacattccaagtggccatatcctaccgaggaagacaaggcaagattggtgcaggaaacgggcttgcaattaaagcagataaataattggttcatcaatcaaaggaagaggaactggcacagtaatccttcaacctcaacagtcttgaaaagcaaacgcaaaagaaaaactctactgagcctgggcaaggaaaaacaaaccctatcgatcaatgcagaagttgaagcaaccagcccccttgaagagggttggtacgcaggagtgactgattccagtcattga

SEQ ID NO: 185 - protein, 452 aa, Populus balsamifera subsp. trichocarpa
  misc_feature: Ceres ANNOT ID no. 1531214
MAYHHNLSSQDLPLHHFTDQQATENHTAPPNWLNTALLRSQQPPQQQTHHHFTDNNNTNNFLNLHTATTTATATTSDSNSHNPVQWLSRSSSSLLNRNHSDVIDDVAAGGDHAIITSISQESSELKNMNKGEGEAMDSGGGESVVNWQNARYKADILTHPLYDQLLSAHVACLRIATPVDQLPRIDAQLAQSQQVVTKYSALGSHQGLVPDDKELDQFMTHYFLLLCSFKEQLQQHVRVHAMEAVMACWEIEQSLQSLTGVSPGEGTGATMSDDDEDQVDSDANLFVGSLEGADTLGFGPLVPTESERSLMERVRQELKHELKQGYKEKIVDIREEILRKRRAGKLPGDTTSVLKAWWQSHSKWPYPTEEDKARLVQETGLQLKQINNWFINQRKRNWHSNPSTSTVLKSKRKRKTLLSLGKEKQTLSINAEVEATSPLEEGWYAGVTDSSH

SEQ ID NO: 186 - protein, 225 aa, Zea mays subsp. mays
  misc_feature: Ceres Clone ID no. 1374536
MPSFAFGSHHHLANPTDSPYTVEISIDGDSSDLDSLSEVDLESGGVTKLHSGGGKKRRTRRRKKKKKRKKKESRDCRICHLPLETTKKADEEGEDSDEQEEQGEEEDEARDGEEEEEYYGLPLQLGCSCKGDLGVAHSKCAETWFKIKGNMTCEICGAMAINVAGEQSNPESTASTHSQVAAGQTQSSQTEPRGIWHGRRVMNFLLAAMVFAFIVSWLFHFKVLK

SEQ ID NO: 187 - DNA, 1126 bp, Gossypium hirsutum
  misc_feature: Ceres Clone ID no. 1896529
aaaaaaaccctaaattctcacttctttcactttcaaaaccaaaactagaagatcggggagagaagatggcggccggcgatcacaccgttttgcagctcagtacgccttcaacggcgaacctatcggccaaggttcatcctctcgtcatctttaacatctgcgattgctacgttagacgccccgaccaagccgagcgcgtaattggcacgctcctcggctccgtccttcctgatggcaccgttgatatccgtaactcctatgccgttcctcacactgaatccgccgaacaggttgctttggatattgaataccatcataatatgttagtctcccaccaaaaagtgaatccaaaggaagtaattgttggatggtattctactggccttggagtcacaggtggtagtgcattgatccacgatttttattctagggaagtcccgaaccctgttcatttgacagtggatacaggatttaggaatggagagggcacgataaaggcctacgtttctgtcaatttagctcttggagaccgacagcttgctgctcaattccaagaaattccccttgatctacgtatggttgaagctgagcgactggggtttgacatcctgaagacaacagcggttgacaaactgccgaatgatttggaaggaatggaagtcacaatgcaaagactgttggctttaatagatgatgtctacaaatacgtagatgatgttgtggaggggcgtgttgctgctgataatagcatcggcagatttatatcagacacagtagcctccttgcctaaactatcgacatccgtatttgataagcttgtgaatgatagtctgcaggatcagttgctgttattatacttatcgagcatcactaggacacaacttggattagctgagaagttgaacacagctgcacagatcctgtgatttgggtccaaagtgatgcatttttgttcttgggcgaatcacgtgctttgattgtgggaaactttagctggagaaagactgactatatgttattattattggcggtttaaaatgacctctatagtttctgtaagttgtccttttctcctaaactacatgtcctacagctgtgaggttgaattaaatttcgcttttaattc

SEQ ID NO: 188 - protein, 286 aa, Gossypium hirsutum
  misc_feature: Ceres Clone ID no. 1896529
MAAGDHTVLQLSTPSTANLSAKVHPLVIFNICDCYVRRPDQAERVIGTLLGSVLPDGTVDIRNSYAVPHTESAEQVALDIEYHHNMLVSHQKVNPKEVIVGWYSTGLGVTGGSALIHDFYSREVPNPVHLTVDTGFRNGEGTIKAYVSVNLALGDRQLAAQFQEIPLDLRMVEAERLGFDILKTTAVDKLPNDLEGMEVTMQRLLALIDDVYKYVDDVVEGRVAADNSIGRFISDTVASLPKLSTSVFDKLVNDSLQDQLLLLYLSSITRTQLGLAEKLNTAAQIL

SEQ ID NO: 189 - DNA, 1077 bp, Gossypium hirsutum
  misc_feature: Ceres Clone ID no. 1932777
atacaaattatattaaaattccactccaaaaccctaaattctttgctcttcttcactcagttcactgaaaccgtaaggagaagatcggagagaaaatatggcggccggcgatcgcaccgttttgcagtttaactcaccgtcatcggcgagcctgtcagcgaaagttcaccctctcgtcatatttaacatctgcgattgctacgtcaggcgtcccgaccaagccgagcgcgtcatcggcacgcttctcggctccgtcctccctgacggaaccgttgatatccgtaactcctatgcagttcctcacacggagtccgctgaacaggttgctttggatattgaataccatcataatatgttagtctcccaccaaaaagtgaatcctaaagaagtcattgttggatggtattctaccgggctaggagtcacaggcggcagtgcattgatccatgatttttattctagggaagtatctaaccctattcatttgacggtggatacggggtttaggaatggagagggtaccgtaaaggcttttgtttctgtcaatttagctcttggagacagacaacttgcagctcaatttcaagaaattcctcttgatctatgtatggttgaagctgagcgagttggatttgacatcctcaagaaaacagcagtcgacaaatttccaaatgatttggaaggaatggaagtcacaatgggaaggctatcggctttgatagacgatgtctacaaatacgttgacgatgttgtggaaggccgtgttccagccgataacaacataggtagatttatagcagaaacagtagcttccttacctaaactatctcccccagctttcgataaactcatgaatgatagcctacaggatcaattgctgttactatacttatcgagcatcgctaggactcaactcgggttagccgaaaagttgaatacggcagctcagattctgtaaacttacttccaagtgattaatttttggatctggcattttcagttttttatttatttttaactaatagcctgtagttttgctaagatgcttcttttcctttaacgaatagcctttgtttc

SEQ ID NO: 190 - protein, 286 aa, Gossypium hirsutum
  misc_feature: Ceres Clone ID no. 1932777
MAAGDRTVLQFNSPSSASLSAKVHPLVIFNICDCYVRRPDQAERVIGTLLGSVLPDGTVDIRNSYAVPHTESAEQVALDIEYHHNMLVSHQKVNPKEVIVGWYSTGLGVTGGSALIHDFYSREVSNPIHLTVDTGFRNGEGTVKAFVSVNLALGDRQLAAQFQEIPLDLCMVEAERVGFDILKKTAVDKFPNDLEGMEVTMGRLSALIDDVYKYVDDVVEGRVPADNNIGRFIAETVASLPKLSPPAFDKLMNDSLQDQLLLLYLSSIARTQLGLAEKLNTAAQIL

SEQ ID NO: 191 - DNA, 864 bp, Populus balsamifera subsp. trichocarpa
  misc_feature: Ceres ANNOT ID no. 1464743
atggcggcgatcgagcagcaaacagtcctccaattcgcaccatcatcttcatcgaccctctccgcgaaggttcaccctcttgtcatcttcaacatctgcgattgctacgttaggcggcctgaccaagctgagcgcgtcattggcactctccttggctccgtcttgcccgatggcaccgttgatatccgcaactcttatgccgtccctcataatgaatcctccgaacaggtggctttggatatcgattaccatcataacttgctgctgtctcatcaaaaagtgaatcctaaggaagttattgttggatggtattcaactgggttaggagttactggcggtagtgctttgattcatgatttctattccagagaagtcccgaaccctattcatctgacggtggatacaggattcagtaatggggagggtaccataaaggcttatgtttctgtgaatctgtctcttggagaccgtccgcttgcggcacaatttcaagaagttcctcttgatcttcgaatggttgaagctgagcgcattggatttgatgttttgaagactacaatggttgacaaaattccaagtgatttggaagggatggaaatctctatgcaacggctactagctctaattgatgatgtgtacaaatatgttgatgatgtggtggaagggcacactgcaccagataataacataggacgttttatatcagacacggtcgcctcccttcccaaactttcaccaccagtgtttgataagcttgtaaatgacaatgtgcaggaccatttactcttgctgtatctgtcaagcatcactaggacacagctcagcttagcagagaagttgaatacagctgctcagatcctctga

SEQ ID NO: 192 - protein, 287 aa, Populus balsamifera subsp. trichocarpa
  misc_feature: Ceres ANNOT ID no. 1464743
MAAIEQQTVLQFAPSSSSTLSAKVHPLVIFNICDCYVRRPDQAERVIGTLLGSVLPDGTVDIRNSYAVPHNESSEQVALDIDYHHNLLLSHQKVNPKEVIVGWYSTGLGVTGGSALIHDFYSREVPNPIHLTVDTGFSNGEGTIKAYVSVNLSLGDRPLAAQFQEVPLDLRMVEAERIGFDVLKTTMVDKIPSDLEGMEISMQRLLALIDDVYKYVDDVVEGHTAPDNNIGRFISDTVASLPKLSPPVFDKLVNDNVQDHLLLLYLSSITRTQLSLAEKLNTAAQIL

SEQ ID NO: 193 - DNA, 1105 bp, Zea mays subsp. mays
  misc_feature: Ceres CLONE ID no. 217004
gagacaaacgccacattccgccacctcctctcccaaaggctcgccctccccggctcccctcgtatcttcgccgtcggcgatcaaaatggcggtgcccatgtccgccatcgagtccgcgtggcagctcctgatcgccaacttcaccgagttccagctcgccaccgtcatcaccttcctgctccacgagaccgtcttctttctctctggccttccctccctcctcttcgagcgcttcggactcttcgccaaatacaagatccagaagaggagcaacacctctgcttaccaaaacagatgtgtcttgcgtcttattctgtaccatgtctctgtgaacctgcctgtcatgattttgtcgtaccctgccttcaaattcatgggtcttaggagctctcttcctctaccacattggacggttgttgtatctcaagttcttttctactttgtccttgaggattttatattctactgggggcacagggcactgcatacgaaatggctatacaaacatgttcacagcgtccaccacgagtacgccacaccctttggtttaacttccgaatatgcccacccagctgaaattttgttcctgggattcgccacagttgttggtcctgctcttactggccctcatctgttcaccctgtggctgtggatggtgttgagggtattggagactgttgaagctcacagcggctatcacttcccatggagcccatcaaatttcctgccactgtacggtggctcggacttccatgactaccatcaccgagtgctgtacacaaagtcagggaactatgcctcgacatttgtttacatggactggttgttccggacggacaatggttatcgcaaggcaaagagaccattgaggagcaagaagtgaagaagaagaagaatctgtaaagtgttgaagctgctcatcaacaggactggcgatagagttgcgcctcatcatggaaggagagaagatggatgcagtcagttattgcctgacgaccaatactataggctcctgagatgttgatttccctgtgttttctatgatcaagaacgaggtcctggcgaccttggtctgtcatgaactgaatttgataaaaaaattgtc

(19) United States
(12) Patent Application Publication
(10) Pub. No.: US 2009/0178160 A1
(43) Pub. Date: Jul. 9, 2009

(54) Modulation of Triterpenoid Content in Plants

(76) Inventors: Joon-Hyun Park, Oak Park, CA (US); Kenneth Feldmann, Newbury Park, CA (US); Amr Saad Ragab, Woodland Hills, CA (US); Steven Craig Bobzin, Malibu, CA (US); Boris Jankowski, Newbury Park, CA (US); Jennifer E. Van Fleet, San Jose, CA (US)

Correspondence Address: FISH & RICHARDSON P.C., PO BOX 1022, MINNEAPOLIS, MN, 55440-1022, US

(21) Appl. No.: 12/091,429

(22) PCT Filed: Oct. 24, 2006
(86) PCT No.: PCT/US06/41516

§ 371 Date: Jul. 29, 2008

Related U.S. Application Data
(60) Provisional application No. 60/730,079, filed on Oct. 25, 2005.

(51) Int. Cl. C12N 15/11; A01H 5/00; C07C 1/00

(52) U.S. Cl. 800/287; 800/278; 585/16; 800/298; 536/23.1; 585/240

(57) ABSTRACT

Compositions and methods for producing triterpenoid compounds, e.g., squalene, are disclosed.

### CROSS-REFERENCE TO RELATED APPLICATIONS

This Application claims priority under 35 U.S.C. § 119 to U.S. Provisional Application No. 60/730,079, filed Oct. 25, 2005, incorporated herein by reference in its entirety.

### TECHNICAL FIELD

This document relates to materials and methods for modulating triterpenoid content in plants. More particularly, the invention relates to materials and methods for modulating the amount of one or more triterpenoid compounds in plants, based on expression of triterpenoid-modulating polypeptides that facilitate changes in the amounts of such compounds in plants.

### INCORPORATION-BY-REFERENCE & TEXTS

The material on the accompanying diskette is hereby incorporated by reference into this application. The accompanying compact discs are identical and contain one file, 11696-176WO1—Sequence.txt, which was created on Oct. 23, 2006. The file named 11696-176WO1—Sequence.txt is 399 KB. The file can be accessed using Microsoft Word on a computer that uses Windows OS.

### BACKGROUND

Triterpenoids are an important class of metabolites distinguished by a wide range of structural diversity, physiological function, and biological activity. Triterpenoid molecules play critical roles in many normal cellular and developmental processes in both plants and animals. In addition, triterpenoids have significant pharmaceutical and neutraceutical applications. Triterpenoids, in both natural and synthetic forms, have been shown to have cholesterol lowering, anticoagulant, anticarcinogenic, hepatoprotective, immunomodulatory, anti-inflammatory and antioxidant activities. Some triterpenoids, for example digoxin and its derivative, digitoxin, are widely used in the treatment of various heat conditions. Other triterpenoids, for example, diosgenin, serve as starting materials in the production of steroids used in contraceptives. Particular plant-derived triterpenoids, the phytosterols, for example, sitostanol, β-sitosterol and stigmasterol, have been shown to have cholesterol lowering properties in humans and so play a valuable role in human nutrition.

Plants can serve as natural sources of triterpenoid molecules. In light of the wide variety of useful applications of these molecules, it is desirable to produce plants having modulated levels of triterpenoids.

### SUMMARY

Disclosed herein are materials and methods for expressing triterpenoid-modulating polypeptides that are capable of modulating amounts of triterpenoids in plants. Modulation can include an increase in the amount of triterpenoids relative to basal or native states (e.g., a control level). In other cases, modulation can include a decrease in the amount of triterpenoids relative to basal or native states, such as the level in a control.

Terpenoids are a diverse class of metabolites derived from five-carbon isoprene units. Terpenoids can be classified according to the number of isoprene units they contain. The triterpenoids generally are built from six isoprene units. Modification of the basic triterpenoid structure can include methylation and demethylation. Depending upon how the isoprene units are assembled, a triterpenoid can be acyclic (e.g. squalene), cyclic or polycyclic including, without limitation, tetra, penta and hexacyclic triterpenoids and their corresponding glycoside derivatives, the triterpene saponins. As used herein, the triterpenoids also include steroids and sterol compounds, as well as their glycoside derivatives, the steroidal saponins.

Provided herein are methods of altering the level of triterpenoid in a plant. The methods can include introducing into a plant cell an exogenous nucleic acid comprising a nucleotide sequence encoding a polypeptide having 80% or greater sequence identity to an amino acid sequence selected from the group consisting of SEQ ID NO: 35, SEQ ID NOS: 37-47, SEQ ID NO: 53, SEQ ID NOS: 55-61, SEQ ID NOS: 49-51, SEQ ID NOs: 2-12, SEQ ID NOs: 14-21, SEQ ID NOs: 23-26, SEQ ID NOS: 28-33, and the consensus sequences set forth in FIG. 2, 4, 5, 6, 7, 8, or 9, where a tissue of a plant produced from the plant cell has a difference in the level of a triterpenoid as compared to the corresponding level in tissue of a control plant that does not comprise the nucleic acid. The sequence identity can be 80%, 85%, 90%, 95% or greater.

In another embodiment, the methods can include introducing into a plant cell an exogenous nucleic acid comprising a nucleotide sequence encoding a polypeptide having 80% or greater sequence identity to an amino acid sequence selected from the group consisting of SEQ ID NO: 35, SEQ ID NO: 37, SEQ ID NO: 42, SEQ ID NO: 53, SEQ ID NO: 55, SEQ ID NOS: 57-60, SEQ ID NOS: 49-50, SEQ ID NO: 2, SEQ ID NO: 14, SEQ ID NO: 23, SEQ ID NO: 28, and the consensus sequences set forth in FIG. 2, 4, 5, 6, 7, 8, or 9, where a tissue of a plant produced from the plant cell has a difference in the level of a triterpenoid as compared to the corresponding level in tissue of a control plant that does not comprise the nucleic acid. The sequence identity can be 80%, 85%, 90%, 95% or greater. In another embodiment, a method of altering the level of a triterpenoid in a plant can include introducing into a plant cell an exogenous nucleic acid comprising a nucleotide sequence can encodes a polypeptide having 80% or greater sequence identity to an amino acid sequence selected from the group consisting of SEQ ID NO: 35, SEQ ID NO: 37, SEQ ID NO: 53, SEQ ID NO: 55, SEQ ID NO: 49, SEQ ID NO: 3, SEQ ID NO: 16, SEQ ID NO: 24, and SEQ ID NO: 29. The sequence identity can be 80%, 85%, 90%, 95% or greater.

In a further embodiment, a method of altering the level of a triterpenoid in a plant is provided, the method comprising introducing into a plant cell: (a) a first exogenous nucleic acid comprising a nucleotide sequence encoding a polypeptide having 80% or greater sequence identity to an amino acid sequence selected from the group consisting of SEQ ID NO: 35, SEQ ID NOS: 37-47, SEQ ID NO: 53, SEQ ID NOS: 55-61, SEQ ID NOS: 49-51, and the consensus sequences set forth in FIG. 2, 4, or 5; and (b) a second exogenous nucleic acid comprising a nucleotide sequence encoding a polypeptide having 80% or greater sequence identity to an amino acid sequence selected from the group consisting of SEQ ID NOs: 2-12, SEQ ID NOs: 14-21, SEQ ID NOs: 23-26, SEQ ID NOS: 28-33, and the consensus sequences set forth in FIG. 6, 7, 8, or 9; where a tissue of a plant produced from the plant cell has a difference in the level of a triterpenoid as compared to the corresponding level in tissue of a control plant that does not comprise the first nucleic acid and the second nucleic acid. The sequence identity can be 80%, 85%, 90%, 95% or greater.

In a further embodiment, a method of altering the level of a triterpenoid in a plant is provided, the method comprising introducing into a plant cell: (a) a first exogenous nucleic acid comprising a nucleotide sequence encoding a polypeptide having 80% or greater sequence identity to an amino acid sequence selected from the group consisting of SEQ ID NO: 35, SEQ ID NOS: 37-47, SEQ ID NO: 53, SEQ ID NOS: 55-61, SEQ ID NOS: 49-51, and the consensus sequences set forth in FIG. 2, 4, or 5; and (b) a second exogenous nucleic acid comprising a nucleotide sequence encoding a polypeptide having 80% or greater sequence identity to an amino acid sequence selected from the group consisting of SEQ ID NO: 35, SEQ ID NOS: 37-47, SEQ ID NO: 53, SEQ ID NOS: 55-61, SEQ ID NOS: 49-51, and the consensus sequences set forth in FIG. 2, 4, or 5; provided that the first exogenous nucleic acid and the second exogenous nucleic acid are not the same, where a tissue of a plant produced from the plant cell has a difference in the level of a triterpenoid as compared to the corresponding level in tissue of a control plant that does not comprise the first nucleic acid and the second nucleic acid.

In a further embodiment, a method of altering the level of a triterpenoid in a plant is provided, the method comprising introducing into a plant cell: (a) a first exogenous nucleic acid comprising a nucleotide sequence encoding a polypeptide having 80% or greater sequence identity to an amino acid sequence selected from the group consisting of SEQ ID NOs: 2-12, SEQ ID NOs: 14-21, SEQ ID NOs: 23-26, SEQ ID NOS: 28-33, and the consensus sequences set forth in FIG. 6, 7, 8, or 9; and (b) a second exogenous nucleic acid comprising a nucleotide sequence encoding a polypeptide having 80% or greater sequence identity to an amino acid sequence selected from the group consisting of SEQ ID NOs: 2-12, SEQ ID NOs: 14-21, SEQ ID NOs: 23-26, SEQ ID NOS: 28-33, and the consensus sequences set forth in FIG. 6, 7, 8, or 9; provided that the first exogenous nucleic acid and the second exogenous nucleic acid are not the same, where a tissue of a plant produced from the plant cell has a difference in the level of a triterpenoid as compared to the corresponding level in tissue of a control plant that does not comprise the first nucleic acid and the second nucleic acid.

Examples of triterpenoids can include squalene, lupeol, α-amyrin, β-amyrin, glycyrrhizin, β-sitosterol, sitostanol, stigmasterol, campesterol, ergosterol, diosgenin, aescin, betulinic acid, cucurbitacin E, ruscogenin, mimusin, avenacin A-1, gracillin, α-tomatine, α-solanine, convallatoxin, acetyldigoxin, digoxin, deslanoside, digitalin, digitoxin, quillaic acid and its glycoside derivatives, squalamine, ouabain, strophanthidin, hydrocortisone, testosterone, and asiaticoside.

Recombinant vectors are also provided. Recombinant vectors can include a described exogenous nucleic acid operably linked to a regulatory region. The regulatory region can be a cell-specific or tissue-specific promoter. The promoter can be a leaf-specific promoter or a seed-specific promoter. A seed-specific promoter can be selected from the group consisting of the promoters YP0092 (SEQ ID NO: 62), PT0676 (SEQ ID NO: 72), PT0708 (SEQ ID NO: 74), PT0613 (SEQ ID NO: 66), PT0672 (SEQ ID NO: 68), PT0678 (SEQ ID NO: 69), PT0688 (SEQ ID NO: 70), PT0837 (SEQ ID NO: 76), the napin promoter, the Arcelin-5 promoter, the phaseolin gene promoter, the soybean trypsin inhibitor promoter, the ACP promoter, the stearoyl-ACP desaturase gene, the soybean α′ subunit of β-conglycinin promoter, the oleosin promoter, the 15 kD zein promoter, the 16 kD zein promoter, the 19 kD zein promoter, the 22 kD zein promoter, the 27 kD zein promoter, the Osgt-1 promoter, the beta-amylase gene promoter, and the barley hordein gene promoter. The promoter can also be a root-specific promoter. A root-specific promoter can be selected from the group consisting of YP0128 (SEQ ID NO: 63), YP0275 (SEQ ID NO: 65), PT0625 (SEQ ID NO: 67), PT0660 (SEQ ID NO: 71), PT0683 (SEQ ID NO: 73), and PT0758 (SEQ ID NO: 75). A regulatory region can be a broadly expressing promoter. A broadly expressing promoter can be selected from the group consisting of p326, YP0158, YP0214, YP0380, PT0848, PT0633, YP0050, YP0144, and YP0190. A regulatory region can also be a constitutive promoter or an inducible promoter. A first nucleic acid and a second nucleic acid can be operably linked to a first and a second regulatory region, respectively.

A plant or plant cell can be a member of one of the following genera: Acokanthera, Aesculus, Ananas, Arachis, Betula, Bixa, Brassica, Calendula, Carthamus, Centella, Chrysanthemum, Cinnamomum, Citrullus, Coffea, Convallaria, Curcuma, Digitalis, Dioscorea, Fragaria, Glycine, Glycyrrhiza, Gossypium, Helianthus, Lactuca, Lavandula, Linum, Luffa, Lycopersicon, Mentha, Musa, Ocimum, Origanum, Oryza, Quillaja, Rosmarinus, Ruscus, Salvia, Sesamum, Solanum, Strophanthus, Theobroma, Thymus, Triticum, Vitis, and Zea.

A plant or plant cell can be a species selected from Acokanthera spp., Ananas comosus, Betula alba, Bixa orellana, Brassica campestris, Brassica napus, Brassica oleracea, Calendula officinalis, Cathamus tinctorius, Centella asiatica, Chrysanthemum parthenium, Cinnamomum camphora, Citrullus spp., Coffea arabica, Convallaria majalis, Digitalis lantana, Digitalis purpurea, Digitalis spp., Dioscorea spp., Glycine max, Glycyrrhiza glabra, Gossypium spp., Lactuca sativa, Luffa spp., Lycopersicon esculentum, Mentha piperita, Mentha spicata, Musa paradisiaca, Oryza sativa, Quillaja saponaria, Rosmarinus officinalis, Ruscus aculeatus, Solanum tuberosum, Strophanthus gratus, Strophanthus spp., Theobroma cacao, Triticum aestivum, Vitis vinifera, and Zea mays.

A plant or plant cell can be selected from the group consisting of peanut, safflower, flax, sugar beet, chick peas, alfalfa, spinach, clover, cabbage, lentils, mustard, soybean, lettuce, castor bean, sesame, carrot, grape, cotton, crambe, strawberry, amaranth, rape, broccoli, peas, pepper, tomato, potato, yam, kidney beans, lima beans, dry beans, green beans, watermelon, cantaloupe, peach, pear, apple, cherry, orange, lemon, grapefruit, plum, mango, soaptree bark, oilseed rape, sunflower, garlic, oil palm, date palm, banana, sweet corn, popcorn, field corn, wheat, rye, barley, oat, onion, pineapple, rice, millet, and sorghum.

A plant tissue can be a leaf, seed, fruit, or tissue culture tissue.

In another aspect, a method of producing plant tissue is provided. The method can include growing a plant cell comprising an exogenous nucleic acid comprising a nucleotide sequence encoding a polypeptide having 80% or greater sequence identity to an amino acid sequence selected from the group consisting of SEQ ID NO: 35, SEQ ID NOS: 37-47, SEQ ID NO: 53, SEQ ID NOS: 55-61, SEQ ID NOS: 49-51, SEQ ID NOs: 2-12, SEQ ID NOs: 14-21, SEQ ID NOs: 23-26, SEQ ID NOS: 28-33, and the consensus sequences set forth in FIG. 2, 4, 5, 6, 7, 8, or 9, wherein the tissue has a difference in the level of a triterpenoid as compared to the corresponding level in tissue of a control plant that does not comprise the nucleic acid. The sequence identity can be 80%, 85%, 90%, 95% or greater.

In a further embodiment, a method of producing a plant tissue is provided. The method can include growing a plant cell comprising (a) a first exogenous nucleic acid comprising a nucleotide sequence encoding a polypeptide having 80% or greater sequence identity to an amino acid sequence selected from the group consisting of SEQ ID NO: 35, SEQ ID NOS: 37-47, SEQ ID NO: 53, SEQ ID NOS: 55-61, SEQ ID NOS: 49-51, and the consensus sequences set forth in FIG. 2, 4, or 5; and (b) a second exogenous nucleic acid comprising a nucleotide sequence encoding a polypeptide having 80% or greater sequence identity to an amino acid sequence selected from the group consisting of SEQ ID NOs: 2-12, SEQ ID NOs: 14-21, SEQ ID NOs: 23-26, SEQ ID NOS: 28-33, and the consensus sequences set forth in FIG. 6, 7, 8, or 9; where the tissue has a difference in the level of a triterpenoid as compared to the corresponding level in tissue of a control plant that does not comprise the first nucleic acid and the second nucleic acid. The sequence identity can be 80%, 85%, 90%, 95% or greater.

In a further embodiment, a method of producing a plant tissue is provided. The method can include growing a plant cell comprising: (a) a first exogenous nucleic acid comprising a nucleotide sequence encoding a polypeptide having 80% or greater sequence identity to an amino acid sequence selected from the group consisting of SEQ ID NO: 35, SEQ ID NOS: 37-47, SEQ ID NO: 53, SEQ ID NOS: 55-61, SEQ ID NOS: 49-51, and the consensus sequences set forth in FIG. 2, 4, or 5; and (b) a second exogenous nucleic acid comprising a nucleotide sequence encoding a polypeptide having 80% or greater sequence identity to an amino acid sequence selected from the group consisting of SEQ ID NO: 35, SEQ ID NOS: 37-47, SEQ ID NO: 53, SEQ ID NOS: 55-61, SEQ ID NOS: 49-51, and the consensus sequences set forth in FIG. 2, 4, or 5; provided that the first exogenous nucleic acid and the second exogenous nucleic acid are not the same, where the tissue has a difference in the level of a triterpenoid as compared to the corresponding level in tissue of a control plant that does not comprise the first nucleic acid and the second nucleic acid. The sequence identity can be 80%, 85%, 90%, 95% or greater.

In a further embodiment, a method of producing a plant tissue is provided. The method can include growing a plant cell comprising: (a) a first exogenous nucleic acid comprising a nucleotide sequence encoding a polypeptide having 80% or greater sequence identity to an amino acid sequence selected from the group consisting of SEQ ID NOs: 2-12, SEQ ID NOs: 14-21, SEQ ID NOs: 23-26, SEQ ID NOS: 28-33, and the consensus sequences set forth in FIG. 6, 7, 8, or 9; and (b) a second exogenous nucleic acid comprising a nucleotide sequence encoding a polypeptide having 80% or greater sequence identity to an amino acid sequence selected from the group consisting of SEQ ID NOs: 2-12, SEQ ID NOs: 14-21, SEQ ID NOs: 23-26, SEQ ID NOS: 28-33, and the consensus sequences set forth in FIG. 6, 7, 8, or 9; provided that the first exogenous nucleic acid and the second exogenous nucleic acid are not the same, where the tissue has a difference in the level of a triterpenoid as compared to the corresponding level in tissue of a control plant that does not comprise the first nucleic acid and the second nucleic acid. The sequence identity can be 80%, 85%, 90%, 95% or greater.

In another aspect, a method of producing a triterpenoid is provided. The method can include extracting a triterpenoid from transgenic plant tissue, the plant tissue including a nucleotide sequence encoding a polypeptide having 80% or greater sequence identity to an amino acid sequence selected from the group consisting of SEQ ID NO: 35, SEQ ID NOS: 37-47, SEQ ID NO: 53, SEQ ID NOS: 55-61, SEQ ID NOS: 49-51, SEQ ID NOs: 2-12, SEQ ID NOs: 14-21, SEQ ID NOs: 23-26, SEQ ID NOS: 28-33, and the consensus sequences set forth in FIG. 2, 4, 5, 6, 7, 8, or 9, where the tissue has a difference in the level of a triterpenoid as compared to the corresponding level in tissue of a control plant that does not comprise the nucleic acid. The sequence identity can be 80%, 85%, 90%, 95% or greater.

In another aspect, the method can include extracting a triterpenoid from transgenic plant tissue, the plant tissue comprising (a) a first exogenous nucleic acid comprising a nucleotide sequence encoding a polypeptide having 80% or greater sequence identity to an amino acid sequence selected from the group consisting of SEQ ID NO: 35, SEQ ID NOS: 37-47, SEQ ID NO: 53, SEQ ID NOS: 55-61, SEQ ID NOS: 49-51, and the consensus sequences set forth in FIG. 2, 4, or 5; and (b) a second exogenous nucleic acid comprising a nucleotide sequence encoding a polypeptide having 80% or greater sequence identity to an amino acid sequence selected from the group consisting of SEQ ID NOs: 2-12, SEQ ID NOs: 14-21, SEQ ID NOs: 23-26, SEQ ID NOS: 28-32, and the consensus sequences set forth in FIG. 6, 7, 8, or 9; wherein the tissue has a difference in the level of a triterpenoid as compared to the corresponding level in tissue of a control plant that does not comprise the first nucleic acid and the second nucleic acid. The sequence identity can be 80%, 85%, 90%, 95% or greater.

In another aspect, the method can include extracting a triterpenoid from transgenic plant tissue, the plant tissue comprising: (a) a first exogenous nucleic acid comprising a nucleotide sequence encoding a polypeptide having 80% or greater sequence identity to an amino acid sequence selected from the group consisting of SEQ ID NO: 35, SEQ ID NOS: 37-47, SEQ ID NO: 53, SEQ ID NOS: 55-61, SEQ ID NOS: 49-51, and the consensus sequences set forth in FIG. 2, 4, or 5; and (b) a second exogenous nucleic acid comprising a nucleotide sequence encoding a polypeptide having 80% or greater sequence identity to an amino acid sequence selected from the group consisting of SEQ ID NO: 35, SEQ ID NOS: 37-47, SEQ ID NO: 53, SEQ ID NOS: 55-61, SEQ ID NOS: 49-51, and the consensus sequences set forth in FIG. 2, 4, or 5; provided that the first exogenous nucleic acid and the second exogenous nucleic acid are not the same, wherein the tissue has a difference in the level of a triterpenoid as compared to the corresponding level in tissue of a control plant that does not comprise the first nucleic acid and the second nucleic acid. The sequence identity can be 80%, 85%, 90%, 95% or greater.

In another aspect, the method can include extracting a triterpenoid from transgenic plant tissue, the plant tissue comprising: (a) a first exogenous nucleic acid comprising a nucleotide sequence encoding a polypeptide having 80% or greater sequence identity to an amino acid sequence selected from the group consisting of SEQ ID NOs: 2-12, SEQ ID NOs: 14-21, SEQ ID NOs: 23-26, SEQ ID NOS: 28-33, and the consensus sequences set forth in FIG. 6, 7, 8, or 9; and (b) a second exogenous nucleic acid comprising a nucleotide sequence encoding a polypeptide having 80% or greater sequence identity to an amino acid sequence selected from the group consisting of SEQ ID NOs: 2-12, SEQ ID NOs: 14-21, SEQ ID NOs: 23-26, SEQ ID NOS: 28-33, and the consensus sequences set forth in FIG. 6, 7, 8, or 9; provided that the first exogenous nucleic acid and the second exogenous nucleic acid are not the same, wherein the tissue has a difference in the level of a triterpenoid as compared to the corresponding level in tissue of a control plant that does not comprise the first nucleic acid and the second nucleic acid. The sequence identity can be 80%, 85%, 90%, 95% or greater.

A difference in the level of a triterpenoid can be a difference in the level of any triterpenoid as described above.

Recombinant vectors are also provided. Recombinant vectors can include a described exogenous nucleic acid operably linked to a regulatory region. The regulatory can be a regulatory region as described above.

A plant or plant cell can be a member of the genera as described above.

A plant or plant cell can be a species selected from the species as described above.

A plant or plant cell can be selected from the group described above.

A plant tissue can be a leaf, seed, fruit or tissue culture tissue.

Plant cells and plants are also provided herein. A plant cell can include an exogenous nucleic acid comprising a nucleotide sequence encoding a polypeptide having 80% or greater sequence identity to an amino acid sequence selected from the group consisting of SEQ ID NO: 35, SEQ ID NOS: 37-47, SEQ ID NO: 53, SEQ ID NOS: 55-61, SEQ ID NOS: 49-51, SEQ ID NOs: 2-12, SEQ ID NOs: 14-21, SEQ ID NOs: 23-26, SEQ ID NOS: 28-33, and the consensus sequences set forth in FIG. 2, 4, 5, 6, 7, 8, or 9, where a tissue of a plant produced from the plant cell has a difference in the level of a triterpenoid as compared to the corresponding level in tissue of a control plant that does not comprise the nucleic acid. The sequence identity can be 80%, 85%, 90%, 95% or greater.

In another embodiment, a plant cell can include (a) a first exogenous nucleic acid comprising a nucleotide sequence encoding a polypeptide having 80% or greater sequence identity to an amino acid sequence selected from the group consisting of SEQ ID NO: 35, SEQ ID NOS: 37-47, SEQ ID NO: 53, SEQ ID NOS: 55-61, SEQ ID NOS: 49-51 and the consensus sequences set forth in FIG. 2, 4, or 5; and (b) a second exogenous nucleic acid comprising a nucleotide sequence encoding a polypeptide having 80% or greater sequence identity to an amino acid sequence selected from the group consisting of SEQ ID NOs: 2-12, SEQ ID NOs: 14-21, SEQ ID NOs: 23-26, SEQ ID NOS: 28-33, and the consensus sequences set forth in FIG. 6, 7, 8, or 9; wherein expression of the exogenous nucleic acids in tissue of a plant produced from the plant cell results in a difference in the level of a triterpenoid as compared to the corresponding level in tissue of a control plant that does not comprise the first nucleic acid and the second nucleic acid. The sequence identity can be 80%, 85%, 90%, 95% or greater.

In another embodiment, a plant cell can include: (a) a first exogenous nucleic acid comprising a nucleotide sequence encoding a polypeptide having 80% or greater sequence identity to an amino acid sequence selected from the group consisting of SEQ ID NO: 35, SEQ ID NOS: 37-47, SEQ ID NO: 53, SEQ ID NOS: 55-61, SEQ ID NOS: 49-51, and the consensus sequences set forth in FIG. 2, 4, or 5; and (b) a second exogenous nucleic acid comprising a nucleotide sequence encoding a polypeptide having 80% or greater sequence identity to an amino acid sequence selected from the group consisting of SEQ ID NO: 35, SEQ ID NOS: 37-47, SEQ ID NO: 53, SEQ ID NOS: 55-61, SEQ ID NOS: 49-51, and the consensus sequences set forth in FIG. 2, 4, or 5; provided that the first exogenous nucleic acid and the second exogenous nucleic acid are not the same, wherein expression of the exogenous nucleic acids in tissue of a plant produced from the plant cell results in a difference in the level of a triterpenoid as compared to the corresponding level in tissue of a control plant that does not comprise the first nucleic acid and the second nucleic acid. The sequence identity can be 80%, 85%, 90%, 95% or greater.

In another embodiment, a plant cell can include: (a) a first exogenous nucleic acid comprising a nucleotide sequence encoding a polypeptide having 80% or greater sequence identity to an amino acid sequence selected from the group consisting of SEQ ID NOs: 2-12, SEQ ID NOs: 14-21, SEQ ID NOs: 23-26, SEQ ID NOS: 28-33, and the consensus sequences set forth in FIG. 6, 7, 8, or 9; and (b) a second exogenous nucleic acid comprising a nucleotide sequence encoding a polypeptide having 80% or greater sequence identity to an amino acid sequence selected from the group consisting of SEQ ID NOs: 2-12, SEQ ID NOs: 14-21, SEQ ID NOs: 23-26, SEQ ID NOS: 28-33, and the consensus sequences set forth in FIG. 6, 7, 8, or 9; provided that the first exogenous nucleic acid and the second exogenous nucleic acid are not the same, where expression of the exogenous nucleic acids in tissue of a plant produced from the plant cell results in a difference in the level of a triterpenoid as compared to the corresponding level in tissue of a control plant that does not comprise the first nucleic acid and the second nucleic acid. The sequence identity can be 80%, 85%, 90%, 95% or greater.

A difference in the level of a triterpenoid can be a difference in the level of any triterpenoid as described above.

Recombinant vectors are also provided. Recombinant vectors can include a described exogenous nucleic acid operably linked to a regulatory region. The regulatory can be a regulatory region as described above.

A plant or plant cell can be a member of the genera as described above.

A plant or plant cell can be a species selected from the species as described above.

A plant or plant cell can be selected from the group described above.

A plant tissue can be a leaf, seed, fruit, or tissue culture tissue.

In another embodiment, transgenic plants having altered levels of a triterpenoid are provided. A transgenic plant can include a plant cell including an exogenous nucleic acid comprising a nucleotide sequence encoding a polypeptide having 80% or greater sequence identity to an amino acid sequence selected from the group consisting of SEQ ID NO: 35, SEQ ID NOS: 37-47, SEQ ID NO: 53, SEQ ID NOS: 55-61, SEQ ID NOS: 49-51, SEQ ID NOs: 2-12, SEQ ID NOs: 14-21, SEQ ID NOs: 23-26, SEQ ID NOS: 28-33, and the consensus sequences set forth in FIG. 2, 4, 5, 6, 7, 8, or 9, where a tissue of a plant produced from the plant cell has a difference in the level of a triterpenoid as compared to the corresponding level in tissue of a control plant that does not comprise the nucleic acid. The sequence identity can be 80%, 85%, 90%, 95% or greater.

In another embodiment, a transgenic plant can include a plant cell including: (a) a first exogenous nucleic acid comprising a nucleotide sequence encoding a polypeptide having 80% or greater sequence identity to an amino acid sequence selected from the group consisting of SEQ ID NO: 35, SEQ ID NOS: 37-47, SEQ ID NO: 53, SEQ ID NOS: 55-61, SEQ ID NOS: 49-51 and the consensus sequences set forth in FIG. 2, 4, or 5; and (b) a second exogenous nucleic acid comprising a nucleotide sequence encoding a polypeptide having 80% or greater sequence identity to an amino acid sequence selected from the group consisting of SEQ ID NOs: 2-12, SEQ ID NOs: 14-21, SEQ ID NOs: 23-26, SEQ ID NOS: 28-33, and the consensus sequences set forth in FIG. 6, 7, 8, or 9; wherein expression of the exogenous nucleic acids in tissue of a plant produced from the plant cell results in a difference in the level of a triterpenoid as compared to the corresponding level in tissue of a control plant that does not comprise the first nucleic acid and the second nucleic acid. The sequence identity can be 80%, 85%, 90%, 95% or greater.

In another embodiment, a transgenic plant can include a plant cell including: (a) a first exogenous nucleic acid comprising a nucleotide sequence encoding a polypeptide having 80% or greater sequence identity to an amino acid sequence selected from the group consisting of SEQ ID NO: 35, SEQ ID NOS: 37-47, SEQ ID NO: 53, SEQ ID NOS: 55-61, SEQ ID NOS: 49-51, and the consensus sequences set forth in FIG. 2, 4, or 5; and (b) a second exogenous nucleic acid comprising a nucleotide sequence encoding a polypeptide having 80% or greater sequence identity to an amino acid sequence selected from the group consisting of SEQ ID NO: 35, SEQ ID NOS: 37-47, SEQ ID NO: 53, SEQ ID NOS: 55-61, SEQ ID NOS: 49-51, and the consensus sequences set forth in FIG. 2, 4, or 5; provided that the first exogenous nucleic acid and the second exogenous nucleic acid are not the same, wherein expression of the exogenous nucleic acids in tissue of a plant produced from the plant cell results in a difference in the level of a triterpenoid as compared to the corresponding level in tissue of a control plant that does not comprise the first nucleic acid and the second nucleic acid. The sequence identity can be 80%, 85%, 90%, 95% or greater.

In another embodiment, a transgenic plant can include a plant cell including: (a) a first exogenous nucleic acid comprising a nucleotide sequence encoding a polypeptide having 80% or greater sequence identity to an amino acid sequence selected from the group consisting of SEQ ID NOs: 2-12, SEQ ID NOs: 14-21, SEQ ID NOs: 23-26, SEQ ID NOS: 28-33, and the consensus sequences set forth in FIG. 6, 7, 8, or 9; and (b) a second exogenous nucleic acid comprising a nucleotide sequence encoding a polypeptide having 80% or greater sequence identity to an amino acid sequence selected from the group consisting of SEQ ID NOs: 2-12, SEQ ID NOs: 14-21, SEQ ID NOs: 23-26, SEQ ID NOS: 28-33, and the consensus sequences set forth in FIG. 6, 7, 8, or 9; provided that the first exogenous nucleic acid and the second exogenous nucleic acid are not the same, where expression of the exogenous nucleic acids in tissue of a plant produced from the plant cell results in a difference in the level of a triterpenoid as compared to the corresponding level in tissue of a control plant that does not comprise the first nucleic acid and the second nucleic acid. The sequence identity can be 80%, 85%, 90%, 95% or greater.

A difference in the level of a triterpenoid can be a difference in the level of any triterpenoid as described above.

Recombinant vectors are also provided. Recombinant vectors can include a described exogenous nucleic acid operably linked to a regulatory region. The regulatory can be a regulatory region as described above.

A plant or plant cell can be a member of the genera as described above.

A plant or plant cell can be a species selected from the species as described above.

A plant or plant cell can be selected from the group described above.

Also provided are progeny of the transgenic plants, where the progeny have a difference in the level of one or more triterpenoids as compared to the corresponding level in tissue of a control plant that does not comprise the exogenous nucleic acid.

In another aspect, the progeny are seeds and the seeds have a difference in the level of one or more triterpenoids as compared to the corresponding level in seeds of a control plant that does not comprise the exogenous nucleic acid.

In another aspect, articles of manufacture are provided including a flour, an oil, or an insoluble fiber product derived from the seeds of the transgenic plants.

In another embodiment, isolated nucleic acid molecules are provided. An isolated nucleic acid molecule can include a nucleotide sequence having 95% or greater sequence identity to the nucleotide sequence set forth in SEQ ID NO: 156; SEQ ID NO: 158; SEQ ID NO: 160; SEQ ID NO: 162; SEQ ID NO: 165; SEQ ID NO: 167; SEQ ID NO: 170; SEQ ID NO: 172; SEQ ID NO: 174; SEQ ID NO: 176; SEQ ID NO: 178; SEQ ID NO: 180; SEQ ID NO: 182; SEQ ID NO: 184; SEQ ID NO: 187; SEQ ID NO: 189; and SEQ ID NO: 191. In another embodiment, an isolated nucleic acid can include a nucleotide sequence encoding a polypeptide having 80% or greater sequence identity to the amino acid sequence set forth in SEQ ID NO: SEQ ID NO: 157; SEQ ID NO: 159; SEQ ID NO: 161; SEQ ID NO: 163; SEQ ID NO: 164; SEQ ID NO: 166; SEQ ID NO: 168; SEQ ID NO: 169; SEQ ID NO: 171; SEQ ID NO: 173; SEQ ID NO: 175; SEQ ID NO: 177; SEQ ID NO: 179; SEQ ID NO: 181; SEQ ID NO: 183; SEQ ID NO: 185; SEQ ID NO: 186; SEQ ID NO: 188; SEQ ID NO: 190; and SEQ ID NO: 192

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting. One or more numeric values in a table herein can be combined with one or more values in another table to describe a range of values for the indicated property or characteristic. If the word “about” is used in conjunction with a numeric value, the exact numeric value is also included as the alternative statement of the numeric value.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 shows the amino acid sequence of a polypeptide designated cDNA ID 23357293, also known as CeresClone 31252, (SEQ ID NO: 35).

FIG. 2 shows an alignment of cDNA ID 23389731 (SEQ ID NO: 37) amino acid sequence with orthologous amino acid sequences gi11463943 (SEQ ID NO:47), gi1805618 (SEQ ID NO:45), gi6016226 (SEQ ID NO:43), gi7446245 (SEQ ID NO:44), CeresClone:515966 (SEQ ID NO:42), gi/946222 (SEQ ID NO:41), and gi1045044 (SEQ ID NO:38).

FIG. 3 shows the amino acid sequence of a polypeptide designated cDNA ID 23543586 (SEQ ID NO: 53).

FIG. 4 shows an alignment cDNA ID 23361365 (SEQ ID NO: 55) amino acid sequence with orthologous amino acid sequences gi9759231 (SEQ ID NO:56), CeresClone642012 (SEQ ID NO:57), CeresClone246572 (SEQ ID NO:60), CeresClone766557 (SEQ ID NO:59), and gi55733851 (SEQ ID NO:61).

FIG. 5 shows an alignment of cDNA ID 23644306 (SEQ ID NO:49) amino acid sequence with orthologous amino acid sequences cDNA CeresClone280200 (SEQ ID NO:50), and gi22165075 (SEQ ID NO:51).

FIG. 6 shows an alignment of cDNA ID 12328487, also known as CeresClone 28635, (SEQ ID NO: 2) amino acid sequence with orthologous amino acid sequences gi/552717 (SEQ ID NO: 11); gi1184109 (SEQ ID NO: 12); gi5360655 (SEQ ID NO: 9); gi4426953 (SEQ ID NO: 10); gi55710094 (SEQ ID NO: 4); gi41224629 (SEQ ID NO: 7); gi27475614 (SEQ ID NO: 8); gi28208268 (SEQ ID NO: 6); gi2144186 (SEQ ID NO: 5); and CeresClone 515962 (SEQ ID NO: 3).

FIG. 7 shows an alignment of cDNA ID 12394143, also known as CeresClone 23439, (SEQ ID NO: 14) amino acid sequence with orthologous amino acid sequences gi51963234 (SEQ ID NO: 19); CeresClone 217004 (SEQ ID NO: 20); CeresClone 977729 (SEQ ID NO: 17); gi34978966 (SEQ ID NO: 18); gi 27448145 (SEQ ID NO: 15); and CeresClone 664026 (SEQ ID NO: 16).

FIG. 8 shows an alignment of cDNA ID 12421417, also known as CeresClone 39378, (SEQ ID NO: 23) amino acid sequence with orthologous amino acid sequences CeresClone 285554 (SEQ ID NO: 25); gi62732798 (SEQ ID NO: 26); and CeresClone 716942 (SEQ ID NO: 24).

FIG. 9 shows an alignment of cDNA ID 13487250, also known as CeresClone 2121, (SEQ ID NO: 28) amino acid sequence with orthologous amino acid sequences gi50900588 (SEQ ID NO: 32); CeresClone 703736 (SEQ ID NO: 33); CeresClone 282337 (SEQ ID NO: 31); CeresClone 592262 (SEQ ID NO: 30); and CeresClone 959258 (SEQ ID NO: 29).

### DETAILED DESCRIPTION

Triterpenoids have diverse functions in all eukaryotes. One such triterpenoid, squalene, is a key precursor in the biosynthesis of a class of triterpenoids termed sterols. Sterols are an important component of eukaryotic cell membranes. The present invention provides materials and methods for modulating the levels of triterpenoids. The materials and methods provided herein permit the modulation of triterpenoids in plants and thereby provide materials for use in nutritional and pharmaceutical products.

The materials and methods provided herein involve the use of triterpenoid-modulating polypeptides to make a plant or plant cell having a modulated level of one or more triterpenoids. Triterpenoid-modulating polypeptides are polypeptides that are effective for modulating the levels of one or more triterpenoids in a cell. A triterpenoid-modulating polypeptide can be a transcription factor, for example, an AP2 domain protein, a zinc-finger containing protein, or a homeodomain-containing protein. A triterpenoid-modulating polypeptide can also be a redox protein, for example, a thioredoxin. A triterpenoid-modulating polypeptide can be a triterpenoid biosynthetic enzyme such as, without limitation, cyclopropyl sterol isomerase or a C-8, 7 sterol isomerase. By using various promoters, it is possible to target the production of various triterpenoids to specific tissues at specific times through development or to have triterpenoid production induced under certain conditions.

Thus, methods for modulating the levels of one or more triterpenoids in a plant are provided. Methods are also provided for producing plants and plant cells having modulated levels of one or more triterpenoids. Methods for producing plant products including seeds, oils, and roots containing modulated levels of one or more triterpenoids are further provided. Such plants may be used to produce foodstuffs having increased nutritional content, which may benefit both food producers and consumers, or can be used as sources from which to extract one or more triterpenoids.

### I. Polypeptides and Polynucleotides

### A. Triterpenoid-Modulating Polypeptides

Provided herein are triterpenoid-modulating polypeptides. A triterpenoid-modulating polypeptide can be effective for modulating the level of one or more triterpenoids in a plant or plant cell. Modulation in the level of a triterpenoid can be either an increase in the level of a triterpenoid or a decrease in the level of a triterpenoid, relative to the corresponding level in a control plant.

A triterpenoid-modulating polypeptide can be a transcription factor. Transcription factors regulate gene expression through specific DNA and protein binding events. It has been well established in both primary and secondary metabolism that transcription factors drive the expression of genes responsible for entire segments of biosynthetic pathways. Transcription factor proteins share common structural features that include a DNA-binding domain, for interacting with nucleic acids, and activation and oligomerization domains that mediate interactions with other proteins. Transcription factors can be classified based on characteristic structural motifs found within these domains.

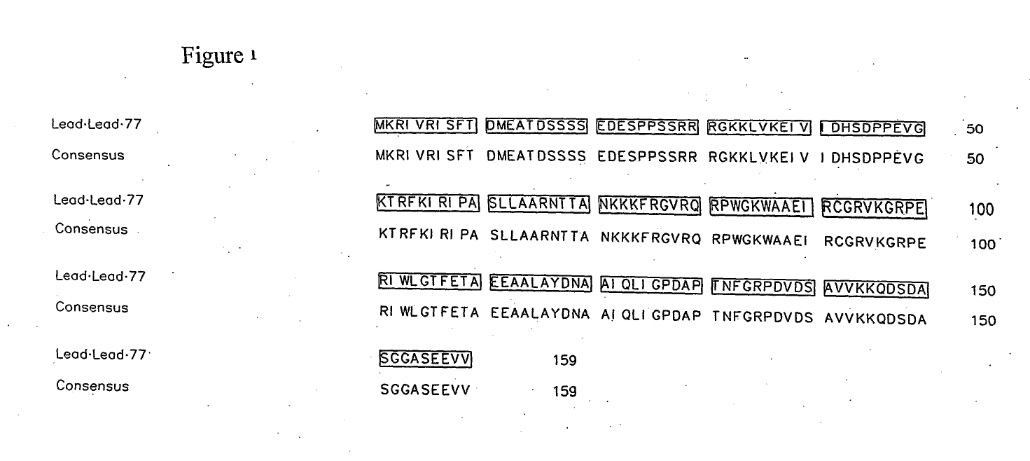

Thus, a triterpenoid-modulating polypeptide can be a transcription factor that contains an AP2 (APETALA2) DNA-binding domain. AP2 is one of the prototypic members of a family of transcription factors unique to plants, whose distinguishing characteristic is that they contain the so-called AP2 DNA-binding domain. cDNA 23357293 (SEQ ID NO: 34) is predicted to encode a transcription factor that contains an AP2 DNA-binding domain. A triterpenoid-modulating polypeptide encoded by a nucleic acid, and useful in the compositions and methods described herein, comprises an amino acid sequence having 80% or greater sequence identity (e.g., 85%, 90%, 95%, 98%, 99%, or 100% sequence identity) to the amino acid sequence encoded by the cDNA ID 23357293 as set forth in FIG. 1 and SEQ ID NO:35. For example, a suitable triterpenoid-modulating polypeptide has 94% or greater sequence identity to the amino acid sequence of SEQ ID NO:35.

A triterpenoid-modulating polypeptide can also be a protein that contains a homeodomain. Homeodomains are evolutionarily conserved DNA-binding regions encoded by a DNA motif of about 180 base-pairs termed a homeobox. Homeobox genes play important roles in regulation of gene expression in development through recognition of specific target genes. The classical homeodomain motif comprises three α helices; different homeodomain proteins have been grouped into separate families based upon either sequence identity within the homeodomain or within conserved protein motifs outside the homeodomain. In plants, several families of homeodomain proteins have been described including the KNOTTED 1-like proteins and the plant homeodomain finger proteins (PHD-finger).

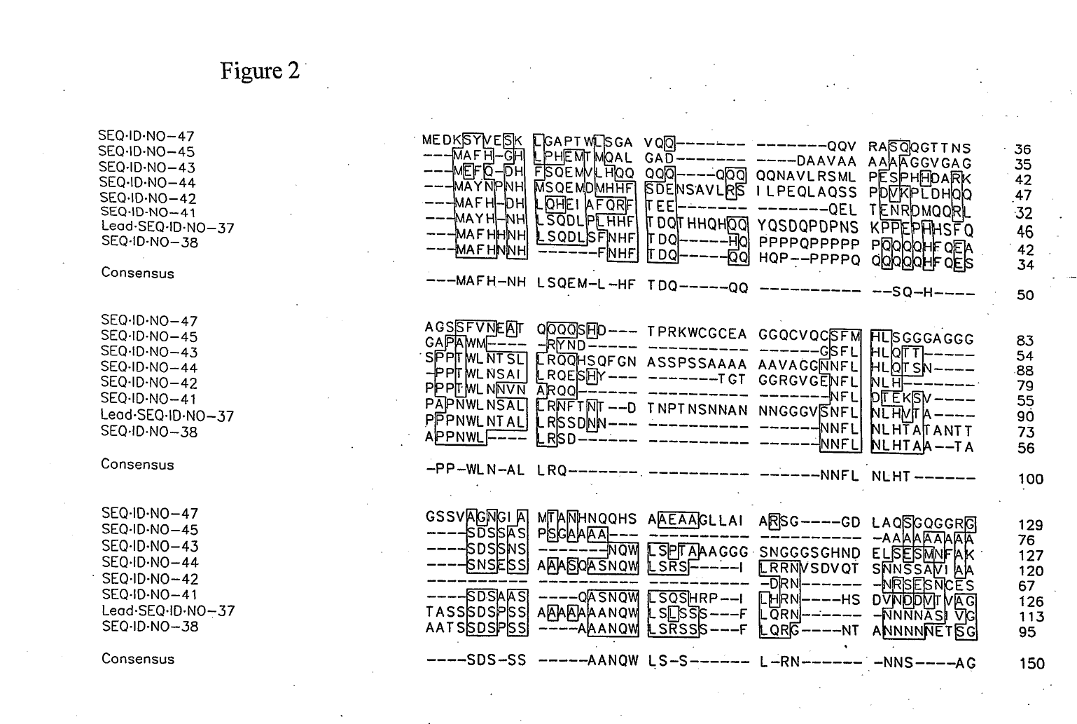

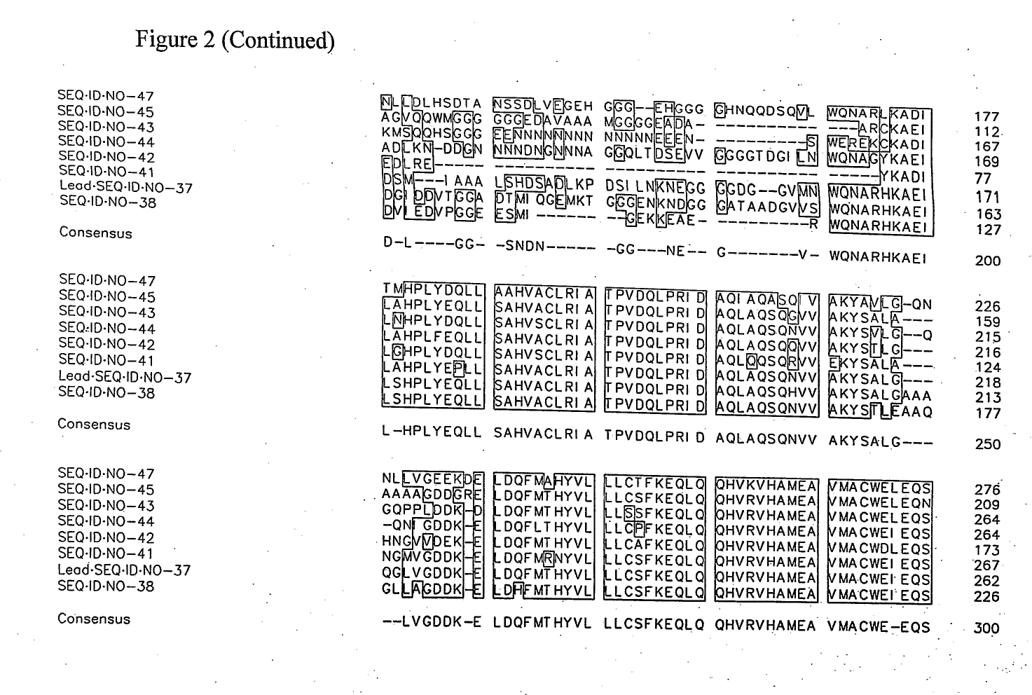

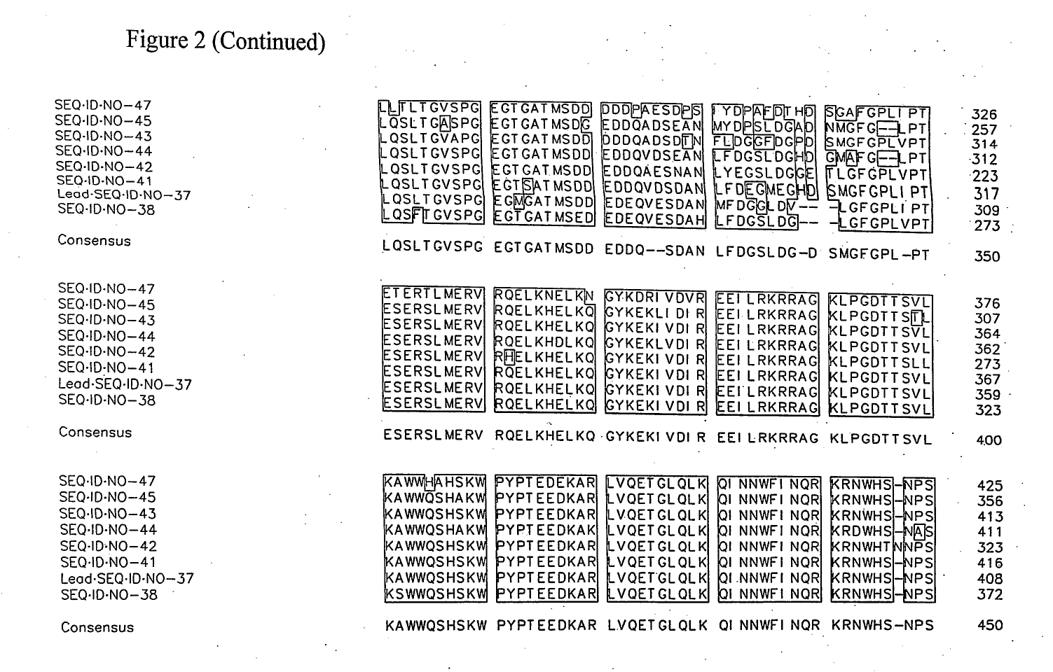

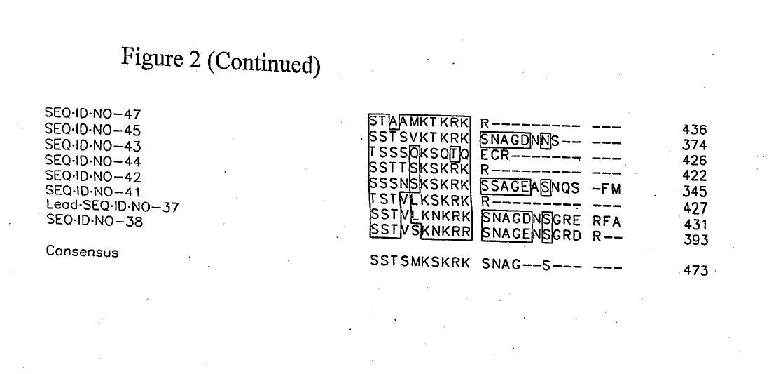

A triterpenoid-modulating polypeptide can have the amino acid sequence encoded by cDNA 23389731 as set forth in FIG. 2 and in SEQ ID NO:37. cDNA 23389731 (SEQ ID NO: 36) is predicted to encode a member of the Arabidopsis KNOTTED 1-like family of proteins, KNAT3. Specifically, SEQ ID NO:36 is predicted to encode KNOX1, KNOX2 and ELK domains. Thus, a triterpenoid-modulating polypeptide can be an Arabidopsis polypeptide having the amino acid sequence set forth in SEQ ID NO:37. Alternatively, a triterpenoid-modulating polypeptide can be an ortholog, homolog, or variant of the polypeptide having the sequence set forth in SEQ ID NO:37. A triterpenoid-modulating polypeptide, as described herein, can have an amino acid sequence with at least 35 percent sequence identity (e.g., 35 percent, 40 percent, 45 percent, 50 percent, 55 percent, 60 percent, 65 percent, 70 percent, 80 percent, 81 percent, 82 percent, 83 percent, 84 percent, 85 percent, 86 percent, 87 percent, 88 percent, 89 percent, 90 percent, 91 percent, 92 percent, 93 percent, 94 percent, 95 percent, 96 percent, 97 percent, 98 percent, or 99 percent sequence identity) to the amino acid sequence set forth in SEQ ID NO:37.

The alignment shown in FIG. 2 sets forth amino acid sequences of SEQ ID NO:37 orthologues and a consensus sequence. A consensus amino acid sequence for such orthologues was determined by aligning amino acid sequences, e.g., amino acid sequences related to SEQ ID NO:37, from a variety of species and determining the most common amino acid or type of amino acid at each position. For example, the alignment in FIG. 2 provides the amino acid sequences of cDNA 23389731 (SEQ ID NO:37), gi/1463943 (SEQ ID NO:47), gi/805618 (SEQ ID NO:45), gi6016226 (SEQ ID NO:43), gi7446245 (SEQ ID NO:44), CeresClone:515966 (SEQ ID NO:42), gi/946222 (SEQ ID NO:41), and gi1045044 (SEQ ID NO:38). Other orthologues include gi26451634 (SEQ ID NO:39), gi9795158 (SEQ ID NO:40) and gi/805617 (SEQ ID NO:46). In certain cases, therefore, a triterpenoid-modulating polypeptide can include an amino acid sequence having about 80% or greater sequence identity to cDNA 23389731 (SEQ ID NO:37), gi11463943 (SEQ ID NO:47), gi/805618 (SEQ ID NO:45), gi6016226 (SEQ ID NO:43), gi7446245 (SEQ ID NO:44), CeresClone:515966 (SEQ ID NO:42), gi/946222 (SEQ ID NO:41), gi1045044 (SEQ ID NO:38), gi26451634 (SEQ ID NO:39), gi9795158 (SEQ ID NO:40) and gi1805617 (SEQ ID NO:46). Eighty percent sequence identity or greater can be about 82, 85, 87, 90, 92, 95, 96, 97, 98, 99, or 100% sequence identity to such a sequence.

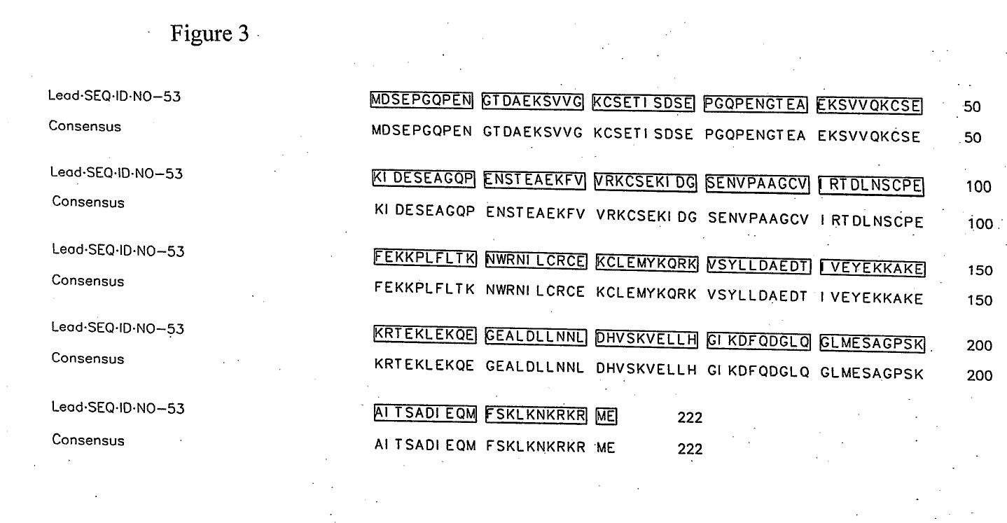

A triterpenoid-modulating polypeptide can have the amino acid sequence encoded by the cDNA 23543586 as set forth in FIG. 3 and in SEQ ID NO:53. cDNA 23543586 (SEQ ID NO:52) is predicted to encode an Arabidopsis PHD-finger containing protein. PHD-fingers are protein domains that are a subclass of zinc finger motifs. Zinc finger motifs typically include one or more cysteine and histidine residues that can bind a zinc atom. Zinc finger motifs can serve as structural platforms for DNA binding; PHD-finger motifs may also function as protein-protein interaction domains. A triterpenoid-modulating polypeptide encoded by a nucleic acid, and useful in the compositions and methods described herein, comprises an amino acid sequence having 80% or greater sequence identity (e.g., 85%, 90%, 95%, 98%, 99%, or 100% sequence identity) to the amino acid sequence of SEQ ID NO:53.

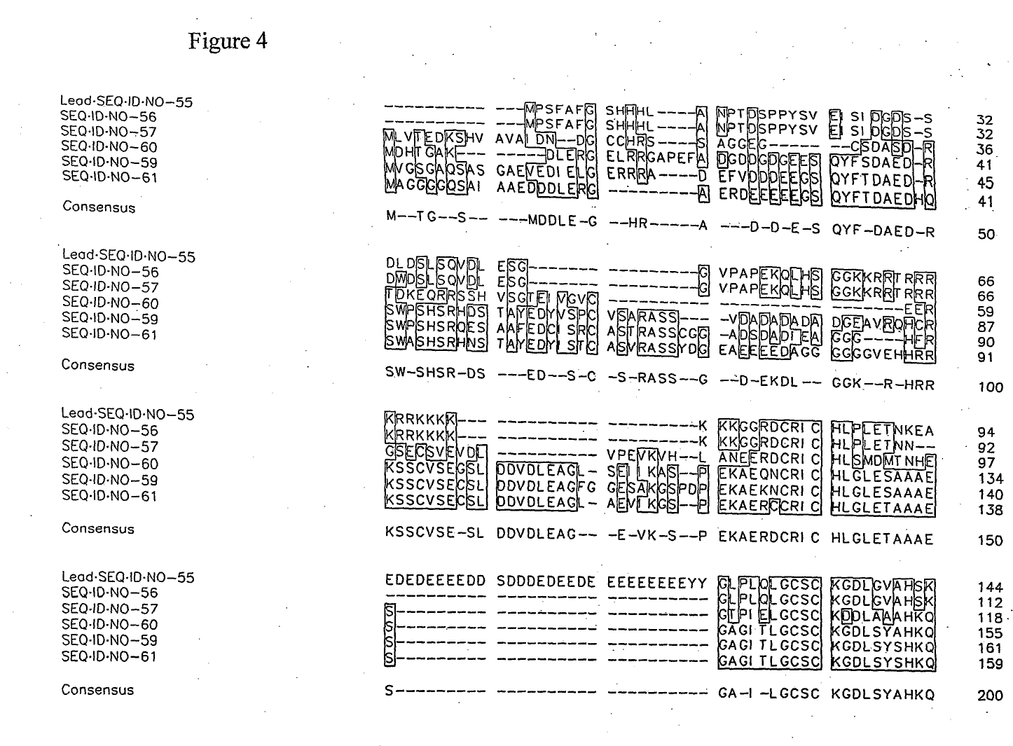

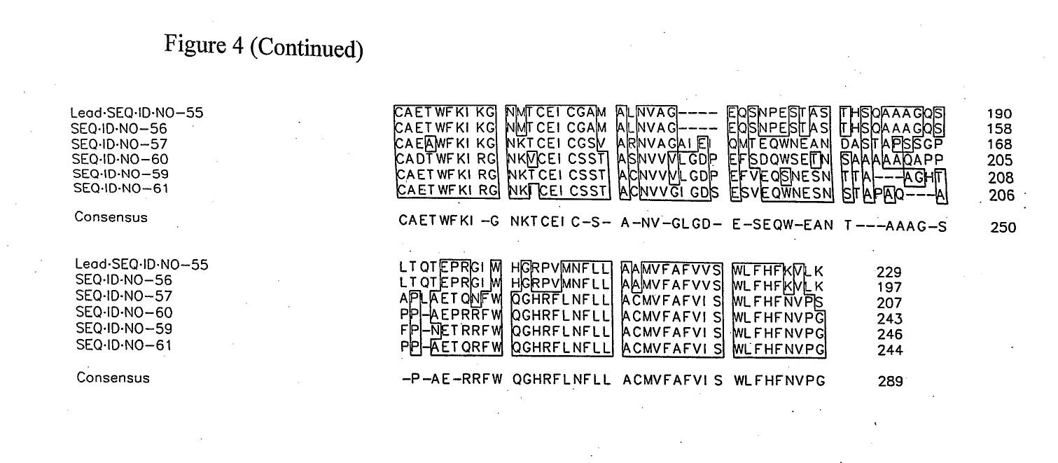

A triterpenoid-modulating polypeptide can have the amino acid sequence encoded by cDNA 23361365 as set forth in FIG. 4 and in SEQ ID NO:55. cDNA 23361365 (SEQ ID NO: 54) is predicted to encode an Arabidopsis C3H4 type RING-finger containing protein. The RING domain is a variant of a zinc finger motif and, like the PHD-finger, has been implicated in a variety of processes that rely upon protein-protein interactions. Thus, a triterpenoid-modulating polypeptide can be an Arabidopsis polypeptide having the amino acid sequence set forth in SEQ ID NO:55. Alternatively, a triterpenoid-modulating polypeptide can be an ortholog, homolog, or variant of the polypeptide having the sequence set forth in SEQ ID NO:55. A triterpenoid-modulating polypeptide, as described herein, can have an amino acid sequence with at least 35 percent sequence identity (e.g., 35 percent, 40 percent, 45 percent, 50 percent, 55 percent, 60 percent, 65 percent, 70 percent, 80 percent, 81 percent, 82 percent, 83 percent, 84 percent, 85 percent, 86 percent, 87 percent, 88 percent, 89 percent, 90 percent, 91 percent, 92 percent, 93 percent, 94 percent, 95 percent, 96 percent, 97 percent, 98 percent, or 99 percent sequence identity) to the amino acid sequence set forth in SEQ ID NO:55.

The alignment shown in FIG. 4 sets forth amino acid sequences of SEQ ID NO:55 orthologues and a consensus sequence. A consensus amino acid sequence for such orthologues was determined by aligning amino acid sequences, e.g., amino acid sequences related to SEQ ID NO:55, from a variety of species and determining the most common amino acid or type of amino acid at each position. For example, the alignment in FIG. 4 provides the amino acid sequences of cDNA 23361365 (SEQ ID NO:55), gi9759231 (SEQ ID NO:56), CeresClone642012 (SEQ ID NO:57), CeresClone246572 (SEQ ID NO:60), CeresClone766557 (SEQ ID NO:59), and gi55733851 (SEQ ID NO:61). Another orthologue can be CeresClone518866 (SEQ ID NO:58). In certain cases, therefore, a triterpenoid-modulating polypeptide can include an amino acid sequence having about 80% or greater sequence identity to cDNA 23361365 (SEQ ID NO:55), gi9759231 (SEQ ID NO:56), CeresClone642012 (SEQ ID NO:57), CeresClone246572 (SEQ ID NO:60), CeresClone766557 (SEQ ID NO:59), gi55733851 (SEQ ID NO:61), and CeresClone518866 (SEQ ID NO:58). Eighty percent sequence identity or greater can be about 82, 85, 87, 90, 92, 95, 96, 97, 98, 99, or 100% sequence identity to such a sequence.

A triterpenoid-modulating polypeptide can also be a thioredoxin. Thioredoxins are an evolutionarily conserved, widely distributed family of small proteins that, by virtue of their ability to undergo reversible oxidation/reduction, help to maintain the redox state of the cell and thus regulate a broad spectrum of cellular processes. Members of the thioredoxin family share a common structural motif termed the thioredoxin fold. Plant thioredoxins fall into three groups based upon their subcellular localization, with thioredoxins m and f found in the chloroplast and thioredoxin h found in the cytosol.

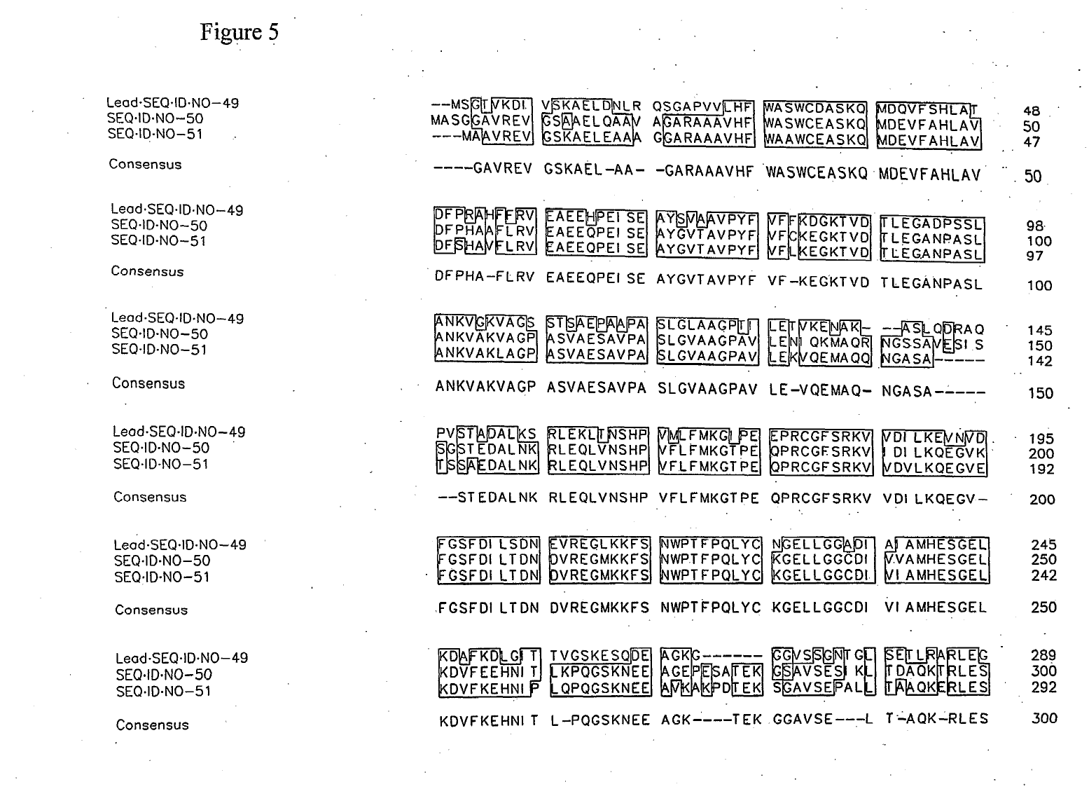

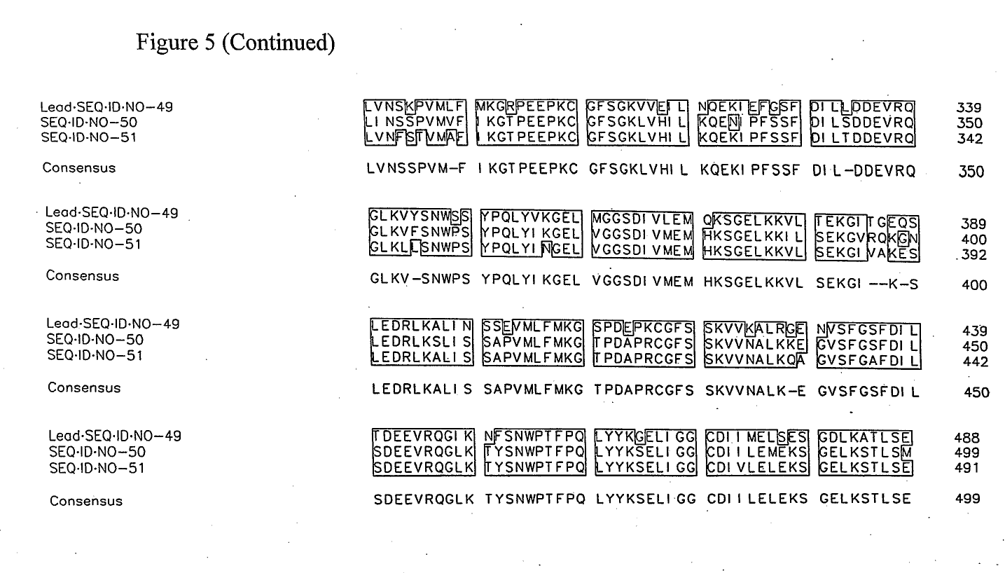

A triterpenoid-modulating polypeptide can have the amino acid sequence encoded by cDNA 23644306 as set forth in FIG. 5 and in SEQ ID NO:49. cDNA 23644306 (SEQ ID NO: 48) is predicted to encode an Arabidopsis thioredoxin m4 protein. Thus, a triterpenoid-modulating polypeptide can be an Arabidopsis polypeptide having the amino acid sequence set forth in SEQ ID NO:49. Alternatively, a triterpenoid-modulating polypeptide can be an ortholog, homolog, or variant of the polypeptide having the sequence set forth in SEQ ID NO:49. A triterpenoid-modulating polypeptide, as described herein, can have an amino acid sequence with at least 35 percent sequence identity (e.g., 35 percent, 40 percent, 45 percent, 50 percent, 55 percent, 60 percent, 65 percent, 70 percent, 80 percent, 81 percent, 82 percent, 83 percent, 84 percent, 85 percent, 86 percent, 87 percent, 88 percent, 89 percent, 90 percent, 91 percent, 92 percent, 93 percent, 94 percent, 95 percent, 96 percent, 97 percent, 98 percent, or 99 percent sequence identity) to the amino acid sequence set forth in SEQ ID NO:49.

The alignment shown in FIG. 5 sets forth amino acid sequences of SEQ ID NO:49 orthologues and a consensus sequence. A consensus amino acid sequence for such orthologues was determined by aligning amino acid sequences, e.g., amino acid sequences related to SEQ ID NO:49, from a variety of species and determining the most common amino acid or type of amino acid at each position. For example, the alignment in FIG. 5 provides the amino acid sequences of cDNA 23644306 (SEQ ID NO:49), CeresClone280200 (SEQ ID NO:50), gi22165075 (SEQ ID NO:51). In certain cases, therefore, a triterpenoid-modulating polypeptide can include an amino acid sequence having about 80% or greater sequence identity to an amino acid sequence set forth in FIG. 5, e.g., 80% or greater amino acid sequence identity to cDNA 23644306 (SEQ ID NO:49), CeresClone280200 (SEQ ID NO:50), gi22165075 (SEQ ID NO:51). Eighty percent sequence identity or greater can be about 82, 85, 87, 90, 92, 95, 96, 97, 98, 99, or 100% sequence identity to such a sequence.

A triterpenoid-modulating polypeptide can be an enzyme involved in triterpenoid biosynthesis. Enzymes involved in triterpenoid biosynthesis can be, for example, farnesyl diphosphate synthase (EC 2.5.1.10), farnesyl-diphosphate:farnesyl-diphosphate farnesyltransferase, also known as presqualene-diphosphate synthase or squalene synthase (EC 2.5.1.21), squalene, hydrogen-donor:oxygen oxidoreductase (2,3-epoxidizing), also known as squalene-2,3-epoxide cyclase (EC 1.14.99.7), cycloartenol synthase (EC5.4.99.8), cyclopropyl sterol isomerase, also known as cycloeucalenol cycloisomerase (EC 5.5.1.9), C-8,7 sterol isomerase, sterol methyl transferase2, sterol methyl oxidase, dammarenediol synthase, α-amyrin synthase, β-amyrin synthase, lupeol synthase, hopene cyclase, sesqueterpene synthases, sesqueterpene cylases, or pentacyclic triterpene synthases.

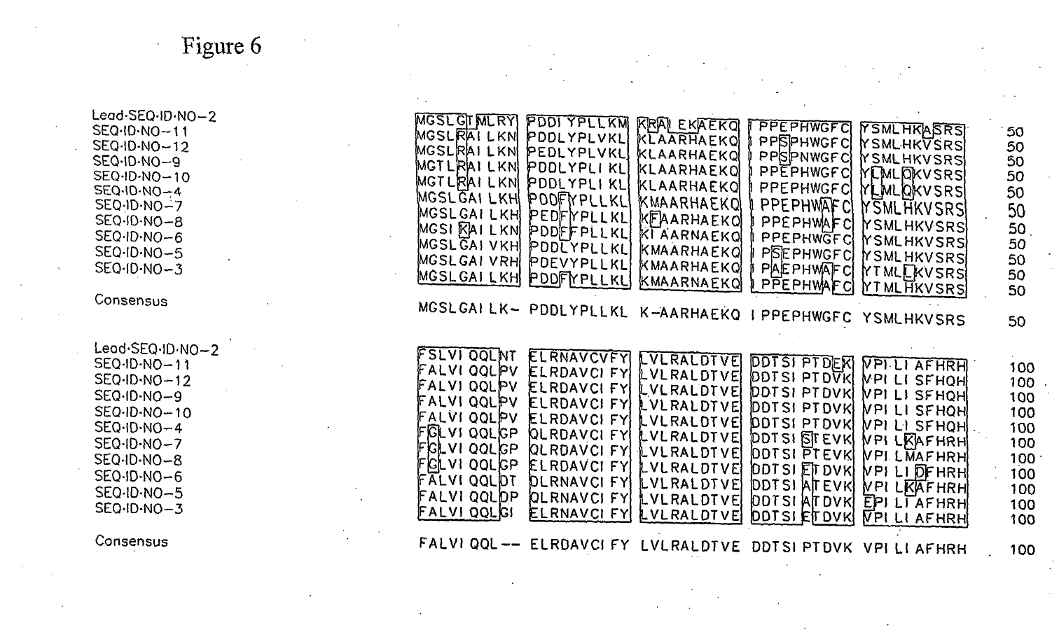

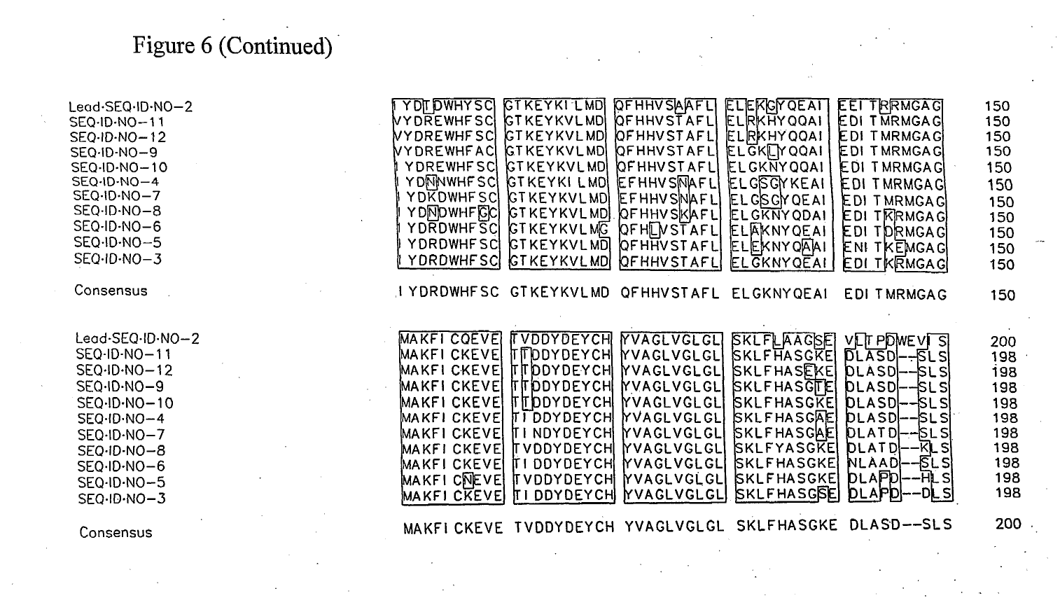

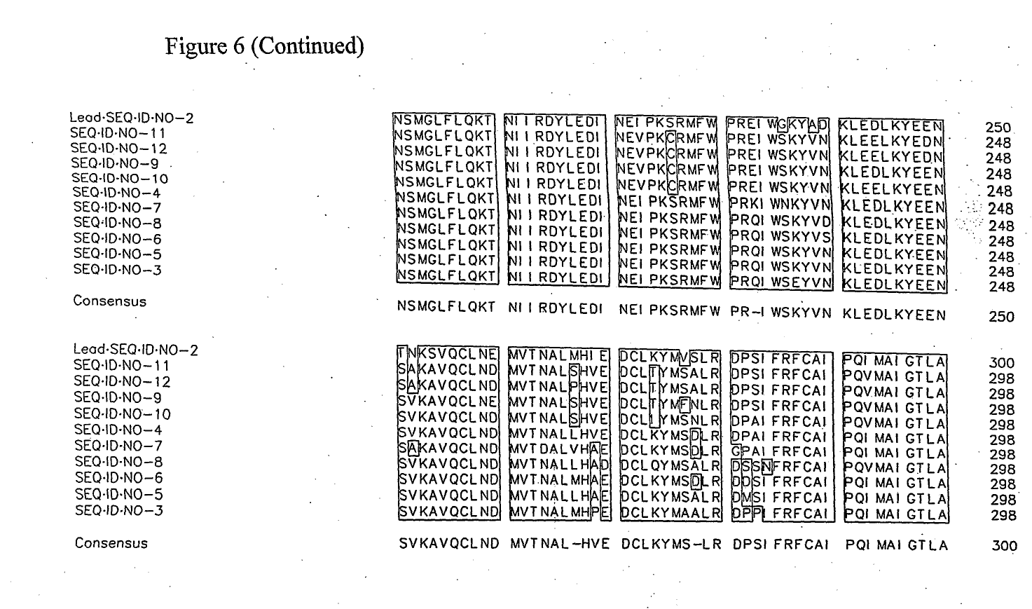

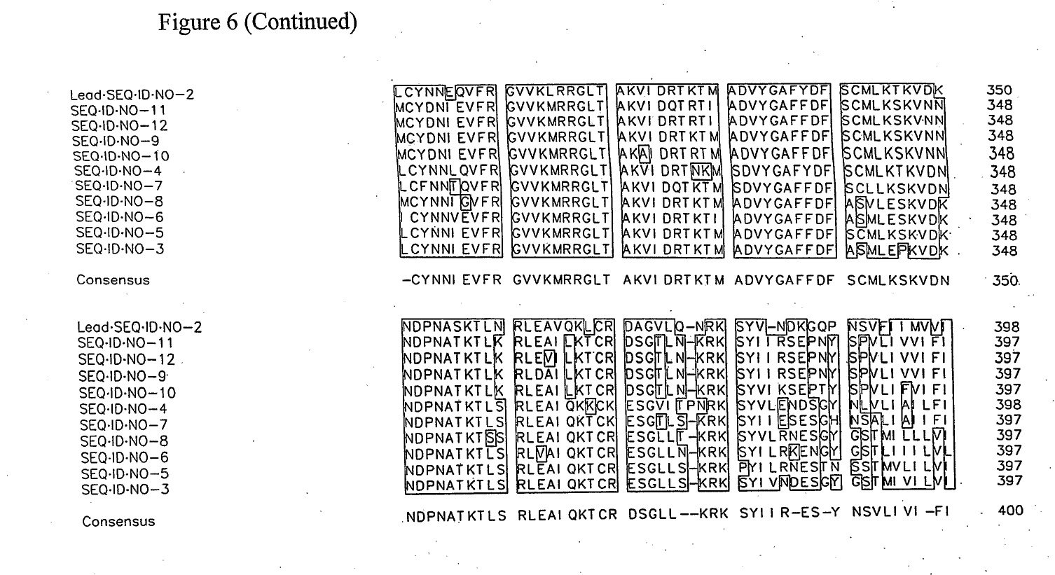

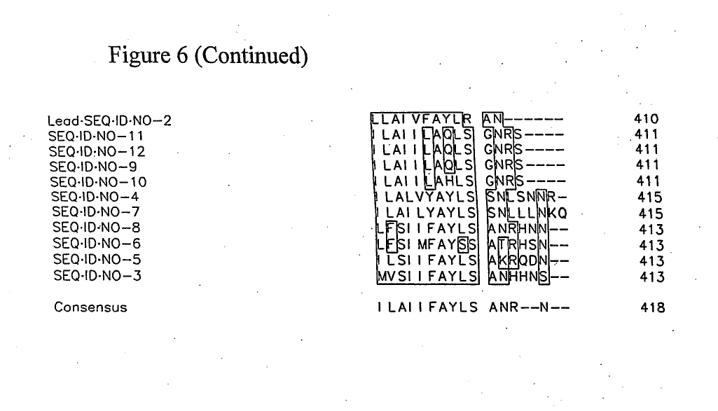

In some embodiments, an enzyme involved in biosynthesis of a triterpenoid compound can be one of the polypeptides whose amino acid sequence is set forth in FIG. 6, 7, 8, or 9, or can correspond to at least one of the consensus sequences as set forth in those figures. Thus, an enzyme involved in triterpenoid biosynthesis can be a squalene synthase. Squalene synthase catalyzes the first committed step in the branch point for diverting carbon specifically to the biosynthesis of triterpenoids. A squalene synthase can have the amino acid sequence encoded by cDNA 12328487 as set forth in FIG. 6 and in SEQ ID NO:2. cDNA 12328487 (SEQ ID NO: 1) is predicted to encode an Arabidopsis squalene/phytoene synthase. Thus, a squalene synthase can be an Arabidopsis polypeptide having the amino acid sequence set forth in SEQ ID NO:2. Alternatively, a squalene synthase can be an ortholog, homolog, or variant of the polypeptide having the sequence set forth in SEQ ID NO:2. A squalene synthase polypeptide, as described herein, can have an amino acid sequence with at least 35 percent sequence identity (e.g., 35 percent, 40 percent, 45 percent, 50 percent, 55 percent, 60 percent, 65 percent, 70 percent, 80 percent, 81 percent, 82 percent, 83 percent, 84 percent, 85 percent, 86 percent, 87 percent, 88 percent, 89 percent, 90 percent, 91 percent, 92 percent, 93 percent, 94 percent, 95 percent, 96 percent, 97 percent, 98 percent, or 99 percent sequence identity) to the amino acid sequence set forth in SEQ ID NO:2.

The alignment shown in FIG. 6 sets forth amino acid sequences of SEQ ID NO:2 orthologues and a consensus sequence. A consensus amino acid sequence for such orthologues was determined by aligning amino acid sequences, e.g., amino acid sequences related to SEQ ID NO:2, from a variety of species and determining the most common amino acid or type of amino acid at each position. For example, the alignment in FIG. 6 provides the amino acid sequences of cDNA 12328487 (SEQ ID NO: 2), Ceres Clone:515962 (SEQ ID NO: 3), gi55710094 (SEQ ID NO: 4), gi2144186 (SEQ ID NO: 5), gi28208268 (SEQ ID NO: 6), gi41224629 (SEQ ID NO: 7), gi27475614 (SEQ ID NO: 8), gi5360655 (SEQ ID NO: 9), gi4426953 (SEQ ID NO: 10), gi1552717 (SEQ ID NO: 11), and gill 184109 (SEQ ID NO: 12). In certain cases, therefore, a squalene synthase polypeptide can include an amino acid sequence having about 80% or greater sequence identity to an amino acid sequence set forth in FIG. 6, e.g., 80% or greater amino acid sequence identity to cDNA 12328487 (SEQ ID NO: 2), Ceres Clone:515962 (SEQ ID NO: 3), gi55710094 (SEQ ID NO: 4), gi2144186 (SEQ ID NO: 5), gi28208268 (SEQ ID NO: 6), gi41224629 (SEQ ID NO: 7), gi27475614 (SEQ ID NO: 8), gi5360655 (SEQ ID NO: 9), gi4426953 (SEQ ID NO: 10), gi/552717 (SEQ ID NO: 11), and gi11184109 (SEQ ID NO: 12). Eighty percent sequence identity or greater can be about 82, 85, 87, 90, 92, 95, 96, 97, 98, 99, or 100% sequence identity to such a sequence.

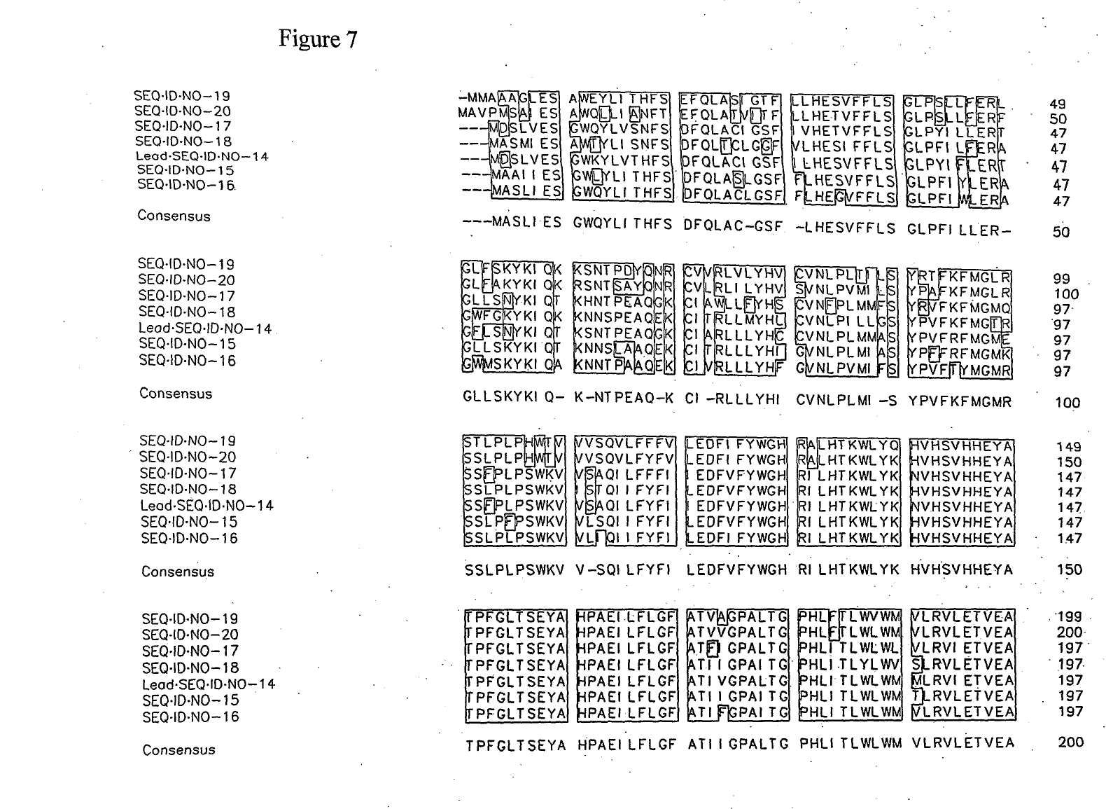

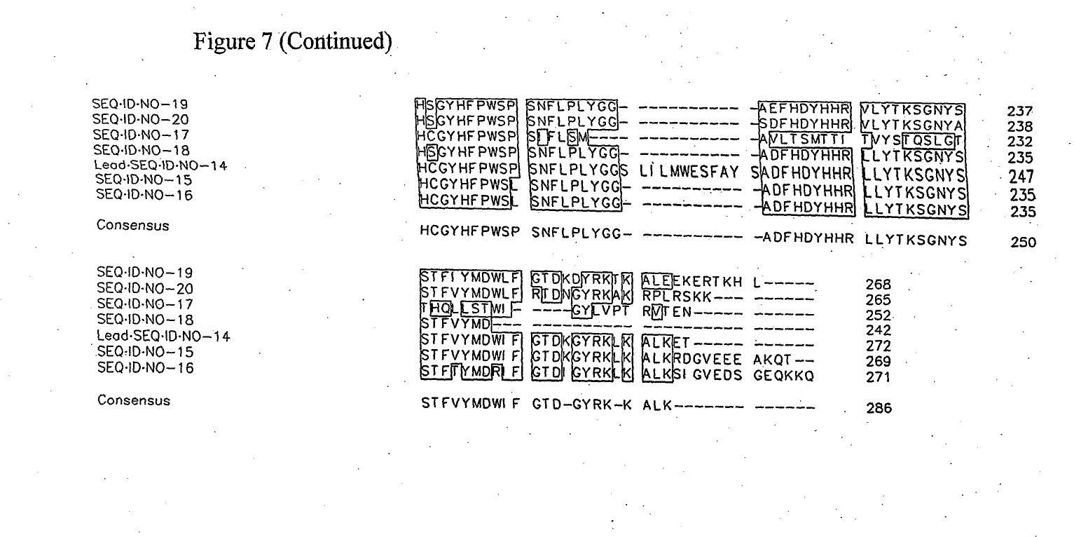

An enzyme involved in triterpenoid biosynthesis can also be a sterol methyl oxidase. Sterol methyl oxidase is a biosynthetic enzyme in the pathway leading to the production of important sterols such as campesterol, β-sitosterol and stigmasterol and catalyzes the conversion of 24-methylene cycloartanol to 4-carboxydimethyl cycloergosenol. A sterol methyl oxidase can have the amino acid sequence encoded by cDNA 12394143 as set forth in FIG. 7 and in SEQ ID NO:14. cDNA 12394143 (SEQ ID NO: 13) is predicted to encode an Arabidopsis sterol methyl oxidase/sterol desaturase. Thus, a sterol methyl oxidase can be an Arabidopsis polypeptide having the amino acid sequence set forth in SEQ ID NO: 14. Alternatively, a sterol methyl oxidase can be an ortholog, homolog, or variant of the polypeptide having the sequence set forth in SEQ ID NO: 14. A sterol methyl oxidase polypeptide, as described herein, can have an amino acid sequence with at least 35 percent sequence identity (e.g., 35 percent, 40 percent, 45 percent, 50 percent, 55 percent, 60 percent, 65 percent, 70 percent, 80 percent, 81 percent, 82 percent, 83 percent, 84 percent, 85 percent, 86 percent, 87 percent, 88 percent, 89 percent, 90 percent, 91 percent, 92 percent, 93 percent, 94 percent, 95 percent, 96 percent, 97 percent, 98 percent, or 99 percent sequence identity) to the amino acid sequence set forth in SEQ ID NO:14.

The alignment shown in FIG. 7 sets forth amino acid sequences of SEQ ID NO: 14 orthologues and a consensus sequence. A consensus amino acid sequence for such orthologues was determined by aligning amino acid sequences, e.g., amino acid sequences related to SEQ ID NO: 14, from a variety of species and determining the most common amino acid or type of amino acid at each position. For example, the alignment in FIG. 7 provides the amino acid sequences of cDNA 12394143, also known as CeresClone 23439, (SEQ ID NO: 14), gi27448145 (SEQ ID NO: 15), CeresClone:664026 (SEQ ID NO: 16), gi34978966 (SEQ ID NO: 18), gi51963234 (SEQ ID NO: 19), and CeresClone:217004 (SEQ ID NO: 20). Another orthologue can be CeresClone:245428 (SEQ ID NO: 21). In certain cases, therefore, a sterol methyl oxidase polypeptide can include an amino acid sequence having about 80% or greater amino acid sequence identity to cDNA 12394143 (SEQ ID NO: 14), gi27448145 (SEQ ID NO: 15), CeresClone:664026 (SEQ ID NO: 16), CeresClone:977729 (SEQ ID NO: 17), gi34978966 (SEQ ID NO: 18), gi51963234 (SEQ ID NO: 19), CeresClone:217004 (SEQ ID NO: 20), and CeresClone:245428 (SEQ ID NO: 21). Eighty percent sequence identity or greater can be about 82, 85, 87, 90, 92, 95, 96, 97, 98, 99, or 100% sequence identity to such a sequence.

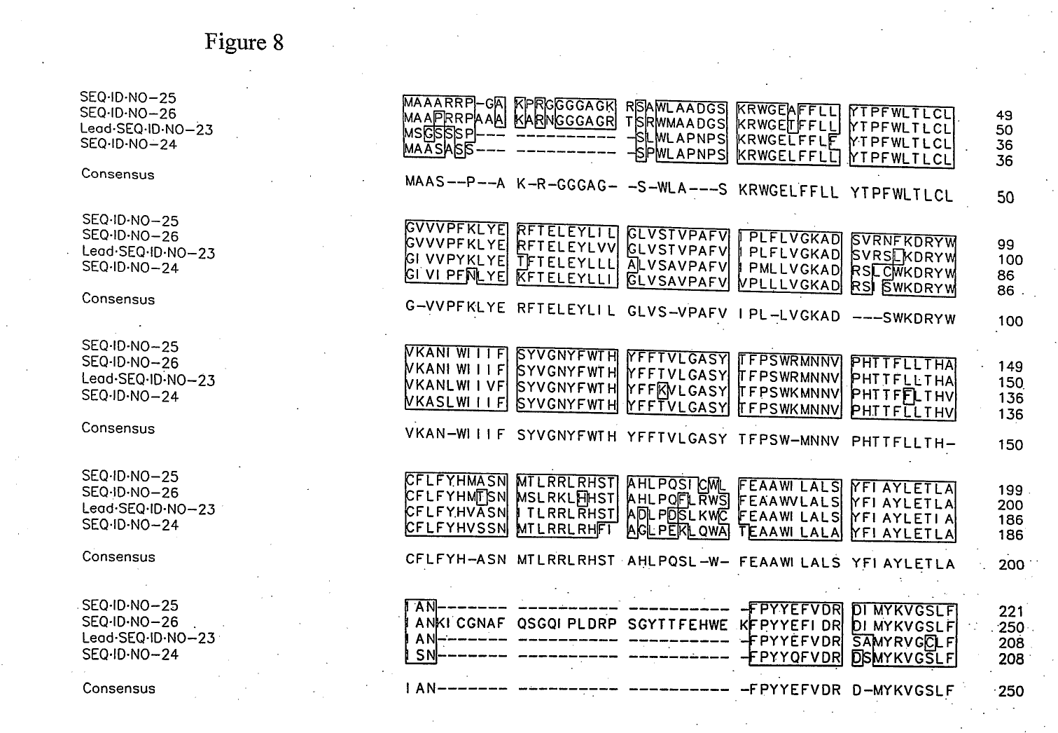

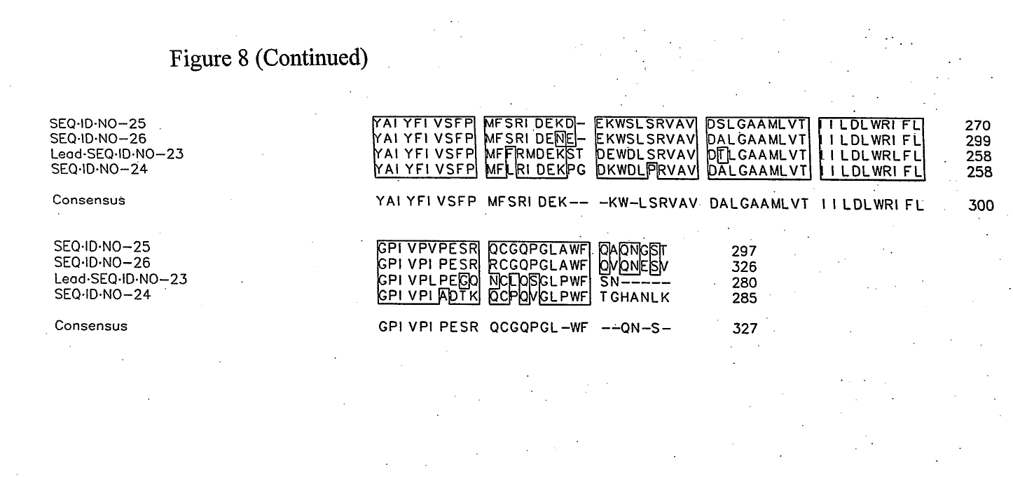

An enzyme involved in triterpenoid biosynthesis can also be a cyclopropyl sterol isomerase. Cyclopropyl sterol isomerase is a biosynthetic enzyme in the pathway leading to the production of important sterols such as campesterol, β-sitosterol and stigmasterol and acts downstream of sterol methyl oxidase to catalyze the conversion of cycloeucalenol to obtusifoliol. A cyclopropyl sterol isomerase can have the amino acid sequence encoded by cDNA 12421417 as set forth in FIG. 8 and in SEQ ID NO:23. cDNA 12421417 (SEQ ID NO: 22) is predicted to encode an Arabidopsis cyclopropyl sterol isomerase. Thus, a cyclopropyl sterol isomerase can be an Arabidopsis polypeptide having the amino acid sequence set forth in SEQ ID NO:23. Alternatively, a cyclopropyl sterol isomerase can be an ortholog, homolog, or variant of the polypeptide having the sequence set forth in SEQ ID NO:23. A cyclopropyl sterol isomerase, as described herein, can have an amino acid sequence with at least 35 percent sequence identity (e.g., 35 percent, 40 percent, 45 percent, 50 percent, 55 percent, 60 percent, 65 percent, 70 percent, 80 percent, 81 percent, 82 percent, 83 percent, 84 percent, 85 percent, 86 percent, 87 percent, 88 percent, 89 percent, 90 percent, 91 percent, 92 percent, 93 percent, 94 percent, 95 percent, 96 percent, 97 percent, 98 percent, or 99 percent sequence identity) to the amino acid sequence set forth in SEQ ID NO:23.

The alignment shown in FIG. 8 sets forth amino acid sequences of SEQ ID NO:23 orthologues and a consensus sequence. A consensus amino acid sequence for such orthologues was determined by aligning amino acid sequences, e.g., amino acid sequences related to SEQ ID NO:23, from a variety of species and determining the most common amino acid or type of amino acid at each position. For example, the alignment in FIG. 8 provides the amino acid sequences of cDNA 12421417 (SEQ ID NO: 23), CeresClone:716942 (SEQ ID NO: 24), CeresClone:285554 (SEQ ID NO: 25), and gi 62732798 (SEQ ID NO: 26). In certain cases, therefore, a cyclopropyl sterol-isomerase polypeptide can include an amino acid sequence having about 80% or greater sequence identity to an amino acid sequence set forth in FIG. 8, e.g., 80% or greater amino acid sequence identity to cDNA 12421417 (SEQ ID NO: 23), CeresClone:716942 (SEQ ID NO: 24), CeresClone:285554 (SEQ ID NO: 25), and gi62732798 (SEQ ID NO: 26). Eighty percent sequence identity or greater can be about 82, 85, 87, 90, 92, 95, 96, 97, 98, 99, or 100% sequence identity to such a sequence.

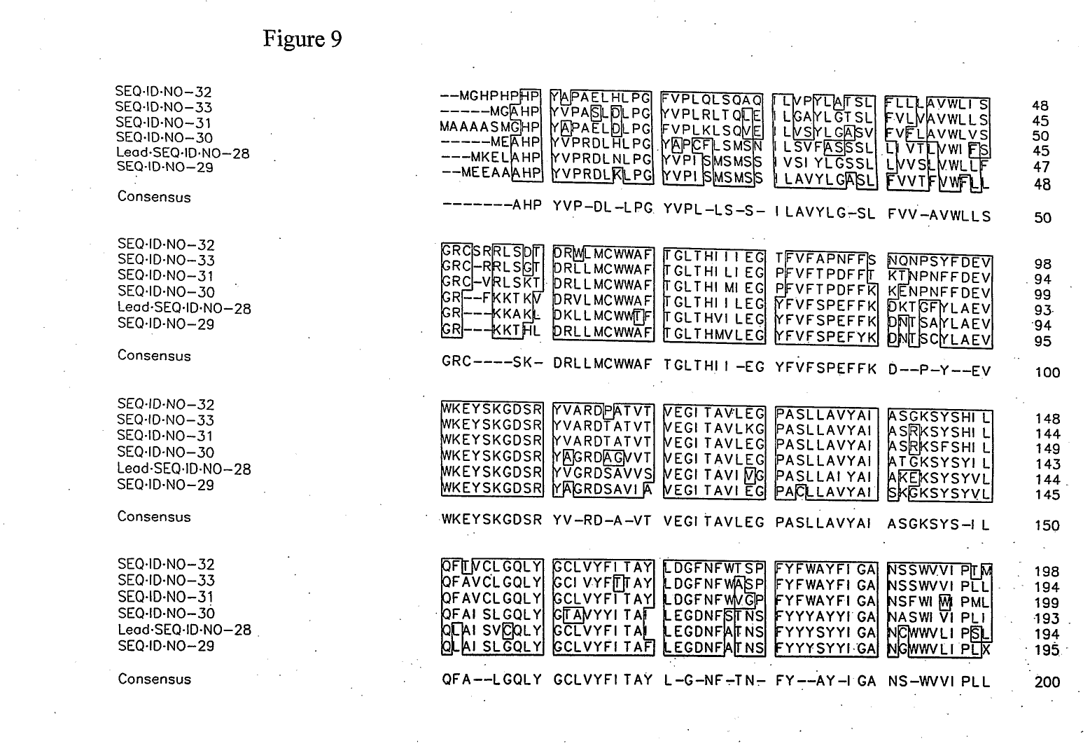

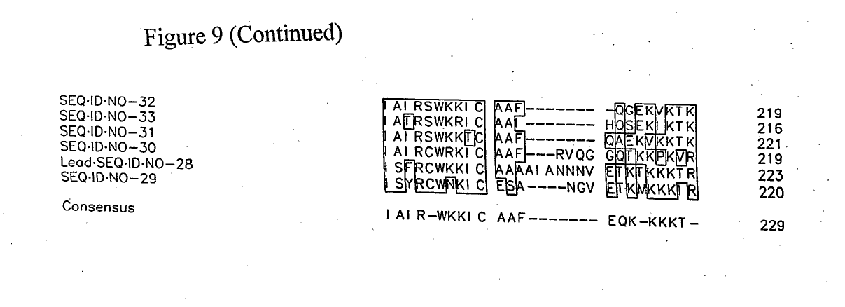

An enzyme involved in triterpenoid biosynthesis can also be a C-8,7 sterol isomerase. C-8,7 sterol isomerase is a biosynthetic enzyme in the pathway leading to the production of important sterols such as campesterol, β-sitosterol and stigmasterol and acts downstream of sterol isomerase to catalyze the conversion of 4-methyl-ergosta-8,24-dienol to 24-methylene lophenol. A C-8,7 sterol isomerase can have the amino acid sequence encoded by cDNA 13487250 as set forth in FIG. 9 and in SEQ ID NO:28. cDNA 13487250 (SEQ ID NO: 27) is predicted to encode an Arabidopsis C-8,7 sterol isomerase. C-8,7 sterol isomerases have region(s) of homology with emopamil binding proteins. Thus, a C-8,7 sterol isomerase can be an Arabidopsis polypeptide having the amino acid sequence set forth in SEQ ID NO:28. Alternatively, a C-8,7 sterol isomerase can be an ortholog, homolog, or variant of the polypeptide having the sequence set forth in SEQ ID NO:28. A C-8,7 sterol isomerase cyclopropyl, as described herein, can have an amino acid sequence with at least 35 percent sequence identity (e.g., 35 percent, 40 percent, 45 percent, 50 percent, 55 percent, 60 percent, 65 percent, 70 percent, 80 percent, 81 percent, 82 percent, 83 percent, 84 percent, 85 percent, 86 percent, 87 percent, 88 percent, 89 percent, 90 percent, 91 percent, 92 percent, 93 percent, 94 percent, 95 percent, 96 percent, 97 percent, 98 percent, or 99 percent sequence identity) to the amino acid sequence set forth in SEQ ID NO:28.

The alignment shown in FIG. 9 sets forth amino acid sequences of SEQ ID NO:28 orthologues and a consensus sequence. A consensus amino acid sequence for such orthologues was determined by aligning amino acid sequences, e.g., amino acid sequences related to SEQ ID NO:28, from a variety of species and determining the most common amino acid or type of amino acid at each position. For example, the alignment in FIG. 9 provides the amino acid sequences of cDNA 13487250 (SEQ ID NO: 28), CeresClone:959258 (SEQ ID NO: 29), CeresClone:592262 (SEQ ID NO: 30), CeresClone:282337 (SEQ ID NO: 31), gi50900588 (SEQ ID NO: 32), and CeresClone:703736 (SEQ ID NO: 33). In certain cases, therefore, a C-8,7 sterol isomerase polypeptide can include an amino acid sequence having about 80% or greater sequence identity to an amino acid sequence set forth in FIG. 9, e.g., 80% or greater amino acid sequence identity to cDNA 13487250 (SEQ ID NO: 28), CeresClone:959258 (SEQ ID NO: 29), CeresClone:592262 (SEQ ID NO: 30), CeresClone:282337 (SEQ ID NO: 31), gi50900588 (SEQ ID NO: 32), and CeresClone:703736 (SEQ ID NO: 33). Eighty percent sequence identity or greater can be about 82, 85, 87, 90, 92, 95, 96, 97, 98, 99, or 100% sequence identity to such a sequence.

It will be appreciated that a number of nucleic acids can encode a polypeptide having a particular amino acid sequence. The degeneracy of the genetic code is well known to the art; i.e., for many amino acids, there is more than one nucleotide triplet that selves as the codon for the amino acid. For example, codons in the coding sequence for a given triterpenoid-modulating polypeptide can be modified such that optimal expression in a particular plant species is obtained, using codon bias tables for that species.

A triterpenoid-modulating polypeptide encoded by a recombinant nucleic acid can be a native triterpenoid-modulating polypeptide, i.e., one or more additional copies of the coding sequence for a triterpenoid-modulating polypeptide that is naturally present in the cell. Alternatively, the triterpenoid-modulating polypeptide can be heterologous to the cell, e.g., a transgenic Lycopersicon plant can contain the coding sequence for a transcription factor from a Glycine plant.

Triterpenoid-modulating polypeptide candidates suitable for use in the invention can be identified by analysis of nucleotide and polypeptide sequence alignments. For example, performing a query on a database of nucleotide or polypeptide sequences can identify orthologs of triterpenoid-modulating polypeptides. Sequence analysis can involve BLAST or PSI-BLAST analysis of nonredundant databases using known triterpenoid-modulating polypeptide amino acid sequences. Those proteins in the database that have greater than 40% sequence identity can be identified as candidates for further evaluation for suitability as a triterpenoid-modulating polypeptide. If desired, manual inspection of such candidates can be carried out in order to narrow the number of candidates to be further evaluated. Manual inspection can be performed by selecting those candidates that appear to have domains suspected of being present in triterpenoid-modulating polypeptides, e.g., conserved functional domains.

The identification of conserved regions in a template or subject polypeptide can facilitate production of variants of wild type triterpenoid-modulating polypeptides. Conserved regions can be identified by locating a region within the primary amino acid sequence of a template polypeptide that is a repeated sequence, forms some secondary structure (e.g., helices and beta sheets), establishes positively or negatively charged domains, or represents a protein motif or domain. See, e.g., the Pfam web site describing consensus sequences for a variety of protein motifs and domains at sanger.ac.uk/Pfam and genome.wustl.edu/Pfam. A description of the information included at the Pfam database is described in Sonnhammer et. al, 1998, Nucl. Acids Res. 26: 320-322; Sonnhammer et. al, 1997, Proteins 28:405-420; and Bateman et. al., 1999, Nucl. Acids Res. 27:260-262.

Conserved regions also can be determined by aligning sequences of the same or related polypeptides from closely related species. Closely related species preferably are from the same family. In some embodiments, alignment of sequences from two different species is adequate. For example, sequences from Arabidopsis and Zea mays can be used to identify one or more conserved regions.

Typically, polypeptides that exhibit at least about 40% amino acid sequence identity are useful to identify conserved regions. Conserved regions of related polypeptides can exhibit at least 45% amino acid sequence identity (e.g., at least 50%, at least 60%, at least 70%, at least 80%, or at least 90% amino acid sequence identity). In some embodiments, a conserved region of target and template polypeptides exhibit at least 92, 94, 96, 98, or 99% amino acid sequence identity. Amino acid sequence identity can be deduced from amino acid or nucleotide sequences. In certain cases, highly conserved domains have been identified within triterpenoid-modulating polypeptides. These conserved regions can be useful in identifying functionally similar (orthologous) triterpenoid-modulating polypeptides.

In some instances, suitable triterpenoid-modulating polypeptides can be synthesized on the basis of consensus functional domains and/or conserved regions in polypeptides that are homologous triterpenoid-modulating polypeptides. Domains are groups of substantially contiguous amino acids in a polypeptide that can be used to characterize protein families and/or parts of proteins. Such domains have a “fingerprint” or “signature” that can comprise conserved (1) primary sequence, (2) secondary structure, and/or (3) three-dimensional conformation. Generally, domains are correlated with specific in vitro and/or in vivo activities. A domain can have a length of from 10 amino acids to 100 amino acids, e.g., 10 to 50 amino acids, or 25 to 100 amino acids, or 35 to 65 amino acids, or 35 to 55 amino acids, or 45 to 60 amino acids.

Representative homologs and/or orthologs are shown in FIGS. 1-9. Each Figure represents an alignment of the amino acid sequence of a query triterpenoid-modulating polypeptide with the amino acid sequences of corresponding homologs and/or orthologs. Amino acid sequences of query triterpenoid-modulating polypeptides and their corresponding homologs and/or orthologs have been aligned to identify conserved amino acids and to determine consensus sequences that contain frequently occurring amino acid residues at particular positions in the aligned sequences, as shown in FIGS. 1-9. A dash in an aligned sequence represents a gap, i.e., a lack of an amino acid at that position. Identical amino acids or conserved amino acid substitutions among aligned sequences are identified by boxes.

Each consensus sequence is comprised of conserved regions. Each conserved region contains a sequence of contiguous amino acid residues. A dash in a consensus sequence indicates that the consensus sequence either lacks an amino acid at that position or includes an amino acid at that position. If an amino acid is present, the residue at that position corresponds to one found in any aligned sequence at that position.

Useful triterpenoid-modulating polypeptides can be constructed based on the consensus sequence in any of FIGS. 1-9. Such a polypeptide includes the conserved regions in the selected consensus sequence, arranged in the order depicted in the Figure from amino-terminal end to carboxy-terminal end. Such a polypeptide may also include zero, one, or more than one amino acid in positions marked by dashes. When no amino acids are present at positions marked by dashes, the length of such a polypeptide is the sum of the amino acid residues in all conserved regions. When amino acids are present at all positions marked by dashes, such a polypeptide has a length that is the sum of the amino acid residues in all conserved regions and all dashes.

Consensus domains and conserved regions can be identified by homologous polypeptide sequence analysis as described herein. The suitability of such synthetic polypeptides for use as triterpenoid-modulating polypeptide can be evaluated by functional complementation of a heterologous regulatory triterpenoid-modulating polypeptide.

A triterpenoid-modulating polypeptide can be a fragment of a naturally occurring triterpenoid-modulating polypeptide. In certain cases, for example, triterpenoid-modulating polypeptides that are transcription factors, a fragment can comprise the DNA-binding and transcription-regulating domains of the naturally occurring transcription factor.

### B. Nucleic Acids

A transgenic plant or plant cell in which the amount and/or rate of biosynthesis of one or more triterpenoids is modulated includes at least one recombinant nucleic acid construct. The construct comprises a nucleic acid encoding a triterpenoid-modulating polypeptide as described herein, operably linked to a regulatory region suitable for expressing the triterpenoid-modulating polypeptide in the plant or cell. Thus, the invention features such recombinant nucleic acid constructs.

Isolated nucleic acids and polypeptides are provided herein. The terms “nucleic acid” and “polynucleotide” are used interchangeably herein, and refer to both RNA and DNA, including cDNA, genomic DNA, synthetic (e.g., chemically synthesized) DNA, and DNA (or RNA) containing nucleic acid analogs. Polynucleotides can have any three-dimensional structure. A nucleic acid can be double-stranded or single-stranded (i.e., a sense strand or an antisense strand). Non-limiting examples of polynucleotides include genes, gene fragments, exons, introns, messenger RNA (mRNA), transfer RNA, ribosomal RNA, siRNA, micro-RNA, ribozymes, cDNA, recombinant polynucleotides, branched polynucleotides, plasmids, vectors, isolated DNA of any sequence, isolated RNA of any sequence, nucleic acid probes, and primers, as well as nucleic acid analogs. As used herein, “isolated,” when in reference to a nucleic acid, refers to a nucleic acid that is separated from other nucleic acids that are present in a genome, e.g., a plant genome, including nucleic acids that normally flank one or both sides of the nucleic acid in the genome. The term “isolated” as used herein with respect to nucleic acids also includes any non-naturally-occurring sequence, since such non-naturally-occurring sequences are not found in nature and do not have immediately contiguous sequences in a naturally-occurring genome.

An isolated nucleic acid can be, for example, a DNA molecule, provided one of the nucleic acid sequences normally found immediately flanking that DNA molecule in a naturally-occurring genome is removed or absent. Thus, an isolated nucleic acid includes, without limitation, a DNA molecule that exists as a separate molecule, independent of other sequences (e.g., a chemically synthesized nucleic acid, or a cDNA or genomic DNA fragment produced by the polymerase chain reaction (PCR) or restriction endonuclease treatment). An isolated nucleic acid also refers to a DNA molecule that is incorporated into a vector, an autonomously replicating plasmid, a virus (e.g., pararetrovirus, retrovirus, lentivirus, adenovirus, adeno-associated virus, or herpesvirus), or into the genomic DNA of a prokaryote or eukaryote. In addition, an isolated nucleic acid can include an engineered nucleic acid such as a DNA molecule that is part of a hybrid or fusion nucleic acid. A nucleic acid existing among hundreds to millions of other nucleic acids within, for example, cDNA libraries or genomic libraries, or gel slices containing a genomic DNA restriction digest, is not to be considered an isolated nucleic acid.

Isolated nucleic acid molecules can be produced by standard techniques. For example, polymerase chain reaction (PCR) techniques can be used to obtain an isolated nucleic acid containing a nucleotide sequence described herein. PCR can be used to amplify specific sequences from DNA as well as RNA, including sequences from total genomic DNA or total cellular RNA. Various PCR methods are described, for example, in PCR Primer: A Laboratory Manual, Dieffenbach and Dveksler, eds., Cold Spring Harbor Laboratory Press, 1995. Generally, sequence information from the ends of the region of interest or beyond is employed to design oligonucleotide primers that are identical or similar in sequence to opposite strands of the template to be amplified. Various PCR strategies also are available by which site-specific nucleotide sequence modifications can be introduced into a template nucleic acid. Isolated nucleic acids also can be chemically synthesized, either as a single nucleic acid molecule (e.g., using automated DNA synthesis in the 3′ to 5′ direction using phosphoramidite technology) or as a series of oligonucleotides. For example, one or more pairs of long oligonucleotides (e.g., >100 nucleotides) can be synthesized that contain the desired sequence, with each pair containing a short segment of complementarity (e.g., about 15 nucleotides) such that a duplex is formed when the oligonucleotide pair is annealed. DNA polymerase is used to extend the oligonucleotides, resulting in a single, double-stranded nucleic acid molecule per oligonucleotide pair, which then can be ligated into a vector. Isolated nucleic acids of the invention also can be obtained by mutagenesis of, e.g., a naturally occurring DNA.

As used herein, the term “percent sequence identity” refers to the degree of identity between any given query sequence and a subject sequence. A subject sequence typically has a length that is more than 80%, e.g., more than 82%, 85%, 87%, 89%, 90%, 93%, 95%, 97%, 99%, 100%, 105%, 110%, 115%, or 120%, of the length of the query sequence. A query nucleic acid or amino acid sequence is aligned to one or more subject nucleic acid or amino acid sequences using the computer program ClustalW (version 1.83, default parameters), which allows alignments of nucleic acid or protein sequences to be carried out across their entire length (global alignment). Chema et al., Nucleic Acids Res., 31(13):3497-500 (2003).

ClustalW calculates the best match between a query and one or more subject sequences, and aligns them so that identities, similarities and differences can be determined. Gaps of one or more residues can be inserted into a query sequence, a subject sequence, or both, to maximize sequence alignments. For fast pairwise alignment of nucleic acid sequences, the following default parameters are used: word size: 2; window size: 4; scoring method: percentage; number of top diagonals: 4; and gap penalty: 5. For multiple alignment of nucleic acid sequences, the following parameters are used: gap opening penalty: 10.0; gap extension penalty: 5.0; and weight transitions: yes. For fast pairwise alignment of protein sequences, the following parameters are used: word size: 1; window size: 5; scoring method: percentage; number of top diagonals: 5; gap penalty: 3. For multiple alignment of protein sequences, the following parameters are used: weight matrix: blosum; gap opening penalty: 10.0; gap extension penalty: 0.05; hydrophilic gaps: on; hydrophilic residues: Gly, Pro, Ser, Asn, Asp, Gln, Glu, Arg, and Lys; residue-specific gap penalties: on. The output is a sequence alignment that reflects the relationship between sequences. ClustalW can be run, for example, at the Baylor College of Medicine Search Launcher site (searchlauncher.bcm.tmc.edu/multi-align/multi-align.html) and at the European Bioinformatics Institute site on the World Wide Web (ebi.ac.ul/clustalw). To determine a “percent identity” between a query sequence and a subject sequence, the number of matching bases or amino acids in the alignment is divided by the total number of matched and mismatched bases or amino acids, followed by multiplying the result by 100. It is noted that the percent identity value can be rounded to the nearest tenth. For example, 78.11, 78.12, 78.13, and 78.14 is rounded down to 78.1, while 78.15, 78.16, 78.17, 78.18, and 78.19 is rounded up to 78.2. It also is noted that the length value will always be an integer.

“Altered level of gene expression” as used herein refers to a comparison of the level of expression of a transcript of a gene or the amount of its corresponding polynucleotide in the presence and absence of a triterpenoid-modulating polypeptide described herein, and refers to a measurable or observable change in the level of expression of a transcript of a gene or the amount of its corresponding polynucleotide relative to a control plant or plant cell under the same conditions (e.g., as measured through a suitable assay such as quantitative RT-PCR, a “northern blot” or through an observable change in phenotype, chemical profile, or metabolic profile). An altered level of gene expression can include increased (activation) or decreased (repression) expression of a transcript of a gene or polynucleotide relative to a control plant or plant cell under the same conditions. Altered expression levels can occur under different environmental or developmental conditions or in different locations than those exhibited by a plant or plant cell in its native state.

The term “exogenous” with respect to a nucleic acid indicates that the nucleic acid is part of a recombinant nucleic acid construct, or is not in its natural environment. For example, an exogenous nucleic acid can be a sequence from one species introduced into another species, i.e., a heterologous nucleic acid. Typically, such an exogenous nucleic acid is introduced into the other species via a recombinant nucleic acid construct. An exogenous nucleic acid can also be a sequence that is native to an organism and that has been reintroduced into cells of that organism. An exogenous nucleic acid that includes a native sequence can often be distinguished from the naturally occurring sequence by the presence of non-natural sequences linked to the exogenous nucleic acid, e.g., non-native regulatory sequences flanking a native sequence in a recombinant nucleic acid construct. In addition, stably transformed exogenous nucleic acids typically are integrated at positions other than the position where the native sequence is found. It will be appreciated that an exogenous nucleic acid may have been introduced into a progenitor and not into the cell under consideration. For example, a transgenic plant containing an exogenous nucleic acid can be the progeny of a cross between a stably transformed plant and a non transgenic plant. Such progeny are considered to contain the exogenous nucleic acid.

### II. Recombinant Constructs and Vectors

Recombinant constructs are also provided herein and can be used to transform plants or plant cells in order to modulate the level of one or more triterpenoids. A recombinant nucleic acid construct comprises a nucleic acid encoding one or more triterpenoid-modulating polypeptides as described herein, operably linked to a regulatory region suitable for expressing the triterpenoid-modulating polypeptide in the plant or cell. Thus, a nucleic acid can comprise a coding sequence that includes any of the triterpenoid-modulating polypeptides as set forth in FIG. 1, 2, 3, 4, or 5. A nucleic acid can also comprise a coding sequence that includes any of the triterpenoid modulating polypeptides involved in triterpenoid biosynthesis as set forth in FIG. 6, 7, 8, or 9.

### A. Vectors

Vectors containing nucleic acids such as those described herein also are provided. A “vector” is a replicon, such as a plasmid, phage, or cosmid, into which another DNA segment may be inserted so as to bring about the replication of the inserted segment. Generally, a vector is capable of replication when associated with the proper control elements. Suitable vector backbones include, for example, those routinely used in the art such as plasmids, viruses, artificial chromosomes, BACs, YACs, or PACs. The term “vector” includes cloning and expression vectors, as well as viral vectors and integrating vectors. An “expression vector” is a vector that includes a regulatory region. Suitable expression vectors include, without limitation, plasmids and viral vectors derived from, for example, bacteriophage, baculoviruses, tobacco mosaic virus and retroviruses. Numerous vectors and expression systems are commercially available from such corporations as Novagen (Madison, Wis.), Clontech (Palo Alto, Calif.), Stratagene (La Jolla, Calif.), and Invitrogen/Life Technologies (Carlsbad, Calif.).

The vectors provided herein also can include, for example, origins of replication, scaffold attachment regions (SARs), and/or markers. A marker gene can confer a selectable phenotype on a plant cell. For example, a marker can confer, biocide resistance, such as resistance to an antibiotic (e.g., kanamycin, G418, bleomycin, or hygromycin), or an herbicide (e.g., chlorosulfuron or phosphinothricin). In addition, an expression vector can include a tag sequence designed to facilitate manipulation or detection (e.g., purification or localization) of the expressed polypeptide. Tag sequences, such as green fluorescent protein (GFP), glutathione S-transferase (GST), polyhistidine, c-myc, hemagglutinin, or Flag™ tag (Kodak, New Haven, Conn.) sequences typically are expressed as a fusion with the encoded polypeptide. Such tags can be inserted anywhere within the polypeptide, including at either the carboxyl or amino terminus.

### B. Regulatory Regions

The term “expression” refers to the process of converting genetic information encoded in a gene or polynucleotide into RNA (e.g., mRNA, rRNA, tRNA, or snRNA) through “transcription” of the gene or polynucleotide (i.e., via the enzymatic action of an RNA polymerase), and into protein, through “translation” of mRNA. Expression may be regulated at many stages in the process. “Up-regulation” or “activation” refers to regulation that increases the production of expression products (i.e., RNA or protein) relative to basal or native states, while “down-regulation” or “repression” refers to regulation that decreases production relative to basal or native states. Molecules (e.g., regulatory proteins) that are involved in up-regulation or down-regulation are often called “activators” and “repressors,” respectively.

The term “regulatory region” refers to nucleotide sequences that influence transcription or translation initiation and rate, and stability and/or mobility of the transcript or polypeptide product. Regulatory regions include, without limitation, promoter sequences, enhancer sequences, response elements, protein recognition sites, inducible elements, promoter control elements, protein binding sequences, 5, and 3, untranslated regions (UTRs), transcriptional start sites, termination sequences, polyadenylation sequences, introns, and other regulatory regions that can reside within coding sequences, such as secretory signals and protease cleavage sites.

As used herein, the term “operably linked” refers to positioning of a regulatory region and a transcribable sequence in a nucleic acid so as to allow or facilitate transcription of the transcribable sequence. For example, to bring a coding sequence under the control of a promoter, it typically is necessary to position the translation initiation site of the translational reading frame of the polypeptide between one and about fifty nucleotides downstream of the promoter. A promoter can, however, be positioned as much as about 5,000 nucleotides upstream of the translation start site, or about 2,000 nucleotides upstream of the transcription start site. A promoter typically comprises at least a core (basal) promoter. A promoter also may include at least one control element such as an upstream element. Such elements include upstream activation regions (UARs) and, optionally, other DNA sequences that affect transcription of a polynucleotide such as a synthetic upstream element. The choice of promoters to be included depends upon several factors, including, but not limited to, efficiency, selectability, inducibility, desired expression level, and cell or tissue specificity. It is a routine matter for one of skill in the art to modulate the expression of a coding sequence by appropriately selecting and positioning promoters and other regulatory regions relative to the coding sequence.

Some suitable promoters initiate transcription only, or predominantly, in certain cell types. For example, a promoter specific to a reproductive tissue (e.g., fruit, ovule, seed, pollen, pistils, female gametophyte, egg cell, central cell, nucellus, suspensor, synergid cell, flowers, embryonic tissue, embryo, zygote, endosperm, integument, seed coat or pollen) can be used. A cell type or tissue-specific promoter, however, may drive expression of operably linked sequences in tissues other than the target tissue. Thus, as used herein a cell type or tissue-specific promoter is one that drives expression preferentially in the target tissue, but may also lead to some expression in other cell types or tissues as well. Methods for identifying and characterizing promoter regions in plant genomic DNA include, for example, those described in the following references: Jordano, et al., Plant Cell, 1:855-866 (1989); Bustos, et al., Plant Cell, 1:839-854 (1989); Green, et al., EMBO J. 7, 4035-4044 (1988); Meier, et al., Plant Cell, 3, 309-316 (1991); and Zhang, et al., Plant Physiology 110: 1069-1079 (1996).

Examples of various classes of promoters are described below. Some of the promoters indicated below as well as additional promoters are described in more detail in U.S. Patent Application Ser. Nos. 60/505,689; 60/518,075; 60/544,771; 60/558,869; 60/583,691; 60/619,181; 60/637,140; 60/757,544; 60/776,307; 10/957,569; 11/058,689; 11/172,703; 11/208,308; 11/274,890; 60/583,609; 60/612,891; 11/097,589; 11/233,726; 11/408,791; 11/414,142; 10/950,321; 11/360,017; PCT/US05/011105; PCT/US05/034308; and PCT/US05/23639. Nucleotide sequences of promoters are set forth in SEQ ID NOs: 62-155. It will be appreciated that a promoter may meet criteria for one classification based on its activity in one plant species, and yet meet criteria for a different classification based on its activity in another plant species.

1. Constitutive Promoters

Constitutive promoters can promote transcription of an operably linked nucleic acid under most, but not necessarily all, environmental conditions and states of development or cell differentiation. Non-limiting examples of constitutive promoters that can be included in the nucleic acid constructs provided herein include the cauliflower mosaic virus (CaMV) 35S transcription initiation region, the mannopine synthase (MAS) promoter, the 1′ or 2′ promoters derived from T-DNA of Agrobacterium tumefaciens, the figwort mosaic virus 35S promoter, actin promoters such as the rice actin promoter, and ubiquitin promoters such as the maize ubiquitin-1 promoter.

2. Broadly Expressing Promoters

A promoter can be said to be “broadly expressing” when it promotes transcription in many, but not all, plant tissues. For example, a broadly expressing promoter can promote transcription of an operably linked sequence in one or more of the stem, shoot, shoot tip (apex), and leaves, but can promote transcription weakly or not at all in tissues such as reproductive tissues of flowers and developing seeds. In certain cases, a broadly expressing promoter operably linked to a sequence can promote transcription of the linked sequence in a plant shoot at a level that is at least two times, e.g., at least 3, 5, 10, or 20 times, greater than the level of transcription in a developing seed. In other cases, a broadly expressing promoter can promote transcription in a plant shoot at a level that is at least two times, e.g., at least 3, 5, 10, or 20 times, greater than the level of transcription in a reproductive tissue of a flower. In view of the above, the CaMV 35S promoter is not considered a broadly expressing promoter. Non-limiting examples of broadly expressing promoters that can be included in the nucleic acid constructs provided herein include the p326, YP0158, YP0214, YP0380, PT0848, PT0633, YP0050, YP0144 and YP0190 promoters. See, e.g., U.S. patent application Ser. No. 11/208,308, filed Aug. 19, 2005.

3. Root-Specific Promoters

Root-specific promoters confer transcription only or predominantly in root tissue. Examples of root-specific promoters include YP0128 (SEQ ID NO: 63), YP0275 (SEQ ID NO: 65), PT0625 (SEQ ID NO: 67), PT0660 (SEQ ID NO: 71), PT0683 (SEQ ID NO: 73), PT0758 (SEQ ID NO: 75), the root specific subdomains of the CaMV 35S promoter (Lam et al., Proc Natl Acad Sci USA 86:7890-7894 (1989)), root cell specific promoters reported by Conkling et. al. Plant Physiol. 93:1203-1211 (1990), and the tobacco RD2 gene promoter.

4. Seed-Specific Promoters

In some embodiments, promoters that are predominantly specific to seeds can be useful. Transcription from a seed-specific promoter can occur primarily in endosperm and cotyledon tissue during seed development. Non-limiting examples of seed-specific promoters that can be included in the nucleic acid constructs provided herein include the promoters YP0092 (SEQ ID NO: 62), PT0676 (SEQ ID NO: 72), PT0708 (SEQ ID NO: 74), PT0613 (SEQ ID NO: 66), PT0672 (SEQ ID NO: 68), PT0678 (SEQ ID NO: 69), PT0688 (SEQ ID NO: 70), PT0837 (SEQ ID NO: 76), the napin promoter, the Arcelin-5 promoter, the phaseolin gene promoter (Bustos et al., Plant Cell 1(9):839-853 (1989)), the soybean trypsin inhibitor promoter (Riggs et al., Plant Cell 1(6):609-621 (1989)), the ACP promoter (Baerson et al., Plant Mol Biol, 22(2):255-267 (1993)), the stearoyl-ACP desaturase gene (Slocombe et al., Plant Physiol 104(4): 167-176 (1994)), the soybean α′ subunit of β-conglycinin promoter (Chen et al., Proc Natl Acad Sci USA 83:8560-8564 (1986)), the oleosin promoter (Hong et al., Plant Mol Biol 34(3):549-555 (1997)), zein promoters such as the 15 kD zein promoter, the 16 kD zein promoter, 19 kD zein promoter, 22 kD zein promoter and 27 kD zein promoter. Also suitable are the Osgt-1 promoter from the rice glutelin-1 gene (Zheng et. al., Mol. Cell Biol. 13:5829-5842 (1993)), the beta-amylase gene promoter, and the barley hordein gene promoter.

5. Non-Seed Fruit Tissue Promoters

Promoters that are active in non-seed fruit tissues can also be useful, e.g., a polygalacturonidase promoter, the banana TRX promoter, and the melon actin promoter.

6. Photosynthetically-Active Tissue Promoters

Photosynthetically-active tissue promoters confer transcription only or predominantly in photosynthetically active tissue. Examples of such promoters include the ribulose-1,5-bisphosphate carboxylase (RbcS) promoters such as the RbcS promoter from eastern larch (Larix laricina), the pine cab6 promoter (Yamamoto et al., Plant Cell Physiol. 35:773-778 (1994)), the Cab-1 gene promoter from wheat (Fejes et al., Plant Mol. Biol. 15:921-932 (1990)), the CAB-1 promoter from spinach (Lubberstedt et al., Plant Physiol. 104:997-1006 (1994)), the cab1R promoter from rice (Luan et al., Plant Cell 4:971-981 (1992)), the pyruvate orthophosphate dikinase (PPDK) promoter from corn (Matsuoka et al., Proc Natl Acad. Sci USA 90:9586-9590 (1993)), the tobacco Lhcb1*2 promoter (Cerdan et al., Plant Mol. Biol. 33:245-255 (1997)), the Arabidopsis thaliana SUC2 sucrose-H+ symporter promoter (Truemit et al., Planta. 196:564-570 (1995)), and thylakoid membrane protein promoters from spinach (psaD, psaF, psaE, PC, FNR, atpC, atpD, cab, rbcS).

7. Basal Promoters

A basal promoter is the minimal sequence necessary for assembly of a transcription complex required for transcription initiation. Basal promoters frequently include a “TATA box” element that may be located between about 15 and about 35 nucleotides upstream from the site of transcription initiation. Basal promoters also may include a “CCAAT box” element (typically the sequence CCAAT) and/or a GGGCG sequence, which can be located between about 40 and about 200 nucleotides, typically about 60 to about 120 nucleotides, upstream from the transcription start site.

8. Other Promoters

Other classes of promoters include, but are not limited to, inducible promoters, such as promoters that confer transcription in response to external stimuli such as chemical agents, developmental stimuli, or environmental stimuli. Promoters designated YP0086 (gDNA ID 7418340), YP0188 (gDNA ID 7418570), YP0263 (gDNA ID 7418658), p13879, p32449, PT0758; PT0743; PT0829; YP0119; and YP0096, as described in the above-referenced patent applications, may also be useful.

9. Other Regulatory Regions

The recombinant constructs provided herein can also encode DNA sequences that are transcribed into RNA, but are not translated. Untranslated regions (UTR's) modulate many aspects of RNA functions including mRNA stability, translational efficiency and mRNA localization. A 5′ UTR lies between the start site of the transcript and the translation initiation codon and may include the +1 nucleotide. Examples of 5′ UTR's include, but are not limited to, internal ribosome entry sequences (IRES), upstream open reading frames (uORF's) and iron-response elements (IRE's). A 3′ UTR can be positioned between the translation termination codon and the end of the transcript. Examples of 3′ UTRs include, but are not limited to, AU-rich elements (ARE's), polyadenylation signals, selenocysteine insertion sequences (SECIS elements), and transcription termination sequences. A polyadenylation region at the 3′-end of a coding region can also be operably linked to a coding sequence. The polyadenylation region can be derived from the natural gene, from various other plant genes, or from an Agrobacterium T-DNA gene.

A suitable enhancer is a cis-regulatory element (−212 to −154) from the upstream region of the octopine synthase (ocs) gene. Fromm et al., The Plant Cell 1:977-984 (1989).

It will be understood that more than one regulatory region may be present in a recombinant polynucleotide, e.g., introns, enhancers, upstream activation regions, and inducible elements. Thus, more than one regulatory region can be operably linked to the sequence for a triterpenoid-modulating polypeptide.

### C. Combinations of Nucleic Acids

A transgenic plant or plant cell in which the amount and/or rate of biosynthesis of one or more triterpenoids is modulated can have one or more exogenous nucleic acids encoding the triterpenoid-modulating polypeptide sequences described herein. In some embodiments, more than one additional exogenous nucleic acid is present in a plant, e.g., two, three, four, five, six, seven, eight, nine, ten or more of such sequences. Each additional exogenous nucleic acid can be present on the same nucleic acid construct, or can be present on one or more separate nucleic acid constructs. For example, two recombinant nucleic acid constructs can be included, where a first construct includes a nucleic acid encoding a first triterpenoid modulating polypeptide, and a second construct includes a nucleic acid encoding a second triterpenoid modulating polypeptide. Of course, regulatory regions such as promoters, introns, enhancers, upstream activation regions, and inducible elements typically can be operably linked to an additional nucleic acid.

Thus, combinations of triterpenoid-modulating polypeptides can be present in a transgenic plant. In one embodiment, a combination can include one- or more triterpenoid modulating polypeptides that are transcription factors in combination with one or more triterpenoid-modulating polypeptides that are enzymes involved in triterpenoid biosynthesis. All permutations of a transcription factor in combination with a triterpenoid-modulating polypeptide that is an enzyme involved in triterpenoid biosynthesis and described herein are encompassed by the previous sentence, as well as any and all subsets of such permutations. For example, a first nucleic acid can encode an AP2 domain containing transcription factor and a second nucleic acid can encode an enzyme involved in triterpenoid biosynthesis, e.g., squalene synthase or sterol methyl oxidase. In another embodiment, a combination can include two or more triterpenoid modulating polypeptides that are transcription factors or redox proteins. All permutations of transcription factors and redox proteins described herein are encompassed by the previous sentence, as well as any and all subsets of such permutations. For example, a first nucleic acid can encode an AP2 domain containing transcription factor and a second nucleic acid can encode a homeodomain containing polypeptide. In another example, a first nucleic acid can encode an AP2 domain containing transcription factor and a second nucleic acid can encode a thioredoxin polypeptide. In another aspect, a combination can include two or more triterpenoid-modulating polypeptides that are enzymes involved in triterpenoid biosynthesis. All permutations of two or more triterpenoid-modulating polypeptides that are enzymes involved in triterpenoid biosynthesis and described herein are encompassed by the previous sentence, as well as any and all subsets of such permutations. For example, two or more of farnesyl diphosphate synthase, farnesyl-diphosphate:farnesyl-diphosphate farnesyltransferase, squalene synthase, squalene, hydrogen-donor:oxygen oxidoreductase (2,3-epoxidizing), also known as squalene-2,3-epoxide cyclase, cycloartenol synthase, cyclopropyl sterol isomerase, also known as cycloeucalenol cycloisomerase, C-8,7 sterol isomerase, sterol methyl transferase2, sterol methyl oxidase, dammarenediol synthase α-amyrin synthase, β-amyrin synthase, lupeol synthase, hopene cyclase, sesqueterpene synthases, sesqueterpene cylases, or pentacyclic triterpene synthases. As another example, a first nucleic acid can encode a squalene synthase enzyme and a second nucleic acid can encode a sterol methyl oxidase.

Alternatively, the polynucleotides and recombinant vectors described herein can be used to suppress or inhibit expression of a triterpenoid-modulating polypeptide in a plant species of interest. For example, inhibition or suppression of transcription or translation of a particular triterpenoid-modulating polypeptide in one branch of a metabolic pathway in triterpenoid biosynthesis may result in increased production of critical intermediates required for the biosynthesis of specific triterpenoids in another branch of the metabolic pathway. Thus, in another embodiment, a construct can have a sequence that is transcribed into a nucleic acid that selectively reduces biosynthesis of a particular triterpenoid.

A number of nucleic-acid based methods, including anti-sense RNA, ribozyme directed RNA cleavage, and interfering RNA (RNAi) can be used to inhibit protein expression in plants. Antisense technology is one well-known method. In this method, a nucleic acid segment from the endogenous gene is cloned and operably linked to a promoter so that the antisense strand of RNA is transcribed. The recombinant vector is then transformed into plants, as described above, and the antisense strand of RNA is produced. The nucleic acid segment need not be the entire sequence of the endogenous gene to be repressed, but typically will be substantially identical to at least a portion of the endogenous gene to be repressed. Generally, higher homology can be used to compensate for the use of a shorter sequence. Typically, a sequence of at least 30 nucleotides is used (e.g., at least 40, 50, 80, 100, 200, 500 nucleotides or more).

Thus, for example, an isolated nucleic acid provided herein can be an antisense nucleic acid to one of the aforementioned nucleic acids encoding a triterpenoid-modulating polypeptide, e.g., SEQ ID NO: 35, SEQ ID NOS: 37-47, SEQ ID NO: 53, SEQ ID NOS: 55-61, SEQ ID NOS: 49-51, SEQ ID NOs: 2-12, SEQ ID NOs: 14-21, SEQ ID NOs: 23-26, SEQ ID NOS: 28-33, and the consensus sequences set forth in FIG. 2, 4, 5, 6, 7, 8, or 9. A nucleic acid that decreases the level of a transcription or translation product of a gene encoding a triterpenoid-modulating polypeptide is transcribed into an antisense nucleic acid similar or identical to the sense coding sequence of an orthologue, homologue or variant, e.g. SEQ ID NO: 35, SEQ ID NOS: 37-47, SEQ ID NO: 53, SEQ ID NOS: 55-61, SEQ ID NOS: 49-51, SEQ ID NOs: 2-12, SEQ ID NOs: 14-21, SEQ ID NOs: 23-26, SEQ ID NOS: 28-33, and the consensus sequences set forth in FIG. 2, 4, 5, 6, 7, 8, or 9. Alternatively, the transcription product of an isolated nucleic acid can be similar or identical to the sense coding sequence of a triterpenoid-modulating polypeptide, but is an RNA that is unpolyadenylated, lacks a 5′ cap structure, or contains an unsplicable intron.

In another method, a nucleic acid can be transcribed into a ribozyme, or catalytic RNA, that affects expression of an mRNA. (See, U.S. Pat. No. 6,423,885). Ribozymes can be designed to specifically pair with virtually any target RNA and cleave the phosphodiester backbone at a specific location, thereby functionally inactivating the target RNA. Heterologous nucleic acids can encode ribozymes designed to cleave particular mRNA transcripts, thus preventing expression of a polypeptide. Hammerhead ribozymes are useful for destroying particular mRNAs, although various ribozymes that cleave mRNA at site-specific recognition sequences can be used. Hammerhead ribozymes cleave mRNAs at locations dictated by flanking regions that form complementary base pairs with the target mRNA. The sole requirement is that the target RNA contain a 5′-UG-3′ nucleotide sequence. The construction and production of hammerhead ribozymes is known in the art. See, for example, U.S. Pat. No. 5,254,678 and WO 02/46449 and references cited therein. Hammerhead ribozyme sequences can be embedded in a stable RNA such as a transfer RNA (tRNA) to increase cleavage efficiency in vivo. Perriman, R. et al., Proc. Natl. Acad. Sci. USA, 92(13):6175-6179 (1995); de Feyter, R. and Gaudron, J., Methods in Molecular Biology, Vol. 74, Chapter 43, “Expressing Ribozymes in Plants”, Edited by Turner, P. C, Humana Press Inc., Totowa, N.J. RNA endoribonucleases such as the one that occurs naturally in Tetrahymena thermophila, and which have been described extensively by Cech and collaborators can be useful. See, for example, U.S. Pat. No. 4,987,071.

Methods based on RNA interference (RNAi) can be used. RNA interference is a cellular mechanism to regulate the expression of genes and the replication of viruses. This mechanism is thought to be mediated by double-stranded small interfering RNA molecules. A cell responds to such a double-stranded RNA by destroying endogenous mRNA having the same sequence as the double-stranded RNA. Methods for designing and preparing interfering RNAs are known to those of skill in the art; see, e.g., WO 99/32619 and WO 01/75164. For example, a construct can be prepared that includes a sequence that is transcribed into an interfering RNA. Such an RNA can be one that can anneal to itself, e.g., a double stranded RNA having a stem-loop structure. One strand of the stem portion of a double stranded RNA comprises a sequence that is similar or identical to the sense coding sequence of the polypeptide of interest, and that is from about 10 nucleotides to about 2,500 nucleotides in length. The length of the sequence that is similar or identical to the sense coding sequence can be from 10 nucleotides to 500 nucleotides, from 15 nucleotides to 300 nucleotides, from 20 nucleotides to 100 nucleotides, or from 25 nucleotides to 100 nucleotides. The other strand of the stem portion of a double stranded RNA comprises an antisense sequence of the triterpenoid-modulating polypeptide of interest, and can have a length that is shorter, the same as, or longer than the corresponding length of the sense sequence. The loop portion of a double stranded RNA can be from 10 nucleotides to 5,000 nucleotides, e.g., from 15 nucleotides to 1,000 nucleotides, from 20 nucleotides to 500 nucleotides, or from 25 nucleotides to 200 nucleotides. The loop portion of the RNA can include an intron. See, e.g., WO 99/53050.

In some nucleic-acid based methods for inhibition of gene expression in plants, a suitable nucleic acid can be a nucleic acid analog. Nucleic acid analogs can be modified at the base moiety, sugar moiety, or phosphate backbone to improve, for example, stability, hybridization, or solubility of the nucleic acid. Modifications at the base moiety include deoxyuridine for deoxythymidine, and 5-methyl-2′-deoxycytidine and 5-bromo-2′-deoxycytidine for deoxycytidine. Modifications of the sugar moiety include modification of the 2′ hydroxyl of the ribose sugar to form 2′-O-methyl or 2′-O-allyl sugars. The deoxyribose phosphate backbone can be modified to produce morpholino nucleic acids, in which each base moiety is linked to a six membered, morpholino ring, or peptide nucleic acids, in which the deoxyphosphate backbone is replaced by a pseudopeptide backbone and the four bases are retained. See, for example, Summerton and Weller, 1997, Antisense Nucleic Acid Drug Dev., 7: 187-195; Hyrup et al., 1996, Bioorgan. Med. Chem., 4: 5-23. In addition, the deoxyphosphate backbone can be replaced with, for example, a phosphorothioate or phosphorodithioate backbone, a phosphoroamidite, or an alkyl phosphotriester backbone.

### III. Transgenic Plant Cells and Organisms

A. Transgenic Plants and Plant Cells

The invention also features transgenic plant cells and plants comprising at least one recombinant nucleic acid construct described herein. Such cells and plants are useful because the amount of a triterpenoid can be modulated in the cells or in one or more tissues of the plants.

Plants or plant cells can be transformed by having a construct integrated into its genome, i.e., be stably transformed. Stably transformed cells typically retain the introduced nucleic acid with each cell division. The plant or plant cells can also be transformed by having the construct not integrated into its genome. Such transformed cells are called transiently transformed cells. Transiently transformed cells typically lose all or some portion of the introduced nucleic acid construct with each cell division such that the introduced nucleic acid cannot be detected in daughter cells after sufficient number of cell divisions. Both transiently transformed and stably transformed transgenic plants and plant cells can be useful in the methods described herein.

A population of transgenic plants can be screened and/or selected for those members of the population that have a desired trait or phenotype conferred by expression of the transgene. Selection and/or screening can be carried out over one or more generations, which can be useful to identify those plants that have a desired trait, such as a modulated level of one or more triterpenoids. Selection and/or screening can also be carried out in more than one geographic location. In some cases, transgenic plants can be grown and selected under conditions which induce a desired phenotype or are otherwise necessary to produce a desired phenotype in a transgenic plant. In addition, selection and/or screening can be carried out during a particular developmental stage in which the phenotype is exhibited by the plant.

Transgenic plant cells used in methods described herein can constitute part or all of a whole plant. Such plants can be grown in a manner suitable for the species under consideration, either in a growth chamber, a greenhouse, or in a field. Transgenic plants can be bred as desired for a particular purpose, e.g., to introduce a recombinant nucleic acid into other lines, to transfer a recombinant nucleic acid to other species or for further selection of other desirable traits. Alternatively, transgenic plants can be propagated vegetatively for those species amenable to such techniques. Progeny includes descendants of a particular plant or plant line. Progeny of an instant plant include seeds formed on F₁, F₂, F₃, F₄, F₅, F₆ and subsequent generation plants, or seeds formed on BC₁, BC₂, BC₃, and subsequent generation plants, or seeds formed on F₁BC₁, F₁BC₂, F₁BC₃, and subsequent generation plants. The designation F₁ refers to the progeny of a cross between two parents that are genetically distinct. The designations F₂, F₃, F₄, F₅ and F₆ refer to subsequent generations of self- or sib-pollinated progeny of an F₁ plant. Seeds produced by a transgenic plant can be grown and then selfed (or outcrossed and selfed) to obtain seeds homozygous for the nucleic acid construct.

Transgenic plant cells growing in suspension culture, or tissue or organ culture, can be useful for extraction of triterpenoid compounds. For the purposes of this invention, solid and/or liquid tissue culture techniques can be used. When using solid medium, transgenic plant cells can be placed directly onto the medium or can be placed onto a filter that is then placed in contact with the medium. When using liquid medium, transgenic plant cells can be placed onto a flotation device, e.g., a porous membrane that contacts the liquid medium. Solid medium typically is made from liquid medium by adding agar. For example, a solid medium can be Murashige and Skoog (MS) medium containing agar and a suitable concentration of an auxin, e.g., 2,4-dichlorophenoxyacetic acid (2,4-D), and a suitable concentration of a cytolcinin, e.g., kinetin.

When transiently transformed plant cells are used, a reporter sequence encoding a reporter polypeptide having a reporter activity can be included in the transformation procedure and an assay for reporter activity or expression can be performed at a suitable time after transformation. A suitable time for conducting the assay typically is about 1-21 days after transformation, e.g., about 1-14 days, about 1-7 days, or about 1-3 days. The use of transient assays is particularly convenient for rapid analysis in different species, or to confirm expression of a heterologous triterpenoid-modulating polypeptide whose expression has not previously been confirmed in particular recipient cells.

Techniques for introducing nucleic acids into monocotyledonous and dicotyledonous plants are known in the art, and include, without limitation, Agrobacterium-mediated transformation, viral vector-mediated transformation, electroporation and particle gun transformation, e.g., U.S. Pat. Nos. 5,538,880, 5,204,253, 6,329,571 and 6,013,863. If a cell or cultured tissue is used as the recipient tissue for transformation, plants can be regenerated from transformed cultures if desired, by techniques known to those skilled in the art.

B. Plant Species

The polynucleotides and vectors described herein can be used to transform a number of monocotyledonous and dicotyledonous plants and plant cell systems, including dicots such as alfalfa, apple, beans (including kidney beans, lima beans, green beans), broccoli, cabbage, carrot, castor bean, cherry, chick peas, chicory, clover, cocoa, coffee, cotton, crambe, flax, foxglove, grape, grapefruit, lemon, lentils, lettuce, linseed, mango, melon (e.g., watermelon, cantaloupe), mustard, orange, peach, peanut, pear, peas, pepper, plum, potato, oilseed rape, rapeseed (high erucic acid (rape) and canola), safflower, sesame, soaptree bark, soybean, spinach, strawberry, sugar beet, sunflower, sweet potatoes, tea, tomato, yams, as well as monocots such as banana, barley, date palm, field corn, garlic, millet, oat, oil palm, onion, pineapple, popcorn, rice, rye, sorghum, sudangrass, sugarcane, sweet corn, switchgrass, and wheat. Brown seaweeds, green seaweeds, red seaweeds, and microalgae can also be used.

Thus, the methods and compositions described herein can be used with dicotyledonous plants belonging, for example, to the orders Apiales, Arecales, Aristochiales, Asterales, Batales, Cainpanulales, Capparales, Caryophyllales, Casuarinales, Celastrales, Cornales, Cucurbitales, Diapensales, Dilleniales, Dipsacales, Ebenales, Ericales, Eucomiales, Euphorbiales, Fabales, Fagales, Gentianales, Geraniales, Haloragales, Hamamelidales, Illiciales, Juglandales, Lamiales, Laurales, Lecythidales, Leitneriales, Linales, Magniolales, Malvales, Myricales, Myrtales, Nymphaeales, Papaverales, Piperales, Plantaginales, Plumbaginales, Podostemales, Polemoniales, Polygalales, Polygonales, Primulales, Proteales, Rafflesiales, Ranunculales, Rhamnales, Rosales, Rubiales, Salicales, Santales, Sapindales, Sarraceniaceae, Scrophulariales, Solanales, Trochodendrales, Theales, Umbellales, Urticales, and Violales. The methods and compositions described herein also can be utilized with monocotyledonous plants such as those belonging to the orders Alisinatales, Arales, Arecales, Asparagales, Bromeliales, Cominelinales, Cyclanthales, Cyperales, Eriocaulales, Hydrocharitales, Juncales, Liliales, Najadales, Orchidales, Pandanales, Poales, Restionales, Triuridales, Typhales, Zingiberales, and with plants belonging to Gymnospermae, e.g., Cycadales, Ginkgoales, Gnetales, and Pinales.

The methods and compositions can be used over a broad range of plant species, including species from the dicot genera Acokanthera, Aesculus, Amaranthus, Anacardium, Angophora, Apium, Arachis, Beta, Betula, Bixa, Brassica, Calendula, Camellia, Capsicum, Carthamus, Centella, Chrysanthemum, Cicer, Cichorium, Cinnamomum, Citrus, Citrullus, Cocculus, Cocos, Coffea, Corylus, Corymbia, Crainbe, Croton, Cucumis, Cucurbita, Cuphea, Daucus, Dianthus, Digitalis, Dioscorea, Duguetia, Ficus, Fragaria, Glaucium, Glycine, Glycyrrhiza, Gossypium, Helianthus, Hyoscyamus, Lactuca, Landolphia, Lavandula, Lens, Linum, Litsea, Luffa, Lupinus, Lycopersicon, Majorana, Malus, Mangifera, Manihot, Medicago, Mentha, Micropus, Nicotiana, Ocimum, Olea, Origanum, Persea, Petunia, Phaseolus, Pistacia, Pisum, Prunus, Pyrus, Quillaja, Rabdosia, Raphanus, Rosa, Rosmarinus, Rubus, Salix, Salvia, Senecio, Sesamum, Sinapis, Solanum, Spinacia, Stephania, Strophanthus, Tagetes, Theobroma, Thymus, Trifolium, Trigonella, Vaccinium, Vicia, Vigna, and Vitis; and the monocot genera Agrostis, Allium, Ananas, Andropogon, Asparagus, Avena, Convallaria, Curcuma, Cynodon, Eragrostis, Festuca, Festulolium, Heterocallis, Hordeum, Lemna, Lolium, Musa, Oryza, Panicum, Pennisetum, Phleuin, Phoenix, Poa, Ruscus, Saccharum, Secale, Sorghum, Triticum, and Zea; and the gymnosperm genera Abies, Cunninghamia, Picea, and Pseudotsuga.

The methods and compositions described herein also can be used with brown seaweeds, e.g., Ascophyllum nodosum, Fucus vesiculosus, Fucus serratus, Himanthalia elongata, and Undaria pinnatifida; red seaweeds, e.g., Chondrus crispus, Cracilaria Verrucosa, Porphyra umbilicalis, and Palmaria palmiata; green seaweeds, e.g., Enteromorpha spp. and Ulva spp.; and microalgae, e.g., Spirulina spp. (S. platensis and S. maxima) and Odontella aurita. In addition, the methods and compositions can be used with Crypthecodinium cohnii, Schizochytrium spp., and Haematococcus pluvialis.

In some embodiments, a plant is a member of the species Acokanthera spp., Ananus comosus, Betula alba, Bixa orellana, Brassica campestris, Brassica napus, Brassica oleracea, Calendula officinalis, Centella asiatica, Chrysanthemum parthenium, Cinnamommum camphora, Citrullus spp., Coffea arabica, Canvallaria majalis, Digitalis lanata, Digitalis purpurea, Digitalis spp., Dioscorea spp., Glycine max, Glycyrrhiza glabra, Gossypium spp., Lactuca sativa, Luffa spp., Lycopersicon esculentum, Musa paradisiaca, Oryza sativa, Quillaja saponaria, Rosmarinus officinalis, Ruscus aculeatus, Solanun tuberosum, Strophanthus gratus, Strophanthus spp., Theobroma cacao, Triticum aestivum, Vitis vinifera, or Zea mays.

C. Other Organisms

In some cases, it may be desirable to produce nucleic acids and/or polypeptides described herein by recombinant production in a prokaryotic or non-plant eukaryotic host cell. To recombinantly produce polypeptides, a nucleic acid encoding the polypeptide of interest can be ligated into an expression vector and used to transform a bacterial, eukaryotic, or plant host cell (e.g., insect, yeast, mammalian, or plant cells). In bacterial systems, a strain of Escherichia coli such as BL-21 can be used. Suitable E. coli vectors include the pGEX series of vectors that produce fusion proteins with glutathione S-transferase (GST). Depending on the vector used, transformed E. coli are typically grown exponentially, then stimulated with isopropylthiogalactopyranoside (IPTG) prior to harvesting. In general, expressed fusion proteins are soluble and can be purified easily from lysed cells by adsorption to glutathione-agarose beads followed by elution in the presence of free glutathione. The pGEX vectors are designed to include thrombin or factor Xa protease cleavage sites so that the cloned target gene product can be released from the GST moiety. Alternatively, 6×His-tags can be used to facilitate isolation.

In eukaryotic animal host cells, a number of viral-based expression systems are often utilized to express polypeptides. A nucleic acid encoding a polypeptide can be cloned into, for example, a baculoviral vector such as pBlueBac (Invitrogen, Carlsbad, Calif.) and then used to co-transfect insect cells such as Spodoptera frugiperda (Sf9) cells with wild type DNA from Autographa californica multiply enveloped nuclear polyhedrosis virus (AcMNPV). Recombinant viruses producing polypeptides of the invention can be identified by standard methodology. Mammalian cell lines that stably express polypeptides can be produced by using expression vectors with the appropriate control elements and a selectable marker. For example, the pcDNA3 eukaryotic expression vector (Invitrogen, Carlsbad, Calif.) is suitable for expression of polypeptides in cell such as, Chinese hamster ovary (CHO) cells, COS-1 cells, human embryonic kidney 293 cells, NIH3T3 cells, BHK21 cells, MDCK cells, ST cells, PK15 cells, or human vascular endothelial cells (HUVEC). In some instances, the pcDNA3 vector can be used to express a polypeptide in BHK21 cells, where the vector includes a CMV promoter and a G418 antibiotic resistance gene. Following introduction of the expression vector, stable cell lines can be selected, e.g., by antibiotic resistance to G418, kanamycin, or hygromycin. Alternatively, amplified sequences can be ligated into a mammalian expression vector such as pcDNA3 (Invitrogen, San Diego, Calif.) and then transcribed and translated in vitro using wheat germ extract or rabbit reticulocyte lysate.

### IV. Triterpenoid Compounds

Compositions and methods described herein are useful for producing one or more triterpenoid compounds, because the triterpenoid-modulating polypeptides described above are effective for modulating the amount of one or more triterpenoid compounds. Thus, a transgenic plant or cell comprising a recombinant nucleic acid expressing such a triterpenoid-modulating polypeptide can be effective for modulating the amount and/or rate of biosynthesis of one or more of such triterpenoids in a plant.

An amount of one or more of any individual triterpenoid compound can be modulated, e.g., increased or decreased, relative to a control plant not transgenic for the particular triterpenoid-modulating polypeptide using the methods described herein. In certain cases, therefore, more than one triterpenoid compound (e.g., two, three, four, five, six, seven, eight, nine, ten or even more triterpenoid compounds) can have its amount modulated relative to a control plant or cell that is not transgenic for a triterpenoid-modulating polypeptide described herein.

Triterpenoid compounds can be produced by the methods and compositions described herein. Exemplary triterpenoids include, without limitation, squalene, lupeol, α-amyrin, β-amyrin, glycyrrhizin, β-sitosterol, sitostanol, stigmasterol, campesterol, ergosterol, diosgenin, aescin, betulinic acid, cucurbitacin E, ruscogenin, mimusin, avenacin A-1, gracillin, α-tomatine, α-solanine, convallatoxin, acetyldigoxin, digoxin, deslanoside, digitalin, digitoxin, quillaic acid and its glycoside derivatives, squalamine, ouabain, strophanthidin, hydrocortisone, testosterone, and asiaticoside. Plants containing a recombinant nucleic acid construct described herein typically have a difference in the amount and/or rate of synthesis of one or more triterpenoid compounds, relative to a corresponding control plant or cell that is not transformed with the recombinant nucleic acid construct.

The amount of one or more triterpenoid compounds can be increased or decreased in transgenic cells expressing a triterpenoid-modulating polypeptide as described herein. An increase can be from about 5% to about 800% on a weight basis (e.g., a fresh or freeze dried weight basis) in such a transgenic cell compared to a corresponding control cell that lacks the recombinant nucleic acid encoding the triterpenoid-modulating polypeptide. In some embodiments, the increase is from about 5% to about 250%, or about 50% to about 500%, or about 100% to about 400%, or about 25% to about 400%, or about 50% to about 350%, or about 75% to about 150%, or about 90% to about 250%, or about 125% to about 375%, or about 150% to about 450%, or about 175% to about 475%, or about 200% to about 500%, or about 250% to about 550%, or about 300% to about 600%, or about 350% to about 650%, or about 400% to about 700%, or about 450% to about 750%, or about 500% to about 800% higher than the amount in a corresponding control cell that lacks the recombinant nucleic acid encoding the triterpenoid-modulating polypeptide. In some embodiments, the increase is from about 1.5-fold to about 800-fold, or about 2-fold to about 22-fold, or about 25-fold to about 50-fold, or about 75-fold to about 130-fold, or about 5-fold to about 50-fold, or about 5-fold to about 10-fold, or about 10-fold to about 20-fold, or about 10-fold to about 25-fold, or about 20-fold to about 75-fold, or about 10-fold to about 100-fold, or about 40-fold to about 100-fold, about 200-fold to about 300-fold, about 100-fold to about 350-fold, or about 200-fold to about 400-fold, about 300-fold to about 500-fold, about 400-fold to about 600-fold, about 500-fold to about 800-fold, about 30-fold to about 50-fold higher than the amount in a corresponding control cell that lacks the recombinant nucleic acid encoding the triterpenoid-modulating polypeptide.

In other embodiments, the triterpenoid compound that is increased in transgenic cells expressing a triterpenoid-modulating polypeptide as described herein is either not produced or is not detectable in a corresponding control cell that lacks the recombinant nucleic acid encoding the triterpenoid-modulating polypeptide. Thus, in such embodiments, the increase in such a triterpenoid compound is infinitely higher in a corresponding control cell that lacks the recombinant nucleic acid encoding the triterpenoid-modulating polypeptide. For example, in certain cases, a triterpenoid-modulating polypeptide described herein may activate a biosynthetic pathway in a plant that is not normally activated or operational in a control plant, and one or more new triterpenoids that were not previously produced in that plant species can be produced.

The increase in amount of one or more triterpenoids can be restricted in some embodiments to particular tissues and/or organs, relative to other tissues and/or organs. For example, a transgenic plant can have an increased amount of a triterpenoid in fruit tissue relative to leaf or root tissue.

In other embodiments, the amounts of one or more triterpenoids are decreased in transgenic cells expressing a triterpenoid-modulating polypeptide as described herein. A decrease ratio can be expressed as the ratio of the triterpenoid in such a transgenic cell on a weight basis (e.g., fresh or freeze dried weight basis) as compared to the triterpenoid in a corresponding control cell that lacks the recombinant nucleic acid encoding the triterpenoid-modulating polypeptide. The decrease ratio can be from about 0.05 to about 0.90. In certain case, the ratio can be from about 0.2 to about 0.6, or from about 0.4 to about 0.6, or from about 0.3 to about 0.5, or from about 0.2 to about 0.4.

In certain embodiments, a triterpenoid compound that is decreased in transgenic cells expressing a triterpenoid-modulating polypeptide as described herein is decreased to an undetectable level as compared to the level in a corresponding control cell that lacks the recombinant nucleic acid encoding the triterpenoid-modulating polypeptide. Thus, in such embodiments, the decrease ratio in such a triterpenoid compound is zero.

The decrease in amount of one or more triterpenoids can be restricted in some embodiments to particular tissues and/or organs, relative to other tissues and/or organs. For example, a transgenic plant can have an decreased amount of a triterpenoid in fruit tissue relative to leaf or root tissue.

In some embodiments, the amounts of two or more triterpenoids are increased and/or decreased, e.g., the amounts of two, three, four, five, six, seven, eight, nine, ten (or more) triterpenoid compounds are independently increased and/or decreased. The amount of a triterpenoid compound can be determined by known techniques, e.g., by extraction of triterpenoid compounds followed by gas chromatography-mass spectrometry (GC-MS) or liquid chromatography-mass spectrometry (LC-MS). If desired, the structure of the triterpenoid compound can be confirmed by GC-MS, LC-MS, nuclear magnetic resonance and/or other known techniques.

Typically, a difference (e.g., an increase) in the amount of any individual triterpenoid compound in a transgenic plant or cell relative to a control plant or cell is considered statistically significant at p≦0.05 with an appropriate parametric or non-parametric statistic, e.g., Chi-square test, Student's t-test, Mann-Whitney test, or F-test. In some embodiments, a difference in the amount of any individual triterpenoid compound is statistically significant at p<0.01, p<0.005, or p<0.001. A statistically significant difference in, for example, the amount of any individual triterpenoid compound in a transgenic plant compared to the amount in cells of a control plant indicates that (1) the recombinant nucleic acid present in the transgenic plant results in altered levels of one or more triterpenoid compounds and/or (2) the recombinant nucleic acid warrants further study as a candidate for altering the amount of a triterpenoid compound in a plant.

### V. Methods of Producing Triterpenoids

Also provided are methods for producing one or more triterpenoids. Such methods can include growing a plant cell that includes a nucleic acid encoding a triterpenoid-modulating polypeptide as described herein, under conditions effective for the expression of the triterpenoid-modulating polypeptide. Also provided herein are methods for modulating (e.g., altering, increasing, or decreasing) the amounts of one or more triterpenoids in a plant cell. The methods can include growing a plant cell as described above, i.e., a plant cell that includes a nucleic acid encoding a triterpenoid-modulating polypeptide as described herein. The one or more triterpenoids produced by these methods can be novel triterpenoids, e.g., not normally produced in a wild-type plant cell.

The methods can further include the step of recovering one or more triterpenoids from the cells. For example, plant cells known or suspected of producing one or more triterpenoids can be subjected to fractionation to recover a desired triterpenoid. Typically, fractionation is guided by in vitro assay of fractions. In some instances, cells containing one or more compounds can be separated from cells not containing, or containing lower amounts of the triterpenoid, in order to enrich for cells or cell types that contain the desired compound(s). A number of methods for separating particular cell types or tissues are known to those having ordinary skill in the art.

Fractionation can be carried out by techniques known in the art. For example, plant tissues or organs can be extracted with 100% MeOH to give a crude oil which is partitioned between several solvents in a conventional manner. As an alternative, fractionation can be carried out on silica gel columns using methylene chloride and ethyl acetate/hexane solvents.

In some embodiments, a fractionated or unfractionated plant tissue or organ is subjected to mass spectrometry in order to identify and/or confirm the presence of a desired triterpenoid(s). See, e.g., WO 02/37111. In some embodiments, electrospray ionization (ESI) mass spectrometry can be used. In other embodiments, atmospheric pressure chemical ionization (APCI) mass spectrometry is used. If it is desired to identify higher molecular weight molecules in an extract, matrix-assisted laser desorption/ionization time-of-flight (MALDI-TOF) mass spectrometry can be useful.

### VI. Seeds, Oils, Vegetative Tissues, Animal Feed, and Articles of Manufacture

Transgenic plants provided herein have particular uses in the agricultural and nutritional industries, e.g., in compositions such as food and feed products. Seeds of transgenic plants describe herein can be conditioned and bagged in packaging material by means known in the art to form an article of manufacture. Packaging material such as paper and cloth are well known in the art. Such a bag of seed preferably has a package label accompanying the bag, e.g., a tag or label secured to the packaging material, a label printed on the packaging material or a label inserted within the bag. The package label may indicate the seed contained therein incorporates transgenes that provide increased amounts of one or more triterpenoids in one or more tissues of plants grown from such seeds.

Transgenic plants described herein can be used to make food products such as fresh, frozen, or canned vegetables and fruits. Suitable plants with which to make such products include bananas, broccoli, grapes, lettuce, mango, melon, spinach, strawberry and tomatoes. Transgenic plants described herein can also be used to make processed food products such as tomato sauce, ketchup, jellies, and jams from the above fruits and vegetables. Such products are useful to provide increased amounts of triterpenoids in a human diet.

Seeds from transgenic plants described herein can be used to make food products such as flours, vegetable oils and insoluble fibers. Suitable plants from which to make such vegetable oils include soybean, canola, corn, cottonseed, flax, oil palm, safflower, and sunflower. Such oils can be used for frying, baking, and spray coating applications. Transgenic plants described herein can also be used as a source of animal feeds.

Seeds or non-seed tissues from transgenic plants described herein can also be used as a source from which to extract triterpenoids, using techniques known in the art. The resulting extract can be included in nutritional supplements as well as processed food products, e.g., snack products, frozen entrees, vegetable oils, breakfast cereals, and baby foods.

The invention is further described in the following examples, which do not limit the scope of the invention described in the claims.

### EXAMPLES

### Example 1

### Generation of Plants Containing a 35S::23357293 Construct

The following symbols are used in the Examples: T1: first generation transformant; T2: second generation, progeny of self-pollinated T1 plants; T3: third generation, progeny of self-pollinated T2 plants; T4: fourth generation, progeny of self-pollinated T3 plants.

cDNA ID 23357293 (SEQ ID No: 34) is predicted to encode an AP2-domain transcription factor. T-DNA binary vector constructs were made using standard molecular biology techniques. A construct was made that contained a nucleic acid designated cDNA ID 23357293 operably linked in the sense orientation to a 35S promoter. The construct also contained a marker gene conferring resistance to the herbicide Finale®. The construct was introduced into Arabidopsis ecotype Wassilewskija (WS) by the floral dip method essentially as described in Bechtold, N. et al., C.R. Acad. Sci. Paris, 316:1194-1199 (1993). Ten independently transformed events were selected and evaluated for their qualitative phenotype in the T₁ generation. Plants from these events were designated as ME01483 events. Control plants contained an empty vector construct having the Finale® marker gene (CRS 338) but lacking the 35S::23357293 sequence. The physical appearance of nine of the ten T1 plants was identical to the controls except for Event-02, which had an abnormal branching pattern, fused inflorescences, a disorganized rosette and was sterile. This phenotype appears sporadically following transformation and is likely an artifact of the transformation process.

T1 seeds were germinated and allowed to self-pollinate. T2 seeds were collected and a portion was germinated, allowed to self-pollinate, and T3 seeds were collected.

### Example 2

### Analysis of Triterpenoids in Arabidopsis ME01483 Events

T2 and T3 seeds of the Arabidopsis thaliana ME01483 screening events described in Example 1 were planted in soil comprising Sunshine LP5 Mix and Thermorock Vermiculite Medium #3 at a ratio of 60:40, respectively containing Marathon insecticide. The seeds were stratified at 4° C. for approximately two to three days. After stratification, the seeds were transferred to the greenhouse and covered with a plastic dome and tarp until most of the seeds had germinated. Plants were grown under long day conditions. Approximately seven to ten days post-germination, plants were sprayed with Finale® herbicide to confirm that the plants were transgenic.

Approximately 10 days post-bolting, aerial tissues from four Finale® resistant plants of each event were pooled, frozen in liquid nitrogen and subsequently lyophilized. Lyophilized tissues were stored at −80° C. for up to four weeks. Tissue samples were removed from the freezer and crushed into a fine powder. About 1.25 ml of ethyl acetate and 20 μl of a 19-OH cholesterol internal standard (1 mg/ml in ethyl acetate) were added to 30±3 mg of ground tissue and the mixture heated at 70° C. for 30 minutes, centrifuged at 14,000 g for 5 minutes, and the supernatant dried in a Speedvac. The dried extract was then derivatized in 80 μl of pyridine using N-Methyl-N-(trimethylsilyl) trifluoroacetamide. Samples of each extract were analyzed in triplicate using a Shimadzu GCMS QP-2010 instrument and a Varian Factor Four Column (30 m×0.25 mm×0.25% film thickness+10 m integrated guard). Compounds were identified via retention time standards and mass spectral libraries. Target peak areas were integrated and the values exported to Excel. All areas were normalized with respect to the internal standard and the initial weight of the sample. All experimental samples were normalized with respect to the control. A calibration curve was generated by plotting the GCMS peak area against serial dilutions of a squalene standard. Values for three independent wild type samples fell within the linear range of the curve. On a dry weight basis, WT1 had 0.0031 gm squalene per gm of sample, WT2 had 0.0035 gm squalene per gm of sample and WT3 had 0.0026 gm squalene per gm of sample.

The results of the squalene analyses are shown in Table 1. In the T2 generation, squalene levels were increased in Events-01, -03 and -04 to 207%, 188%, and 138% respectively, of those found in transgenic control plants. In the T3 generation, squalene levels were increased in Events-01, -03 and -04 to 139%, 145%, and 130% respectively, of those found in transgenic control plants transfected with vector alone. No statistically significant differences were detected in α-tocopherol, β-tocopherol, γ-tocopherol, and δ-tocopherol, campesterol, stigmasterol, β-sitosterol, cycloartenol, α-amyrin, β-amyrin or lupeol in any of the T2 and T3 generation samples. P-values were determined using a Student's t-test.

TABLE 1
Squalene Levels in ME01483 T₂ and T₃ Generations^{a}

| | | | |
|---|---|---|---|
| ME01483-01 | ME01483-03 | ME01483-04 | Control |

| | | | | |
|---|---|---|---|---|
| T₂ | 207 ± 43 | 188 ± 32 | 138 ± 4 | 100 ± 30 |
| p-value^{b} | <0.01 | 0.02 | <0.01 | NA |
| T₃ | 139 ± 12 | 145 ± 5 | 130 ± 15 | 100 ± 10 |
| p-valueb | 0.01 | <0.01 | 0.05 | NA |

^{a}Values for ME01483 plants are expressed as percent relative to control.

T2 seeds and plants of ME01483 events-01, -03 or -04 exhibited no statistically significant reduction in germination rate, days to flowering, rosette area 7 days post-bolting, or fertility (silique number and seed fill).

### Example 3

### Analysis of Triterpenoid Content in Plants Containing a 35S::KNAT3 Homeobox Protein cDNA 23389731 Construct

cDNA ID 23389731 (SEQ ID NO: 36) is predicted to encode a KNAT3 homeobox protein. Transgenic plants containing a 35S::23389731 cDNA construct were made according to the protocol described in Example 1, using a construct that contained a nucleic acid designated cDNA ID 23389731 operably linked in the sense orientation to a 35S promoter. Ten independent transformation events were selected and evaluated for their qualitative phenotype in the T1 generation. Plants from these events were designated ME06492 events.

T1 plants were allowed to self-pollinate and T2 seeds were collected. A portion of the T2 seeds were germinated, allowed to self-pollinate, and T3 seeds collected. T2 and T3 seeds of the Arabidopsis thaliana ME06492 events were planted and grown as described in Example 1 to confirm that the plants were transgenic for the Finale® marker.

Qualitative analyses of the ME06492 plants indicated that 8 out of 10 T1 plants were morphologically identical to control plants transformed with vector alone. A reduction in height and fertility levels was noted for events-04 and -07, but this phenotype did not persist in the T2 generation. No negative phenotypes were observed in the T2 plants.

Approximately 10 days post-bolting, all aerial tissues were collected from four Finale® resistant T2 plants of each event and analyzed for triterpenoid content as described in Example 2. Aerial tissues from Finale® resistant T3 plants from 5 events were analyzed in the same manner.

The results of this analysis are shown in Tables 2 and 3. Analyses of four T2 plants indicated that Events-02, -03, and -04 had statistically significant increases in levels of both α- and β-amyrin compared to those in the transgenic controls. Event-07 had a significant increase in levels of β-amyrin. Analyses of four T3 plants indicated significant increases in the levels of both α- and β-amyrin in Event-02 (167% and 129% of control respectively) and in Event-04 (163% and 135% of control respectively). Event-03 had a significant increase in levels of β-amyrin. Separate calibration curves, prepared with known concentrations of α- and β-amyrin standards respectively, were used to confirm that all α- and β-amyrin measurements on plant tissues were within the linear range of detection by GC-MS.

TABLE 2
α-Amyrin Levels in ME06492 T2 and T3 Generations^{a}

| | ME06492-02 | ME06492-03 | ME06492-04 | ME06492-06 | ME06492-07 | Control |
|---|---|---|---|---|---|---|
| T2 | 141 ± 28 | 148 ± 18 | 168 ± 19 | 118 ± 20 | 123 ± 21 | 100 ± 20 |
| p-value^{b} | 0.01 | <0.01 | <0.01 | 0.57 | 0.09 | N/A |
| T3 | 167 ± 7 | 146 ± 10 | 163 ± 12 | 99 ± 12 | 101 ± 12 | 100 ± 26 |
| p-value^{b} | <0.01 | 0.01 | <0.01 | 0.97 | 0.94 | N/A |

^{a}Values for ME06492 plants are expressed as percent relative to control.
^{b}P-values were determined using a Student's t-test.

TABLE 3
β-Amyrin Levels in ME06492 T2 and T3 Generations^{a}

| | ME06492-02 | ME06492-03 | ME06492-04 | ME06492-06 | ME06492-07 | Control |
|---|---|---|---|---|---|---|
| T2 | 154 ± 13 | 146 ± 8 | 153 ± 13 | 125 ± 6 | 142 ± 4 | 100 ± 14 |
| p-value^{b} | <0.01 | <0.01 | <0.01 | 0.01 | <0.01 | N/A |
| T3 | 129 ± 9 | 121 ± 10 | 135 ± 5 | 142 ± 16 | 106 ± 6 | 100 ± 17 |
| p-value | 0.02 | 0.07 | <0.01 | <0.01 | 0.55 | N/A |

^{a}Values for ME06492 plants are expressed as percent relative to control.
^{b}P-values were determined using a Student's t-test.

### Example 4

### Analysis of Triterpenoid Content in Plants Containing a 35S::PHD Finger Transcription Factor cDNA 23543586 Construct

cDNA ID 23543586 (SEQ ID NO: 52) is predicted to encode a PHD finger domain containing protein. Transgenic plants containing a 35S::cDNA 23543586 construct were made according to the protocol described in Example 1, using a construct that contained a nucleic acid designated cDNA ID 23543586 operably linked in the sense orientation to a 35S promoter. Three independent transformation events were selected and evaluated for their qualitative phenotype in the T1 generation. No observable differences in morphology were noted between T1 plants and controls. Plants from these events were designated ME11013 events.

T1 plants were allowed to self-pollinate and T2 seeds were collected. A portion of the T2 seeds were germinated, allowed to self-pollinate, and T3 seeds collected. T2 and T3 seeds of the Arabidopsis thaliana ME11013 events were planted and grown as described in Example 1 to confirm that the plants were transgenic for the Finale® marker.

Approximately 10 days post-bolting, all aerial tissues were collected from four Finale® resistant T2 plants of three events and analyzed for triterpenoid content as described in Example 2. Aerial tissues from four Finale® resistant T3 plants from each of three events were analyzed in the same manner.

The results of these experiments are shown in Table 4. Arabidopsis plants ME07139-02 and ME07139-07 in the T2 generation showed an increase in squalene levels of 222% and 229%, respectively, relative to those of control plants.

TABLE 4
Stigmasterol Levels in ME11013 T2 and T3 Generations^{a}

| | ME11013-01 | ME11013-02 | ME11013-07 | Control |
|---|---|---|---|---|
| T2 | 127 ± 37 | 222 ± 92 | 229 ± 61 | 100 ± 47 |
| p-value^{b} | 0.39 | 0.01 | <0.01 | N/A |
| T3 | 127 ± 4 | 103 ± 18 | 101 ± 14 | 100 ± 28 |
| p-value^{b} | 0.1 | 0.87 | 0.37 | N/A |

^{a}Values for ME11013 plants are expressed as percent relative to control.
^{b}P-values were determined using a Student's t-test.

### Example 5

### Analysis of Triterpenoid Content in Plants Containing a 35S::RING Finger Transcription Factor cDNA 23361365 Construct

cDNA 23361365 (SEQ ID NO: 54) is predicted to encode a RING finger domain containing protein. Transgenic plants containing a 35S::cDNA 23361365 construct were made according to the protocol described in Example 1, using a construct that contained a nucleic acid designated cDNA ID 23361365 operably linked in the sense orientation to a 35S promoter. Five independent transformation events were selected and evaluated for their qualitative phenotype in the T1 generation. No observable differences in morphology were noted between T1 plants and controls. Plants from these events were designated ME07139 events.

T1 plants were allowed to self-pollinate and T2 seeds were collected. A portion of the T2 seeds were germinated, allowed to self-pollinate, and T3 seeds collected. T2 and T3 seeds of the Arabidopsis thaliana ME07139 events were planted and grown as described in Example 1 to confirm that the plants were transgenic for the Finale® marker.

Approximately 10 days post-bolting, all aerial tissues were collected from four Finale® resistant T2 plants of five events and analyzed for triterpenoid content as described in Example 2. Aerial tissues from four Finale® resistant T3 plants from 5 events were analyzed in the same manner. The results of these experiments are shown in Table 5. In Arabidopsis plants ME07139-02 and ME07139-04 in the T2 generation, squalene levels were increased to 450% and 270% of those of control plants.

TABLE 5
Squalene levels in ME07139 T2 and T3 Generations^{a}

| | ME07139-01 | ME07139-02 | ME07139-03 | ME07139-04 | ME07139-05 | Control |
|---|---|---|---|---|---|---|
| T2 | 102 ± 5 | 450 ± 2 | 108 ± 7 | 270 ± 6 | 93 ± 7 | 100 ± 2 |
| p-value^{b} | 0.5 | <0.01 | 0.18 | <0.01 | 0.27 | N/A |
| T3 | 32 ± 20 | 66 ± 1 | 32 ± 2 | 63 ± 17 | 123 ± 78 | 100 ± 2 |
| p-value^{b} | <0.01 | <0.01 | <0.01 | 0.29 | 0.51 | N/A |

^{a}Values for ME07139 plants are expressed as percent relative to control.
^{b}P-values were determined using a Student's t-test.

### Example 6

### Analysis of Triterpenoid Content in Plants Containing a 35S::thioredoxin m4 cDNA 23644306 Construct

cDNA ID 23644306 (SEQ ID NO: 48) is predicted to encode a thioredoxin m4 protein. Transgenic plants containing a 35S::cDNA 23644306 construct were made according to the protocol described in Example 1, using a construct that contained a nucleic acid designated cDNA ID 23644306 operably linked in the sense orientation to a 35S-promoter. Five independent transformation events were selected and evaluated for their qualitative phenotype in the T1 generation. No observable differences in morphology were noted between T1 plants and controls. Plants from these events were designated ME09883 events.

T1 plants were allowed to self-pollinate and T2 seeds were collected. A portion of the T2 seeds were germinated, allowed to self-pollinate, and T3 seeds collected. T2 and T3 seeds of the Arabidopsis thaliana ME09883 events were planted and grown as described in Example 1 to confirm that the plants were transgenic for the Finale® marker.

Approximately 10 days post-bolting, all aerial tissues were collected from four Finale® resistant T2 plants of five events and analyzed for triterpenoid content as described in Example 2. Aerial tissues from four Finale® resistant T3 plants from five events were analyzed in the same manner.

The results of these experiments are shown in Table 6. Arabidopsis plants ME09883-01 and -05 had statistically significant increases of stigmasterol in the T2 generation relative to control plants (177% and 167% of control, respectively).

TABLE 6
Stigmasterol Levels in ME09883 T2 and T3 Generations^{a}

| | ME09883-01 | ME09883-02 | ME09883-03 | ME09883-04 | ME09883-05 | Control |
|---|---|---|---|---|---|---|
| T2 | 177 ± 40 | 96 ± 34 | 130 ± 11 | 95 ± 16 | 167 ± 39 | 100 ± 37 |
| p-value^{b} | 0.01 | 0.86 | 0.02 | 0.57 | 0.02 | N/A |
| T3 | 120 ± 10 | 120 ± 30 | 120 ± 20 | 70 ± 20 | 70 ± 20 | 100 ± 24 |
| p-value^{b} | 0.2 | 0.3 | 0.3 | 0.09 | 0.4 | N/A |

^{a}Values for ME09883 plants are expressed as percent relative to control.
^{b}P-values were determined using a Student's t-test.

### Example 7

### Analysis of Triterpenoid Content in Plants Containing an 35S::SQS1 12328487 cDNA Construct

Squalene synthase (SQS) catalyzes the conversion of the first committed step in the branch point for diverting carbon specifically to the biosynthesis of triterpenoids in the isoprenoid biosynthetic pathway. cDNA 12328487 (SEQ ID NO: 1) encodes a squalene synthase. Wild type Arabidopsis Wassilewskija (WS) plants were transformed with a T1 plasmid containing a nucleic acid designated cDNA ID 12328487 operably linked in the sense orientation to a CaMV 35S constitutive promoter according the protocol described in Example 1. The construct also contained a marker gene conferring resistance to the herbicide Basta®. Two independent transformation events were selected and evaluated for their qualitative phenotype in the T1 generation. No observable differences in morphology were noted between T1 plants and controls. Plants from these events were designated SQS1 events.

T1 plants were allowed to self-pollinate and T2 seeds were collected. A portion of the T2 seeds were germinated, allowed to self-pollinate, and T3 seeds collected. T2 and T3 seeds of the Arabidopsis thaliana SQS1 events were planted and grown as described in Example 1 to confirm that the plants were transgenic for the Basta® marker.

Approximately 14 days post-bolting, leaves and cauline leaves from 10-20 Basta® resistant T2 and T3 plants of each event were pooled, frozen in liquid nitrogen and subsequently lyophilized. In addition, stems, siliques, floral and meristematic tissues were separately collected, pooled, frozen and lyophilized from the same plants. Lyophilized tissues were analyzed for triterpenoid content as follows. Lyophilized tissues were ground, using a spatula, into a powder fine enough to pass through a 1000 μm seed sieve. Approximately 100 mg finely ground tissue was placed into a Dionex ASE-200 extraction cell according to the manufacturer's directions. One-hundred μg of a 2 mg/ml solution of 19OH-cholesterol that had been dissolved in ethyl acetate was added to the plant tissue; the tissue was then subjected to 3 cycles of extraction with 100% ethyl acetate for 5 minutes each at 10° C. (1500 psi). The total extract volume per cycle was 5 mL. The extract was reduced to dryness in a SpeedVac at ambient temperature. The dried extract was resuspended in 1 mL ethyl acetate, sonicated until completely dissolved and stored at −80° C. until GCMS analysis was performed.

T2 plants from both events had statistically significant increases in β-sitosterol levels in leaf tissue and in stem/silique tissue. T3 plants from both events also exhibited statistically significant increases in β-sitosterol levels in leaf tissue and in stem/silique tissue. No qualitative alterations in phenotype were noted in either the T2 or T3 plants.

### Example 8

### Analysis of Triterpenoid Content in Plants Containing a 35S::SMO 12394143 cDNA Construct

Sterol methyl oxidase (SMO) catalyzes the conversion of 24-methylene cycloartenol to 4-carboxydimethyl cycloergosenol. cDNA ID 12394143 (SEQ ID NO: 13) encodes a sterol methyl oxidase. Arabidopsis Wassilewslija (WS) plants were transformed with a T1 plasmid containing a nucleic acid designated cDNA ID 12394143 operably linked in the sense orientation relative to the CaMV 35S constitutive promoter according to the protocol in Example 1.

Six independent transformation events were selected and evaluated for their qualitative phenotype in the T1 generation. No observable differences in morphology were noted between T1 plants and controls. Plants from these events were designated ME01999 events.

T1 plants were allowed to self-pollinate and T2 seeds were collected. A portion of the T2 seeds were germinated, allowed to self-pollinate, and T3 seeds collected. T2 and T3 seeds of the Arabidopsis thaliana ME01999 events were planted and grown as described in Example 1 to confirm that the plants were transgenic for the Finale® marker.

Approximately two weeks post-bolting, all aerial tissues were collected from six Finale® resistant T2 plants of each event and analyzed for triterpenoid content as described in Example 7, except that the specific number of extractions and injections for each experiment was as described in the legend for Table 7. Aerial tissues from Finale® resistant heterozygous and homozygous T3 plants from 5 events were analyzed in the same manner.

The results of the analysis are shown in Table 7. Arabidopsis plants containing the 35S::SMO construct had increased sterol levels relative to control plants. Both campesterol and β-sitosterol levels were increased to 150% of control in the aerial tissues of six T2 events. In the T3 generation, the levels of campesterol and β-sitosterol were increased to 140% and 134% respectively, of control plants in the aerial tissues. No qualitative alterations in phenotype were noted in the T2 or T3 plants.

TABLE 7
Campesterol and β-Sitosterol levels in ME01999
T2 and T3 Generations^{a}

| | | | |
|---|---|---|---|
| Campesterol | Campesterol | β-Sitosterol | β-Sitosterol |
| (avg.) | S.D. | (avg.) | S.D. |

T2

| | | | | |
|---|---|---|---|---|
| control^{b} | 100.0 | 8.3 | 100.0 | 14.3 |
| ME01999^{c} | 150.4 | 20.1 | 150.9 | 17.1 |

T3

| | | | | |
|---|---|---|---|---|
| control^{b} | 100.0 | 5.3 | 100.0 | 11.6 |
| ME01999^{d} | 142.0 | 20.8 | 134.7 | 10.3 |

^{a}Values for ME01999 plants are expressed as percent relative to control.
^{b}Results obtained from 4 extractions and a single injection for each extraction.
^{c}Results obtained from 6 independent events with a single extraction and injection per event
^{d}Results obtained from 5 independent events with a single extraction and injection per event.

### Example 9

### Analysis of Triterpenoid Content in Plants Containing a 35S::CPI 12421417 cDNA Construct

Cyclopropyl sterol isomerase (CPI) catalyzes the conversion of cycloeucalenol to obtusifoliol. cDNA ID 12421417 (SEQ ID NO: 22) encodes a cyclopropyl sterol isomerase. Arabidopsis Wassilewskija (WS) plants were transformed with a T1 plasmid containing a nucleic acid designated cDNA ID 12421417 operably linked in the sense orientation relative to the CaMV 35S constitutive promoter according to the protocol in Example 1.

Five independent transformation events were selected and evaluated for their qualitative phenotype in the T1 generation. No observable differences in morphology were noted between T1 plants and controls. Plants from these events were designated ME01768 events.

Generation of T2 and T3 plants containing 35S::CPI cDNA 12421417 was performed as described in Example 1. Tissue extraction and triterpenoid analysis was carried out as described in Example 7, except that the number of extractions and injections was a described in the legend to Table 8.

The results of this experiment are shown in Table 8. Arabidopsis plants containing the 35S::CPI construct had increased sterol levels relative to control plants, In aerial tissues from T2 plants, campesterol levels were increased to 159% of control and β-sitosterol levels were increased to 146% of control. In aerial tissues from T3 plants, campesterol levels were increased to 138% of control and β-sitosterol levels were increased to 125% of control. No qualitative alterations in phenotype were noted in the T2 or T3 plants.

TABLE 8
Campesterol and β-Sitosterol Levels in ME01768
T2 and T3 Generations^{a}

| | | | |
|---|---|---|---|
| Campesterol | Campesterol | β-Sitosterol | β-Sitosterol |
| (avg.) | S.D. | (avg.) | S.D. |

T2

| | | | | |
|---|---|---|---|---|
| control^{b} | 100.0 | 8.3 | 100.0 | 14.3 |
| ME01768^{c} | 159.8 | 13.3 | 146.3 | 16.6 |

T3

| | | | | |
|---|---|---|---|---|
| control^{b} | 100.0 | 5.3 | 100.0 | 11.6 |
| ME01768^{d} | 138.2 | 16.8 | 125.3 | 13.7 |

^{a}Values for ME01768 plants are expressed as percent relative to control.
^{b}Results obtained from 4 extractions and a single injection for each extraction.
^{c}Results obtained from 6 independent events with a single extraction and injection per event
^{d}Results obtained from 4 independent events with a single extraction and injection per event.

### Example 10

### Analysis of Triterpenoid Content in Plants Containing a 35S::SI 13487250 cDNA Construct

C-8,7 sterol isomerase (SI) catalyzes the conversion of 4-methyl-ergosta-8,24-dienol to 24-methylene lophenol. cDNA ID 13487250 (SEQ ID NO: 27) encodes a C-8,7 sterol isomerase. Arabidopsis Wassilewslcija (WS) plants were transformed with a T1 plasmid containing a nucleic acid designated cDNA ID 13487250 operably linked in the sense orientation relative to the CaMV 35S constitutive promoter according to the protocol in Example 1. Two independent transformations were carried out with this construct resulting in two independent sets of events, ME01923 and ME02046. Ten independent transformation events of ME01923 and ME02046 were selected and evaluated for their qualitative phenotype in the T1 generation. No observable differences in morphology were noted between T1 plants and controls. Generation of T2 and T3 plants containing 35S::SI cDNA 13487250 was performed as described in Example 1. No qualitative alterations in phenotype were noted in the T2 or T3 plants. Tissue extraction and triterpenoid analysis was carried out as described in Example 7, except the number of extractions and injections was a described in the legend to Table 9.

For analysis of triterpenoid content in aerial tissues of T2 plants, 5 plants ME01923 plants and 3 ME02046 plants were extracted separately and analyzed by GC/MS. The data from all 8 plants were averaged and are shown in Table 9. Levels of β-sitosterol in the SI-transformed plants were 138% of those in control plants. In the T3 generation, Finale® resistant heterozygous and homozygous plants from ME01923 events-02 and -03 were analyzed. The level of β-sitosterol in aerial tissues was 158% and 128% respectively in Events ME01923-02 and ME01923, respectively, of those in control plants.

TABLE 9
β-Sitosterol levels in ME01923 and ME02046 T2
and ME01923 T3 Generations^{a}

| | |
|---|---|
| β-Sitosterol (avg.) | β-Sitosterol S.D. |

T2

| | | |
|---|---|---|
| control^{b} | 100.0 | 14.3 |
| M1E01923 and ME02046^{c} | 138.8 | 10.6 |

T3

| | | |
|---|---|---|
| control^{d} | 100.0 | 11.6 |
| ME01923-02^{e} | 158.5 | 19.6 |
| ME01923-03^{e} | 128.7 | 4.8 |

^{a}Values for ME01768 plants are expressed as percent relative to control.
^{b}Results obtained from 4 extractions and a single injection for each extraction.
^{C}Results obtained from 8 independent events with a single extraction and injection per event
^{d}Results obtained from 8 extractions and duplicate injections for each extraction.
^{e}Results obtained from duplicated extraction and duplicate injections for each extraction.

### Example 11

### Analysis of cDNA ID 23357293 (SEQ ID NO: 34) Activity In Vivo

The 35S::23357293 construct of Example 1 was introduced into tobacco plants, along with a construct containing an Arabidopsis squalene synthase promoter operably linked to a luciferase reporter. Treated intact leaves were collected five days after infection, and placed in a square Petri dish. Each leaf was sprayed with 10 uM luciferin in 0.01% triton-X-100. Leaves were then incubated in the dark for at least a minute prior to imaging with a Night Owl™ CCD camera from Berthold Technology. The exposure time was typically between 2 to 5 minutes. Qualitative scoring of luciferase reporter activity from each infected leaf was done by visual inspection and comparison of images, based on the following criteria: (1) whether the luminescence signal was higher in the treated leaf than in the 35S-GFP-treated reference control (considered as the background activity of the regulatory region), and (2) whether the elevated signal occurred in at least two independent transformation events carrying the regulatory region-luciferase reporter construct.

The results showed that luciferase reporter activity was detected when the Arabidopsis squalene synthase promoter::luciferase reporter construct was introduced along with the 35S::23357293 construct.

### Example 12

### Generation of Transgenic Tomato Plants Containing a 35S::ring Finger Transcription Factor cDNA 23361365 Construct

The 35S::23361365 cDNA construct of Example 5 was used to generate transgenic tomato plants. Explants of cotyledons from 7-9 day old seedlings were transfected using an Agrobacterium-mediated transformation method essentially as described in Park et al., J. Plant Physiol. 160:1253-1257 (2003). Transformants were selected using a bialaphos resistance gene as a selectable marker and selecting on a bialaphos containing medium. After selection for transformed tissues, plants were regenerated in the greenhouse, allowed to self pollinate, and seeds were collected. Seeds were germinated and grown and fruit tissues were analyzed for triterpenoid content essentially as described in Example 2.

### Example 13

### Determination of Ortholog/Functional Homology Sequences

A subject sequence was considered a functional homolog and/or ortholog of a query sequence if the subject and query sequences encode proteins having a similar function and/or activity. A process known as Reciprocal BLAST (Rivera et al, Proc. Natl Acad. Sci. USA, 1998, 95:6239-6244) was used to identify potential functional homolog and/or ortholog sequences from databases consisting of all available public and proprietary peptide sequences, including NR from NCBI and peptide translations from Ceres clones.

Before starting a Reciprocal BLAST process, a specific query polypeptide was searched against all peptides from its source species using BLAST in order to identify polypeptides having sequence identity of 80% or greater to the query polypeptide and an alignment length of 85% or greater along the shorter sequence in the alignment. The query polypeptide and any of the aforementioned identified polypeptides were designated as a cluster.

The main Reciprocal BLAST process consists of two rounds of BLAST searches; forward search and reverse search. In the forward search step, a query polypeptide sequence, “polypeptide A,” from source species S^{A} was BLASTed against all protein sequences from a species of interest. Top hits were determined using an E-value cutoff of 10⁻⁵ and an identity cutoff of 35%. Among the top hits, the sequence having the lowest E-value was designated as the best hit, and considered a potential functional homolog and/or ortholog. Any other top hit that had a sequence identity of 80% or greater to the best hit or to the original query polypeptide was considered a potential functional homolog and/or ortholog as well. This process was repeated for all species of interest.

In the reverse search round, the top hits identified in the forward search from all species were BLASTed against all protein sequences from the source species SA. A top hit from the forward search that returned a polypeptide from the aforementioned cluster as its best hit was also considered as a potential functional homolog and/or ortholog.

Functional homologs and/or orthologs were identified by manual inspection of potential functional homolog and/or ortholog sequences. Representative functional homologs and/or orthologs are shown in FIGS. 2, 4, 5, 6, 7, 8 and 9 for Arabidopsis cDNA 23389731, cDNA 23361365, cDNA 23644306, cDNA 12328487 SQS1, cDNA 12394143 SMO, cDNA 12421417 CPI, and cDNA 13487250 SI, respectively. The percent identity to Arabidopsis cDNA 23389731, cDNA 23361365, cDNA 23644306, cDNA 12328487, cDNA 12394143, cDNA 12421417, and cDNA 13487250 (SEQ ID NOS: 37, 55, 49, 2, 14, 23, and 28, respectively) are shown in Tables 10, 11, 12, 13, 14, 15, and 16, respectively, below.

TABLE 10
Percent identity to cDNA 23389731 SEQ ID NO:37

| | | SEQ | | |
|---|---|---|---|---|
| | | ID | % | |
| Designation | Species | NO: | Identity | e-value |
| gi\|1045044 | Arabidopsis thaliana | 38 | 87.9 | 0 |
| gi\|9795158 | Arabidopsis thaliana | 40 | 87.8 | 0 |
| gi\|26451634 | Arabidopsis thaliana | 39 | 87.7 | 0 |
| CeresClone:515966 | Glycine max | 42 | 86.1 | 3.4E−125 |
| gi\|1946222 | Malus x domestica | 41 | 75.7 | 0 |
| gi\|1805618 | Oryza sativa subsp. | 45 | 73.2 | 4.7E−125 |
| | japonica | | | |
| gi\|1805617 | Oryza sativa subsp. | 46 | 72.4 | 7.1E−122 |
| | japonica | | | |
| gi\|7446245 | Nicotiana tabacum | 44 | 72 | 0 |
| gi\|11463943 | Ceratopteris richardii | 47 | 66.2 | 1.8E−116 |
| gi\|6016226 | Lycopersicon | 43 | 64.4 | 6.5E−128 |
| | esculentum | | | |

TABLE 11
Percent identity to cDNA 23361365 (SEQ ID NO:55)

| | | SEQ | | |
|---|---|---|---|---|
| | | ID | % | |
| Designation | Species | NO: | Identity | e-value |
| gi\|9759231 | Arabidopsis thaliana | 56 | 98.6 | 6.4E−66 |
| CeresClone:642012 | Glycine max | 57 | 71.7 | 1.1E−19 |
| CeresClone:518866 | Glycine max | 58 | 69.6 | 7.2E−19 |
| CeresClone:766557 | Triticum aestivum | 59 | 65.9 | 7.1E−17 |
| CeresClone:246572 | Zea mays | 60 | 63.6 | 1.2E−13 |
| gi\|55733851 | Oryza sativa subsp. | | | |
| | japonica | 61 | 63 | 2.6E−15 |

TABLE 12
Percent identity to cDNA 23644306 (SEQ ID NO:49)

| | | SEQ ID | | |
|---|---|---|---|---|
| Designation | Species | NO: | % Identity | e-value |
| CeresClone:280200 | Zea mays | 50 | 68.4 | 0 |
| gi\|122 165075 | Oriyza sativa subsp. | 51 | 69.4 | 0 |
| | japonica | | | |

TABLE 13
Percent identity to cDNA 12328487 SQS1 (SEQ ID NO:2)

| | | SEQ | | |
|---|---|---|---|---|
| | | ID | % | |
| Designation | Species | NO: | Identity | e-value |
| Ceres Clone:515962 | Glycine max | 3 | 80.4 | 0 |
| gi\|55710094 | Centella asiatica | 4 | 79.6 | 0 |
| gi\|2144186 | Glycyrrhiza glabra | 5 | 78.6 | 0 |
| gi\|28208268 | Lotus japonicus | 6 | 77.9 | 0 |
| gi\|41224629 | Panax ginseng | 7 | 77.6 | 0 |
| gi\|27475614 | Medicago truncatula | 8 | 77.6 | 0 |
| gi\|5360655 | Solanum tuberosum | 9 | 76.4 | 0 |
| gi\|4426953 | Capsicum annuum | 10 | 76.2 | 0 |
| gi\|1552717 | Nicotiana tabacum | 11 | 75.9 | 0 |
| gi\|1184109 | Nicotiana benthamiana | 12 | 75.2 | 0 |

TABLE 14
Percent identity to cDNA 12394143 SMO (SEQ ID NO:14)

| | | SEQ | % | |
|---|---|---|---|---|
| | | ID | Iden- | |
| Designation | Species | NO: | tity | e-value |
| gi\|27448145 | Gossypium arboreum | 15 | 85.7 | 2.7E−122 |
| CeresClone:664026 | Glycine max | 16 | 84.1 | 5.8E−120 |
| CeresClone:977729 | Brassica napus | 17 | 81.8 | 5.6E−106 |
| gi\|34978966 | Nicotiana benthamiana | 18 | 80.2 | 9.5E−111 |
| gi\|51963234 | Oryza sativa subsp. | 19 | 76.1 | 4E−112 |
| | japonica | | | |
| CeresClone:217004 | Zea mays | 20 | 73.9 | 4.9E−107 |
| CeresClone:245428 | Zea mays | 21 | 73.5 | 3.7E−109 |

TABLE 15
Percent identity to cDNA 12421417 CPI (SEQ ID NO:23)

| | | SEQ ID | % | |
|---|---|---|---|---|
| Designation | Species | NO: | Identity | e-value |
| CeresClone:716942 | Glycine max | 24 | 80.3 | 1.4E−127 |
| CeresClone:285554 | Zea mays | 25 | 79.6 | 9.1E−122 |
| gi\|62732798 | Oryza sativa subsp. | 26 | 76.4 | 6.5E−112 |
| | japonica | | | |

TABLE 16
Percent identity to cDNA 13487250 SI (SEQ ID NO:28)

| | | SEQ ID | % | |
|---|---|---|---|---|
| Designation | Species | NO: | Identity | e-value |
| CeresClone:959258 | Brassica napus | 29 | 81.3 | 2.5E−94 |
| CeresClone:592262 | Glycine max | 30 | 71.8 | 7.1E−83 |
| CeresClone:282337 | Zea mays | 31 | 64.9 | 7.9E−52 |
| gi\|50900588 | Oryza sativa subsp. | | | |
| | japonica | 32 | 60.0 | 1.5E−66 |
| CeresClone:703736 | Triticum aestivum | 33 | 59.4 | 1.5E−66 |

Nucleic acids encoding other functional homologs and/or orthologs are shown in SEQ ID NO: 156; SEQ ID NO: 158; SEQ ID NO: 160; SEQ ID NO: 162; SEQ ID NO: 165; SEQ ID NO: 167; SEQ ID NO: 170; SEQ ID NO: 172; SEQ ID NO: 174; SEQ ID NO: 176; SEQ ID NO: 178; SEQ ID NO: 180; SEQ ID NO: 182; SEQ ID NO: 184; SEQ ID NO: 187; SEQ ID NO: 189 and SEQ ID NO: 191. Amino acid sequences for the encoded polypeptides are shown in SEQ ID NO: 157; SEQ ID NO: 159; SEQ ID NO: 161; SEQ ID NO: 163; SEQ ID NO: 164; SEQ ID NO: 166; SEQ ID NO: 168; SEQ ID NO: 169; SEQ ID NO: 171; SEQ ID NO: 173; SEQ ID NO: 175; SEQ ID NO: 177; SEQ ID NO: 179; SEQ ID NO: 181; SEQ ID NO: 183; SEQ ID NO: 185; SEQ ID NO: 186; SEQ ID NO: 188; SEQ ID NO: 190 and SEQ ID NO: 192.

### Example 14

### Generation and Analysis of Triterpenoid Content in Transgenic Tomato Plants Containing a 35S::SMO 12394143 cDNA Construct

The Arabidopsis 35S::SMO 12394143 cDNA (SEQ ID NO: 13) (AtSMO) construct of Example 8 was used to generate transgenic tomato plants. Explants of cotyledons from 7-9 day old seedlings were transfected using an Agrobacterium-mediated transformation method essentially as described in Park et al., J. Plant Physiol. 160:1253-1257 (2003). Transformants were selected using a bialaphos resistance gene as a selectable marker and selecting on a bialaphos containing medium. After selection for transformed tissues, T₀ plants were regenerated in the greenhouse, allowed to self pollinate, and fruit tissues were analyzed for triterpenoid content essentially as described in Example 2.

As shown in Tables 17-20, the levels of one or more of stigmasterol, sitosterol, β-amyrin and α-amyrin were significantly increased relative to the corresponding amounts in transgenic control plants in fruit tissues of T₀ events. For example, as shown in Table 17, the stigmasterol content in fruits from T₀ events SMO-01, SMO-03, and SMO-X was increased to 160%, 123%, and 210%, respectively, of the stigmasterol content in transgenic control plants. In one event, SMO-Y, the stigmasterol content was decreased to 65% of the stigmasterol content in transgenic control plants.

TABLE 17
Stigmasterol levels in 35S::AtSMO T₀ Tomato Plants^{a}

| | SMO-01 | SMO-02 | SMO-03 | SMO-04 | SMO-06 | SMO-18 | SMO-20 | SMO-X | SMO-Y | Control |
|---|---|---|---|---|---|---|---|---|---|---|
| T₀ | 160 ± 10 | 104 ± 32 | 123 ± 7 | 91 | 86 ± 7 | 93 ± 62 | 81 | 210 ± 14 | 65 ± 8 | 100 ± 7 |
| p-value^{b} | 0.02 | 0.75 | p < 0.01 | — | 0.56 | 0.14 | — | p < 0.01 | p < 0.01 | N/A |

^{a}Values for 35S::AtSMO T₀ tomato plants are expressed as percent relative to control.
^{b}P-values were determined using a Student's t-test.
— = p-value not determined.

As shown in Table 18, the sitosterol content in fruits from T₀ events SMO-01, SMO-03, and SMO-X was increased to 224%, 138%, and 234%, respectively, of the sitosterol content in transgenic control plants.

TABLE 18
Sitosterol levels in 35S::AtSMO T₀ Tomato Plants^{a}

| | SMO-01 | SMO-02 | SMO-03 | SMO-04 | SMO-06 | SMO-18 | SMO-20 | SMO-X | SMO-Y | Control |
|---|---|---|---|---|---|---|---|---|---|---|
| T₀ | 224 ± 8 | 113 ± 17 | 138 ± 1 | 93 | 90 ± 07 | 122 ± 29 | 104 | 234 ± 41 | 77 ± 17 | 100 ± 10 |
| p-value^{b} | p < 0.01 | 0.45 | p < 0.01 | — | 0.56 | 0.48 | — | 0.05 | 0.24 | N/A |

^{a}Values for 35S::AtSMO T₀ tomato plants are expressed as percent relative to control.
^{b}P-values were determined using a Student's t-test.
— = p-value not determined.

As shown in Table 19, the β-amyrin content in fruits from T₀ events SMO-X and SMO-Y was increased to 296% and 152%, respectively, of the β-amyrin content in transgenic control plants.

TABLE 19
β-amyrin levels in 35S::AtSMO T₀ Tomato Plants^{a}

| | SMO-01 | SMO-02 | SMO-03 | SMO-04 | SMO-06 | SMO-18 | SMO-20 | SMO-X | SMO-Y | Control |
|---|---|---|---|---|---|---|---|---|---|---|
| T₀ | 127 ± 20 | 129 ± 17 | 131 ± 24 | 133 | 148 ± 7 | 100 ± 02 | 98 | 296 ± 41 | 152 ± 15 | 100 ± 3 |
| p-value^{b} | 0.17 | 0.45 | 0.11 | — | 0.56 | 0.90 | — | p < 0.01 | 0.02 | N/A |

^{a}Values for 35S::AtSMO T0 tomato plants are expressed as percent relative to control.
^{b}P-values were determined using a Student's t-test.
— = p-value not determined.

As shown in Table 20, the α-amyrin content in fruits from T₀ events SMO-03 and SMO-X was increased to 212% and 157%, respectively, of the α-amyrin content in transgenic control plants. In one event, SMO-18, the α-amyrin content was decreased to 68% of the α-amyrin content in transgenic control plants.

TABLE 20
α-amyrin levels in 35S::AtSMO T₀ Tomato Plants^{a}

| | SMO-01 | SMO-02 | SMO-03 | SMO-04 | SMO-06 | SMO-18 | SMO-20 | SMO-X | SMO-Y | Control |
|---|---|---|---|---|---|---|---|---|---|---|
| T₀ | 71 ± 11 | 62 ± 17 | 212 ± 4 | 53 | 61 ± 7 | 68 ± 3 | 106 | 157 ± 12 | 80 ± 13 | 100 ± 5 |
| p-value^{b} | 0.45 | 0.45 | p < 0.01 | — | 0.56 | 0.01 | — | 0.01 | 0.11 | N/A |

^{a}Values for 35S::AtSMO T₀ tomato plants are expressed as percent relative to control.
^{b}P-values were determined using a Student's t-test.
— = p-value not determined.

### Example 15

### Analysis of Triterpenoid Content in Plants Containing a 35S::SMO 217004 cDNA Construct

CeresClone 217004 (SEQ ID NO: 193) is predicted to encode a Zea mays sterol methyl oxidase. Transgenic Arabidopsis thaliana plants containing a 35S::CeresClone 217004 construct were made according to the protocol described in Example 1, using a construct that contained a nucleic acid designated clone ID 217004 operably linked in the sense orientation to a 35S promoter. Independent transformation events were selected and evaluated for their qualitative phenotype in the T1 generation. No observable differences in morphology were noted between T1 plants and controls. Plants from these events were designated ME13726 events.

T1 plants were allowed to self-pollinate and T2 seeds were collected. T2 seeds of ME13726 events were planted and grown as described in Example 1 to confirm that the plants were transgenic for the Finale® marker. Approximately 10 days post-bolting, all aerial tissues were collected from Finale® resistant T2 plants of five events and analyzed for triterpenoid content as described in Example 2.

As shown in Tables 21-24, the levels of one or more of squalene, campesterol, stigmasterol and β-amyrin were significantly increased relative to the corresponding amounts in “transgenic control” plants in T2 aerial tissues of ME13726 events. For example, as shown in Table 21, the squalene content in aerial tissues from T2 events ME13726-01, ME13726-02, ME13726-03, and ME13726-04 was increased to 169%, 185%, 191%, and 181%, respectively, of the squalene content in transgenic control plants.

TABLE 21
Squalene Levels in ME13726 T2 Generation^{a}

| | ME013726-01 | ME013726-02 | ME013726-03 | ME013726-04 | ME013726-05 | Control |
|---|---|---|---|---|---|---|
| T2 | 169 ± 12 | 185 ± 11 | 191 ± 0.05 | 181 ± 6 | 116 ± 11 | 100 ± 21 |
| p-value^{b} | 0.01 | <0.01 | 0.01 | 0.01 | 0.25 | N/A |

^{a}Values for ME13726 plants are expressed as percent relative to control.
^{b}P-values were determined using a Student's t-test.

As shown in Table 22, the campesterol content in aerial tissues from T2 events ME13726-01, ME13726-02, ME13726-03, ME13726-04, and ME13726-05 was increased to 158%, 124%, 111%, 111% and 131%, respectively, of the campesterol content in transgenic control plants.

TABLE 22
Campesterol Levels in ME13726 T2 Generation^{a}

| | ME013726-01 | ME013726-02 | ME013726-03 | ME013726-04 | ME013726-05 | Control |
|---|---|---|---|---|---|---|
| T2 | 158 ± 5 | 124 ± 2 | 111 ± 4 | 111 ± 1 | 131 ± 11 | 100 ± 2 |
| p-value^{b} | <0.01 | <0.01 | 0.02 | 0.01 | <0.01 | N/A |

^{a}Values for ME13726 plants are expressed as percent relative to control.
^{b}P-values were determined using a Student's t-test.

As shown in Table 23, the stigmasterol content in aerial tissues from the T2 event ME13726-02 was increased to 174% of the stigmasterol content in transgenic control plants.

TABLE 23
Stigmasterol Levels in ME13726 T2 Generation^{a}

| | ME013726-01 | ME013726-02 | ME013726-03 | ME013726-04 | ME013726-05 | Control |
|---|---|---|---|---|---|---|
| T2 | 127 ± 26 | 174 ± 4 | 127 ± 5 | 108 ± 44 | 92 ± 23 | 100 ± 23 |
| p-value^{b} | 0.2 | 0.01 | 0.06 | 0.79 | 0.02 | N/A |

^{a}Values for ME13726 plants are expressed as percent relative to control.
^{b}P-values were determined using a Student's t-test.

As shown in Table 24, the β-amyrin content in aerial tissues from T2 events ME13726-01, ME13726-02, ME13726-03, ME13726-04, and ME13726-05 was increased to 176%, 243%, 201%, 201% and 134%, respectively, of the β-amyrin content in transgenic control plants.

TABLE 24
β-Amyrin Levels in ME13726 T2 Generation^{a}

| | ME013726-01 | ME013726-02 | ME013726-03 | ME013726-04 | ME013726-05 | Control |
|---|---|---|---|---|---|---|
| T2 | 176 ± 8 | 243 ± 17 | 201 ± 16 | 201± 6 | 134 ± 5 | 100 ± 3 |
| p-value^{b} | <0.01 | <0.01 | 0.01 | <0.01 | <0.01 | N/A |

^{a}Values for ME13726 plants are expressed as percent relative to control.
^{b}P-values were determined using a Student's t-test.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of altering the level of a triterpenoid in a plant, said method comprising introducing into a plant cell an exogenous nucleic acid comprising a nucleotide sequence encoding a polypeptide having 80% or greater sequence identity to an amino acid sequence selected from the group consisting of SEQ ID NO: 35, SEQ ID NOS: 37-47, SEQ ID NO: 53, SEQ ID NOS: 55-61, SEQ ID NOS: 49-51, SEQ ID NOs: 2-12, SEQ ID NOs: 14-21, SEQ ID NOs: 23-26, and SEQ ID NOS: 28-33, wherein a tissue of a plant produced from said plant cell has a difference in the level of a triterpenoid as compared to the corresponding level in tissue of a control plant that does not comprise said nucleic acid.

2. A method of altering the level of a triterpenoid in a plant, said method comprising introducing into a plant cell an exogenous nucleic acid comprising a nucleotide sequence encoding a polypeptide having 95% or greater sequence identity to an amino acid sequence selected from the group consisting of SEQ ID NO: 35, SEQ ID NO: 37, SEQ ID NO: 42, SEQ ID NO: 53, SEQ ID NO: 55, SEQ ID NOS: 57-60, SEQ ID NOS: 49-50, SEQ ID NO: 2, SEQ ID NO: 14, SEQ ID NO: 23, and SEQ ID NO: 28, wherein a tissue of a plant produced from said plant cell has a difference in the level of a triterpenoid as compared to the corresponding level in tissue of a control plant that does not comprise said nucleic acid

3. The method of claims 1 or 2, wherein said nucleotide sequence encodes a polypeptide having 80% or greater sequence identity to an amino acid sequence selected from the group consisting of SEQ ID NO: 35, SEQ ID NO: 37, SEQ ID NO: 53, SEQ ID NO: 55, SEQ ID NO: 49, SEQ ID NO: 3, SEQ ID NO: 16, SEQ ID NO: 24, and SEQ ID NO: 29.

4. The method of claim 1 wherein said sequence identity is 85% or greater.

5. The method of claim 1 wherein said sequence identity is 90% or greater.

6. The method of claim 3, wherein said sequence identity is 95% or greater.

7. The method of claims 1 or 2 wherein said nucleotide sequence encodes a polypeptide comprising an amino acid sequence corresponding to SEQ ID NO: 35.

8. The method of claims 1 or 2 wherein said nucleotide sequence encodes a polypeptide comprising an amino acid sequence corresponding to SEQ ID NO: 37.

9. The method of claims 1 or 2 wherein said nucleic acid sequence encodes a polypeptide comprising an amino acid sequence corresponding to SEQ ID NO: 53.

10. The method of claims 1 or 2 wherein said nucleic acid sequence encodes a polypeptide comprising an amino acid sequence corresponding to SEQ ID NO: 55.

11. The method of claims 1 or 2 wherein said nucleic acid sequence encodes a polypeptide comprising an amino acid sequence corresponding to SEQ ID NO: 49.

12. (canceled)

13. A method of altering the level of a triterpenoid in a plant, said method comprising introducing into a plant cell: (a) a first exogenous nucleic acid comprising a nucleotide sequence encoding a polypeptide having 80% or greater sequence identity to an amino acid sequence selected from the group consisting of SEQ ID NO: 35, SEQ ID NOS: 37-47, SEQ ID NO: 53, SEQ ID NOS: 55-61, and SEQ ID NOS: 49-51; and (b) a second exogenous nucleic acid comprising a nucleotide sequence encoding a polypeptide having 80% or greater sequence identity to an amino acid sequence selected from the group consisting of SEQ ID NOs: 2-12, SEQ ID NOs: 14-21, SEQ ID NOs: 23-26, and SEQ ID NOS: 28-33, wherein a tissue of a plant produced from said plant cell has a difference in the level of a triterpenoid as compared to the corresponding level in tissue of a control plant that does not comprise said first nucleic acid and said second nucleic acid.

14. The method of claim 13, wherein said second nucleic acid comprises a nucleic acid sequence encoding a polypeptide having 80% or greater sequence identity to an amino acid sequence selected from the group consisting of SEQ ID NOS: 2-12.

15. The method of claim 13, wherein said second nucleic acid comprises a nucleotide sequence encoding a polypeptide having 80% or greater sequence identity to an amino acid sequence selected from the group consisting of SEQ ID NOS: 14-21.

16. The method of claim 13, wherein said second nucleic acid comprises a nucleic acid sequence encoding a polypeptide having 80% or greater sequence identity to an amino acid sequence selected from the group consisting of SEQ ID NOS: 23-26.

17. The method of claim 13, wherein said second nucleic acid comprises a nucleic acid sequence encoding a polypeptide having 80% or greater sequence identity to an amino acid sequence selected from the group consisting of SEQ ID NOS: 28-33.

18. A method of altering the level of a triterpenoid in a plant, said method comprising introducing into a plant cell: (a) a first exogenous nucleic acid comprising a nucleotide sequence encoding a polypeptide having 80% or greater sequence identity to an amino acid sequence selected from the group consisting of SEQ ID NO: 35, SEQ ID NOS: 37-47, SEQ ID NO: 53, SEQ ID NOS: 55-61, and SEQ ID NOS: 49-51; and (b) a second exogenous nucleic acid comprising a nucleotide sequence encoding a polypeptide having 80% or greater sequence identity to an amino acid sequence selected from the group consisting of SEQ ID NO: 35, SEQ ID NOS: 37-47, SEQ ID NO: 53, SEQ ID NOS: 55-61, and SEQ ID NOS: 49-51; provided that the said first exogenous nucleic acid and the said second exogenous nucleic acid are not the same, wherein a tissue of a plant produced from said plant cell has a difference in the level of a triterpenoid as compared to the corresponding level in tissue of a control plant that does not comprise said first nucleic acid and said second nucleic acid.

19. A method of altering the level of a triterpenoid in a plant, said method comprising introducing into a plant cell: (a) a first exogenous nucleic acid comprising a nucleotide sequence encoding a polypeptide having 80% or greater sequence identity to an amino acid sequence selected from the group consisting of SEQ ID NOs: 2-12, SEQ ID NOs: 14-21, SEQ ID NOs: 23-26, and SEQ ID NOS: 28-33; and (b) a second exogenous nucleic acid comprising a nucleotide sequence encoding a polypeptide having 80% or greater sequence identity to an amino acid sequence selected from the group consisting of SEQ ID NOs: 2-12, SEQ ID NOs: 14-21, SEQ ID NOs: 23-26, and SEQ ID NOS: 28-33; provided that the said first exogenous nucleic acid and the said second exogenous nucleic acid are not the same, wherein a tissue of a plant produced from said plant cell has a difference in the level of a triterpenoid as compared to the corresponding level in tissue of a control plant that does not comprise said first nucleic acid and said second nucleic acid.

20. The method of any of claims 1, 2, 13, 18 and 19, wherein said difference is an increase in the level of an acyclic triterpenoid.

21. The method of any of claims 1, 2, 13, 18 and 19, wherein said triterpenoid is selected from the group consisting of squalene, β-sitosterol, sitostanol, stigmasterol, campesterol, □-amyrin, and β-amyrin

22. The method of any of claims 1, 2, 13, 18 and 19, wherein said difference is an increase in the level of a triterpenoid selected from the group consisting of squalene, lupeol, α-amyrin, β-amyrin, glycyrrhizin, β-sitosterol, sitostanol, stigmasterol, campesterol, ergosterol, diosgenin, aescin, betulinic acid, cucurbitacin E, ruscogenin, mimusin, avenacin A-1, gracillin, α-tomatine, α-solanine, convallatoxin, acetyldigoxin, digoxin, deslanoside, digitalin, digitoxin, quillaic acid and its glycoside derivatives, squalamine, ouabain, strophanthidin, hydrocortisone, testosterone, and asiaticoside.

23. The method of any of claims 1, 2, 13, 18 and 19, wherein said difference is an increase in the level of a sterol.

24. The method of claim 23, wherein said difference is an increase in the level of β-sitosterol.

25. The method of any of claims 1 or 2, wherein said exogenous nucleic acid is operably linked to a regulatory region.

26. The method of claim 25, wherein said regulatory region is a cell-specific or tissue-specific promoter.

27. The method of claim 26, wherein said promoter is a leaf-specific promoter.

28. The method of claim 26, wherein said promoter is a seed-specific promoter.

29. The method of claim 28, wherein said seed-specific promoter is selected from the group consisting of the promoters YP0092 (SEQ ID NO: 62), PT0676 (SEQ ID NO: 72), PT0708 (SEQ ID NO: 74), PT0613 (SEQ ID NO: 66), PT0672 (SEQ ID NO: 68), PT0678 (SEQ ID NO: 69), PT0688 (SEQ ID NO: 70), PT0837 (SEQ ID NO: 76), the napin promoter, the Arcelin-5 promoter, the phaseolin gene promoter, the soybean trypsin inhibitor promoter, the ACP promoter, the stearoyl-ACP desaturase gene, the soybean α′ subunit of β-conglycinin promoter, the oleosin promoter, the 15 kD zein promoter, the 16 kD zein promoter, the 19 kD zein promoter, the 22 kD zein promoter, the 27 kD zein promoter, the Osgt-1 promoter, the beta-amylase gene promoter, and the barley hordein gene promoter.

30. The method of claim 26, wherein said promoter is a root-specific promoter.

31. The method of claim 30, wherein said root-specific promoter is selected from the group consisting of YP0128 (SEQ ID NO: 63), YP0275 (SEQ ID NO: 65), PT0625 (SEQ ID NO: 67), PT0660 (SEQ ID NO: 71), PT0683 (SEQ ID NO: 73), and PT0758 (SEQ ID NO: 75).

32. The method of claim 25, wherein said regulatory region is a broadly expressing promoter.

33. The method of claim 32, wherein said broadly expressing promoter is selected from the group consisting of p326, YP0158, YP0214, YP0380, PT0848, PT0633, YP0050, YP0144, and YP0190.

34. The method of claim 25, wherein said regulatory region is a constitutive promoter.

35. The method of claim 25, wherein said regulatory region is an inducible promoter.

36. The method of any of claims 13, 18 or 19, wherein said first nucleic acid and said second nucleic acid are operably linked to a first and a second regulatory region, respectively.

37. The method of claim 36, wherein said regulatory regions are cell-specific or tissue-specific promoters.

38. The method of claim 36, wherein said regulatory regions are seed-specific promoters.

39. The method of claim 36, wherein said regulatory regions are leaf-specific promoters.

40. The method of claim 36, wherein said regulatory regions are broadly expressing promoters.

41. The method of claim 36, wherein said regulatory regions are constitutive promoters.

42. The method of claim 36, wherein said regulatory regions are inducible promoters.

43. The method of any of claims 1, 2, 13, 18, or 19, wherein said plant is from a genus selected from the group consisting of Acokanthera, Aesculus, Ananas, Arachis, Betula, Bixa, Brassica, Calendula, Carthamus, Centella, Chrysanthemum, Cinnamomum, Citrullus, Coffea, Convallaria, Curcuma, Digitalis, Dioscorea, Fragaria, Glycine, Glycyrrhiza, Gossypium, Helianthus, Lactuca, Lavandula, Linum, Luffa, Lycopersicon, Mentha, Musa, Ocimum, Origanum, Oryza, Quillaja, Rosmarinus, Ruscus, Salvia, Sesamum, Solanum, Strophanthus, Theobroma, Thymus, Triticum, Vitis, and Zea.

44. The method of any of claims 1, 12, 13, 18, or 19, wherein said plant is a species selected from Acokanthera spp., Ananas comosus, Betula alba, Bixa orellana, Brassica campestris, Brassica napus, Brassica oleracea, Calendula officinalis, Cathamus tinctorius, Centella asiatica, Chrysanthemum parthenium, Cinnamomum camphora, Citrullus spp., Coffea arabica, Convallaria majalis, Digitalis lantana, Digitalis purpurea, Digitalis spp., Dioscorea spp., Glycine max, Glycyrrhiza glabra, Gossypium spp., Lactuca sativa, Luffa spp., Lycopersicon esculentum, Mentha piperita, Mentha spicata, Musa paradisiaca, Oryza sativa, Quillaja saponaria, Rosmarinus officinalis, Ruscus aculeatus, Solanum tuberosum, Strophanthus gratus, Strophanthus spp., Theobroma cacao, Triticum aestivum, Vitis vinifera, and Zea mays.

45. The method of any of claims 1, 12, 13, 18, or 19, wherein said plant is selected from the group consisting of peanut, safflower, flax, sugar beet, chick peas, alfalfa, spinach, clover, cabbage, lentils, mustard, soybean, lettuce, castor bean, sesame, carrot, grape, cotton, crambe, strawberry, amaranth, rape, broccoli, peas, pepper, tomato, potato, yam, kidney beans, lima beans, dry beans, green beans, watermelon, cantaloupe, peach, pear, apple, cherry, orange, lemon, grapefruit, plum, mango, soaptree bark, oilseed rape, sunflower, garlic, oil palm, date palm, banana, sweet corn, popcorn, field corn, wheat, rye, barley, oat, onion, pineapple, rice, millet, and sorghum.

46. The method of any of claims 1, 12, 13, 18, or 19, wherein said tissue is leaf tissue.

47. The method of any of claims 1, 12, 13, 18, or 19, wherein said tissue is seed tissue.

48. A method of producing a plant tissue, said method comprising growing a plant cell an exogenous nucleic acid comprising a nucleotide sequence encoding a polypeptide having 80% or greater sequence identity to an amino acid sequence selected from the group consisting of SEQ ID NO: 35, SEQ ID NOS: 37-47, SEQ ID NO: 53, SEQ ID NOS: 55-61, SEQ ID NOS: 49-51, SEQ ID NOs: 2-12, SEQ ID NOs: 14-21, SEQ ID NOs: 23-26, and SEQ ID NOS: 28-33, wherein said tissue has a difference in the level of a triterpenoid as compared to the corresponding level in tissue of a control plant that does not comprise said nucleic acid.

49. A method of producing a plant tissue, said method comprising growing a plant cell comprising (a) a first exogenous nucleic acid comprising a nucleotide sequence encoding a polypeptide having 80% or greater sequence identity to an amino acid sequence selected from the group consisting of SEQ ID NO: 35, SEQ ID NOS: 37-47, SEQ ID NO: 53, SEQ ID NOS: 55-61, and SEQ ID NOS: 49-51; and (b) a second exogenous nucleic acid comprising a nucleotide sequence encoding a polypeptide having 80% or greater sequence identity to an amino acid sequence selected from the group consisting of SEQ ID NOs: 2-12, SEQ ID NOs: 14-21, SEQ ID NOs: 23-26, and SEQ ID NOS: 28-33; wherein said tissue has a difference in the level of a triterpenoid as compared to the corresponding level in tissue of a control plant that does not comprise said first nucleic acid and said second nucleic acid.

50. The method of claim 49, wherein said second nucleic acid comprises a nucleic acid sequence encoding a polypeptide having 80% or greater sequence identity to an amino acid selected from the group consisting of SEQ ID NOS: 2-12.

51. The method of claim 49, wherein said second nucleic acid comprises a nucleotide sequence encoding a polypeptide having 80% or greater sequence identity to an amino acid sequence selected from the group consisting of SEQ ID NOS: 14-21.

52. The method of claim 49, wherein said second nucleic acid comprises a nucleic acid sequence encoding a polypeptide having 80% or greater sequence identity to an amino acid sequence selected from the group consisting of SEQ ID NOS: 23-26.

53. The method of claim 49, wherein said second nucleic acid comprises a nucleic acid sequence encoding a polypeptide having 80% or greater sequence identity to an amino acid sequence selected from the group consisting of SEQ ID NOS: 28-33.

54. A method of producing a plant tissue, said method comprising growing a plant cell comprising: (a) a first exogenous nucleic acid comprising a nucleotide sequence encoding a polypeptide having 80% or greater sequence identity to an amino acid sequence selected from the group consisting of SEQ ID NO: 35, SEQ ID NOS: 37-47, SEQ ID NO: 53, SEQ ID NOS: 55-61, and SEQ ID NOS: 49-51; and (b) a second exogenous nucleic acid comprising a nucleotide sequence encoding a polypeptide having 80% or greater sequence identity to an amino acid sequence selected from the group consisting of SEQ ID NO: 35, SEQ ID NOS: 37-47, SEQ ID NO: 53, SEQ ID NOS: 55-61, and SEQ ID NOS: 49-51; provided that the said first exogenous nucleic acid and the said second exogenous nucleic acid are not the same, wherein said tissue has a difference in the level of a triterpenoid as compared to the corresponding level in tissue of a control plant that does not comprise said first nucleic acid and said second nucleic acid.

55. A method of producing a plant tissue, said method comprising growing a plant cell comprising: (a) a first exogenous nucleic acid comprising a nucleotide sequence encoding a polypeptide having 80% or greater sequence identity to an amino acid sequence selected from the group consisting of SEQ ID NOs: 2-12, SEQ ID NOs: 14-21, SEQ ID NOs: 23-26, and SEQ ID NOS: 28-33; and (b) a second exogenous nucleic acid comprising a nucleotide sequence encoding a polypeptide having 80% or greater sequence identity to an amino acid sequence selected from the group consisting of SEQ ID NOs: 2-12, SEQ ID NOs: 14-21, SEQ ID NOs: 23-26, and SEQ ID NOS: 28-33; provided that the said first exogenous nucleic acid and the said second exogenous nucleic acid are not the same, wherein said tissue has a difference in the level of a triterpenoid as compared to the corresponding level in tissue of a control plant that does not comprise said first nucleic acid and said second nucleic acid.

56. A method of producing a triterpenoid, said method comprising extracting a triterpenoid from transgenic plant tissue, said plant tissue comprising a nucleotide sequence encoding a polypeptide having 80% or greater sequence identity to an amino acid sequence selected from the group consisting of SEQ ID NO: 35, SEQ ID NOS: 37-47, SEQ ID NO: 53, SEQ ID NOS: 55-61, SEQ ID NOS: 49-51, SEQ ID NOs: 2-12, SEQ ID NOs: 14-21, SEQ ID NOs: 23-26, and SEQ ID NOS: 28-33, wherein said tissue has a difference in the level of a triterpenoid as compared to the corresponding level in tissue of a control plant that does not comprise said nucleic acid.

57. A method of producing a triterpenoid, said method comprising extracting a triterpenoid from transgenic plant tissue, said plant tissue comprising (a) a first exogenous nucleic acid comprising a nucleotide sequence encoding a polypeptide having 80% or greater sequence identity to an amino acid sequence selected from the group consisting of SEQ ID NO: 35, SEQ ID NOS: 37-47, SEQ ID NO: 53, SEQ ID NOS: 55-61, and SEQ ID NOS: 49-51; and (b) a second exogenous nucleic acid comprising a nucleotide sequence encoding a polypeptide having 80% or greater sequence identity to an amino acid sequence selected from the group consisting of SEQ ID NOs: 2-12, SEQ ID NOs: 14-21, SEQ ID NOs: 23-26, and SEQ ID NOS: 28-32; wherein said tissue has a difference in the level of a triterpenoid as compared to the corresponding level in tissue of a control plant that does not comprise said first nucleic acid and said second nucleic acid.

58. The method of claim 57, wherein said second nucleic acid comprises a nucleic acid sequence encoding a polypeptide having 80% or greater sequence identity to an amino acid selected from the group consisting of SEQ ID NOS: 2-12.

59. The method of claim 57, wherein said second nucleic acid comprises a nucleotide sequence encoding a polypeptide having 80% or greater sequence identity to an amino acid sequence selected from the group consisting of SEQ ID NOS: 14-21.

60. The method of claim 57, wherein said second nucleic acid comprises a nucleic acid sequence encoding a polypeptide having 80% or greater sequence identity to an amino acid sequence selected from the group consisting of SEQ ID NOS: 23-26.

61. The method of claim 57, wherein said second nucleic acid comprises a nucleic acid sequence encoding a polypeptide having 80% or greater sequence identity to an amino acid sequence selected from the group consisting of SEQ ID NOS: 28-33.

62. A method of producing a triterpenoid, said method comprising extracting a triterpenoid from transgenic plant tissue, said plant tissue comprising: (a) a first exogenous nucleic acid comprising a nucleotide sequence encoding a polypeptide having 80% or greater sequence identity to an amino acid sequence selected from the group consisting of SEQ ID NO: 35, SEQ ID NOS: 37-47, SEQ ID NO: 53, SEQ ID NOS: 55-61, and SEQ ID NOS: 49-51; and (b) a second exogenous nucleic acid comprising a nucleotide sequence encoding a polypeptide having 80% or greater sequence identity to an amino acid sequence selected from the group consisting of SEQ ID NO: 35, SEQ ID NOS: 37-47, SEQ ID NO: 53, SEQ ID NOS: 55-61, and SEQ ID NOS: 49-51; provided that the said first exogenous nucleic acid and the said second exogenous nucleic acid are not the same, wherein said tissue has a difference in the level of a triterpenoid as compared to the corresponding level in tissue of a control plant that does not comprise said first nucleic acid and said second nucleic acid.

63. A method of producing a triterpenoid, said method comprising extracting a triterpenoid from transgenic plant tissue, said plant tissue comprising: (a) a first exogenous nucleic acid comprising a nucleotide sequence encoding a polypeptide having 80% or greater sequence identity to an amino acid sequence selected from the group consisting of SEQ ID NOs: 2-12, SEQ ID NOs: 14-21, SEQ ID NOs: 23-26, and SEQ ID NOS: 28-33; and (b) a second exogenous nucleic acid comprising a nucleotide sequence encoding a polypeptide having 80% or greater sequence identity to an amino acid sequence selected from the group consisting of SEQ ID NOs: 2-12, SEQ ID NOs: 14-21, SEQ ID NOs: 23-26, and SEQ ID NOS: 28-33; provided that the said first exogenous nucleic acid and the said second exogenous nucleic acid are not the same, wherein said tissue has a difference in the level of a triterpenoid as compared to the corresponding level in tissue of a control plant that does not comprise said first nucleic acid and said second nucleic acid.

64. The method of any of claims 48, 49, 54, 55, 56, 57, 62 or 63, wherein said sequence identity is 95% or greater.

65-66. (canceled)

67. The method of claims 48 or 56, wherein said nucleotide sequence encodes a polypeptide comprising an amino acid sequence corresponding to SEQ ID NO: 35.

68. The method of claims 48 or 56, wherein said nucleotide sequence encodes a polypeptide comprising an amino acid sequence corresponding to SEQ ID NO: 37.

69. The method of claims 48 or 56, wherein said nucleic acid sequence encodes a polypeptide comprising an amino acid sequence corresponding to SEQ ID NO: 53.

70. The method of claims 48 or 56, wherein said nucleic acid sequence encodes a polypeptide comprising an amino acid sequence corresponding to SEQ ID NO: 55.

71. The method of claims 48 or 56, wherein said nucleic acid sequence encodes a polypeptide comprising an amino acid sequence corresponding to SEQ ID NO: 49.

72.-74. (canceled)

75. The method of any of claims 48, 49, 54, 55, 56, 57, 62 or 63, wherein said difference is an increase in the level of a triterpenoid selected from the group consisting of squalene, lupeol, α-amyrin, β-amyrin, glycyrrhizin, β-sitosterol, sitostanol, stigmasterol, campesterol, ergosterol, diosgenin, aescin, betulinic acid, cucurbitacin E, ruscogenin, mimusin, avenacin A-1, gracillin, α-tomatine, α-solanine, convallatoxin, acetyldigoxin, digoxin, deslanoside, digitalin, digitoxin, quillaic acid and its glycoside derivatives, squalamine, ouabain, strophanthidin, hydrocortisone, testosterone, and asiaticoside.

76.-95. (canceled)

96. The method of any of claims 48, 63, 48, 49, 54, 55, 56, 57, 62 or 63, wherein said plant is from a genus selected from the group consisting of Acokanthera, Aesculus, Ananas, Arachis, Betula, Bixa, Brassica, Calendula, Carthamus, Centella, Chrysanthemum, Cinnamomum, Citrullus, Coffea, Convallaria, Curcuma, Digitalis, Dioscorea, Fragaria, Glycine, Glycyrrhiza, Gossypium, Helianthus, Lactuca, Lavandula, Linum, Luffa, Lycopersicon, Mentha, Musa, Ocimum, Origanum, Oryza, Quillaja, Rosmarinus, Ruscus, Salvia, Sesamum, Solanum, Strophanthus, Theobroma, Thymus, Triticum, Vitis, and Zea.

97.-101. (canceled)

102. A plant cell comprising an exogenous nucleic acid comprising a nucleotide sequence encoding a polypeptide having 80% or greater sequence identity to an amino acid sequence selected from the group consisting of SEQ ID NO: 35, SEQ ID NOS: 37-47, SEQ ID NO: 53, SEQ ID NOS: 55-61, SEQ ID NOS: 49-51, SEQ ID NOs: 2-12, SEQ ID NOs: 14-21, SEQ ID NOs: 23-26, and SEQ ID NOS: 28-33, wherein a tissue of a plant produced from said plant cell has a difference in the level of a triterpenoid as compared to the corresponding level in tissue of a control plant that does not comprise said nucleic acid.

103.-107. (canceled)

108. A plant cell comprising (a) a first exogenous nucleic acid comprising a nucleotide sequence encoding a polypeptide having 80% or greater sequence identity to an amino acid sequence selected from the group consisting of SEQ ID NO: 35, SEQ ID NOS: 37-47, SEQ ID NO: 53, SEQ ID NOS: 55-61, and SEQ ID NOS: 49-51; and (b) a second exogenous nucleic acid comprising a nucleotide sequence encoding a polypeptide having 80% or greater sequence identity to an amino acid sequence selected from the group consisting of SEQ ID NOs: 2-12, SEQ ID NOs: 14-21, SEQ ID NOs: 23-26, and SEQ ID NOS: 28-33; wherein expression of said exogenous nucleic acids in tissue of a plant produced from said plant cell results in a difference in the level of a triterpenoid as compared to the corresponding level in tissue of a control plant that does not comprise the said first nucleic acid and the said second nucleic acid.

109.-112. (canceled)

113. A plant cell comprising: (a) a first exogenous nucleic acid comprising a nucleotide sequence encoding a polypeptide having 80% or greater sequence identity to an amino acid sequence selected from the group consisting of SEQ ID NO: 35, SEQ ID NOS: 37-47, SEQ ID NO: 53, SEQ ID NOS: 55-61, and SEQ ID NOS: 49-51; and (b) a second exogenous nucleic acid comprising a nucleotide sequence encoding a polypeptide having 80% or greater sequence identity to an amino acid sequence selected from the group consisting of SEQ ID NO: 35, SEQ ID NOS: 37-47, SEQ ID NO: 53, SEQ ID NOS: 55-61, and SEQ ID NOS: 49-51; provided that the said first exogenous nucleic acid and the said second exogenous nucleic acid are not the same, wherein expression of said exogenous nucleic acids in tissue of a plant produced from said plant cell results in a difference in the level of a triterpenoid as compared to the corresponding level in tissue of a control plant that does not comprise the said first nucleic acid and the said second nucleic acid.

114. A plant cell comprising: (a) a first exogenous nucleic acid comprising a nucleotide sequence encoding a polypeptide having 80% or greater sequence identity to an amino acid sequence selected from the group consisting of SEQ ID NOs: 2-12, SEQ ID NOs: 14-21, SEQ ID NOs: 23-26, and SEQ ID NOS: 28-33; and (b) a second exogenous nucleic acid comprising a nucleotide sequence encoding a polypeptide having 80% or greater sequence identity to an amino acid sequence selected from the group consisting of SEQ ID NOs: 2-12, SEQ ID NOs: 14-21, SEQ ID NOs: 23-26, and SEQ ID NOS: 28-33; provided that the said first exogenous nucleic acid and the said second exogenous nucleic acid are not the same, wherein expression of said exogenous nucleic acids in tissue of a plant produced from said plant cell results in a difference in the level of a triterpenoid as compared to the corresponding level in tissue of a control plant that does not comprise the said first nucleic acid and the said second nucleic acid.

115.-148. (canceled)

149. A transgenic plant comprising the plant cell of any of claims 113 or 114.

150. Progeny of the plant of claim 149, wherein said progeny have a difference in the level of one or more triterpenoids as compared to the corresponding level in tissue of a control plant that does not comprise said exogenous nucleic acid.

151. Progeny of the plant of claim 149, wherein said progeny are seeds.

152. A flour, an oil, or an insoluble fiber product derived from the seeds of claim 151.

153. An isolated nucleic acid molecule comprising a nucleotide sequence having 95% or greater sequence identity to the nucleotide sequence set forth in SEQ ID NO: 156; SEQ ID NO: 158; SEQ ID NO: 160; SEQ ID NO: 162; SEQ ID NO: 165; SEQ ID NO: 167; SEQ ID NO: 170; SEQ ID NO: 172; SEQ ID NO: 174; SEQ ID NO: 176; SEQ ID NO: 178; SEQ ID NO: 180; SEQ ID NO: 182; SEQ ID NO: 184; SEQ ID NO: 187; SEQ ID NO: 189; or SEQ ID NO: 191.

154. An isolated nucleic acid comprising a nucleotide sequence encoding a polypeptide having 80% or greater sequence identity to the amino acid sequence set forth in SEQ ID NO: SEQ ID NO: 157; SEQ ID NO: 159; SEQ ID NO: 161; SEQ ID NO: 163; SEQ ID NO: 164; SEQ ID NO: 166; SEQ ID NO: 168; SEQ ID NO: 169; SEQ ID NO: 171; SEQ ID NO: 173; SEQ ID NO: 175; SEQ ID NO: 177; SEQ ID NO: 179; SEQ ID NO: 181; SEQ ID NO: 183; SEQ ID NO: 185; SEQ ID NO: 186; SEQ ID NO: 188; SEQ ID NO: 190; or SEQ ID NO: 192.
